(19) United States
(12) Patent Application Publication
(10) Pub. No.: US 2024/0409976 A1
(43) Pub. Date: Dec. 12, 2024

(54) METHOD FOR THE BIOSYNTHESIS OF DIOSMIN AND/OR HESPERIDIN IN A MICROORGANISM

(71) Applicant: LES LABORATOIRES SERVIER, SURESNES (FR)

(72) Inventors: CYRILLE PAUTHENIER, JUVISY-SUR-ORGE (FR); ANDRÉ LE JEUNE, DRAVEIL (FR); HÉLÈNE SCORNEC, LONGJUMEAU (FR); CÉLIA ROUSSEL, SAINTRY SUR SEINE (FR); LAETITIA JOUBERT, PALAISEAU (FR)

(21) Appl. No.: 18/668,751

(22) Filed: May 20, 2024

Related U.S. Application Data
(62) Division of application No. 17/429,459, filed on Aug. 9, 2021, now Pat. No. 11,987,829.

(30) Foreign Application Priority Data
Feb. 11, 2019 (EP) 19305163.8

(51) Int. Cl. C12P 19/60; C12N 9/00; C12N 9/02; C12N 9/10; C12N 9/88; C12N 9/90

(52) U.S. Cl. CPC C12P 19/60; C12N 9/0042; C12N 9/0071; C12N 9/0073; C12N 9/1007; C12N 9/1051; C12N 9/1085; C12N 9/88; C12N 9/90; C12N 9/93; C12Y 106/02004; C12Y 114/11022; C12Y 114/13011; C12Y 114/14; C12Y 204/01; C12Y 204/01185; C12Y 205/01006; C12Y 402/01076; C12Y 403/01023; C12Y 403/01024; C12Y 505/01006; C12Y 602/01012

(57) ABSTRACT

The present invention relates to a recombinant microorganism which is modified to be capable of producing diosmin and hesperidin and to the use thereof for producing diosmin and/or hesperidin.

### CROSS-REFERENCE TO RELATED APPLICATIONS

This application is a divisional of U.S. application Ser. No. 17/429,459, filed Aug. 9, 2021, now U.S. Pat. No. 11,987,829, which is the U.S. national stage application of International Patent Application No. PCT/EP2020/053503, filed Feb. 11, 2020.

The Sequence Listing for this application is labeled “Seq-List.xml” which was created on May 17, 2024 and is 331,375 bytes. The entire content of the sequence listing is incorporated herein by reference in its entirety.

### FIELD OF THE INVENTION

The present invention relates to a method for producing diosmin and hesperidin.

### TECHNOLOGICAL BACKGROUND

Daflon is a mixture of flavonoids with vasotonic and vascular-protective effects. This mixture is mainly composed of ˜85% diosmin, ˜8% hesperidin and traces of various flavones and the respective oxidized forms thereof.

The current method for producing the medicament is acid/base extraction, from the peel of small oranges, of a mixture of flavonoids mainly containing hesperidin (94%) and a mixture of isonaringin (˜3%), neoponcirin (˜2%) and hesperetin (<1%). This mixture then undergoes controlled oxidation via a chemical process, transforming about 90% of the hesperidin into diosmin, and minor flavonoids in their oxidized form.

Thus, the production of Daflon is associated with the supply of purified extract of orange flavonoids, which may vary as a result of climatic variations, fluctuations in currency movements and the difficulty of being supplied from dozens of sites in several countries (mainly Mexico, the countries of the Mediterranean basin and China).

It would thus be valuable to have available an alternative method for producing Daflon which is not dependent on the vagaries of being supplied with purified extract of orange flavonoids. Thus, there is an unsatisfied need for a process for the biosynthesis of diosmin and hesperidin.

### SUMMARY OF THE INVENTION

The inventors have developed a method for the biosynthesis of diosmin and hesperidin in a recombinant microorganism.

Thus, the present invention relates to a recombinant microorganism comprising:
a heterologous nucleic acid sequence coding for a flavanone 7-O-beta-D-glucosyltransferase (UGT) which is capable of adding a glucose in position 7 of hesperetin and/or diosmetin; and
a heterologous nucleic acid sequence coding for a 6″-O-rhamnosyltransferase (RhaT) which is capable of transferring a rhamnose into position 6 of the glucose of hesperetin-7-O-glucoside and/or of diosmetin-7-O-glucoside; and
a heterologous nucleic acid sequence coding for a UDP-glucose 4,6-dehydratase/UDP-4-keto-6-deoxy-D-glucose 3,5-epimerase/UDP-4-keto-L-rhamnose-reductase (RHM) which is capable of producing UDP-rhamnose.

Preferably, the flavanone 7-O-beta-D-glucosyltransferase (UGT) is an enzyme from Citrus sinensis, Citrus clementina, Arabidopsis thaliana, Scutellaria baicalensis or Homo sapiens. In particular, the flavanone 7-O-beta-D-glucosyltransferase (UGT) may be an enzyme from Arabidopsis thaliana, Scutellaria baicalensis or Homo sapiens, preferably from Arabidopsis thaliana or Scutellaria baicalensis. Preferably, the flavanone 7-O-beta-D-glucosyltransferase (UGT) is from Citrus sinensis or Scutellaria baicalensis.

The flavanone 7-O-beta-D-glucosyltransferase may be selected from enzymes comprising a sequence chosen from SEQ ID NOs: 113, 115, 91, 93, 95, 97, 99 and 101 and polypeptides comprising a sequence having at least 60, 70, 80, 85, 90 or 95% sequence identity with one of these sequences and having flavanone 7-O-beta-D-glucosyltransferase activity, preferably selected from enzymes comprising a sequence chosen from SEQ ID NOs: 113, 115, 91, 93, 95 and 97 and polypeptides comprising a sequence having at least 60, 70, 80, 85, 90 or 95% sequence identity with one of these sequences and having flavanone 7-O-beta-D-glucosyltransferase activity. In particular, it may be selected from enzymes comprising a sequence chosen from SEQ ID NOs: 91, 93, 95, 97, 99 and 101 and polypeptides comprising a sequence having at least 60, 70, 80, 85, 90 or 95% sequence identity with one of these sequences and having flavanone 7-O-beta-D-glucosyltransferase activity, preferably selected from enzymes comprising a sequence chosen from SEQ ID NOs: 91, 93, 95 and 97 and polypeptides comprising a sequence having at least 60, 70, 80, 85, 90 or 95% sequence identity with one of these sequences and having flavanone 7-O-beta-D-glucosyltransferase activity. Preferably, the flavanone 7-O-beta-D-glucosyltransferase is selected from enzymes comprising a sequence chosen from SEQ ID NOs: 113 and 95 and polypeptides comprising a sequence having at least 60, 70, 80, 85, 90 or 95% sequence identity with one of these sequences and having flavanone 7-O-beta-D-glucosyltransferase activity, preferably selected from enzymes comprising a sequence chosen from SEQ ID NO: 113 and polypeptides comprising a sequence having at least 60, 70, 80, 85, 90 or 95% sequence identity with this sequence and having flavanone 7-O-beta-D-glucosyltransferase activity.

Preferably, 6″-O-rhamnosyltransferase (RhaT) is a plant enzyme, preferably of the genus Citrus or Petunia hybrida, preferably Citrus sinensis, Citrus maxima, or Citrus clementina, more preferably Citrus sinensis or Citrus clementina. Preferably, 6″-O-rhamnosyltransferase is selected from enzymes comprising a sequence chosen from SEQ ID NOs: 103, 105 and polypeptides comprising a sequence having at least 60, 70, 80, 85, 90 or 95% sequence identity with one of these sequences and having 6″-O-rhamnosyltransferase activity. More particularly preferably, 6″-O-rhamnosyltransferase is selected from enzymes comprising a sequence chosen from SEQ ID NO: 103 and polypeptides comprising a sequence having at least 60, 70, 80, 85, 90 or 95% sequence identity with this sequence and having 6″-O-rhamnosyltransferase activity.

Preferably, UDP-glucose 4,6-dehydratase/UDP-4-keto-6-deoxy-D-glucose 3,5-epimerase/UDP-4-keto-L-rhamnose-reductase (RHM) is a plant enzyme, preferably from Citrus sinensis or Arabidopsis thaliana. Preferably, UDP-glucose 4,6-dehydratase/UDP-4-keto-6-deoxy-D-glucose 3,5-epimerase/UDP-4-keto-L-rhamnose-reductase is selected from enzymes comprising a sequence chosen from SEQ ID NOs: 107, 109 and 111 and polypeptides comprising a sequence having at least 60, 70, 80, 85, 90 or 95% sequence identity with one of these sequences and having UDP-glucose 4,6-dehydratase/UDP-4-keto-6-deoxy-D-glucose 3,5-epimerase/UDP-4-keto-L-rhamnose-reductase activity. More particularly preferably, UDP-glucose 4,6-dehydratase/UDP-4-keto-6-deoxy-D-glucose 3,5-epimerase/UDP-4-keto-L-rhamnose-reductase is selected from enzymes comprising a sequence chosen from SEQ ID NO: 107 and polypeptides comprising a sequence having at least 60, 70, 80, 85, 90 or 95% sequence identity with this sequence and having UDP-glucose 4,6-dehydratase/UDP-4-keto-6-deoxy-D-glucose 3,5-epimerase/UDP-4-keto-L-rhamnose-reductase activity.

In one embodiment, the microorganism according to the invention also comprises:
a heterologous nucleic acid sequence coding for a tyrosine ammonia lyase (TAL);
and/or a heterologous nucleic acid sequence coding for a phenylalanine ammonia lyase (PAL) and a heterologous nucleic acid sequence coding for a cinnamate 4-hydroxylase (C4H);
a heterologous nucleic acid sequence coding for a 4-coumaroyl-CoA ligase (4CL);
a heterologous nucleic acid sequence coding for a naringenin-chalcone synthase (CHS);
a heterologous nucleic acid sequence coding for a chalcone isomerase (CHI);
a heterologous nucleic acid sequence coding for a flavonoid 3′-monooxygenase (F3′H);
a heterologous nucleic acid sequence coding for an O-methyl-transferase (OMT), and
optionally, a heterologous nucleic acid sequence coding for a 4-methoxybenzoate O-demethylase which is capable of converting tyrosine into L-DOPA and also p-coumaric acid into caffeic acid; or a heterologous nucleic acid sequence coding for a p-coumarate 3-hydroxylase which is capable of converting p-coumaric acid into caffeic acid.

Preferably, the microorganism comprises:
a heterologous nucleic acid sequence coding for a tyrosine ammonia lyase (TAL) comprising a sequence chosen from SEQ ID NOs: 41 and 39 and polypeptides comprising a sequence having at least 60, 70, 80, 85, 90 or 95% sequence identity with one of these sequences and having tyrosine ammonia lyase activity, and preferably a tyrosine ammonia lyase (TAL) comprising a sequence chosen from SEQ ID NO: 41 and polypeptides comprising a sequence having at least 60, 70, 80, 85, 90 or 95% sequence identity with this sequence and having tyrosine ammonia lyase activity; and
a heterologous nucleic acid sequence coding for a 4-coumaroyl-CoA ligase (4CL) comprising a sequence chosen from SEQ ID NOs: 123, 125, 43, 45, 47 and 49 and polypeptides comprising a sequence having at least 60, 70, 80, 85, 90 or 95% sequence identity with one of these sequences and having 4-coumarate-CoA ligase activity, preferably a 4CL comprising a sequence selected from SEQ ID NOs: 123, 125 and 45 and polypeptides comprising a sequence having at least 60, 70, 80, 85, 90 or 95% sequence identity with one of these sequences and having 4-coumarate-CoA ligase activity, and in particular a heterologous nucleic acid sequence coding for a 4-coumaroyl-CoA ligase (4CL) comprising a sequence selected from SEQ ID NO: 45 and polypeptides comprising a sequence having at least 60, 70, 80, 85, 90 or 95% sequence identity with this sequence and having 4-coumarate-CoA ligase activity; and
a heterologous nucleic acid sequence coding for a chalcone synthase (CHS) comprising a sequence chosen from SEQ ID NOs: 53, 51, 55 and 57 and polypeptides comprising a sequence having at least 60, 70, 80, 85, 90 or 95% sequence identity with one of these sequences and having chalcone synthase activity, preferably a CHS comprising a sequence chosen from SEQ ID NO: 53 and polypeptides comprising a sequence having at least 60, 70, 80, 85, 90 or 95% sequence identity with this sequence and having chalcone synthase activity; and
a heterologous nucleic acid sequence coding for a chalcone isomerase (CHI) comprising a sequence chosen from SEQ ID NOs: 61 and 59 and polypeptides comprising a sequence having at least 60, 70, 80, 85, 90 or 95% sequence identity with one of these sequences and having chalcone isomerase activity, and preferably a CHI comprising a sequence chosen from SEQ ID NO: 61 and polypeptides comprising a sequence having at least 60, 70, 80, 85, 90 or 95% sequence identity with this sequence and having chalcone isomerase activity.

Preferably, the microorganism comprises a heterologous nucleic acid sequence coding for a flavonoid 3′-monooxygenase (F3′H) from Callistephus chinensis, Perilla frutescens var. crispa, Petunia x hybrida, Gerbera hybrida, Citrus sinensis, Arabidopsis thaliana, Pilosella officinarum, Osteospermum hybrid cultivar, Phanerochaete chrysosporium, Citrus clementina or Streptomyces avermitilis, in particular from Callistephus chinensis, Perilla frutescens var. crispa, Petunia x hybrida, Gerbera hybrida, Citrus sinensis, Arabidopsis thaliana or Pilosella officinarum, preferably an enzyme comprising a sequence chosen from SEQ ID NOs: 7, 1, 3, 5, 9, 11, 13, 15, 17, 19, 21 and 121 and polypeptides comprising a sequence having at least 60, 70, 80, 85, 90 or 95% sequence identity with one of these sequences and having flavonoid 3′-monooxygenase activity, preferably selected from enzymes having the SEQ ID NOs: 7, 11, 17 and 121 and polypeptides having at least 60, 70, 80, 85, 90 or 95% identity with one of these sequences and having flavonoid 3′-monooxygenase activity.

More particularly preferably, the microorganism comprises a heterologous nucleic acid sequence coding for a flavonoid 3′-monooxygenase (F3′H) comprising a sequence chosen from SEQ ID NOs: 7, 17 and 121 and polypeptides having at least 60, 70, 80, 85, 90 or 95% identity with one of these sequences and having flavonoid 3′-monooxygenase activity, preferably a flavonoid 3′-monooxygenase (F3′H) comprising a sequence chosen from SEQ ID NO: 7 and polypeptides having at least 60, 70, 80, 85, 90 or 95% identity with this sequence and having flavonoid 3′-monooxygenase activity.

Preferably, the microorganism comprises a heterologous nucleic acid sequence coding for an O-methyl-transferase (OMT) from Citrus, in particular Citrus clementina or Citrus sinensis, from Homo sapiens or from Arabidopsis thaliana, preferably an enzyme comprising a sequence chosen from SEQ ID NOs: 119, 117, 87 and 89 and polypeptides comprising a sequence having at least 60, 70, 80, 85, 90 or 95% sequence identity with one of these sequences and having O-methyltransferase activity. More particularly preferably, it comprises a heterologous nucleic acid sequence coding for an O-methyltransferase (OMT) comprising a sequence chosen from SEQ ID NOs: 119, 117 and 89 and polypeptides comprising a sequence having at least 60, 70, 80, 85, 90 or 95% sequence identity with one of these sequences and having O-methyltransferase activity, preferably a sequence chosen from SEQ ID NOs: 119 and 117 and polypeptides comprising a sequence having at least 60, 70, 80, 85, 90 or 95% sequence identity with one of these sequences and having O-methyltransferase activity.

Preferably, the microorganism comprises:
a heterologous nucleic acid sequence coding for a phenylalanine ammonia lyase (PAL), in particular a PAL comprising a sequence chosen from SEQ ID NOs: 63, 65 and 77, preferably SEQ ID NOs: 65 and 77, and polypeptides comprising a sequence having at least 60, 70, 80, 85, 90 or 95% sequence identity with one of these sequences and having phenylalanine ammonia lyase activity, and more particularly preferably a phenylalanine ammonia lyase (PAL) comprising a sequence chosen from SEQ ID NO: 65 and polypeptides comprising a sequence having at least 60, 70, 80, 85, 90 or 95% sequence identity with this sequence and having phenylalanine ammonia lyase activity; and
a heterologous nucleic acid sequence coding for a cinnamate 4-hydroxylase (C4H), in particular a C4H comprising a sequence chosen from SEQ ID NOs: 67, 69 and 79 and polypeptides comprising a sequence having at least 60, 70, 80, 85, 90 or 95% sequence identity with one of these sequences and having cinnamate 4-hydroxylase activity; and most particularly preferably a cinnamate 4-hydroxylase (C4H) comprising a sequence chosen from SEQ ID NO: 79 and polypeptides comprising a sequence having at least 60, 70, 80, 85, 90 or 95% sequence identity with this sequence and having cinnamate 4-hydroxylase activity.

The microorganism may also comprise a heterologous nucleic acid sequence coding for a flavone synthase (FNS), in particular a flavone synthase which is capable of producing luteolin from eriodictyol, preferably from Arabidopsis thaliana, Petroselinum crispum, Zea mays, Lonicera japonica, Lonicera macranthoides, Callistephus chinensis, Apium graveolens, Medicago truncatula, Cuminum cyminum, Aethusa cynapium, Angelica archangelica, Conium maculatum, Camellia sinensis, Cynara cardunculus var scolymus, Saussurea medusa, Plectranthus barbatus, Scutellaria baicalensis, Dorcoceras hygrometricum, Antirrhinum majus, Perilla frutescens var crispa, Dahlia pinnata or Erythranthe lewisii. The flavone synthase (FNS) may be selected from enzymes comprising a sequence chosen from SEQ ID NOs: 33, 35, 37, 127, 129, 131, 133, 135, 137, 139, 141, 143, 145, 147, 149, 151, 153, 155, 157 and 159 and polypeptides comprising a sequence having at least 60, 70, 80, 85, 90 or 95% sequence identity with one of these sequences and having flavone synthase activity. In particular, the FNS may be an FNS comprising a sequence chosen from SEQ ID NOs: 33, 35 and 37 and polypeptides comprising a sequence having at least 60, 70, 80, 85, 90 or 95% sequence identity with one of these sequences and having flavone synthase activity, and preferably a flavone synthase (FNS) comprising a sequence chosen from SEQ ID NO: 37 and a polypeptide comprising a sequence having at least 60, 70, 80, 85, 90 or 95% sequence identity with this sequence and having flavone synthase activity.

Moreover, it may also comprise:
a heterologous nucleic acid sequence coding for a cytochrome P450 reductase (CPR); and/or
a heterologous or endogenous nucleic acid sequence coding for an S-adenosylmethionine synthetase (SAMT).

Preferably, the CPR comprises a sequence chosen from SEQ ID NOs: 25, 23, 27, 29 and 31 and polypeptides comprising a sequence having at least 60, 70, 80, 85, 90 or 95% sequence identity with one of these sequences and having cytochrome P450 reductase activity, preferably a sequence chosen from SEQ ID NOs: 23, 25 and 29 and polypeptides comprising a sequence having at least 60, 70, 80, 85, 90 or 95% sequence identity with one of these sequences and having cytochrome P450 reductase activity, and particularly a sequence chosen from SEQ ID NO: 25 and polypeptides comprising a sequence having at least 60, 70, 80, 85, 90 or 95% sequence identity with this sequence and having cytochrome P450 reductase activity.

Preferably, the microorganism is a yeast or a bacterium, preferably a yeast of the genus Saccharomyces, in particular Saccharomyces cerevisiae, or a bacterium such as Escherichia coli.

The present invention also relates to the use of a microorganism as described in the present document for producing diosmin and/or hesperidin.

In addition, the invention relates to a method for producing diosmin and/or hesperidin comprising the cultivation of a microorganism as described in the present document, and optionally the harvesting of diosmin and/or hesperidin.

Preferably, during the use of the microorganism according to the invention for producing diosmin and/or hesperidin there is no naringenin, apigenin, eriodictyol, luteolin, hesperetin and/or diosmetin supplied to the culture medium.

### DETAILED DESCRIPTION OF THE INVENTION

The pathways for the biosynthesis of hesperetin in the peel of small oranges are poorly understood. The inventors thus explored several biosynthetic pathways and succeeded in developing the biosynthesis of diosmin and of hesperidin in a recombinant microorganism.

### Definition

The term “microorganism” refers to a unicellular organism. Preferably, the microorganism is a bacterium or a yeast.

The term “recombinant microorganism” refers to a microorganism which is not found in nature and which contains a genome modified following insertion, modification or deletion of one or more heterologous genetic elements.

The term “recombinant nucleic acid” refers to a nucleic acid which has been modified and does not exist in a natural microorganism. For example, this term may denote a coding sequence or gene which is operatively linked to a promoter which is not the natural promoter. This may also denote a coding sequence in which the introns have been deleted for genes comprising exons and introns.

The term “heterologous” means that the gene has been introduced by genetic engineering into the cell. It may be present therein in episomal or chromosomal form. The origin of the gene may be different from that of the cell into which it is introduced. However, the gene may also originate from the same species as the cell into which it is introduced, but it is considered as heterologous on account of its unnatural environment. For example, the gene or the nucleic acid sequence is heterologous since it is under the control of a promoter other than its natural promoter, it is introduced into a position different from that in which it is naturally located. The host cell may contain a copy of the endogenous gene prior to the introduction of the heterologous gene or it may not contain an endogenous copy. Moreover, the nucleic acid sequence may be heterologous in the sense that the coding sequence has been optimized for expression in the host microorganism. Preferably, in the present document, a heterologous nucleic acid sequence codes for a protein which is heterologous to the host cell, i.e. which is not naturally present in the yeast.

As used herein, the term “native” or “endogenous”, relative to the host microorganism, refers to a genetic element or to a protein that is naturally present in said microorganism.

The term “gene” denotes any nucleic acid coding for a protein. The term “gene” covers DNA, such as cDNA or gDNA, and also RNA. The gene may first be prepared via recombinant, enzymatic and/or chemical techniques, and subsequently replicated in a host cell or a system in vitro. The gene typically comprises an open reading frame coding for a desired protein. The gene may contain additional sequences such as a transcription terminator or a signal peptide.

As a result of degeneracy of the genetic code, several nucleic acids may code for a particular polypeptide. Thus, the codons in the coding sequence for a given polypeptide may be modified such that optimum expression in a particular microorganism is obtained, for example by using suitable codon translation tables for this microorganism. The nucleic acids may also be optimized according to a preferable GC content for the particular yeast and/or to reduce the number of repeat sequences. In certain embodiments, the heterologous nucleic acids were codon-optimized for expression in the microorganism concerned. Codon optimization may be performed via routine processes known in the art (see, for example, Welch, M., et al. (2011), Methods in Enzymology 498: 43-66).

The term “operatively linked” denotes a configuration in which a control sequence is placed in a suitable position relative to a coding sequence, such that the control sequence controls the expression of the coding sequence.

The term “control sequences” denotes the nucleic acid sequences required for the expression of a gene. The control sequences may be native or heterologous. Control sequences that are well known and currently used by those skilled in the art will be preferred. Such control sequences comprise, but without being limited thereto, a leader, a polyadenylation sequence, a propeptide sequence, a promoter, a signal peptide sequence and a transcription terminator. Preferably, the control sequences comprise a promoter and a transcription terminator.

The term “expression cassette” denotes a nucleic acid construct comprising a coding region, i.e. a gene, and a regulating region, i.e. a region comprising one or more control sequences, which are operatively linked. Preferably, the control sequences are suitable for use in the host microorganism.

As used herein, the term “expression vector” denotes a DNA or RNA molecule which comprises an expression cassette. Preferably, the expression vector is a linear or circular double-stranded DNA molecule. The vector may also comprise an origin of replication, a selection marker, etc.

For the purposes of the present invention, the term “percentage of identity” between two nucleic acid sequences or amino acid sequences is intended to denote a percentage of nucleotides or of amino acid residues that are identical between the two sequences to be compared, obtained after the best alignment, this percentage being purely statistical and the differences between the two sequences being distributed randomly and over their entire length. The best alignment or optimum alignment is the alignment for which the percentage of identity between the two sequences to be compared, as calculated below, is the highest. Sequence comparisons between two nucleic acid or amino acid sequences are conventionally performed by comparing these sequences after they have been optimally aligned, said comparison being performed by segment or by comparison window to identify and compare the local regions with sequence similarity. The alignment for the purposes of determining the percentage of amino acid sequence identity may be performed in various ways that are well known in the field, for example by using computer software available on the Internet, such as http://blast.ncbi.nlm. Nih.gov/or http://www.ebi.ac.uk/Tools/emboss/). A person skilled in the art can determine the appropriate parameters for measuring the alignment, including any algorithm necessary to obtain a maximum alignment over the entire length of the sequences compared. For the purposes of the present invention, the values of the percentage of amino acid sequence identity refer to values generated using the EMBOSS Needle pair sequence alignment program which creates an optimum global alignment of two sequences by means of the Needleman-Wunsch algorithm, in which all the search parameters are defined by default Notation matrix=BLOSUM62, Open gap=10, Extended gap=0.5, end gap penalty=false, open end gap=10 and extended end gap=0.5. In certain embodiments, all the percentages of identity mentioned in the present patent application may be set at at least 60%, at least 70%, at least 80%, at least 85%, preferably at at least 90% identity, more preferably at at least 95% identity. In particular, the embodiments in which all the percentages of sequence identity of the enzymes are at least 80% or at least 85%, preferably at least 90% or at least 95% sequence identity are considered as described.

In one embodiment, the polypeptides may contain 1, 2, 3, 4, 5, 6, 7, 8, 9 or 10 additions, substitutions or deletions relative to the sequences described in the SEQ ID NOs. In particular, these additions, substitutions or deletions are introduced at the N-terminal end, the C-terminal end or at both ends.

The polypeptides may optionally be in the form of a fusion protein.

The terms “overexpression” and “increased expression” as used herein are used interchangeably and mean that the expression of a gene or of an enzyme is increased relative to an unmodified microorganism, for example a wild-type microorganism or a microorganism not comprising the genetic modifications described herein. The term “wild-type” refers to an unmodified microorganism existing in nature. The increased expression of an enzyme is usually obtained by increasing the expression of the gene coding for said enzyme. In embodiments in which the gene or the enzyme is not naturally present in the microorganism of the invention, i.e. a heterologous gene or enzyme, the terms “overexpression” and “expression” may be used interchangeably. To increase the expression of a gene, a person skilled in the art can use any known technique such as increasing the number of copies of the gene in the microorganism, by using a promoter inducing a high level of expression of the gene, i.e. a strong promoter, by using elements which stabilize the corresponding messenger RNA or sequences which sequester the ribosomal binding site (RBS) and the sequences surrounding same. In particular, overexpression may be obtained by increasing the number of copies of the gene in the microorganism. One or more copies of the gene may be introduced into the genome via recombination processes, known to those skilled in the art, including the replacement of the genes or multi-copy integration (see, for example, the international patent application WO 2015/092013). Preferably, an expression cassette comprising the gene, preferably placed under the control of a strong promoter, is integrated into the genome. As a variant, the gene may be carried by an expression vector, preferably a plasmid, comprising an expression cassette with the gene of interest preferably placed under the control of a strong promoter. The expression vector may be present in the microorganism in one or more copies, depending on the nature of the origin of replication. Overexpression of the gene may also be obtained by using a promoter which induces a high level of expression of the gene. For example, the promoter of an endogenous gene may be replaced with a stronger promoter, i.e. a promoter which induces a higher level of expression. The endogenous gene under the control of a promoter which is not the natural promoter is termed a heterologous nucleic acid. The promoters that are suitable for use in the present invention are known to those skilled in the art and may be constitutive or inducible, and may be endogenous or heterologous.

The term “comprising” also means “consisting of” or “consisting essentially of”. The term “consisting essentially of” means that the sequence may contain 1, 2, 3, 4, 5, 6, 7, 8, 9 or 10 additions, substitutions or deletions relative to the sequences described in the SEQ ID NOs.

### Recombinant Microorganisms

The microorganism according to the present invention may be a eukaryotic or prokaryotic microorganism.

In a first embodiment, the microorganism is a eukaryote. Preferably, it is a yeast of the Saccharomycetales, Sporidiobolales and Schizosaccharomycetales orders. The yeast may be selected, for example, from Pichia, Kluyveromyces, Saccharomyces, Schizosaccharomyces, Candida, Lipomyces, Rhodotorula, Rhodosporidium, Yarrowia, or Debaryomyces. In one embodiment, the yeast is chosen from Pichia pastoris, Kluyveromyces lactis, Kluyveromyces marxianus, Saccharomyces cerevisiae, Saccharomyces carlsbergensis, Saccharomyces diastaticus, Saccharomyces douglasii, Saccharomyces kluyveri, Saccharomyces norbensis, Saccharomyces oviformis, Schizosaccharomyces pombe, Candida albicans, Candida tropicalis, Rhodotorula glutinis, Rhodosporidium toruloides, Yarrowia lipolytica, Debaryomyces hansenii and Lipomyces starkeyi. In a preferred embodiment, the microorganism is a Saccharomyces yeast, preferably a Saccharomyces cerevisiae yeast. Alternatively, the microorganism may be a fungus, preferably a filamentous fungus. Preferably, it is chosen from Aspergillus, Trichoderma, Neurospora, Podospora, Endothia, Mucor, Cochiobolus or Pyricularia. Preferentially, the fungus is chosen from Aspergillus nidulans, Aspergillus niger, Aspergillus awomari, Aspergillus oryzae, Aspergillus terreus, Neurospora crassa, Trichoderma reesei and Trichoderma viride.

In a second embodiment, the microorganism is a prokaryote. Preferably, it is a bacterium, notably chosen from the phylum Acidobacteria, Actinobacteria, Aquificae, Bacterioidetes, Chlamydiae, Chlorobi, Chloroflexi, Chrysiogenetes, Cyanobacteria, Deferribacteres, Deinococcus-Thermus, Dictyoglomi, Fibrobacteres, Firmicutes, Fusobacteria, Gemmatimonadetes, Nitrospirae, Planctomycetes, Proteobacteria, Spirochaetes, Thermodesulfobacteria, Thermomicrobia, Thermotogae or Verrucomicrobia. Preferably, the bacterium belongs to the genus Acaryochloris, Acetobacter, Actinobacillus, Agrobacterium, Alicyclobacillus, Anabaena, Anacystis, Anaerobiospirillum, Aquifex, Arthrobacter, Arthrospira, Azobacter, Bacillus, Brevibacterium, Burkholderia, Chlorobium, Chromatium, Chlorobaculum, Clostridium, Corynebacterium, Cupriavidus, Cyanothece, Enterobacter, Deinococcus, Erwinia, Escherichia, Geobacter, Gloeobacter, Gluconobacter, Hydrogenobacter, Klebsiella, Lactobacillus, Lactococcus, Mannheimia, Mesorhizobium, Methylobacterium, Microbacterium, Microcystis, Nitrobacter, Nitrosomonas, Nitrospina, Nitrospira, Nostoc, Phormidium, Prochlorococcus, Pseudomonas, Ralstonia, Rhizobium, Rhodobacter, Rhodococcus, Rhodopseudomonas, Rhodospirillum, Salmonella, Scenedesmun, Serratia, Shigella, Staphylococcus, Streptomyces, Synechoccus, Synechocystis, Thermosynechococcus, Trichodesmium or Zymomonas. More preferably, the bacterium is chosen from the species Agrobacterium tumefaciens, Anaerobiospirillum succiniciproducens, Actinobacillus succinogenes, Aquifex aeolicus, Aquifex pyrophilus, Bacillus subtilis, Bacillus amyloliquefacines, Brevibacterium ammoniagenes, Brevibacterium immariophilum, Clostridium pasteurianum, Clostridium ljungdahlii, Clostridium acetobutylicum, Clostridium beigerinckii, Corynebacterium glutamicum, Cupriavidus necator, Cupriavidus metallidurans, Enterobacter sakazakii, Escherichia coli, Gluconobacter oxydans, Hydrogenobacter thermophilus, Klebsiella oxytoca, Lactococcus lactis, Lactobacillus plantarum, Mannheimia succiniciproducens, Mesorhizobium loti, Pseudomonas aeruginosa, Pseudomonas mevalonii, Pseudomonas pudica, Pseudomonas putida, Pseudomonas fluorescens, Rhizobium etli, Rhodobacter capsulatus, Rhodobacter sphaeroides, Rhodospirillum rubrum, Salmonella enterica, Salmonella typhi, Salmonella typhimurium, Shigella dysenteriae, Shigella flexneri, Shigella sonnei, Staphylococcus aureus, Streptomyces coelicolor, Zymomonas mobilis, Acaryochloris marina, Anabaena variabilis, Arthrospira platensis, Arthrospira maxima, Chlorobium tepidum, Chlorobaculum sp., Cyanothece sp., Gloeobacter violaceus, Microcystis aeruginosa, Nostoc punctiforme, Prochlorococcus marinus, Synechococcus elongatus, Synechocystis sp., Thermosynechococcus elongatus, Trichodesmium erythraeum and Rhodopseudomonas palustris. In a preferred embodiment, the microorganism is an Escherichia coli bacterium, for example E. coli BL21, E. coli BL21 (DE3), E. coli MG1655 or E. coli W31 10 and derivatives thereof. In an alternative embodiment, the microorganism is a bacterium of the Streptomyces genus, in particular Streptomyces venezuelae.

The microorganisms may have been modified to increase the production of tyrosine and/or phenylalanine, preferably tyrosine. Notably, the genes responsible for the feedback inhibition of the production of tyrosine and/or phenylalanine, preferably of tyrosine, may be inactivated. Alternatively or cumulatively, the pathway for the biosynthesis of tyrosine and/or phenylalanine, preferably of tyrosine, may be optimized, notably by redirecting the flow of carbon from other metabolic pathways toward that of tyrosine and/or phenylalanine, preferably of tyrosine. These modifications and these genes are well known to those skilled in the art (see U.S. Pat. No. 8,809,028; Pandey et al., 2016, Biotechnol. Adv., 34, 634-662).

Thus, in one embodiment, the microorganism produces large amounts of tyrosine and/or of phenylalanine, in particular from a simple carbon source such as glucose.

Modifications Enabling the Production of Hesperidin and/or Diosmin

The recombinant microorganism according to the present invention was modified to produce hesperidin and/or diosmin. Notably, to enable the microorganism to synthesize hesperidin and/or diosmin from hesperetin and/or from diosmetin, respectively, the microorganism was modified to introduce the enzymes required for the glycosylation of hesperetin and/or of diosmetin in position 7 and for the transfer of a rhamnose in position 6 of the glucose of hesperetin-7-O-glucoside and/or diosmetin-7-O-glucoside.

In a first embodiment, the recombinant microorganism is capable of producing hesperetin and/or diosmetin: in particular, it has been modified for this purpose. In an alternative embodiment, hesperetin and/or diosmetin may be provided to the microorganism, for example by adding these compounds to the culture medium.

In one particular embodiment, the microorganism produces hesperidin. Diosmin may then be prepared from hesperidin by chemical conversion, notably by oxidation.

In a preferred embodiment, the microorganism produces hesperidin and diosmin.

Thus, the recombinant microorganism comprises:
a. a heterologous nucleic acid sequence coding for a flavanone 7-O-beta-D-glucosyltransferase (UGT) which is capable of adding a glucose in position 7 of hesperetin and/or diosmetin;
b. a heterologous nucleic acid sequence coding for a 6-O-rhamnosyltransferase (RhaT) which is capable of transferring a rhamnose into position 6 of the glucose of hesperetin-7-O-glucoside and/or diosmetin-7-O-glucoside; and
c. a heterologous nucleic acid sequence coding for a UDP-glucose 4,6-dehydratase/UDP-4-keto-6-deoxy-D-glucose 3,5-epimerase/UDP-4-keto-L-rhamnose-reductase (RHM) which is capable of producing UDP-rhamnose.

In one embodiment, the flavanone 7-O-beta-D-glucosyltransferase (UGT), 6″-O-rhamnosyltransferase (RhaT) and UDP-glucose 4,6-dehydratase/UDP-4-keto-6-deoxy-D-glucose 3,5-epimerase/UDP-4-keto-L-rhamnose-reductase (RHM) are enzymes that are heterologous to the microorganism.

### UGT: Flavanone 7-O-Beta-Glucosyltransferase

UGT is an enzyme which performs the transfer of a glucose into position 7 of hesperetin and/or diosmetin. The name of UGT is UDP-glucose: flavanone 7-O-beta-D-glucosyltransferase or flavanone 7-O-beta-D-glucosyltransferase. It is also referred to by the following names: uridine diphosphoglucose-flavanone 7-O-glucosyltransferase, naringenin 7-O-glucosyltransferase, and hesperetin 7-O-glucosyl-transferase. This enzyme belongs to the class EC 2.4.1.185.

The inventors had to identify and select enzymes that are capable of accepting hesperetin and/or diosmetin as a substrate and of adding a glucose in position 7 of these compounds. Preferably, the enzyme is selected so as to have a preference for glycosylation in position 7 of hesperetin and/or diosmetin. In a preferred embodiment, the enzyme is specific for position 7 of hesperetin and/or diosmetin.

The microorganism thus comprises a heterologous nucleic acid sequence coding for a flavanone 7-O-beta-D-glucosyltransferase (UGT) which is capable of adding a glucose in position 7 of hesperetin and diosmetin.

The term “7-O-beta-glycosyltransferase activity” refers to a UGT enzyme which is capable of adding a glucose in position 7 of a flavonoid. To determine whether there is 7-0-beta-glycosyltransferase activity, an enzymatic test may be performed, which consists of the in vitro incubation of the flavanone 7-O-beta-D-glucosyltransferase enzyme in the presence of NAD(P)H, O₂, and of a flavonoid, under optimum conditions (pH, ions, etc.), and observation by UPLC-MS and comparison with the standard expected for the appearance of a flavonoid containing an additional glucose in position 7. Preferably, the flavonoid is hesperetin or diosmetin and the flavonoid containing an additional glucose in position 7 is their form with an additional glucose in position 7, i.e. hesperetin 7-O-glucoside and diosmetin 7-O-glucoside.

This enzyme is present only in higher eukaryotes, in particular in plants. For example, the enzyme may originate from plants of the genus Citrus, in particular Citrus maxima, Citrus sinensis, Citrus clementina, Citrus mitis and Citrus x paradisi, Lysium, in particular Lysium barbarum, Petunia, in particular Petunia x hybrida, Arabidopsis, in particular Arabidopsis thaliana, or Scutellaria, in particular Scutellaria baicalensis.

In one embodiment, the UGT is an enzyme from Arabidopsis thaliana, Scutellaria baicalensis or Homo sapiens. Preferably, the UGT is an enzyme from Arabidopsis thaliana or from Scutellaria baicalensis.

In a preferred embodiment, the UGT is an enzyme from Citrus sinensis, from Citrus clementina, from Arabidopsis thaliana, from Scutellaria baicalensis or from Homo sapiens, preferably from Citrus sinensis or from Scutellaria baicalensis.

In a particular embodiment, the UGT is selected from enzymes comprising a sequence chosen from SEQ ID NOs: 91, 93, 95, 97, 99 and 101 and polypeptides comprising a sequence having at least 60, 70, 80, 85, 90 or 95% sequence identity with one of these sequences and having flavanone 7-O-beta-D-glucosyltransferase activity, preferably selected from enzymes comprising a sequence chosen from SEQ ID NOs: 91, 93, 95 and 97 and polypeptides comprising a sequence having at least 60, 70, 80, 85, 90 or 95% sequence identity with one of these sequences and having flavanone 7-O-beta-D-glucosyltransferase activity, notably with hesperetin and/or diosmetin as substrate.

In a preferred embodiment, the UGT is selected from enzymes comprising a sequence chosen from SEQ ID NOs: 113, 115, 91, 93, 95, 97, 99 and 101 and polypeptides comprising a sequence having at least 60, 70, 80, 85, 90 or 95% sequence identity with one of these sequences and having flavanone 7-O-beta-D-glucosyltransferase activity, preferably selected from enzymes comprising a sequence chosen from SEQ ID NOs: 113, 115, 91, 93, 95 and 97 and polypeptides comprising a sequence having at least 60, 70, 80, 85, 90 or 95% sequence identity with one of these sequences and having flavanone 7-O-beta-D-glucosyltransferase activity. More particularly preferably, the UGT is selected from enzymes comprising a sequence chosen from SEQ ID NOs: 113, 115 and 95 and polypeptides comprising a sequence having at least 60, 70, 80, 85, 90 or 95% sequence identity with one of these sequences and having flavanone 7-O-beta-D-glucosyltransferase activity.

Thus, the UGT may be from Arabidopsis thaliana. The nucleic acid sequences coding for this enzyme and protein sequences are described in NCBI under the reference numbers NM_119576 and NP_567995.1, respectively, and more particularly in SEQ ID NO: 91. The protein is also described in UniProtKB/Swiss Prot under the reference number UGT73B1.

Alternatively, the UGT is from Scutellaria baicalensis. In a first aspect, the nucleic acid sequences coding for a first UGT and protein sequences are described in NCBI under the reference numbers KU712253 and AMK52071.1, respectively, and more particularly in SEQ ID NO: 93. The protein is described in UniProtKB/Swiss Prot under the reference number AOA140DPB7. In a second aspect, the nucleic acid sequences coding for a second UGT and protein sequences are described in NCBI under the reference numbers KU712254 and AMK52072.1, respectively, and more particularly in SEQ ID NO: 95. The protein is described in UniProtKB/Swiss Prot under the reference number AOA140DPB8. In a third aspect, the nucleic acid sequences coding for a third UGT and protein sequences are described in NCBI under the reference numbers KU712255 and AMK52073.1, respectively, and more particularly in SEQ ID NO: 97. The protein is described in UniProtKB/Swiss Prot under the reference number AOA140DPB9.

Moreover, the UGT may be from Homo sapiens. In a first aspect, the UGT is UGT1A6 (UDP glucuronosyltransferase family 1 member A6). The protein is described in UniProtKB/Swiss Prot under the reference number P19224. The consensus coding sequence is described in NCBI under the number CCDS2507.1. The sequence of this enzyme is described in SEQ ID NO: 99. In a second aspect, the UGT is UGT1A7 (UDP glucuronosyltransferase family 1 member A7). The protein is described in UniProtKB/Swiss Prot under the reference number Q9HAW7. The consensus coding sequence is described in NCBI under the number CCDS2506.1. The sequence of this enzyme is described in SEQ ID NO: 101.

The UGT may also be from Citrus, in particular from Citrus sinensis or Citrus clementina. In particular, the UGT from Citrus sinensis is described in SEQ ID NO: 113. A nucleotide sequence coding for this enzyme is described in SEQ ID NO: 114. The UGT from Citrus clementina is described in SEQ ID NO: 115. A nucleotide sequence coding for this enzyme is described in SEQ ID NO: 116.

In a preferred embodiment, the UGT is selected from enzymes comprising a sequence chosen from SEQ ID NOs: 113 and 95 and polypeptides comprising a sequence having at least 60, 70, 80, 85, 90 or 95% sequence identity with one of these sequences and having flavanone 7-O-beta-D-glucosyltransferase activity, preferably selected from enzymes comprising a sequence chosen from SEQ ID NO: 113 and polypeptides comprising a sequence having at least 60, 70, 80, 85, 90 or 95% sequence identity with this sequence and having flavanone 7-O-beta-D-glucosyltransferase activity.

### RhaT: 6-O-Rhamnosyltransferase

RhaT is an enzyme which performs the transfer of a rhamnose into position 6 of the glucose of hesperetin-7-O-glucoside and/or diosmetin-7-O-glucoside. RhaT is a 6-O-rhamnosyltransferase. This enzyme belongs to the class EC 2.4.1.B53.

The inventors had to identify and select enzymes that are capable of accepting hesperetin-7-O-glucoside and/or diosmetin-7-O-glucoside as a substrate and of adding a rhamnose in position 6 of the glucose of these compounds.

The microorganism thus comprises a heterologous nucleic acid sequence coding for a 6-O-rhamnosyltransferase (RhaT) which is capable of transferring a rhamnose into position 6 of the glucose of hesperetin-7-O-glucoside and/or diosmetin-7-O-glucoside. This enzyme is present only in higher eukaryotes, in particular in plants.

The term “6-O-rhamnosyltransferase activity” means the addition of a rhamnose in position 6 of the glucose by the enzyme RhaT. To determine whether there is 6-O-rhamnosyltransferase activity, an enzymatic test may be performed, which consists of the in vitro incubation of the 6-O-rhamnosyltransferase enzyme in the presence of NAD(P)H, O₂, and of a flavonoid, under optimum conditions (pH, ions, etc.), and observation by UPLC-MS and comparison with the standard expected for the appearance of a flavonoid in which a rhamnose is added in position 6 of the glucose. Preferably, the flavonoid is hesperetin 7-O-glucoside or diosmetin 7-O-glucoside and the flavonoids in which a rhamnose is added in position 6 of the glucose are hesperidin and diosmin.

Preferably, this enzyme is an enzyme produced by a plant of the genus Citrus or Petunia hybrida, preferably of the species Citrus sinensis, Citrus maxima, or Citrus clementina. Preferably, the enzyme is an enzyme originating from Citrus sinensis or Citrus clementina.

In a particular embodiment, the 6-O-rhamnosyltransferase is selected from enzymes comprising a sequence chosen from SEQ ID NOs: 103, 105 and polypeptides comprising a sequence having at least 60, 70, 80, 85, 90 or 95% sequence identity with one of these sequences and having 6-O-rhamnosyltransferase activity.

Thus, the RhaT may be from Citrus clementina. It is described in the GenBank database from NCBI under the number XM_006420965 for the nucleic acid sequence and under the number XP_006421028 for the protein sequence, and more particularly in SEQ ID NO: 103. The protein is described in UniProtKB/Swiss Prot under the reference number V4RJL6.

The RhaT may also be from Citrus sinensis. It is described in the GenBank database from NCBI under the number DQ119035 for the nucleic acid sequence and under the number ABA18631.1 for the protein sequence, and more particularly in SEQ ID NO: 105. The protein is described in UniProtKB/Swiss Prot under the reference number A7ISD3.

In a particular embodiment, the RhaT is from Citrus sinensis and is an enzyme comprising the sequence SEQ ID NO: 105 or a polypeptide comprising a sequence having at least 60, 70, 80, 85, 90 or 95% sequence identity therewith and having 6″-O-rhamnosyltransferase activity.

In a preferred embodiment, the RhaT is selected from enzymes comprising a sequence chosen from SEQ ID NO: 103 and polypeptides comprising a sequence having at least 60, 70, 80, 85, 90 or 95% sequence identity with this sequence and having 6″-O-rhamnosyltransferase activity.

### RHM: UDP-Glucose 4,6-Dehydratase/UDP-4-Keto-6-Deoxy-D-Glucose 3,5-Epimerase/UDP-4-Keto-L-Rhamnose-Reductase

RHM is a trifunctional enzyme UDP-glucose 4,6-dehydratase/UDP-4-keto-6-deoxy-D-glucose 3,5-epimerase/UDP-4-keto-L-rhamnose-reductase. This enzyme is capable of producing UDP-rhamnose from UDP-glucose. This enzyme belongs to the class EC 4.2.1.76. UDP-rhamnose is necessary for 6-O-rhamnosyltransferase (RhaT) activity.

The microorganism thus comprises a heterologous nucleic acid sequence coding for a UDP-glucose 4,6-dehydratase/UDP-4-keto-6-deoxy-D-glucose 3,5-epimerase/UDP-4-keto-L-rhamnose-reductase (RHM) which is capable of producing UDP-rhamnose. This enzyme is present only in higher eukaryotes, in particular in plants.

The term “UDP-glucose 4,6-dehydratase/UDP-4-keto-6-deoxy-D-glucose 3,5-epimerase/UDP-4-keto-L-rhamnose-reductase activity” means the transformation of UDP-glucose into UDP-rhamnose. To determine whether there is UDP-glucose 4,6-dehydratase/UDP-4-keto-6-deoxy-D-glucose 3,5-epimerase/UDP-4-keto-L-rhamnose-reductase activity, an enzymatic test may be performed, which consists of the in vitro incubation of the UDP-glucose enzyme, NAD(P)H and O₂, under optimum conditions (pH, ions, etc.), and observation by UPLC-MS and comparison with the standard expected for the appearance of a UDP-rhamnose.

Preferably, this enzyme is an enzyme produced by a plant of the genus Citrus, in particular Citrus sinensis, or by Arabidopsis thaliana.

In a particular embodiment, the UDP-glucose 4,6-dehydratase/UDP-4-keto-6-deoxy-D-glucose 3,5-epimerase/UDP-4-keto-L-rhamnose-reductase is selected from enzymes comprising a sequence chosen from SEQ ID NOs: 107, 109 and 111 and polypeptides comprising a sequence having at least 60, 70, 80, 85, 90 or 95% sequence identity with one of these sequences and having UDP-glucose 4,6-dehydratase/UDP-4-keto-6-deoxy-D-glucose 3,5-epimerase/UDP-4-keto-L-rhamnose-reductase activity.

The RHM may be from Citrus sinensis. It is described in the GenBank database from NCBI under the number XM_006477756 for the nucleic acid sequence and under the number XP_006477819.1 for the protein sequence, and more particularly in SEQ ID NO: 107.

The RHM may also be from Arabidopsis thaliana. In a first aspect, the protein is described in the GenBank database from NCBI under the number AY081471 for the nucleic acid sequence and under the number AAM10033.1 for the protein sequence, and more particularly in SEQ ID NO: 109. The protein is described in UniProtKB/Swiss Prot under the reference number Q9SYM5. In a second aspect, the protein is described in the GenBank database from NCBI under the number AJ565874 for the nucleic acid sequence and under the number CAD92667.1 for the protein sequence, and more particularly in SEQ ID NO: 111. The protein is described in UniProtKB/Swiss Prot under the reference number Q9LPG6.

In a particular embodiment, the RHM is an enzyme comprising a sequence chosen from SEQ ID NOs: 107 and 109 and polypeptides comprising a sequence having at least 60, 70, 80, 85, 90 or 95% sequence identity with one of these sequences and having UDP-glucose 4,6-dehydratase/UDP-4-keto-6-deoxy-D-glucose 3,5-epimerase/UDP-4-keto-L-rhamnose-reductase activity.

In a preferred embodiment, the RHM is selected from enzymes comprising a sequence chosen from SEQ ID NO: 107 and polypeptides comprising a sequence having at least 60, 70, 80, 85, 90 or 95% sequence identity with this sequence and having UDP-glucose 4,6-dehydratase/UDP-4-keto-6-deoxy-D-glucose 3,5-epimerase/UDP-4-keto-L-rhamnose-reductase activity.

### Combination of Enzymes

In a particular embodiment, the recombinant microorganism comprises:
a heterologous nucleic acid sequence coding for a flavanone 7-O-beta-D-glucosyltransferase (UGT) from Arabidopsis thaliana, Scutellaria baicalensis or Homo sapiens, preferably from Arabidopsis thaliana or from Scutellaria baicalensis, preferably a flavanone 7-O-beta-D-glucosyltransferase (UGT) selected from enzymes comprising a sequence chosen from SEQ ID NOs: 113, 115, 91, 93, 95, 97, 99 and 101 and polypeptides comprising a sequence having at least 60, 70, 80, 85, 90 or 95% sequence identity with one of these sequences and having flavanone 7-O-beta-D-glucosyltransferase activity; in particular a flavanone 7-O-beta-D-glucosyltransferase (UGT) comprising a sequence chosen from SEQ ID NOs: 113, 115, 91, 93, 95 and 97 and polypeptides comprising a sequence having at least 60, 70, 80, 85, 90 or 95% sequence identity with one of these sequences and having flavanone 7-O-beta-D-glucosyltransferase activity, and most particularly preferably comprising a sequence chosen from SEQ ID NOs: 113, 115 and 95 and polypeptides comprising a sequence having at least 60, 70, 80, 85, 90 or 95% sequence identity with one of these sequences and having flavanone 7-O-beta-D-glucosyltransferase activity, in particular comprising a sequence chosen from SEQ ID NOs: 113 and 95 and polypeptides comprising a sequence having at least 60, 70, 80, 85, 90 or 95% sequence identity with one of these sequences and having flavanone 7-O-beta-D-glucosyltransferase activity;
a heterologous nucleic acid sequence coding for a 6-O-rhamnosyltransferase (RhaT) of the genus Citrus or Petunia hybrida, preferably Citrus sinensis, Citrus maxima, or Citrus clementina, even more preferably Citrus sinensis or Citrus clementina, preferably a 6-O-rhamnosyltransferase (RhaT) comprising a sequence chosen from SEQ ID NOs: 103 and 105 and polypeptides comprising a sequence having at least 60, 70, 80, 85, 90 or 95% sequence identity with one of these sequences and having 6-O-rhamnosyltransferase activity, and preferably an RhaT comprising a sequence chosen from SEQ ID NO: 103 and polypeptides comprising a sequence having at least 60, 70, 80, 85, 90 or 95% sequence identity with this sequence and having 6″-O-rhamnosyltransferase activity; and
a heterologous nucleic acid sequence coding for a UDP-glucose 4,6-dehydratase/UDP-4-keto-6-deoxy-D-glucose 3,5-epimerase/UDP-4-keto-L-rhamnose-reductase (RHM) from Citrus sinensis or from Arabidopsis thaliana, preferably an RHM comprising a sequence chosen from SEQ ID NOs: 107, 109 and 111 and polypeptides comprising a sequence having at least 60, 70, 80, 85, 90 or 95% sequence identity with one of these sequences and having UDP-glucose 4,6-dehydratase/UDP-4-keto-6-deoxy-D-glucose 3,5-epimerase/UDP-4-keto-L-rhamnose-reductase activity, in particular an RHM comprising a sequence selected from SEQ ID NOs: 107 and 109 and polypeptides comprising a sequence having at least 60, 70, 80, 85, 90 or 95% sequence identity with this sequence and having UDP-glucose 4,6-dehydratase/UDP-4-keto-6-deoxy-D-glucose 3,5-epimerase/UDP-4-keto-L-rhamnose-reductase activity, and preferably an RHM comprising a sequence selected from SEQ ID NO: 107 and polypeptides comprising a sequence having at least 60, 70, 80, 85, 90 or 95% sequence identity with this sequence and having UDP-glucose 4,6-dehydratase/UDP-4-keto-6-deoxy-D-glucose 3,5-epimerase/UDP-4-keto-L-rhamnose-reductase activity.

In another particular embodiment, the recombinant microorganism comprises a heterologous nucleic acid sequence coding for a 6-O-rhamnosyltransferase (RhaT) and a heterologous nucleic acid sequence coding for a UDP-glucose 4,6-dehydratase/UDP-4-keto-6-deoxy-D-glucose 3,5-epimerase/UDP-4-keto-L-rhamnose-reductase (RHM) as defined in the preceding embodiment and a heterologous nucleic acid sequence coding for a flavanone 7-O-beta-D-glucosyltransferase (UGT) from Arabidopsis thaliana, Scutellaria baicalensis or Homo sapiens, preferably from Arabidopsis thaliana or from Scutellaria baicalensis, preferably a flavanone 7-O-beta-D-glucosyltransferase (UGT) selected from enzymes comprising a sequence chosen from SEQ ID NOs: 91, 93, 95, 97, 99 and 101 and polypeptides comprising a sequence having at least 60, 70, 80, 85, 90 or 95% sequence identity with one of these sequences and having flavanone 7-O-beta-D-glucosyltransferase activity; in particular a flavanone 7-O-beta-D-glucosyltransferase (UGT) comprising a sequence chosen from SEQ ID NOs: 91, 93, 95 and 97 and polypeptides comprising a sequence having at least 60, 70, 80, 85, 90 or 95% sequence identity with one of these sequences and having flavanone 7-O-beta-D-glucosyltransferase activity.

In a preferred embodiment, the microorganism comprises:
a heterologous nucleic acid sequence coding for a flavanone 7-O-beta-D-glucosyltransferase (UGT) selected from enzymes comprising a sequence chosen from SEQ ID NOs: 113, 115 and 95 and polypeptides comprising a sequence having at least 60, 70, 80, 85, 90 or 95% sequence identity with one of these sequences and having flavanone 7-O-beta-D-glucosyltransferase activity, preferably selected from enzymes comprising a sequence chosen from SEQ ID NOs: 113 and 95 and polypeptides comprising a sequence having at least 60, 70, 80, 85, 90 or 95% sequence identity with one of these sequences and having flavanone 7-O-beta-D-glucosyltransferase activity, and more particularly preferably selected from enzymes comprising a sequence chosen from SEQ ID NO: 113 and polypeptides comprising a sequence having at least 60, 70, 80, 85, 90 or 95% sequence identity with this sequence and having flavanone 7-O-beta-D-glucosyltransferase activity; and
a heterologous nucleic acid sequence coding for a 6″-O-rhamnosyltransferase selected from enzymes comprising a sequence chosen from SEQ ID NOs: 103 and polypeptides comprising a sequence having at least 60, 70, 80, 85, 90 or 95% sequence identity with this sequence and having 6″-O-rhamnosyltransferase activity; and
a heterologous nucleic acid sequence coding for a UDP-glucose 4,6-dehydratase/UDP-4-keto-6-deoxy-D-glucose 3,5-epimerase/UDP-4-keto-L-rhamnose-reductase selected from enzymes comprising a sequence chosen from SEQ ID NO: 107 and polypeptides comprising a sequence having at least 60, 70, 80, 85, 90 or 95% sequence identity with this sequence and having UDP-glucose 4,6-dehydratase/UDP-4-keto-6-deoxy-D-glucose 3,5-epimerase/UDP-4-keto-L-rhamnose-reductase activity.

In a particular embodiment, the enzymes described above are enzymes from higher eukaryotes, preferably plant enzymes. In a particular embodiment, the enzymes originate from plants of the same genus, for example of the same species.

Modifications Enabling the Production of Hesperetin and/or Diosmetin

As indicated previously, either hesperetin and/or diosmetin are supplied to the microorganism, or the microorganism is capable of producing hesperetin and/or diosmetin. In particular, the microorganism is capable or has been modified to be capable of producing hesperetin and/or diosmetin. The inventors thus also developed a biosynthetic pathway enabling the microorganism to produce hesperetin and/or diosmetin.

### From Naringenin/Apigenin to Hesperetin/Diosmetin

Hesperetin and/or diosmetin may be obtained from naringenin and apigenin. Several biosynthetic strategies were possible. To prepare hesperetin and/or diosmetin, it is necessary to make two modifications: methylation of the hydroxyl in position 4′ and hydroxylation of position 3′. Thus, to increase the specificity of methylation of the hydroxyl in position 4′, it appears logical to first perform methylation of the hydroxyl group already present before adding a second hydroxyl group in position 3′. On the contrary, the inventors arrived at the conclusion that it was necessary first to perform the hydroxylation and then the methylation, despite the risk of the problem of methylation specificity due to the introduction of the second hydroxyl.

### F3′H: Flavonoid 3′-Monooxygenase

Flavonoid 3′-monooxygenase (F3′H) is an enzyme which performs the addition of a hydroxyl group in position 3′ of naringenin and/or apigenin. This enzyme belongs to the class EC 1.14.14.82. It is also known as flavone 3′-hydroxylase.

The inventors had to identify and select enzymes that are capable of accepting naringenin and/or apigenin as a substrate and of adding a hydroxyl group in position 3′ of these compounds. Preferably, the enzyme is selected so as to have a preference for hydroxylation in position 3′ of naringenin and/or apigenin. In a preferred embodiment, the enzyme is specific for the 3′ position of naringenin and/or apigenin, in particular relative to the 5′ position so as to avoid a double hydroxylation in positions 3′ and 5′, and preferably also to avoid hydroxylation in position 5′.

Flavonoid 3′-monooxygenase (F3′H) is an enzyme which performs the addition of a hydroxyl group in position 3′ of naringenin and/or apigenin. This enzyme belongs to the class EC 1.14.14.82. It is also known as flavonoid 3′-hydroxylase.

The term “flavonoid 3′-monooxygenase activity” means the transformation of a flavonoid into a 3′-hydroxylated flavonoid by a CPR-dependent F3′H enzyme. To determine whether there is flavonoid 3′-monooxygenase activity, an enzymatic test may be performed, which consists of the in vitro incubation of the flavonoid 3′-monooxygenase enzyme in the presence of NAD(P)H, O₂, and of a flavonoid, under optimum conditions (pH, ions, etc.), and observation by UPLC-MS and comparison with the standard expected for the appearance of a 3′-hydroxylated flavonoid. Preferably, the flavonoid is naringenin or apigenin and the 3′-hydroxylated flavonoid is the 3′-hydroxylated form thereof, i.e. eriodictyol or luteolin.

The microorganism may thus comprise a heterologous nucleic acid sequence coding for a flavonoid 3′-monooxygenase (F3′H) which is capable of adding a hydroxyl in position 3′ of naringenin and/or apigenin.

In one embodiment, the F3′H is a plant enzyme, notably from plants of the genus Allium, Arabidopsis, Brassica, Callistephus, Columnea, Citrus, Dianthus, Gentiana, Gerbera, Glycine, Fragaria, Ipomoea, Malus, Matthiola, Osteospermum, Oryza, Phanerochaete, Perilla, Petroselinum, Pelargonium, Pilosella, Petunia, Sinningia, Sorghum, Torenia, Vitis or Zea, for example Allium cepa, Arabidopsis thaliana, Brassica napus, Columnea hybrida, Callistephus chinensis, Citrus sinensis, Citrus clementina, Dianthus caryophyllus, Fragaria vesca, Fragaria x ananassa, Gerbera hybrida, Glycine max, Gentiana triflora, Ipomoea nil, Ipomoea purpurea, Ipomoea tricolor, Matthiola incana, Malus domestica, Osteospermum hybrid cultivar, Oryza sativa, Phanerochaete chrysosporium, Pilosella officinarum, Petroselinum crispum, Pelargonium x hortorum, Perilla frutescens var. crispa, Petunia x hybrida, Sinningia cardinalis, Sorghum bicolor, Torenia sp, Torenia hybrid cultivar, Vitis vinifera or Zea mays. In a more specific embodiment, the F3′H is an enzyme from plants of the genus Allium, Brassica, Callistephus, Columnea, Citrus, Dianthus, Gentiana, Gerbera, Glycine, Fragaria, Ipomoea, Malus, Matthiola, Osteospermum, Oryza, Phanerochaete, Perilla, Petroselinum, Pelargonium, Pilosella, Petunia, Sinningia, Sorghum, Torenia, Vitis or Zea, for example Allium cepa, Brassica napus, Columnea hybrida, Callistephus chinensis, Citrus sinensis, Citrus clementina, Dianthus caryophyllus, Fragaria vesca, Fragaria x ananassa, Gerbera hybrida, Glycine max, Gentiana triflora, Ipomoea nil, Ipomoea purpurea, Ipomoea tricolor, Matthiola incana, Malus domestica, Osteospermum hybrid cultivar, Oryza sativa, Phanerochaete chrysosporium, Pilosella officinarum, Petroselinum crispum, Pelargonium x hortorum, Perilla frutescens var. crispa, Petunia x hybrida, Sinningia cardinalis, Sorghum bicolor, Torenia sp, Torenia hybrid cultivar, Vitis vinifera or Zea mays.

Preferably, the F3′H is an enzyme from Perilla frutescens var. crispa, Petunia x hybrida, Callistephus chinensis, Gerbera hybrida, Citrus clementina, Osteospermum hybrid cultivar, Phanerochaete chrysosporium, Streptomyces avermitilis, Citrus sinensis, Arabidopsis thaliana or Pilosella officinarum. In particular, the F3′H may be an enzyme from Perilla frutescens var. crispa, Petunia x hybrida, Callistephus chinensis, Gerbera hybrida, Citrus sinensis and Pilosella officinarum.

In a particular embodiment, the F3′H is selected from enzymes comprising a sequence chosen from SEQ ID NOs: 1, 3, 5, 7, 9, 11, 13, 15, 17, 19, 21 and 121, in particular from SEQ ID NOs: 1, 3, 5, 7, 9, 11, 13, 15, 17, 19 and 21 and polypeptides comprising a sequence having at least 60, 70, 80, 85, 90 or 95% sequence identity with one of these sequences and having flavonoid 3′-monooxygenase activity, preferably selected from enzymes comprising a sequence chosen from SEQ ID NOs: 1, 5, 7, 11, 17, 19 and 121, in particular from SEQ ID NOs: 1, 5, 7, 11, 17 and 19 and polypeptides comprising a sequence having at least 60, 70, 80, 85, 90 or 95% sequence identity with one of these sequences and having flavonoid 3′-monooxygenase activity, notably with naringenin and/or apigenin as substrate and with hydroxylation in position 3′. In a particular embodiment, the F3′H is an enzyme comprising a sequence selected from SEQ ID NOs: 5, 7 and 17 and polypeptides comprising a sequence having at least 60, 70, 80, 85, 90 or 95% identity with one of these sequences and having flavonoid 3′-monooxygenase activity.

In a preferred embodiment, the F3′H is an enzyme comprising a sequence selected from SEQ ID NOs: 7, 11, 17 and 121 and polypeptides comprising a sequence having at least 60, 70, 75, 80, 85, 90 or 95% identity with one of these sequences and having flavonoid 3′-monooxygenase activity. Most particularly preferably, the F3′H may be an enzyme comprising a sequence selected from SEQ ID NOs: 7, 17 and 121 and polypeptides comprising a sequence having at least 60, 70, 75, 80, 85, 90 or 95% identity with one of these sequences and having flavonoid 3′-monooxygenase activity.

Thus, the F3′H may be from Perilla frutescens var. crispa. The nucleic acid sequences coding for this enzyme and protein sequences are described in NCBI under the reference numbers AB045593.1 and BAB59005.1, respectively, and more particularly in SEQ ID NOs: 2 and 1.

The F3′H may be from Phanerochaete chrysosporium. The nucleic acid sequences coding for this enzyme and protein sequences are described in NCBI under the reference numbers AB597870.1 and BAL05157.1, respectively, and more particularly in SEQ ID NOs: 4 and 3.

The F3′H may be from Petunia x hybrida. The nucleic acid sequences coding for this enzyme and protein sequences are described in NCBI under the reference numbers AF155332.1 and AAD56282.1, respectively, and more particularly in SEQ ID NOs: 6 and 5.

The F3′H may be from Callistephus chinensis. In one embodiment, the nucleic acid sequences coding for this enzyme and protein sequences are described in NCBI under the reference numbers AF313488.1 and AAG49298.1, respectively, and more particularly in SEQ ID NOs: 8 and 7. In another embodiment, the nucleic acid sequences coding for this enzyme and protein sequences are described in NCBI under the reference numbers AF313489.1 and AAG49299.1, respectively, and more particularly in SEQ ID NOs: 10 and 9.

The F3′H may be from Gerbera hybrida. The nucleic acid sequences coding for this enzyme and protein sequences are described in NCBI under the reference numbers DQ218417.1 and ABA64468.1, respectively, and more particularly in SEQ ID NOs: 12 and 11.

The F3′H may be from Osteospermum hybrid cultivar. The nucleic acid sequences coding for this enzyme and protein sequences are described in NCBI under the reference numbers DQ250711.1 and ABB29899.1, respectively, and more particularly in SEQ ID NOs: 14 and 13.

The F3′H may be from Citrus clementina. The nucleic acid sequences coding for this enzyme and protein sequences are described in NCBI under the reference numbers XM_006440673.1 and XP_006440736.1, respectively, and more particularly in SEQ ID NOs: 16 and 15.

The F3′H may be from Citrus sinensis. The nucleic acid sequences coding for this enzyme and protein sequences are described in NCBI under the reference numbers XM_006477592.2 et XP_006477655.1, respectively, and more particularly in SEQ ID NOs: 18 and 17.

The F3′H may be from Pilosella officinarum. The nucleic acid sequences coding for this enzyme and protein sequences are described in NCBI under the reference numbers DQ319866.2 and ABC47161.1, respectively, and more particularly in SEQ ID NOs: 20 and 19.

The F3′H may be from Streptomyces avermitilis. The nucleic acid sequences coding for this enzyme and protein sequences are described in NCBI under the reference numbers SAV_4539 et WP_010985964.1, respectively, and more particularly in SEQ ID NOs: 22 and 21.

The F3′H may be from Arabidopsis thaliana. A nucleic acid sequence coding for this enzyme and the protein sequence are described in NCBI under the reference numbers NM_120881.2 and NP_196416.1, respectively, and more particularly in SEQ ID NOs: 122 and 121.

Preferably, the F3′H is an enzyme comprising a sequence selected from SEQ ID NOs: 7, 11, 17 and 121 and polypeptides comprising a sequence having at least 75%, at least 80%, at least 85%, at least 90% or at least 95% identity with one of these sequences and having flavonoid 3′-monooxygenase activity. Most particularly preferably, the F3′H is an enzyme comprising a sequence selected from SEQ ID NOs: 7, 17 and 121 and polypeptides comprising a sequence having at least 75%, at least 80%, at least 85%, at least 90% or at least 95% identity with one of these sequences and having flavonoid 3′-monooxygenase activity.

According to a preferred embodiment, the F3′H is an enzyme comprising a sequence chosen from SEQ ID NO: 7 and polypeptides comprising a sequence having at least 75%, at least 80%, at least 85%, at least 90% or at least 95% sequence identity with the sequence SEQ ID NO: 7 and having flavonoid 3′-monooxygenase activity.

According to another particular embodiment, the F3′H is an enzyme comprising a sequence chosen from SEQ ID NO: 17 and polypeptides comprising a sequence having at least 75%, at least 80%, at least 85%, at least 90% or at least 95% sequence identity with the sequence SEQ ID NO: 17 and having flavonoid 3′-monooxygenase activity.

According to another particular embodiment, the F3′H is an enzyme comprising a sequence chosen from SEQ ID NO: 121 and polypeptides comprising a sequence having at least 70%, at least 75%, at least 80%, at least 85%, at least 90% or at least 95% sequence identity with the sequence SEQ ID NO: 121 and having flavonoid 3′-monooxygenase activity.

According to another particular embodiment, the F3′H is an enzyme comprising a sequence chosen from SEQ ID NO: 11 and polypeptides comprising a sequence having at least 75%, at least 80%, at least 85%, at least 90% or at least 95% sequence identity with the sequence SEQ ID NO: 11 and having flavonoid 3′-monooxygenase activity.

CPR: cytochrome P450 reductase Flavonoid 3′-monooxygenase (F3′H) requires the presence of NADPH to perform the addition of the hydroxyl group.

Thus, in a preferred embodiment, the microorganism comprises a heterologous nucleic acid coding for a cytochrome P450 reductase, an NADPH-cytochrome P450 reductase. This enzyme belongs to the class EC 1.6.2.4.

Cytochrome P450 reductase originates from a eukaryote, notably from a yeast, for example of the genus Saccharomycetales, or from a plant, for example a plant of the genus Arabidopsis, Ammi, Avicennia, Camellia, Camptotheca, Catharanthus, Citrus, Glycine, Helianthus, Lotus, Mesembryanthemum, Phaseolus, Physcomitrella, Pinus, Populus, Ruta, Saccharum, Solanum, Vigna, Vitis or Zea.

In a preferred embodiment, the cytochrome P450 reductase originates from a eukaryote, for example from yeast, in particular from Saccharomyces cerevisiae, or from a plant, for example from Catharanthus roseus or Arabidopsis thaliana.

In a particular embodiment, the cytochrome P450 reductase is selected from enzymes comprising a sequence chosen from SEQ ID NOs: 23, 25, 27, 29 and 31 and polypeptides comprising a sequence having at least 60, 70, 80, 85, 90 or 95% sequence identity with one of these sequences and having cytochrome P450 reductase activity, preferably from enzymes comprising a sequence chosen from SEQ ID NOs: 23, 25, 29 and 31 and polypeptides comprising a sequence having at least 60, 70, 80, 85, 90 or 95% sequence identity with one of these sequences and having cytochrome P450 reductase activity. In a very particular embodiment, the cytochrome P450 reductase may be selected from enzymes comprising a sequence chosen from SEQ ID NO: 25 and polypeptides comprising a sequence having at least 60, 70, 80, 85, 90 or 95% sequence identity with this sequence and having cytochrome P450 reductase activity.

For example, the cytochrome P450 reductase may be from Catharanthus roseus. It is described in the GenBank database from NCBI under the number X69791.1 for the nucleic acid sequence and under the number CAA49446.1 for the protein sequence, and more particularly in SEQ ID NOs: 24 and 23, respectively. The protein is described in UniProtKB/Swiss Prot under the reference number Q05001.

The cytochrome P450 reductase may be from Saccharomyces cerevisiae. It is described in the GenBank database from NCBI under the number NM_001179172.1 for the nucleic acid sequence and under the number NP_011908.1 for the protein sequence, and more particularly in SEQ ID NOs: 26 and 25, respectively. The protein is described in UniProtKB/Swiss Prot under the reference number P16603.

The cytochrome P450 reductase may be chimeric. It is described in the article by Aigrain et al. (2009, EMBO Reports, 10, 742-747). The nucleic acid sequence coding for this enzyme and the protein sequence are described in SEQ ID NOs: 28 and 27, respectively.

Moreover, the cytochrome P450 reductase may be from Arabidopsis thaliana. When the cytochrome P450 originates from Arabidopsis thaliana, it may be named ATR. It is described in the GenBank database from NCBI under the number NM_118585.4 for the nucleic acid sequence and under the number NP_194183.1 for the protein sequence, and more particularly in SEQ ID NOs: 30 and 29, respectively. The protein is described in UniProtKB/Swiss Prot under the reference number Q9SB48.

In addition, the cytochrome P450 reductase may be from Arabidopsis thaliana and may be described in the GenBank database from NCBI under the number NM_179141.2 for the nucleic acid sequence and under the number NP_849472.2 for the protein sequence, and more particularly in SEQ ID NOs: 32 and 31, respectively. The protein is described in UniProtKB/Swiss Prot under the reference number Q9SUM3.

In one embodiment, a new copy of a sequence coding for CPR as defined above is introduced into the yeast. In another embodiment, when the yeast is Saccharomyces cerevisiae and when the CPR originates from the same yeast, the promoter of the endogenous gene coding for CPR is replaced with a strong promoter. Thus, the expression of the CPR is increased relative to the wild-type yeast; the CPR is thus overexpressed in the modified yeast.

In one particular embodiment, the F3′H and the CPR are from the same origin, the same species.

### OMT: O-Methyltransferase

O-methyltransferases (OMT) are a very large family of enzymes having targets that are difficult to define. The inventors had to identify and select O-methyltransferases that are capable of methylating eriodictyol and/or luteolin in position 4′ (para position).

Preferably, the enzyme was selected so as to have a preference for methylation in position 4′ of eriodictyol and/or luteolin. In a preferred embodiment, the enzyme is specific for position 4′ of eriodictyol and/or luteolin. The term “specific” means that the methyl group introduced by the enzyme onto eriodictyol and/or luteolin is found in position 4′ in 60% of the cases, the remainder being introduced into position 3′, preferably in 70% of the cases, and even more preferably in 80% of the cases.

The term “4′-O-methyltransferase activity” means the transformation of a 4′-hydroxyflavonoid into a 4′-methoxyflavonoid by a 4′-O-methyltransferase enzyme. To determine whether there is 4′-O-methyltransferase activity, an enzymatic test may be performed, which consists of the in vitro incubation of a mixture composed of the 4′-O-methyltransferase enzyme, a 4′-hydroxyflavonoid and S-adenosyl-L-methionine, under optimum conditions (pH, temperature, ions, etc.). After a certain incubation time, the appearance of the 4′-methoxyflavonoid is observed in UPLC-MS in comparison with the expected standard.

In the present case, the 4′-hydroxyflavonoid is eriodictyol or luteolin, which will be transformed, respectively, into their 4′-methoxyflavonoid form, i.e. into hesperetin or diosmetin.

The microorganism may thus comprise a heterologous nucleic acid sequence coding for an O-methyltransferase which is capable of methylating eriodictyol and/or luteolin in position 4′.

This enzyme is present only in higher eukaryotes, in particular in plants.

In one embodiment, the O-methyltransferase (OMT) is an enzyme from Arabidopsis thaliana. In another embodiment, the O-methyltransferase (OMT) originates from a higher eukaryote, preferably from a mammal. In particular, the O-methyltransferase (OMT) is of human origin (Homo sapiens).

In a particular embodiment, the OMT is selected from enzymes comprising a sequence chosen from SEQ ID NOs: 87 and 89 and polypeptides comprising a sequence having at least 60, 70, 80, 85, 90 or 95% identity with one of these sequences and having O-methyltransferase activity, notably with eriodictyol and/or luteolin as substrate and with methylation in position 4′.

In one embodiment, the OMT is selected from the enzyme comprising a sequence chosen from SEQ ID NO: 89 and polypeptides comprising a sequence having at least 60, 70, 80, 85, 90 or 95% identity with this sequence and having O-methyltransferase activity.

In another embodiment, the OMT is selected from the enzyme comprising a sequence chosen from SEQ ID NO: 87 and polypeptides comprising a sequence having at least 60, 70, 80, 85, 90 or 95% identity with this sequence and having O-methyltransferase activity.

Thus, the OMT may be from Arabidopsis thaliana. The nucleic acid sequences coding for this enzyme and protein sequences are described in NCBI under the reference numbers NM_118755.4 and NP_567739.1, respectively. The protein is also described in UniProtKB/Swiss Prot under the reference number Q9C5D7, and more particularly in SEQ ID NO: 87.

Alternatively, the OMT is from Homo sapiens. The nucleic acid sequences coding for this enzyme and protein sequences are described in NCBI under the reference numbers NM_007310.2 and NP_009294.1, respectively. The protein is also described in UniProtKB/Swiss Prot under the reference number P21964, and more particularly in SEQ ID NO: 89.

The OMT from Homo sapiens has the advantage of accepting eriodictyol and luteolin as substrate for the methylation, whereas the OMT from Arabidopsis thaliana has a strong preference for eriodictyol. Conversely, if the synthesis of hesperetin is to be favored relative to that of diosmetin, the OMT from Arabidopsis thaliana might have an advantage.

In a preferred embodiment, the OMT is an OMT from Citrus, in particular Citrus clementina or Citrus sinensis. In a particularly preferred embodiment, the OMT is selected from an enzyme comprising a sequence chosen from SEQ ID NOs 117 and 119 and polypeptides comprising a sequence having at least 60, 70, 80, 85, 90 or 95% identity with one of these sequences and having O-methyltransferase activity.

Preferably, the OMT is selected from an enzyme comprising a sequence chosen from SEQ ID NO: 117 and polypeptides comprising a sequence having at least 60, 70, 80, 85, 90 or 95% identity with this sequence and having O-methyltransferase activity.

Alternatively, the OMT is selected from an enzyme comprising a sequence chosen from SEQ ID NO: 119 and polypeptides comprising a sequence having at least 60, 70, 80, 85, 90 or 95% identity with this sequence and having O-methyltransferase activity.

The OMTs from Citrus and from Arabidopsis thaliana described above have the advantage of specifically methylating eriodictyol in position 4′.

During the design of the microorganism, the inventors observed that this methylation step constituted one of the limiting steps. Surprisingly, despite the presence of the cofactor S-adenosyl-L-methionine in the microorganism, in particular the yeast, the addition of an enzyme which increases the synthesis of this cofactor made it possible to dispel the limiting aspect of this step. Thus, in a preferred embodiment, the microorganism also comprises a heterologous or endogenous sequence coding for an enzyme which synthesizes S-adenosyl-L-methionine, an S-adenosylmethionine synthetase (SAMT). This enzyme belongs to the class EC 2.5.1.6.

In one embodiment, the microorganism comprises a heterologous nucleic acid sequence coding for an O-methyltransferase (OMT), which is in particular capable of methylating eriodictyol and/or luteolin in position 4′ and a heterologous or endogenous nucleic acid sequence coding for an S-adenosylmethionine synthetase (SAMT).

In one embodiment, the SAMT originates from a yeast, in particular from Saccharomyces cerevisiae, most particularly when the microorganism is a yeast.

In a particular embodiment, the S-adenosylmethionine synthetase is an enzyme comprising a sequence chosen from SEQ ID NO: 81 and a polypeptide comprising a sequence having at least 60, 70, 80, 85, 90 or 95% identity with this sequence and having S-adenosylmethionine synthetase activity.

For example, the S-adenosylmethionine synthase may be from Saccharomyces cerevisiae. It is described in the GenBank database from NCBI under the number NM_001180810.3 for the nucleic acid sequence and under the number NP_010790.3 for the protein sequence. The protein is described in UniProtKB/Swiss Prot under the reference number P19358.

In one embodiment, a new copy of a sequence coding for SAMT as defined above is introduced into the microorganism. In another embodiment, when the microorganism is Saccharomyces cerevisiae, the promoter of the endogenous gene coding for SAMT is replaced with a strong promoter. Thus, the expression of the SAMT is increased relative to the wild-type microorganism; the SAMT is thus overexpressed in the modified microorganism.

Thus, in a preferred embodiment, the microorganism comprises a heterologous nucleic acid sequence coding for an O-methyltransferase which is capable of methylating eriodictyol and/or luteolin in position 4′ and a heterologous or endogenous nucleic acid sequence coding for an S-adenosylmethionine synthetase (SAMT) which is capable of producing S-adenosyl-L-methionine.

### FNS: Flavone Synthase

Diosmetin may be produced from luteolin. It may also be obtained from eriodictyol, either by transforming it into luteolin and then preparing diosmetin from luteolin, or by transforming it into hesperetin and then preparing diosmetin from hesperetin. The enzyme that is capable of transforming eriodictyol into luteolin and/or hesperetin into diosmetin is a flavone synthase (FNS). In a particular embodiment, the flavone synthase is also capable of transforming eriodictyol into luteolin.

Thus, the microorganism may comprise a heterologous nucleic acid sequence coding for a flavone synthase, in particular a flavone synthase which is capable of producing luteolin from eriodictyol and/or diosmetin from hesperetin.

The term “flavone synthase activity” means the transformation of a flavanone into flavone by an FNSI enzyme (CPR-independent) or an FNSII enzyme (CPR-dependent).

To determine if there is flavone synthase activity, an enzymatic test may be performed, which consists of the in vitro incubation in the case of FNSI of a mixture composed of the flavone synthase enzyme (FNSI), a flavanone, 2-oxoglutarate and O₂, under optimum conditions (pH, temperature, ions, etc.) and in the case of FNSII of a mixture composed of the enzyme FNSII, a flavanone, NAD(P)H and O₂, under optimum conditions (pH, temperature, ions, etc.). After a certain incubation time, the appearance of the flavone corresponding to the flavanone is observed in UPLC-MS in comparison with the expected standard. Preferably, the flavanone is eriodictyol or hesperetin, which will be transformed, respectively, into their flavone form, i.e. into luteolin or diosmetin.

Thus, in a particular embodiment, the microorganism comprises a heterologous nucleic acid sequence coding for an O-methyltransferase (OMT), which is in particular capable of methylating eriodictyol and/or luteolin in position 4′; and a heterologous nucleic acid sequence coding for a flavone synthase, in particular a flavone synthase which is capable of producing luteolin from eriodictyol and/or diosmetin from hesperetin.

Preferably, the flavone synthase is an enzyme originating from a plant, for example of the genus Aethusa, Angelica, Antirrhinum, Apium, Arabidopsis, Callistephus, Camellia, Conium, Cuminum, Cynara, Dahlia, Dorcoceras, Erythranthe, Lonicera, Medicago, Oryza, Perilla, Petroselinum, Plectranthus, Populus, Saussurea, Scutellaria or Zea, in particular of the genus Arabidopsis, Lonicera, Medicago, Oryza, Petroselinum, Populus or Zea, notably of Arabidopsis thaliana, Lonicera japonica, Lonicera macranthoides, Medicago truncatula, Oryza sativa, Petroselinum crispum, Populus deltoides, Zea mays, Callistephus chinensis, Apium graveolens, Cuminum cyminum, Aethusa cynapium, Angelica archangelica, Conium maculatum, Camellia sinensis, Cynara cardunculus var scolymus, Saussurea medusa, Plectranthus barbatus, Scutellaria baicalensis, Dorcoceras hygrometricum, Antirrhinum majus, Perilla frutescens var crispa, Dahlia pinnata or Erythranthe lewisii, in particular of Arabidopsis thaliana, Lonicera japonica, Lonicera macranthoides, Medicago truncatula, Oryza sativa, Petroselinum crispum, Populus deltoides or Zea mays, preferably of Petroselinum crispum, or of the genus Lonicera for instance Lonicera japonica and Lonicera macranthoides.

In a particular embodiment, the flavone synthase (FNS) is selected from enzymes comprising a sequence chosen from SEQ ID NOs: 33, 35, 37, 127, 129, 131, 133, 135, 137, 139, 141, 143, 145, 147, 149, 151, 153, 155, 157 and 159 and polypeptides comprising a sequence having at least 60, 70, 80, 85, 90 or 95% sequence identity with one of these sequences and having flavone synthase activity. In particular, the flavone synthase (FNS) is selected from enzymes comprising a sequence chosen from SEQ ID NOs: 33, 35 and 37 and polypeptides comprising a sequence having at least 60, 70, 80, 85, 90 or 95% sequence identity with one of these sequences and having flavone synthase activity. Preferably, the FNS is selected from enzymes comprising a sequence chosen from SEQ ID NO: 37 and polypeptides comprising a sequence having at least 60, 70, 80, 85, 90 or 95% sequence identity with this sequence and having flavone synthase activity.

There are two types of flavone synthase (FNS): flavone synthase 1 (FNSI) and flavone synthase 2 (FNSII). Starting with a flavanone and 2-oxoglutarate, FNSI is capable of producing the corresponding flavone. The enzyme FNSI belongs to the class EC 1.14.11.22. FNSII belongs to the P450 group and requires the presence of a cytochrome P450 reductase. The enzyme FNSII belongs to the class EC 1.14.13.

In one embodiment, the FNS is a type I flavone synthase. In another embodiment, the FNS is a type II flavone synthase. In an additional embodiment, the microorganism comprises a type I flavone synthase and a type II flavone synthase.

In a preferred embodiment, the microorganism comprises a heterologous nucleic acid sequence coding for a type I flavone synthase (FNSI). The advantage of FNSI is that it functions without cytochrome P450 reductase.

The FNSI may be a flavone synthase from a plant such as Petroselinum crispum, Oryza sativa, Populus deltoides, Medicago truncatula, Apium graveolens, Cuminum cyminum, Aethusa cynapium, Angelica archangelica, or Conium maculatum, in particular from Petroselinum crispum, Oryza sativa, Populus deltoides or Medicago truncatula, preferably from Petroselinum crispum.

The FNSI may be an enzyme comprising a sequence chosen from SEQ ID NOs: 37, 127, 137, 141, 143 and 145 and polypeptides comprising a sequence having at least 60, 70, 80, 85, 90 or 95% sequence identity with one of these sequences and having flavone synthase activity. In a particular aspect, the FNSI may be an enzyme comprising a sequence chosen from SEQ ID NO: 37 and a polypeptide comprising a sequence having at least 60, 70, 80, 85, 90 or 95% sequence identity with this sequence and having flavone synthase activity.

For example, the FNSI may be from Petroselinum crispum. It is described in the GenBank database from NCBI under the number AY817680.1 for the nucleic acid sequence and under the number AAX21541.1 for the protein sequence. The protein is described in UniProtKB/Swiss Prot under the reference number Q7XZQ8. The amino acid and nucleic acid sequences are described in SEQ ID NOs: 37 and 38, respectively.

The FNSI may also be from Angelica archangelica. It is described in the GenBank database from NCBI under the number DQ683352.1 for the nucleic acid sequence and under the number ABG78793.1 for the protein sequence. The amino acid and nucleic acid sequences are described in SEQ ID NOs: 127 and 128, respectively.

The FNSI may also be from Apium graveolens. It is described in the GenBank database from NCBI under the number AY817676.1 for the nucleic acid sequence and under the number AAX21537.1 for the protein sequence. The amino acid and nucleic acid sequences are described in SEQ ID NOs: 137 and 138, respectively.

The FNSI may also be from Cuminum cyminum. It is described in the GenBank database from NCBI under the number DQ683349.1 for the nucleic acid sequence and under the number ABG78790.1 for the protein sequence. The amino acid and nucleic acid sequences are described in SEQ ID NOs: 141 and 142, respectively.

The FNSI may also be from Aethusa cynapium. It is described in the GenBank database from NCBI under the number DQ683350.1 for the nucleic acid sequence and under the number DQ683350.1 for the protein sequence. The amino acid and nucleic acid sequences are described in SEQ ID NOs: 143 and 144, respectively.

The FNSI may also be from Conium maculatum. It is described in the GenBank database from NCBI under the number DQ683354.1 for the nucleic acid sequence and under the number ABG78795.1 for the protein sequence. The amino acid and nucleic acid sequences are described in SEQ ID NOs: 145 and 146, respectively.

In another embodiment, the microorganism comprises a heterologous nucleic acid sequence coding for a type II flavone synthase (FNSII).

The FNSII may be a flavone synthase from a plant, for example from Arabidopsis thaliana, Zea mays, of the genus Lonicera for instance Lonicera japonica and Lonicera macranthoides, Callistephus chinensis, Medicago truncatula, Camellia sinensis, Cynara cardunculus var scolymus, Saussurea medusa, Plectranthus barbatus, Scutellaria baicalensis, Dorcoceras hygrometricum, Antirrhinum majus, Perilla frutescens var crispa, Dahlia pinnata or Erythranthe lewisii, in particular a flavone synthase from Arabidopsis thaliana, Zea mays or of the genus Lonicera, for instance Lonicera japonica and Lonicera macranthoides.

In a particular embodiment, the flavone synthase (FNSII) is selected from enzymes comprising a sequence chosen from SEQ ID NOs: 33, 35, 129, 131, 133, 135, 139, 147, 149, 151, 153, 155, 157 and 159 and polypeptides comprising a sequence having at least 60, 70, 80, 85, 90 or 95% sequence identity with one of these sequences and having flavone synthase activity, preferably from enzymes comprising a sequence chosen from SEQ ID NOs: 33 and 35 and polypeptides comprising a sequence having at least 60, 70, 80, 85, 90 or 95% sequence identity with one of these sequences and having flavone synthase activity.

In one embodiment, the flavone synthase FNS is an FNSII originating from Lonicera japonica. In this embodiment, the enzyme may be an enzyme described in the GenBank database from NCBI under the number KU127576.1 for the nucleic acid sequence and under the number AMQ91109.1 for the protein sequence, and more particularly in SEQ ID NOs: 34 and 33, respectively.

In another embodiment, the flavone synthase FNS is an FNSII originating from Lonicera macranthoides. The nucleic acid sequences coding for this enzyme and protein sequences are described in NCBI under the reference numbers KU127580.1 and AMQ91113.1, respectively, and more particularly in SEQ ID NOs: 36 and 35, respectively.

In another embodiment, the flavone synthase FNS is an FNSII originating from Cynara cardunculus var scolymus. The nucleic acid sequences coding for this enzyme and protein sequences are described in NCBI under the reference numbers JN825735.1 and AFG31000.1, respectively, and more particularly in SEQ ID NOs: 130 and 129, respectively.

In another embodiment, the flavone synthase FNS is an FNSII originating from Perilla frutescens var crispa. The nucleic acid sequences coding for this enzyme and protein sequences are described in NCBI under the reference numbers AB045592.1 and BAB59004.1, respectively, and more particularly in SEQ ID NOs: 132 and 131, respectively.

In another embodiment, the flavone synthase FNS is an FNSII originating from Dahlia pinnata. The nucleic acid sequences coding for this enzyme and protein sequences are described in NCBI under the reference numbers AB769842.1 and BAM72335.1, respectively, and more particularly in SEQ ID NOs: 134 and 133, respectively.

In another embodiment, the flavone synthase FNS is an FNSII originating from Callistephus chinensis. The nucleic acid sequences coding for this enzyme and protein sequences are described in NCBI under the reference numbers AF188612.1 and AAF04115.1, respectively, and more particularly in SEQ ID NOs: 136 and 135, respectively.

In another embodiment, the flavone synthase FNS is an FNSII originating from Medicago truncatula. The nucleic acid sequences coding for this enzyme and protein sequences are described in NCBI under the reference numbers DQ354373.1 and ABC86159.1, respectively, and more particularly in SEQ ID NOs: 140 and 139, respectively.

In another embodiment, the flavone synthase FNS is an FNSII originating from Camellia sinensis. The nucleic acid sequences coding for this enzyme and protein sequences are described in NCBI under the reference numbers FJ169499.1 and ACH99109.1, respectively, and more particularly in SEQ ID NOs: 148 and 147, respectively.

In another embodiment, the flavone synthase FNS is an FNSII originating from Saussurea medusa. The nucleic acid sequences coding for this enzyme and protein sequences are described in NCBI under the reference numbers KF170286.1 and AGV40781.1, respectively, and more particularly in SEQ ID NOs: 150 and 149, respectively.

In another embodiment, the flavone synthase FNS is an FNSII originating from Plectranthus barbatus. The nucleic acid sequences coding for this enzyme and protein sequences are described in NCBI under the reference numbers KF606861.1 and AHJ89438.1, respectively, and more particularly in SEQ ID NOs: 152 and 151, respectively.

In another embodiment, the flavone synthase FNS is an FNSII originating from Scutellaria baicalensis. The nucleic acid sequences coding for this enzyme and protein sequences are described in NCBI under the reference numbers KT963454.1 and AMW91729.1, respectively, and more particularly in SEQ ID NOs: 154 and 153, respectively.

In another embodiment, the flavone synthase FNS is an FNSII originating from Dorcoceras hygrometricum. The nucleic acid sequences coding for this enzyme and protein sequences are described in NCBI under the reference numbers KV013332.1 and KZV23934.1, respectively, and more particularly in SEQ ID NOs: 156 and 155, respectively.

In another embodiment, the flavone synthase FNS is an FNSII originating from Antirrhinum majus. The nucleic acid sequences coding for this enzyme and protein sequences are described in NCBI under the reference numbers AB028151.1 and BAA84071.1, respectively, and more particularly in SEQ ID NOs: 158 and 157, respectively.

In another embodiment, the flavone synthase FNS is an FNSII originating from Erythranthe lewisii. The nucleic acid sequences coding for this enzyme and protein sequences are described in NCBI under the reference numbers KX710102.1 and AOR81894.1, respectively, and more particularly in SEQ ID NOs: 160 and 159, respectively.

In a particular embodiment, the microorganism comprises a heterologous nucleic acid sequence coding for a type II flavone synthase (FNSII) and a type I flavone synthase, for example a sequence chosen from SEQ ID NOs: 33, 35, 129, 131, 133, 135, 139, 147, 149, 151, 153, 155, 157 and 159 and polypeptides comprising a sequence having at least 60, 70, 80, 85, 90 or 95% sequence identity with one of these sequences and having flavone synthase activity, and an enzyme comprising a sequence chosen from SEQ ID NOs: 37, 127, 137, 141, 143 and 145 and a polypeptide comprising a sequence having at least 60, 70, 80, 85, 90 or 95% sequence identity with this sequence and having flavone synthase activity, preferably a sequence chosen from SEQ ID NOs: 33 and 35 and polypeptides comprising a sequence having at least 60, 70, 80, 85, 90 or 95% sequence identity with one of these sequences and having flavone synthase activity and an enzyme comprising a sequence chosen from SEQ ID NO: 37 and a polypeptide comprising a sequence having at least 60, 70, 80, 85, 90 or 95% sequence identity with this sequence and having flavone synthase activity.

The type II FNSs, FNSII, require the presence of a cytochrome P450 reductase (CPR). If the microorganism does not comprise cytochrome P450 reductase, it will thus be necessary to introduce a heterologous cytochrome P450 reductase. If the microorganism already comprises one, it is possible to envisage either the overexpression of an endogenous cytochrome P450 reductase (for example by replacing the promoter with a strong promoter or by adding one or more copies of the coding sequence) or by also introducing a heterologous cytochrome P450 reductase.

In a particular embodiment, the type II FNS and the CPR are from the same origin, the same species.

### Combination of Enzymes

Thus, besides the enzymes required for the biosynthesis of hesperidin and/or diosmin from hesperetin and/or diosmetin, respectively, as described previously, the microorganism preferably comprises enzymes for producing hesperetin and/or diosmetin from naringenin and/or apigenin.

In a first particular embodiment, the recombinant microorganism comprises:
a heterologous nucleic acid sequence coding for a flavanone 7-O-beta-D-glucosyltransferase (UGT) which is capable of adding a glucose in position 7 of hesperetin and/or diosmetin; preferably a flavanone 7-O-beta-D-glucosyltransferase (UGT) from Arabidopsis thaliana, Citrus sinensis, Citrus clementina, Scutellaria baicalensis or Homo sapiens, preferably from Citrus sinensis or Scutellaria baicalensis; preferably a flavanone 7-O-beta-D-glucosyltransferase (UGT) selected from enzymes comprising a sequence chosen from SEQ ID NOs: 113, 115, 91, 93, 95, 97, 99 and 101 and polypeptides comprising a sequence having at least 60, 70, 80, 85, 90 or 95% sequence identity with one of these sequences and having flavanone 7-O-beta-D-glucosyltransferase activity; in particular a flavanone 7-O-beta-D-glucosyltransferase (UGT) comprising a sequence chosen from SEQ ID NOs: 113, 115, 91, 93, 95 and 97 and polypeptides comprising a sequence having at least 60, 70, 80, 85, 90 or 95% sequence identity with one of these sequences and having flavanone 7-O-beta-D-glucosyltransferase activity;
a heterologous nucleic acid sequence coding for a 6-O-rhamnosyltransferase (RhaT) which is capable of transferring a rhamnose into position 6 of the glucose of hesperetin-7-O-glucoside and/or diosmetin-7-O-glucoside; preferably an RhaT of the genus Citrus or Petunia hybrida, preferably Citrus sinensis, Citrus maxima, or Citrus clementina, even more preferably Citrus sinensis or Citrus clementina, preferably a 6-O-rhamnosyltransferase (RhaT) comprising a sequence chosen from SEQ ID NOs: 103 and 105 and polypeptides comprising a sequence having at least 60, 70, 80, 85, 90 or 95% sequence identity with one of these sequences and having 6-O-rhamnosyltransferase activity;
a heterologous nucleic acid sequence coding for a UDP-glucose 4,6-dehydratase/UDP-4-keto-6-deoxy-D-glucose 3,5-epimerase/UDP-4-keto-L-rhamnose-reductase (RHM) which is capable of producing a UDP-rhamnose; preferably an RHM from Citrus sinensis or from Arabidopsis thaliana, preferably an RHM comprising a sequence chosen from SEQ ID NOs: 107, 109 and 111 and polypeptides comprising a sequence having at least 60, 70, 80, 85, 90 or 95% sequence identity with one of these sequences and having UDP-glucose 4,6-dehydratase/UDP-4-keto-6-deoxy-D-glucose 3,5-epimerase/UDP-4-keto-L-rhamnose-reductase activity, in particular an RHM comprising a sequence selected from SEQ ID NOs: 107 and 109 and polypeptides comprising a sequence having at least 60, 70, 80, 85, 90 or 95% sequence identity with this sequence and having UDP-glucose 4,6-dehydratase/UDP-4-keto-6-deoxy-D-glucose 3,5-epimerase/UDP-4-keto-L-rhamnose-reductase activity;
a heterologous nucleic acid sequence coding for a flavonoid 3′-monooxygenase (F3′H) which is capable of hydroxylating naringenin and/or apigenin in position 3′; in particular which is capable of hydroxylating naringenin and/or apigenin in position 3′; preferably from Perilla frutescens var. crispa, Petunia x hybrida, Callistephus chinensis, Gerbera hybrida, Citrus sinensis, Citrus clementina, Osteospermum hybrid cultivar, Phanerochaete chrysosporium, Streptomyces avermitilis or Pilosella officinarum, in particular from Perilla frutescens var. crispa, Petunia x hybrida, Callistephus chinensis, Gerbera hybrida, Citrus sinensis or Pilosella officinarum, preferably an F3′H comprising a sequence chosen from SEQ ID NOs: 1, 3, 5, 7, 9, 11, 13, 15, 17, 19, 21 and 121 and polypeptides comprising a sequence having at least 60, 70, 80, 85, 90 or 95% sequence identity with one of these sequences and having flavonoid 3′-monooxygenase activity, preferably selected from enzymes comprising a sequence chosen from SEQ ID NOs: 1, 5, 7, 11, 17, 19 and 121 and polypeptides comprising a sequence having at least 60, 70, 80, 85, 90 or 95% sequence identity with one of these sequences and having flavonoid 3′-monooxygenase activity, in particular an F3′H comprising a sequence selected from SEQ ID NOs: 7, 11, 17 and 121 and polypeptides comprising a sequence having at least 60, 70, 80, 85, 90 or 95% sequence identity with one of these sequences and having flavonoid 3′-monooxygenase activity;
optionally, a heterologous nucleic acid sequence coding for a cytochrome P450 reductase (CPR); preferably a CPR from Saccharomyces cerevisiae, or from a plant, for example from Catharanthus roseus or Arabidopsis thaliana; preferably a CPR comprising a sequence chosen from SEQ ID NOs: 23, 25, 27, 29 and 31 and polypeptides comprising a sequence having at least 60, 70, 80, 85, 90 or 95% sequence identity with one of these sequences and having cytochrome P450 reductase activity, preferably from enzymes comprising a sequence chosen from SEQ ID NOs: 23, 25, 29 and 31 and polypeptides comprising a sequence having at least 60, 70, 80, 85, 90 or 95% sequence identity with one of these sequences and having cytochrome P450 reductase activity;
a heterologous nucleic acid sequence coding for an O-methyltransferase (OMT) which is capable of methylating eriodictyol and/or luteolin in position 4′; preferably an OMT from Arabidopsis thaliana or Homo sapiens, preferably an OMT comprising a sequence chosen from SEQ ID NOs: 117, 119, 87 and 89 and polypeptides comprising a sequence having at least 60, 70, 80, 85, 90 or 95% sequence identity with one of these sequences and having O-methyltransferase activity, notably with eriodictyol and/or luteolin as substrate and with methylation in position 4′, preferably selected from the enzyme comprising a sequence chosen from SEQ ID NOs: 117, 119 and 89 and polypeptides comprising a sequence having at least 60, 70, 80, 85, 90 or 95% sequence identity with one of these sequences and having O-methyltransferase activity; and
optionally, a heterologous nucleic acid sequence coding for a flavone synthase (FNS) which is capable of producing a flavone from a flavanone, in particular which is capable of transforming naringenin into apigenin, and/or eriodictyol into luteolin, preferably of transforming eriodictyol into luteolin; preferably an FNS from Arabidopsis thaliana, Lonicera japonica, Lonicera macranthoides, Medicago truncatula, Oryza sativa, Petroselinum crispum, Populus deltoides, Zea mays, Callistephus chinensis, Apium graveolens, Cuminum cyminum, Aethusa cynapium, Angelica archangelica, Conium maculatum, Camellia sinensis, Cynara cardunculus var scolymus, Saussurea medusa, Plectranthus barbatus, Scutellaria baicalensis, Dorcoceras hygrometricum, Antirrhinum majus, Perilla frutescens var crispa, Dahlia pinnata or Erythranthe lewisii, in particular from Arabidopsis thaliana, Lonicera japonica, Lonicera macranthoides, Medicago truncatula, Oryza sativa, Petroselinum crispum, Populus deltoides or Zea mays, preferably from Lonicera japonica, Lonicera macranthoides and Petroselinum crispum; preferably an FNS comprising a sequence chosen from SEQ ID NOs: 33, 35, 37, 127, 129, 131, 133, 135, 137, 139, 141, 143, 145, 147, 149, 151, 153, 155, 157 and 159 and polypeptides comprising a sequence having at least 60, 70, 80, 85, 90 or 95% sequence identity with one of these sequences and having flavone synthase activity, preferably from enzymes comprising a sequence chosen from SEQ ID NOs: 33, 35 and 37 and polypeptides comprising a sequence having at least 60, 70, 80, 85, 90 or 95% sequence identity with one of these sequences and having flavone synthase activity, preferably a flavone synthase (FNS) comprising a sequence chosen from SEQ ID NO: 37 and a polypeptide comprising a sequence having at least 60, 70, 80, 85, 90 or 95% sequence identity with this sequence and having flavone synthase activity.

In another particular embodiment, the recombinant microorganism comprises:
a heterologous nucleic acid sequence coding for a flavanone 7-O-beta-D-glucosyltransferase (UGT) which is capable of adding a glucose in position 7 of hesperetin and/or diosmetin; preferably a flavanone 7-O-beta-D-glucosyltransferase (UGT) from Arabidopsis thaliana, Scutellaria baicalensis or Homo sapiens, preferably from Arabidopsis thaliana or Scutellaria baicalensis; preferably a flavanone 7-O-beta-D-glucosyltransferase (UGT) selected from enzymes comprising a sequence chosen from SEQ ID NOs: 91, 93, 95, 97, 99 and 101 and polypeptides comprising a sequence having at least 60, 70, 80, 85, 90 or 95% sequence identity with one of these sequences and having flavanone 7-O-beta-D-glucosyltransferase activity; in particular a flavanone 7-O-beta-D-glucosyltransferase (UGT) comprising a sequence chosen from SEQ ID NOs: 91, 93, 95 and 97 and polypeptides comprising a sequence having at least 60, 70, 80, 85, 90 or 95% sequence identity with one of these sequences and having flavanone 7-O-beta-D-glucosyltransferase activity;
a heterologous nucleic acid sequence coding for a 6-O-rhamnosyltransferase (RhaT) which is capable of transferring a rhamnose into position 6 of the glucose of hesperetin-7-O-glucoside and/or diosmetin-7-O-glucoside; preferably an RhaT of the genus Citrus or Petunia hybrida, preferably Citrus sinensis, Citrus maxima, or Citrus clementina, even more preferably Citrus sinensis or Citrus clementina, preferably a 6-O-rhamnosyltransferase (RhaT) comprising a sequence chosen from SEQ ID NOs: 103 and 105 and polypeptides comprising a sequence having at least 60, 70, 80, 85, 90 or 95% sequence identity with one of these sequences and having 6-O-rhamnosyltransferase activity;
a heterologous nucleic acid sequence coding for a UDP-glucose 4,6-dehydratase/UDP-4-keto-6-deoxy-D-glucose 3,5-epimerase/UDP-4-keto-L-rhamnose-reductase (RHM) which is capable of producing a UDP-rhamnose; preferably an RHM from Citrus sinensis or from Arabidopsis thaliana, preferably an RHM comprising a sequence chosen from SEQ ID NOs: 107, 109 and 111 and polypeptides comprising a sequence having at least 60, 70, 80, 85, 90 or 95% sequence identity with one of these sequences and having UDP-glucose 4,6-dehydratase/UDP-4-keto-6-deoxy-D-glucose 3,5-epimerase/UDP-4-keto-L-rhamnose-reductase activity, in particular an RHM comprising a sequence selected from SEQ ID NOs: 107 and 109 and polypeptides comprising a sequence having at least 60, 70, 80, 85, 90 or 95% sequence identity with this sequence and having UDP-glucose 4,6-dehydratase/UDP-4-keto-6-deoxy-D-glucose 3,5-epimerase/UDP-4-keto-L-rhamnose-reductase activity;
a heterologous nucleic acid sequence coding for a flavonoid 3′-monooxygenase (F3′H) which is capable of hydroxylating naringenin and/or apigenin in position 3′; in particular which is capable of hydroxylating naringenin and/or apigenin in position 3′; preferably from Perilla frutescens var. crispa, Petunia x hybrida, Callistephus chinensis, Gerbera hybrida, Citrus sinensis and Pilosella officinarum, preferably an F3′H comprising a sequence chosen from SEQ ID NOs: 1, 3, 5, 7, 9, 11, 13, 15, 17, 19 and 21 and polypeptides comprising a sequence having at least 60, 70, 80, 85, 90 or 95% sequence identity with one of these sequences and having flavonoid 3′-monooxygenase activity, preferably selected from enzymes comprising a sequence chosen from SEQ ID NOs: 1, 5, 7, 11, 17 and 19 and polypeptides comprising a sequence having at least 60, 70, 80, 85, 90 or 95% sequence identity with one of these sequences and having flavonoid 3′-monooxygenase activity, in particular an F3′H comprising a sequence selected from SEQ ID NOs: 5, 7 and 17 and polypeptides comprising a sequence having at least 60, 70, 80, 85, 90 or 95% sequence identity with this sequence and having flavonoid 3′-monooxygenase activity;
optionally, a heterologous nucleic acid sequence coding for a cytochrome P450 reductase (CPR); preferably a CPR from Saccharomyces cerevisiae, or from a plant, for example from Catharanthus roseus or Arabidopsis thaliana; preferably a CPR comprising a sequence chosen from SEQ ID NOs: 23, 25, 27, 29 and 31 and polypeptides comprising a sequence having at least 60, 70, 80, 85, 90 or 95% sequence identity with one of these sequences and having cytochrome P450 reductase activity, preferably from enzymes comprising a sequence chosen from SEQ ID NOs: 23, 25, 29 and 31 and polypeptides comprising a sequence having at least 60, 70, 80, 85, 90 or 95% sequence identity with one of these sequences and having cytochrome P450 reductase activity;
a heterologous nucleic acid sequence coding for an O-methyltransferase (OMT) which is capable of methylating eriodictyol and/or luteolin in position 4′; preferably an OMT from Arabidopsis thaliana or Homo sapiens, preferably an OMT comprising a sequence chosen from SEQ ID NOs: 87 and 89 and polypeptides comprising a sequence having at least 60, 70, 80, 85, 90 or 95% sequence identity with one of these sequences and having O-methyltransferase activity, notably with eriodictyol and/or luteolin as substrate and with methylation in position 4′, preferably selected from the enzyme comprising the sequence SEQ ID NO: 89 and polypeptides comprising a sequence having at least 60, 70, 80, 85, 90 or 95% sequence identity with this sequence and having O-methyltransferase activity; and
optionally, a heterologous nucleic acid sequence coding for a flavone synthase (FNS) which is capable of producing a flavone from a flavanone, in particular capable of transforming naringenin into apigenin, and/or eriodictyol into luteolin, preferably of transforming eriodictyol into luteolin; preferably an FNS from Arabidopsis thaliana, Lonicera japonica, Lonicera macranthoides, Medicago truncatula, Oryza sativa, Petroselinum crispum, Populus deltoides or Zea mays, preferably from Lonicera japonica, Lonicera macranthoides and Petroselinum crispum; preferably an FNS comprising a sequence chosen from SEQ ID NOs: 33, 35 and 37 and polypeptides comprising a sequence having at least 60, 70, 80, 85, 90 or 95% sequence identity with one of these sequences and having flavone synthase activity, preferably a flavone synthase (FNS) comprising a sequence chosen from SEQ ID NO: 37 and a polypeptide comprising a sequence having at least 60, 70, 80, 85, 90 or 95% sequence identity with this sequence and having flavone synthase activity.

In a preferred embodiment, the microorganism comprises:
a heterologous nucleic acid sequence coding for a flavanone 7-O-beta-D-glucosyltransferase selected from enzymes comprising a sequence chosen from SEQ ID NOs: 113, 115 and 95 and polypeptides comprising a sequence having at least 60, 70, 80, 85, 90 or 95% sequence identity with one of these sequences and having flavanone 7-O-beta-D-glucosyltransferase activity, preferably selected from enzymes comprising a sequence chosen from SEQ ID NOs: 113 and 95 and polypeptides comprising a sequence having at least 60, 70, 80, 85, 90 or 95% sequence identity with one of these sequences and having flavanone 7-O-beta-D-glucosyltransferase activity, and most particularly preferably selected from enzymes comprising a sequence chosen from SEQ ID NO: 113 and polypeptides comprising a sequence having at least 60, 70, 80, 85, 90 or 95% sequence identity with this sequence and having flavanone 7-O-beta-D-glucosyltransferase activity; and
a heterologous nucleic acid sequence coding for a 6″-O-rhamnosyltransferase selected from enzymes comprising a sequence chosen from SEQ ID NO: 103 and polypeptides comprising a sequence having at least 60, 70, 80, 85, 90 or 95% sequence identity with this sequence and having 6″-O-rhamnosyltransferase activity; and
a heterologous nucleic acid sequence coding for a UDP-glucose 4,6-dehydratase/UDP-4-keto-6-deoxy-D-glucose 3,5-epimerase/UDP-4-keto-L-rhamnose-reductase selected from enzymes comprising a sequence chosen from SEQ ID NO: 107 and polypeptides comprising a sequence having at least 60, 70, 80, 85, 90 or 95% sequence identity with this sequence and having UDP-glucose 4,6-dehydratase/UDP-4-keto-6-deoxy-D-glucose 3,5-epimerase/UDP-4-keto-L-rhamnose-reductase activity; and
a heterologous nucleic acid sequence coding for a flavonoid 3′-monooxygenase (F3′H) selected from enzymes comprising a sequence chosen from SEQ ID NOs: 7, 11, 17 and 121 and polypeptides comprising a sequence having at least 60, 70, 80, 85, 90 or 95% sequence identity with one of these sequences and having flavonoid 3′-monooxygenase activity, preferably a flavonoid 3′-monooxygenase (F3′H) comprising a sequence chosen from SEQ ID NOs: 7, 17 and 121 and polypeptides having at least 60, 70, 80, 85, 90 or 95% identity with one of these sequences and having flavonoid 3′-monooxygenase activity, and most particularly preferably a flavonoid 3′-monooxygenase (F3′H) comprising a sequence chosen from SEQ ID NO: 7 and polypeptides having at least 60, 70, 80, 85, 90 or 95% identity with this sequence and having flavonoid 3′-monooxygenase activity;
optionally, a heterologous nucleic acid sequence coding for a cytochrome P450 reductase (CPR) comprising a sequence chosen from SEQ ID NOs: 23, 25, 29 and 31 and polypeptides comprising a sequence having at least 60, 70, 80, 85, 90 or 95% sequence identity with one of these sequences and having cytochrome P450 reductase activity, preferably from enzymes comprising a sequence chosen from SEQ ID NOs: 23, 25 and 29 and polypeptides comprising a sequence having at least 60, 70, 80, 85, 90 or 95% sequence identity with one of these sequences and having cytochrome P450 reductase activity, and most particularly preferably from enzymes comprising a sequence chosen from SEQ ID NO: 25 and polypeptides comprising a sequence having at least 60, 70, 80, 85, 90 or 95% sequence identity with this sequence and having cytochrome P450 reductase activity; and
a heterologous nucleic acid sequence coding for an O-methyltransferase (OMT) which is capable of methylating eriodictyol and/or luteolin in position 4′ and comprising a sequence chosen from SEQ ID NOs: 117, 119 and 89 and polypeptides comprising a sequence having at least 60, 70, 80, 85, 90 or 95% sequence identity with one of these sequences and having O-methyltransferase activity, notably with eriodictyol and/or luteolin as substrate and with methylation in position 4′, preferably selected from the enzyme comprising a sequence chosen from SEQ ID NOs: 117 and 119 and polypeptides comprising a sequence having at least 60, 70, 80, 85, 90 or 95% sequence identity with one of these sequences and having O-methyltransferase activity; and
optionally, a heterologous nucleic acid sequence coding for a flavone synthase (FNS) which is capable of producing a flavone from a flavanone, in particular which is capable of transforming naringenin into apigenin, and/or eriodictyol into luteolin, preferably of transforming eriodictyol into luteolin and comprising a sequence chosen from SEQ ID NO: 37 and a polypeptide comprising a sequence having at least 60, 70, 80, 85, 90 or 95% sequence identity with this sequence and having flavone synthase activity.

Preferably, the microorganism comprises each of these heterologous nucleic acid sequences.

In another particular embodiment, the recombinant microorganism comprises:
a heterologous nucleic acid sequence coding for a flavanone 7-O-beta-D-glucosyltransferase (UGT) which is capable of adding a glucose in position 7 of hesperetin and/or diosmetin;
a heterologous nucleic acid sequence coding for a 6″-O-rhamnosyltransferase (RhaT) which is capable of transferring a rhamnose into position 6 of the glucose of hesperetin-7-O-glucoside and/or diosmetin-7-O-glucoside;
a heterologous nucleic acid sequence coding for a UDP-glucose 4,6-dehydratase/UDP-4-keto-6-deoxy-D-glucose 3,5-epimerase/UDP-4-keto-L-rhamnose-reductase (RHM) which is capable of producing UDP-rhamnose;
a heterologous nucleic acid sequence coding for a flavonoid 3′-monooxygenase (F3′H) which is capable of hydroxylating naringenin and/or apigenin in position 3′;
a heterologous nucleic acid sequence coding for a cytochrome P450 reductase;
a heterologous nucleic acid sequence coding for an O-methyltransferase which is capable of methylating eriodictyol and/or luteolin in position 4′; and
a heterologous nucleic acid sequence coding for a flavone synthase (FNS) which is capable of producing a flavone from a flavanone, in particular which is capable of transforming naringenin into apigenin, eriodictyol into luteolin and/or hesperetin into diosmetin, preferably of transforming eriodictyol into luteolin.

In another particular embodiment, the recombinant microorganism also comprises a heterologous or endogenous nucleic acid sequence coding for an S-adenosylmethionine synthetase (SAMT), in particular from Saccharomyces cerevisiae, for example an SAMT comprising a sequence chosen from SEQ ID NO: 81 and a polypeptide comprising a sequence having at least 60, 70, 80, 85, 90 or 95% sequence identity with this sequence and having S-adenosylmethionine synthetase activity.

Each enzyme may be chosen from the enzymes described above.

### Up to Naringenin and Apigenin

Various pathways for the biosynthesis of naringenin and apigenin are known in plants, in particular from glucose, tyrosine or phenylalanine. Microorganisms, notably E. coli and Saccharomyces cerevisiae, have been modified to produce naringenin and/or apigenin (Hwang E I, et al. 2003. Appl. Environ. Microbiol. 2003, 69(5): 2699-2706; Jiang H1, et al. 2005. Appl. Environ. Microbiol. 2005, 71(6): 2962-9; Pandey et al., 2016, Biotechnol. Adv., 34, 634-662).

For example, the pathway for the biosynthesis of naringenin and apigenin may be that described in FIG. 1.

In a first embodiment, the microorganism comprises the enzymes required for the synthesis of naringenin and/or apigenin from tyrosine.

In a second embodiment, the microorganism comprises the enzymes required for the synthesis of naringenin and/or apigenin from phenylalanine.

In a third embodiment, the microorganism comprises the enzymes required for the synthesis of naringenin and/or apigenin from tyrosine and phenylalanine.

### TAL: Tyrosine Ammonia Lyase

TAL is a tyrosine ammonia lyase. This enzyme is capable of producing p-coumaric acid from tyrosine. This enzyme belongs to the class EC 4.3.1.23.

The term “phenylalanine ammonia lyase activity” means the transformation of phenylalanine into trans-cinnamic acid by means of the enzyme phenylalanine ammonia lyase. To determine whether there is phenylalanine ammonia lyase activity, an enzymatic test may be performed, which consists of the in vitro incubation of a mixture composed of the phenylalanine ammonia lyase enzyme and phenylalanine, under optimum conditions (pH, temperature, ions, etc.). After a certain incubation time, the appearance of trans-cinnamic acid is observed in UPLC-MS in comparison with the expected standard.

A tyrosine ammonia lyase (TAL) may also have phenylalanine ammonia lyase (PAL) activity as defined above and/or dihydroxyphenylalanine ammonia-lyase (DAL) activity.

The microorganism may thus comprise a heterologous nucleic acid sequence coding for a tyrosine ammonia lyase.

Preferably, this enzyme is an enzyme produced by a bacterium of the genus Rhodobacter or a bacterium of the genus Flavobacteriaceae. In a particular embodiment, this enzyme is produced by a Rhodobacter capsulatus or Rhodobacter sphaeroides bacterium. In another particular embodiment, this enzyme is produced by a Flavobacterium johnsoniae bacterium. In another embodiment, this enzyme is an enzyme produced by a yeast, in particular a yeast of the genus Rhodotorula, for example Rhodotorula glutinis. Other organisms also produce such an enzyme, for example Camellia sinensis, Fragaria x ananassa, Ralstonia metallidurans or Zea mays.

In a particular embodiment, the tyrosine ammonia lyase is selected from enzymes comprising a sequence chosen from SEQ ID NOs: 39 and 41 and polypeptides comprising a sequence having at least 60, 70, 80, 85, 90 or 95% sequence identity with one of these sequences and having tyrosine ammonia lyase activity.

In one embodiment, the TAL is from Flavobacterium johnsoniae. It is described in the GenBank database from NCBI under the number KR095306.1 for the nucleic acid sequence and under the number AKE50827.1 for the protein sequence, and more particularly in SEQ ID NOs: 40 and 39.

In a particularly preferred embodiment, the TAL is from Rhodotorula glutinis. It is described in the GenBank database from NCBI under the number KF765779.1 for the nucleic acid sequence and under the number AGZ04575.1 for the protein sequence, and more particularly in SEQ ID NOs: 42 and 41, respectively.

In a particularly preferred embodiment, the tyrosine ammonia lyase is selected from the enzyme comprising a sequence chosen from SEQ ID NO: 41 and polypeptides comprising a sequence having at least 60, 70, 80, 85, 90 or 95% sequence identity with this sequence and having tyrosine ammonia lyase activity.

### 4CL: 4-Coumarate-CoA Ligase

4CL is a 4-coumarate-CoA ligase. This enzyme is capable of producing 4-coumaroyl-CoA from p-coumaric acid and Coenzyme A and of producing caffeoyl-CoA from caffeic acid and Coenzyme A. This enzyme belongs to the class EC 6.2.1.12.

The term “4-coumarate-CoA ligase activity” means the transformation of p-coumaric acid into p-coumaroyl-CoA or of caffeic acid into caffeoyl-CoA by the enzyme 4-coumarate CoA ligase. To determine whether there is 4-coumarate CoA ligase activity, an enzymatic test may be performed, which consists of the in vitro incubation of a mixture composed of the 4-coumarate CoA ligase enzyme, p-coumaric acid or caffeic acid, ATP and CoA under optimum conditions (pH, temperature, ions, etc.). After a certain incubation time, the appearance of p-coumaroyl-CoA or of caffeoyl-CoA is observed on the UV spectrophotometer at a wavelength of 333 nm and 346 nm, respectively, in comparison with the expected standard.

The microorganism may thus comprise a heterologous nucleic acid sequence coding for a 4-coumarate-CoA ligase.

Preferably, this enzyme is an enzyme produced by a plant, for example Abies, Arabidopsis, Agastache, Amorpha, Brassica, Citrus, Cathaya, Cedrus, Crocus, Larix, Festuca, Glycine, Juglans, Keteleeria, Lithospermum, Lolium, Lotus, Lycopersicon, Malus, Medicago, Mesembryanthemum, Nicotiana, Nothotsuga, Oryza, Phaseolus, Pelargonium, Petroselinum, Physcomitrella, Picea, Prunus, Pseudolarix, Pseudotsuga, Rosa, Rubus, Ryza, Saccharum, Suaeda, Pinus, Populus, Solanum, Thellungiella, Triticum, Tsuga, Vitis or Zea. Alternatively, this enzyme is an enzyme produced by a microorganism, for example Aspergillus, Mycosphaerella, Mycobacterium, Neisseria, Neurospora, Streptomyces, Rhodobacter or Yarrowia.

In a preferred embodiment, this enzyme is an enzyme produced by a plant, preferably Arabidopsis thaliana, Citrus clementina or Petroselinum crispum, in particular Arabidopsis thaliana or Petroselinum crispum, or by a bacterium, preferably of the genus Streptomyces, in particular Streptomyces clavuligerus.

In a particular embodiment, the 4-coumarate-CoA ligase is selected from enzymes comprising a sequence chosen from SEQ ID NOs: 43, 45, 47, 49, 123 and 125 and polypeptides comprising a sequence having at least 60, 70, 80, 85, 90 or 95% sequence identity with one of these sequences and having 4-coumarate-CoA ligase activity.

In another particular embodiment, the 4-coumarate-CoA ligase is an enzyme comprising a sequence selected from SEQ ID NOs: 43, 45, 47 and 49, preferably SEQ ID NOs: 45 and 49 and polypeptides comprising a sequence having at least 60, 70, 80, 85, 90 or 95% sequence identity with one of these sequences and having 4-coumarate-CoA ligase activity.

In a first particular embodiment, the 4CL is from Arabidopsis thaliana. It is described in the GenBank database from NCBI under the number AY099747.1 for the nucleic acid sequence and under the number AAM20598.1 for the protein sequence, and more particularly in SEQ ID NOs: 44 and 43, respectively.

In a second particular embodiment, the 4CL is from Petroselinum crispum. It is described in the GenBank database from NCBI under the number X13324.1 or X13325.1 for the nucleic acid sequence and under the number CAA31696.1 or CAA31697.1 for the protein sequence, respectively. The proteins are described in UniProtKB/Swiss Prot under the reference numbers P14912 and P14913, respectively, and more particularly in SEQ ID NOs: 46 and 45, 48 and 47, respectively. Preferably, the 4CL is from Petroselinum crispum and is described in the GenBank database from NCBI under the number X13324.1 for the nucleic acid sequence and under the number CAA31696.1 for the protein sequence, and in UniProtKB/Swiss Prot under the reference number P14912, and more particularly in SEQ ID NOs: 46 and 45, respectively.

In a third particular embodiment, the 4CL is from Streptomyces clavuligerus. It is described in the GenBank database from NCBI under the number CP016559.1 for the nucleic acid sequence and under the number ANW18832.1 for the protein sequence, and more particularly in SEQ ID NOs: 50 and 49, respectively.

In a fourth particular embodiment, the 4CL is from Arabidopsis thaliana. A nucleotide sequence and the protein sequence of this enzyme are described, respectively, in SEQ ID NOs: 124 and 123.

In a fifth particular embodiment, the 4CL is from Citrus clementina and a nucleotide sequence and the protein sequence of this enzyme are described, respectively, in SEQ ID NOs: 126 and 125.

In a preferred embodiment, the 4CL is an enzyme comprising a sequence selected from SEQ ID NOs: 45, 123 and 125, preferably SEQ ID NOs: 123 and 45 and polypeptides comprising a sequence having at least 60, 70, 80, 85, 90 or 95% sequence identity with one of these sequences and having 4-coumarate-CoA ligase activity. Most particularly preferably, the 4CL is an enzyme comprising a sequence selected from SEQ ID NO: 45 and polypeptides comprising a sequence having at least 60, 70, 80, 85, 90 or 95% sequence identity with this sequence and having 4-coumarate-CoA ligase activity.

### CHS: Chalcone Synthase

CHS is a chalcone synthase. This enzyme is capable of producing naringenin-chalcone from 4-coumaroyl-CoA and from malonyl-CoA and of producing eriodictyol-chalcone from caffeoyl-CoA and from malonyl-CoA. This enzyme belongs to the class EC 2.3.1.74.

The term “chalcone synthase activity” means the transformation of p-coumaroyl-CoA and of malonyl-CoA into naringenin chalcone or of caffeoyl-CoA and of malonyl-CoA-into eriodictyol chalcone by means of the chalcone synthase enzyme. To determine whether there is chalcone synthase activity, an enzymatic test may be performed, which consists of the in vitro incubation of a mixture composed of the chalcone synthase enzyme, coumaroyl-CoA or caffeoyl-CoA and malonyl-CoA, under optimum conditions (pH, temperature, ions, etc.). After a certain incubation time, the appearance of naringenin chalcone or of eriodictyol chalcone, respectively, is observed in HPLC-MS in comparison with the expected standard.

The microorganism thus comprises a heterologous nucleic acid sequence coding for a chalcone synthase.

This enzyme may be an enzyme produced by a plant, notably of the genus Arabidopsis, Avena, Cosmos, Citrus, Daucus, Fagopyrum, Freesia, Glycine, Glycyrrhiza, Humulus, Hypericum, Hordeum, Juglans, Medicago, Phaseolus, Physcomitrella, Plagiochasma, Petroselinum, Pueraria, Rubus, Secale, Scutellaria, Silene, Sinapis, Spinacia, Stellaria, Triticum, Tulipa, Verbena, Vitis or Xanthisma, for example Arabidopsis thaliana, Avena sativa, Cosmos sulphureus, Citrus sinensis, Daucus carota, Fagopyrum esculentum, Freesia hybrid cultivar, Glycine max, Glycyrrhiza echinata, Humulus lupulus, Hypericum androsaemum, Hordeum vulgare, Juglans sp., Medicago sativa, Phaseolus vulgaris, Physcomitrella patens, Plagiochasma appendiculatum, Petroselinum crispum, Pueraria montana var. lobata, Rubus idaeus, Secale cereale, Scutellaria baicalensis, Silene sp., Sinapis alba, Spinacia oleracea, Stellaria longipes, Triticum aestivum, Tulipa hybrid cultivar, Verbena sp., Vitis vinifera or Xanthisma gracile.

Preferably, this enzyme is an enzyme produced by a plant, for example of the genus Citrus, in particular Citrus sinensis, or Hordeum vulgare or by a bacterium, preferably of the genus Streptomyces, in particular Streptomyces clavuligerus.

In a particular embodiment, the chalcone synthase is selected from enzymes comprising a sequence chosen from SEQ ID NOs: 51, 53, 55 and 57 and polypeptides comprising a sequence having at least 60, 70, 80, 85, 90 or 95% sequence identity with one of these sequences and having chalcone synthase activity, preferably from enzymes comprising a sequence chosen from SEQ ID NOs: 53 and 55 and polypeptides comprising a sequence having at least 60, 70, 80, 85, 90 or 95% sequence identity with one of these sequences and having chalcone synthase activity.

In a particularly preferred embodiment, the chalcone synthase is an enzyme comprising a sequence chosen from SEQ ID NO: 53 and polypeptides comprising a sequence having at least 60, 70, 80, 85, 90 or 95% sequence identity with this sequence and having chalcone synthase activity.

In a first particular embodiment, the CHS is from Hordeum vulgare. It is described in the GenBank database from NCBI under the number Y09233.1 for the nucleic acid sequence and under the number CAA70435.1 for the protein sequence, and more particularly in SEQ ID NOs: 52 and 51, respectively. The protein is described in UniProtKB/Swiss Prot under the reference number Q96562.

In a second particular embodiment, the CHS is from Citrus sinensis. It is described in the GenBank database from NCBI under the number AB009351.1 for the nucleic acid sequence and under the number BAA81664.1 for the protein sequence, and more particularly in SEQ ID NOs: 54 and 53, respectively.

In a third particular embodiment, the CHS is from Citrus sinensis. It is described in the GenBank database from NCBI under the number XM 006489733.1 for the nucleic acid sequence and under the number XP_006489796.1 for the protein sequence, and more particularly in SEQ ID NOs: 56 and 55, respectively.

In a fourth particular embodiment, the CHS is from Streptomyces clavuligerus. It is described in the GenBank database from NCBI under the number CP016559.1 for the nucleic acid sequence and under the number ANW16917.1 for the protein sequence, and more particularly in SEQ ID NOs: 58 and 57, respectively.

Since the reaction catalyzed by chalcone synthase requires the presence of malonyl-CoA, the microorganism can be modified to increase the synthesis of malonyl-CoA.

### CHI: Chalcone Isomerase

CHI is a chalcone isomerase. It is capable of producing naringenin from naringenin chalcone and of producing eriodictyol from eriodictyol chalcone. This enzyme belongs to the class EC 5.5.1.6.

The term “chalcone isomerase activity” means the transformation of naringenin chalcone or of eriodictyol chalcone into naringenin or eriodictyol by a chalcone isomerase enzyme. To determine whether there is chalcone isomerase activity, an enzymatic test may be performed, which consists of the in vitro incubation of a mixture composed of the chalcone isomerase enzyme, naringenin chalcone or eriodictyol chalcone under optimum conditions (pH, temperature, ions, etc.). After a certain incubation time, the appearance of naringenin or of eriodictyol, respectively, is observed in HPLC-MS in comparison with the expected standard.

The microorganism thus comprises a heterologous nucleic acid sequence coding for a chalcone isomerase.

This enzyme may originate from a plant, notably of the genus Arabidopsis, Ginkgo, Oncidium, Perilla, Citrus or Trigonella, for example Arabidopsis thaliana, Ginkgo biloba, Oncidium Gower Ramsey, Perilla frutescens, Citrus Sinensis or Trigonella foenum-graecum.

Preferably, this enzyme is an enzyme produced by a plant, for example Arabidopsis thaliana or by a bacterium, preferably of the genus Streptomyces, in particular Streptomyces clavuligerus.

In a particular embodiment, the chalcone isomerase is selected from enzymes comprising a sequence chosen from SEQ ID NOs: 59 and 61 and polypeptides comprising a sequence having at least 60, 70, 80, 85, 90 or 95% sequence identity with one of these sequences and having chalcone isomerase activity.

In a preferred embodiment, the chalcone isomerase is selected from enzymes comprising a sequence chosen from SEQ ID NO: 61 and polypeptides comprising a sequence having at least 60, 70, 80, 85, 90 or 95% sequence identity with this sequence and having chalcone isomerase activity.

In a first particular embodiment, the CHI is from Streptomyces clavuligerus. It is described in the GenBank database from NCBI under the number CP016559.1 for the nucleic acid sequence and under the number ANW16918.1 for the protein sequence, and more particularly in SEQ ID NOs: 60 and 59, respectively.

In a second particular embodiment, the CHI is from Arabidopsis thaliana. It is described in the GenBank database from NCBI under the number NM_115370.4 for the nucleic acid sequence and under the number NP_191072.1 for the protein sequence, and more particularly in SEQ ID NOs: 62 and 61, respectively.

### FNS: Flavone Synthase

Apigenin may be prepared from naringenin using a flavone synthase (FNS). It is capable of producing apigenin from naringenin.

The microorganism may thus comprise a heterologous nucleic acid sequence coding for a flavone synthase, which is in particular capable of producing apigenin from naringenin and/or a heterologous nucleic acid sequence coding for a flavone synthase, which is in particular capable of producing luteolin from eriodictyol, and/or a heterologous nucleic acid sequence coding for a flavone synthase, which is in particular capable of producing diosmetin from hesperetin.

The FNS may be chosen from those described previously.

Starting with Phenylalanine

Alternatively or in addition, the microorganism may also comprise the enzymes required for the synthesis of p-coumaric acid from phenylalanine.

In this context, the microorganism may also comprise a heterologous nucleic acid sequence coding for a phenylalanine ammonia lyase (PAL) and a heterologous nucleic acid sequence coding for a cinnamate 4-hydroxylase (C4H).

The PAL belongs to the class EC 4.3.1.24. It is capable of producing cinnamic acid from phenylalanine.

The term “phenylalanine ammonia lyase activity” means the transformation of phenylalanine into trans-cinnamic acid by means of the enzyme phenylalanine ammonia lyase. To determine whether there is phenylalanine ammonia lyase activity, an enzymatic test may be performed, which consists of the in vitro incubation of a mixture composed of the phenylalanine ammonia lyase enzyme and phenylalanine, under optimum conditions (pH, temperature, ions, etc.). After a certain incubation time, the appearance of trans-cinnamic acid is observed in UPLC-MS in comparison with the expected standard.

Several enzymes have already been described in the prior art. Preferably, the enzyme originates from a plant, example a plant of the genus Arabidopsis, Agastache, Ananas, Asparagus, Brassica, Bromheadia, Bambusa, Beta, Betula, Citrus, Cucumis, Camellia, Capsicum, Cassia, Catharanthus, Cicer, Citrullus, Coffea, Cucurbita, Cynodon, Daucus, Dendrobium, Dianthus, Digitalis, Dioscorea, Eucalyptus, Gallus, Ginkgo, Glycine, Hordeum, Helianthus, Ipomoea, Lactuca, Lithospermum, Lotus, Lycopersicon, Medicago, Malus, Manihot, Medicago, Mesembryanthemum, Nicotiana, Olea, Oryza, Phaseolus, Pinus, Populus, Pisum, Persea, Petroselinum, Phalaenopsis, Phyllostachys, Physcomitrella, Picea, Pyrus, Prunus, Quercus, Raphanus, Rehmannia, Rubus, Solanum, Sorghum, Sphenostylis, Stellaria, Stylosanthes, Triticum, Trifolium, Vaccinium, Vigna, Vitis, Zea or Zinnia. For example, mention may be made of those from Arabidopsis thaliana or from Petroselinum crispum. In a preferred embodiment, the PAL is from Citrus sinensis.

In addition, phenylalanine ammonia lyase (PAL) may also have tyrosine ammonia lyase (TAL) activity and/or dihydroxyphenylalanine ammonia-lyase (DAL) activity as defined below.

In a particular embodiment, the phenylalanine ammonia lyase (PAL) is selected from enzymes comprising a sequence chosen from SEQ ID NOs: 63, 65 and 77 and polypeptides comprising a sequence having at least 60, 70, 80, 85, 90 or 95% sequence identity with one of these sequences and having phenylalanine ammonia lyase activity.

In a preferred embodiment, the PAL is selected from enzymes comprising a sequence chosen from SEQ ID NOs: 65 and 77 and polypeptides comprising a sequence having at least 60, 70, 80, 85, 90 or 95% sequence identity with one of these sequences and having phenylalanine ammonia lyase activity. Most particularly preferably, the PAL is selected from enzymes comprising a sequence chosen from SEQ ID NOs: 65 and polypeptides comprising a sequence having at least 60, 70, 80, 85, 90 or 95% sequence identity with this sequence and having phenylalanine ammonia lyase activity.

In a particular embodiment, the PAL from Citrus sinensis is described in the GenBank database from NCBI under the number XM 006481431.2 for the nucleic acid sequence and under the number XP_006481494.1 for the protein sequence, and more particularly in SEQ ID NOs: 64 and 63, respectively.

In another particular embodiment, the PAL from Citrus sinensis is described in the GenBank database from NCBI under the number XM_006488000.2 for the nucleic acid sequence and under the number XP_006488063.1 for the protein sequence, and more particularly in SEQ ID NOs: 66 and 65, respectively.

In another particular embodiment, the PAL from Arabidopsis thaliana is described in the GenBank database from NCBI under the number NM_115186.4 for the nucleic acid sequence and under the number NP_190894.1 for the protein sequence, and more particularly in SEQ ID NOs: 78 and 77, respectively.

Optionally, if biosynthesis starting with tyrosine and phenylalanine is envisaged, the PAL and the TAL may be replaced or supplemented with a phenylalanine/tyrosine ammonia lyase (PTAL). PTAL belongs to the class EC 4.3.1.25.

C4H belongs to the class EC 1.14.13.11. It is capable of producing p-coumaric acid from cinnamic acid.

The term “trans-cinnamate 4-monooxygenase activity” means the transformation of trans-cinnamic acid into p-coumaric acid by a trans-cinnamate 4-monooxygenase enzyme (CPR-dependent). To determine whether there is trans-cinnamate 4-monooxygenase activity, an enzymatic test may be performed, which consists of the in vitro incubation of a mixture composed of the trans-cinnamate 4-monooxygenase enzyme, cinnamic acid, NADPH, H⁺ and O₂, under optimum conditions (pH, temperature, ions, etc.). After a certain incubation time, the appearance of the 4-hydroxycinnamate (p-coumaric acid) is observed in UPLC-MS in comparison with the expected standard.

Several enzymes have already been described in the prior art. Preferably, the enzyme originates from a plant, for example a plant of the genus Arabidopsis, Ammi, Avicennia, Camellia, Camptotheca, Catharanthus, Citrus, Glycine, Helianthus, Lotus, Mesembryanthemum, Physcomitreila, Phaseolus, Pinus, Populus, Ruta, Saccharum, Solanum, Vitis, Vigna or Zea. In a preferred embodiment, the cinnamate 4-hydroxylase (C4H) is from Citrus sinensis or from Arabidopsis thaliana.

In a particular embodiment, the cinnamate 4-hydroxylase (C4H) is selected from enzymes comprising a sequence chosen from SEQ ID NOs: 67, 69 and 79 and polypeptides comprising a sequence having at least 60, 70, 80, 85, 90 or 95% sequence identity with one of these sequences and having cinnamate 4-hydroxylase activity.

In a preferred embodiment, the C4H is selected from enzymes comprising a sequence chosen from SEQ ID NO: 79 and polypeptides comprising a sequence having at least 60, 70, 80, 85, 90 or 95% sequence identity with this sequence and having cinnamate 4-hydroxylase activity.

In a particular embodiment, the C4H from Citrus sinensis is described in the GenBank database from NCBI under the number NM_001288840.1 for the nucleic acid sequence and under the number NP_001275769.1 for the protein sequence, and more particularly in SEQ ID NOs: 68 and 67, respectively.

In another particular embodiment, the C4H from Citrus sinensis is described in the GenBank database from NCBI under the number NM_001288895.1 for the nucleic acid sequence and under the number NP_001275824.1 for the protein sequence, and more particularly in SEQ ID NOs: 70 and 69, respectively.

In another particular embodiment, the C4H from Arabidopsis thaliana is described in the GenBank database from NCBI under the number NM_128601.3 for the nucleic acid sequence and under the number NP_180607.1 for the protein sequence, and more particularly in SEQ ID NOs: 80 and 79, respectively.

### Proceeding Via Caffeic Acid

In an additional embodiment, the biosynthesis of eriodictyol may also comprise the synthesis of L-DOPA (3,4-dihydroxy-L-phenylalanine) from tyrosine and then of caffeic acid from L-DOPA (3,4-dihydroxy-L-phenylalanine). To do this, the following enzymes are necessary. To convert tyrosine into L-DOPA (3,4-dihydroxy-L-phenylalanine), two subunits are necessary, HpaB and HpaC.

HpaB is a 4-hydroxyphenylacetate 3-monooxygenase oxygenase subunit (HpaB).

Preferably, this enzyme is an enzyme produced by a bacterium, preferably Escherichia coli.

In a particular embodiment, the 4-hydroxyphenylacetate 3-monooxygenase oxygenase (HpaB) is an enzyme comprising a sequence chosen from SEQ ID NO: 83 and polypeptides comprising a sequence having at least 60, 70, 80, 85, 90 or 95% sequence identity with this sequence and having 4-hydroxyphenylacetate 3-monooxygenase activity.

In a particular embodiment, the HpaB is from Escherichia coli. It is described in the GenBank database from NCBI under the number CAQ34705.1 for the protein sequence, and more particularly in SEQ ID NO: 83. A nucleic acid sequence coding for this enzyme is described in SEQ ID NO: 84. The protein is described in UniProtKB/Swiss Prot under the reference number AOA140NG21.

HpaC is a 4-hydroxyphenylacetate 3-monooxygenase reductase subunit. The microorganism may thus comprise a heterologous nucleic acid sequence coding for a 4-hydroxyphenylacetate 3-monooxygenase reductase subunit (HpaC).

The term “p-coumarate 3-hydroxylase activity” means the transformation of p-coumaric acid into caffeic acid and/or of L-tyrosine into L-DOPA using an enzymatic complex composed of HpaB (4-hydroxyphenylacetate 3-hydroxylase oxidase) and HpaC (4-hydroxyphenylacetate 3-hydroxylase reductase). To determine whether there is p-coumarate 3-hydroxylase activity, an enzymatic test may be performed, which consists of the in vitro incubation of a mixture composed of the enzymes HpaB, HpaC, p-coumaric acid or L-tyrosine, FAD and NADH under optimum conditions (pH, temperature, ions, etc.). After a certain incubation time, the appearance of caffeic acid or of L-DOPA is observed in HPLC-MS in comparison with the expected standard.

Preferably, this enzyme is an enzyme produced by a bacterium, preferably Escherichia coli.

In a particular embodiment, the 4-hydroxyphenylacetate 3-monooxygenase reductase (HpaC) is an enzyme comprising a sequence chosen from SEQ ID NO: 85 and polypeptides comprising a sequence having at least 60, 70, 80, 85, 90 or 95% sequence identity with this sequence and having 4-hydroxyphenylacetate 3-monooxygenase activity.

In a particular embodiment, the HpaC is from Escherichia coli. It is described in the GenBank database from NCBI under the number CAQ34704.1 for the protein sequence, and more particularly in SEQ ID NO: 85. A nucleic acid sequence coding for this enzyme is described in SEQ ID NO: 86. The protein is described in UniProtKB/Swiss Prot under the reference number AOA140NG67.

Together, HpaB and HpaC are capable of producing L-DOPA (3,4-dihydroxy-L-phenylalanine) from tyrosine.

Thus, the microorganism may comprise a heterologous nucleic acid sequence coding for a 4-hydroxyphenylacetate 3-monooxygenase oxygenase (HpaB) and a heterologous nucleic acid sequence coding for 4-hydroxyphenylacetate 3-monooxygenase reductase (HpaC).

Moreover, this pathway also requires the presence of an enzyme that is capable of synthesizing caffeic acid from L-DOPA (3,4-dihydroxy-L-phenylalanine), a dihydroxyphenylalanine ammonia-lyase (DAL). This enzyme belongs to the class EC 4.3.1.11.

The term “dihydroxyphenylalanine ammonia lyase activity” means the transformation of L-DOPA into trans-caffeic acid by means of a dihydroxyphenylalanine ammonia lyase enzyme. To determine whether there is dihydroxyphenylalanine ammonia lyase activity, an enzymatic test may be performed, which consists of the in vitro incubation of a mixture composed of the dihydroxyphenylalanine ammonia lyase enzyme and L-DOPA (levodopa) under optimum conditions (pH, temperature, ions, etc.). After a certain incubation time, the appearance of trans-caffeic acid is observed in UPLC-MS in comparison with the expected standard.

In addition, dihydroxyphenylalanine ammonia lyase (DAL) may also have tyrosine ammonia lyase (TAL) activity and/or phenylalanine ammonia-lyase (PAL) activity.

The microorganism may thus comprise a heterologous nucleic acid sequence coding for a 4-hydroxyphenylacetate 3-monooxygenase oxygenase subunit (HpaB), a heterologous nucleic acid sequence coding for a 4-hydroxyphenylacetate 3-monooxygenase reductase subunit (HpaC) and a heterologous nucleic acid sequence coding for a dihydroxyphenylalanine ammonia-lyase (DAL).

As an alternative to the use of HpaB and HpaC or in combination therewith, it is possible to use an enzyme for converting tyrosine into L-DOPA and an enzyme for converting p-coumaric acid into caffeic acid.

These are, respectively, a 4-methoxybenzoate O-demethylase, also known as 4-methoxybenzoate monooxygenase (0-demethylating) which has L-tyrosine hydroxylase activity, belonging to the class EC 1.14.99.15, and a p-coumarate 3-hydroxylase having p-coumarate 3-hydroxylase activity, belonging to the class EC 1.14.13.

These various enzymes both form part of the cytochrome P450 (CYP) family.

The term “L-tyrosine hydroxylase activity” means the transformation of p-coumaric acid into caffeic acid and/or of L-tyrosine into L-DOPA using a p-coumarate 3-hydroxylase enzyme (CPR-dependent). To determine whether there is L-tyrosine hydroxylase activity, an enzymatic test may be performed, which consists of the in vitro incubation of a mixture composed of the p-coumarate 3-hydroxylase enzyme, p-coumaric acid or L-tyrosine and the necessary cofactors, under optimum conditions (pH, temperature, ions, etc.). After a certain incubation time, the appearance of caffeic acid or of L-DOPA is observed in HPLC-MS in comparison with the expected standard.

The term “p-coumarate 3-hydroxylase activity” means the transformation of p-coumaric acid into caffeic acid and/or of L-tyrosine into L-DOPA using a p-coumarate 3-hydroxylase enzyme (CPR-dependent). To determine whether there is p-coumarate 3-hydroxylase activity, an enzymatic test may be performed, which consists of the in vitro incubation of a mixture composed of the p-coumarate 3-hydroxylase enzyme, p-coumaric acid or L-Tyrosine under optimum conditions (pH, temperature, ions, etc.). After a certain incubation time, the appearance of caffeic acid or of L-DOPA is observed in HPLC-MS in comparison with the expected standard.

The recombinant microorganism may thus comprise a heterologous nucleic acid sequence coding for a 4-methoxybenzoate O-demethylase (CYP) which is capable of converting tyrosine into L-DOPA and also p-coumaric acid into caffeic acid.

In one embodiment, the 4-methoxybenzoate O-demethylase is a bacterial enzyme, notably from Rhodopseudomonas palustris, Pseudomonas putida or Escherichia coli, a plant enzyme, notably from Beta vulgaris, a mammalian enzyme, notably from Oryctolagus cuniculus or a fungal enzyme, notably from Rhodotorula glutinis. In a particular embodiment, the 4-methoxybenzoate O-demethylase is an enzyme from Rhodopseudomonas palustris, Saccharothrix espanaensis or Beta vulgaris.

In a particular embodiment, the 4-methoxybenzoate O-demethylase is selected from enzymes comprising a sequence chosen from SEQ ID NOs: 73 and 75 and polypeptides comprising a sequence having at least 60, 70, 80, 85, 90 or 95% sequence identity with one of these sequences and having L-tyrosine hydrolase activity.

The 4-methoxybenzoate O-demethylase may also be from Beta vulgaris. The nucleic acid sequences coding for this enzyme and the protein sequences are described in SEQ ID NOs: 74 and 73, respectively. The protein is described in UniProtKB/Swiss Prot under the reference number P0DKI2.

In addition, the 4-methoxybenzoate O-demethylase may be from Saccharothrix espanaensis. The nucleic acid sequences coding for this enzyme and protein sequences are described in NCBI under the reference numbers NC_005296.1 and WP_011157377.1, respectively, and more particularly in SEQ ID NOs: 76 and 75. The protein is described in UniProtKB/Swiss Prot under the reference number Q6N8N2.

In one embodiment, the microorganism may comprise a heterologous nucleic acid sequence coding for a 4-methoxybenzoate O-demethylase and a heterologous nucleic acid sequence coding for a dihydroxyphenylalanine ammonia-lyase (DAL).

The recombinant microorganism may thus comprise a heterologous nucleic acid sequence coding for a coumarate 3-hydroxylase (Coum3H) which is capable of converting p-coumaric acid into caffeic acid.

In one embodiment, the coumarate 3-hydroxylase is a bacterial enzyme, notably from Saccharothrix.

In a particular embodiment, the 4-methoxybenzoate O-demethylase is selected from the enzyme comprising a sequence chosen from SEQ ID NO: 71 and polypeptides comprising a sequence having at least 60, 70, 80, 85, 90 or 95% sequence identity with one of these sequences and having coumarate 3-hydroxylase activity.

The nucleic acid sequence coding for this enzyme and the protein sequence are described in NCBI under the reference numbers DQ357071.1 and ABC88666.1, respectively, and more particularly in SEQ ID NOs: 72 and 71.

In one embodiment, the microorganism may comprise a heterologous nucleic acid sequence coding for a coumarate 3-hydroxylase and a heterologous nucleic acid sequence coding for a dihydroxyphenylalanine ammonia-lyase (DAL).

### Additional Combination of Enzymes

Thus, besides the enzymes required for the biosynthesis of hesperidin and/or diosmin from naringenin and/or apigenin as described previously, the microorganism preferably comprises enzymes for producing naringenin and/or apigenin from tyrosine and/or phenylalanine, preferably from tyrosine.

Thus, according to particular embodiments, the microorganism comprises
a heterologous nucleic acid sequence coding for a flavanone 7-O-beta-D-glucosyltransferase (UGT) which is capable of adding a glucose in position 7 of hesperetin and/or diosmetin,
a heterologous nucleic acid sequence coding for a 6″-O-rhamnosyltransferase (RhaT) which is capable of transferring a rhamnose into position 6 of the glucose of hesperetin-7-O-glucoside and/or diosmetin-7-O-glucoside,
a heterologous nucleic acid sequence coding for a UDP-glucose 4,6-dehydratase/UDP-4-keto-6-deoxy-D-glucose 3,5-epimerase/UDP-4-keto-L-rhamnose-reductase (RHM) which is capable of producing UDP-rhamnose,
a heterologous nucleic acid sequence coding for an O-methyltransferase (OMT) which is capable of methylating eriodictyol and/or luteolin in position 4′,
a heterologous nucleic acid sequence coding for an F3′H enzyme,
and optionally a heterologous nucleic acid sequence coding for an FNS enzyme and a heterologous nucleic acid sequence coding for a CPR enzyme,
and also comprises
a heterologous nucleic acid sequence coding for a tyrosine ammonia lyase (TAL), a heterologous nucleic acid sequence coding for a 4-coumaroyl-CoA ligase (4CL), a heterologous nucleic acid sequence coding for a chalcone synthase (CHS) and a heterologous nucleic acid sequence coding for a chalcone isomerase (CHI).

In one embodiment, the microorganism comprises:
a heterologous nucleic acid sequence coding for an O-methyltransferase (OMT) which is capable of methylating eriodictyol and/or luteolin in position 4′; preferably an OMT from Citrus clementina, Citrus sinensis, Arabidopsis thaliana or Homo sapiens, preferably an OMT comprising a sequence chosen from SEQ ID NOs: 117, 119, 87 and 89 and polypeptides comprising a sequence having at least 60, 70, 80, 85, 90 or 95% sequence identity with one of these sequences and having O-methyltransferase activity, notably with eriodictyol and/or luteolin as substrate and methylation in position 4′, preferably an OMT comprising a sequence chosen from SEQ ID NOs: 117 and 119 and polypeptides comprising a sequence having at least 60, 70, 80, 85, 90 or 95% sequence identity with one of these sequences and having O-methyltransferase activity, and most particularly preferably an OMT comprising a sequence chosen from SEQ ID NO: 117 and polypeptides comprising a sequence having at least 60, 70, 80, 85, 90 or 95% sequence identity with this sequence and having O-methyltransferase activity; and
a heterologous nucleic acid sequence coding for a flavonoid 3′-monooxygenase (F3′H) which is capable of hydroxylating naringenin and/or apigenin in position 3′ and comprising a sequence selected from SEQ ID NOs: 7, 11, 17 and 121 and polypeptides comprising a sequence having at least 75%, at least 80%, at least 85%, at least 90% or at least 95% identity with one of these sequences and having flavonoid 3′-monooxygenase activity, preferably an enzyme comprising a sequence selected from SEQ ID NOs: 7, 17 and 121 and polypeptides comprising a sequence having at least 75%, at least 80%, at least 85%, at least 90% or at least 95% identity with one of these sequences and having flavonoid 3′-monooxygenase activity, and most particularly preferably an enzyme comprising a sequence selected from SEQ ID NO: 7 and polypeptides comprising a sequence having at least 75%, at least 80%, at least 85%, at least 90% or at least 95% identity with this sequence and having flavonoid 3′-monooxygenase activity; and
optionally, a heterologous nucleic acid sequence coding for a cytochrome P450 reductase (CPR) and comprising a sequence chosen from SEQ ID NOs: 23, 25, 27, 29 and 31 and polypeptides comprising a sequence having at least 60, 70, 80, 85, 90 or 95% sequence identity with one of these sequences and having cytochrome P450 reductase activity, preferably from enzymes comprising a sequence chosen from SEQ ID NOs: 23, 25 and 29 and polypeptides comprising a sequence having at least 60, 70, 80, 85, 90 or 95% sequence identity with one of these sequences and having cytochrome P450 reductase activity, and particularly from enzymes comprising a sequence chosen from SEQ ID NO: 25 and polypeptides comprising a sequence having at least 60, 70, 80, 85, 90 or 95% sequence identity with this sequence and having cytochrome P450 reductase activity; and
optionally, a heterologous nucleic acid sequence coding for a flavone synthase (FNS) and comprising a sequence chosen from SEQ ID NOs: 33, 35, 37, 127, 129, 131, 133, 135, 137, 139, 141, 143, 145, 147, 149, 151, 153, 155, 157 and 159 and polypeptides comprising a sequence having at least 60, 70, 80, 85, 90 or 95% sequence identity with one of these sequences and having flavone synthase activity, in particular for a flavone synthase (FNS) and comprising a sequence chosen from SEQ ID NOs: 33, 35 and 37 and polypeptides comprising a sequence having at least 60, 70, 80, 85, 90 or 95% sequence identity with one of these sequences and having flavone synthase activity, preferably a flavone synthase (FNS) comprising a sequence chosen from SEQ ID NO: 37 and a polypeptide comprising a sequence having at least 60, 70, 80, 85, 90 or 95% sequence identity with this sequence and having flavone synthase activity; and
a heterologous nucleic acid sequence coding for a tyrosine ammonia lyase (TAL) from Rhodotorula glutinis or Flavobacterium johnsoniae; in particular a TAL comprising a sequence chosen from SEQ ID NOs: 41 and 39 and polypeptides comprising a sequence having at least 60, 70, 80, 85, 90 or 95% sequence identity with one of these sequences and having tyrosine ammonia lyase activity; preferably a TAL comprising a sequence chosen from SEQ ID NO: 41 and polypeptides comprising a sequence having at least 60, 70, 80, 85, 90 or 95% sequence identity with this sequence and having tyrosine ammonia lyase activity; and
a heterologous nucleic acid sequence coding for a 4-coumaroyl-CoA ligase (4CL) from Arabidopsis thaliana, Citrus clementina, Petroselinum crispum or Streptomyces clavuligerus; in particular a 4CL comprising a sequence chosen from SEQ ID NOs: 123, 125, 45, 43, 47 and 49 and polypeptides comprising a sequence having at least 60, 70, 80, 85, 90 or 95% sequence identity with one of these sequences and having 4-coumarate-CoA ligase activity; preferably a 4CL comprising a sequence selected from SEQ ID NOs: 123, 125 and 45 and polypeptides comprising a sequence having at least 60, 70, 80, 85, 90 or 95% sequence identity with one of these sequences and having 4-coumarate-CoA ligase activity; most particularly preferably a 4CL comprising a sequence selected from SEQ ID NO: 45 and polypeptides comprising a sequence having at least 60, 70, 80, 85, 90 or 95% sequence identity with this sequence and having 4-coumarate-CoA ligase activity, and
a heterologous nucleic acid sequence coding for a chalcone synthase (CHS) from Citrus sinensis, Hordeum vulgare or Streptomyces clavuligerus, in particular a CHS comprising a sequence chosen from SEQ ID NOs: 53, 51, 55 and 57 and polypeptides comprising a sequence having at least 60, 70, 80, 85, 90 or 95% sequence identity with one of these sequences and having chalcone synthase activity, preferably a CHS comprising a sequence chosen from SEQ ID NO: 53 and polypeptides comprising a sequence having at least 60, 70, 80, 85, 90 or 95% sequence identity with this sequence and having chalcone synthase activity; and
a heterologous nucleic acid sequence coding for a chalcone isomerase (CHI) from Arabidopsis thaliana or Streptomyces clavuligerus, in particular a CHI comprising a sequence chosen from SEQ ID NOs: 61 and 59 and polypeptides comprising a sequence having at least 60, 70, 80, 85, 90 or 95% sequence identity with one of these sequences and having chalcone isomerase activity; preferably a CHI comprising a sequence chosen from SEQ ID NO: 61 and polypeptides comprising a sequence having at least 60, 70, 80, 85, 90 or 95% sequence identity with this sequence and having chalcone isomerase activity.

Preferably, in this embodiment, the microorganism comprises one of the combinations of enzymes UGT, RhaT and RHM described above, in particular
the flavanone 7-O-beta-D-glucosyltransferase selected from enzymes comprising a sequence chosen from SEQ ID NOs: 113 and 95 and polypeptides comprising a sequence having at least 60, 70, 80, 85, 90 or 95% sequence identity with one of these sequences and having flavanone 7-O-beta-D-glucosyltransferase activity, preferably selected from enzymes comprising a sequence chosen from SEQ ID NO: 113 and polypeptides comprising a sequence having at least 60, 70, 80, 85, 90 or 95% sequence identity with this sequence and having flavanone 7-O-beta-D-glucosyltransferase activity; and
the 6″-O-rhamnosyltransferase selected from enzymes comprising a sequence chosen from SEQ ID NO: 103 and polypeptides comprising a sequence having at least 60, 70, 80, 85, 90 or 95% sequence identity with this sequence and having 6″-O-rhamnosyltransferase activity; and
the UDP-glucose 4,6-dehydratase/UDP-4-keto-6-deoxy-D-glucose 3,5-epimerase/UDP-4-keto-L-rhamnose-reductase selected from enzymes comprising a sequence chosen from SEQ ID NO: 107 and polypeptides comprising a sequence having at least 60, 70, 80, 85, 90 or 95% sequence identity with this sequence and having UDP-glucose 4,6-dehydratase/UDP-4-keto-6-deoxy-D-glucose 3,5-epimerase/UDP-4-keto-L-rhamnose-reductase activity.

Preferably, in this embodiment, the microorganism also comprises:
a heterologous nucleic acid sequence coding for a phenylalanine ammonia lyase (PAL), in particular a PAL comprising a sequence chosen from SEQ ID NOs: 63, 65 and 77, preferably SEQ ID NOs: 65 and 77 and polypeptides comprising a sequence having at least 60, 70, 80, 85, 90 or 95% sequence identity with one of these sequences and having phenylalanine ammonia lyase activity, and more particularly preferably a phenylalanine ammonia lyase (PAL) comprising a sequence chosen from SEQ ID NO: 65 and polypeptides comprising a sequence having at least 60, 70, 80, 85, 90 or 95% sequence identity with this sequence and having phenylalanine ammonia lyase activity; and
a heterologous nucleic acid sequence coding for a cinnamate 4-hydroxylase (C4H), in particular a C4H comprising a sequence chosen from SEQ ID NOs: 67, 69 and 79 and polypeptides comprising a sequence having at least 60, 70, 80, 85, 90 or 95% sequence identity with one of these sequences and having cinnamate 4-hydroxylase activity; and most particularly preferably a cinnamate 4-hydroxylase (C4H) comprising a sequence chosen from SEQ ID NO: 79 and polypeptides comprising a sequence having at least 60, 70, 80, 85, 90 or 95% sequence identity with this sequence and having cinnamate 4-hydroxylase activity.

In another embodiment, the microorganism comprises:
a heterologous nucleic acid sequence coding for an O-methyltransferase (OMT) comprising a sequence chosen from SEQ ID NOs: 117 and 119 and polypeptides comprising a sequence having at least 60, 70, 80, 85, 90 or 95% sequence identity with one of these sequences and having O-methyltransferase activity, and most particularly preferably an OMT comprising a sequence chosen from SEQ ID NO: 117 and polypeptides comprising a sequence having at least 60, 70, 80, 85, 90 or 95% sequence identity with this sequence and having O-methyltransferase activity; and
a heterologous nucleic acid sequence coding for a flavonoid 3′-monooxygenase (F3′H) which is capable of hydroxylating naringenin and/or apigenin in position 3′ and comprising a sequence selected from SEQ ID NOs: 7, 17 and 121 and polypeptides comprising a sequence having at least 75%, at least 80%, at least 85%, at least 90% or at least 95% identity with one of these sequences and having flavonoid 3′-monooxygenase activity, preferably an enzyme comprising a sequence selected from SEQ ID NO: 7 and polypeptides comprising a sequence having at least 75%, at least 80%, at least 85%, at least 90% or at least 95% identity with this sequence and having flavonoid 3′-monooxygenase activity; and
optionally, a heterologous nucleic acid sequence coding for a cytochrome P450 reductase (CPR) and comprising a sequence chosen from SEQ ID NOs: 23, 25 and 29 and polypeptides comprising a sequence having at least 60, 70, 80, 85, 90 or 95% sequence identity with one of these sequences and having cytochrome P450 reductase activity, preferably chosen from enzymes comprising a sequence chosen from SEQ ID NO: 25 and polypeptides comprising a sequence having at least 60, 70, 80, 85, 90 or 95% sequence identity with this sequence and having cytochrome P450 reductase activity; and
optionally, a heterologous nucleic acid sequence coding for a flavone synthase (FNS) and comprising a sequence chosen from SEQ ID NO: 37 and a polypeptide comprising a sequence having at least 60, 70, 80, 85, 90 or 95% sequence identity with this sequence and having flavone synthase activity; and
   a heterologous nucleic acid sequence coding for a tyrosine ammonia lyase (TAL) comprising a sequence chosen from SEQ ID NOs: 41 and polypeptides comprising a sequence having at least 60, 70, 80, 85, 90 or 95% sequence identity with this sequence and having tyrosine ammonia lyase activity;
   a heterologous nucleic acid sequence coding for a 4-coumaroyl-CoA ligase (4CL) comprising a sequence selected from SEQ ID NOs: 123, 125 and 45 and polypeptides comprising a sequence having at least 60, 70, 80, 85, 90 or 95% sequence identity with these sequences and having 4-coumarate-CoA ligase activity, most particularly preferably a 4CL comprising a sequence selected from SEQ ID NO: 45 and polypeptides comprising a sequence having at least 60, 70, 80, 85, 90 or 95% sequence identity with this sequence and having 4-coumarate-CoA ligase activity; and
   a heterologous nucleic acid sequence coding for a chalcone synthase (CHS) comprising a sequence chosen from SEQ ID NO: 53 and polypeptides comprising a sequence having at least 60, 70, 80, 85, 90 or 95% sequence identity with this sequence and having chalcone synthase activity; and
   a heterologous nucleic acid sequence coding for a chalcone isomerase (CHI) comprising a sequence chosen from SEQ ID NO: 61 and polypeptides comprising a sequence having at least 60, 70, 80, 85, 90 or 95% sequence identity with this sequence and having chalcone isomerase activity.

Preferably, in this embodiment, the microorganism comprises one of the combinations of enzymes UGT, RhaT and RHM described above, in particular
the flavanone 7-O-beta-D-glucosyltransferase selected from enzymes comprising a sequence chosen from SEQ ID NOs: 113 and 95 and polypeptides comprising a sequence having at least 60, 70, 80, 85, 90 or 95% sequence identity with one of these sequences and having flavanone 7-O-beta-D-glucosyltransferase activity, preferably selected from enzymes comprising a sequence chosen from SEQ ID NO: 113 and polypeptides comprising a sequence having at least 60, 70, 80, 85, 90 or 95% sequence identity with this sequence and having flavanone 7-O-beta-D-glucosyltransferase activity; and
the 6″-O-rhamnosyltransferase selected from enzymes comprising a sequence chosen from SEQ ID NO: 103 and polypeptides comprising a sequence having at least 60, 70, 80, 85, 90 or 95% sequence identity with this sequence and having 6″-O-rhamnosyltransferase activity; and
the UDP-glucose 4,6-dehydratase/UDP-4-keto-6-deoxy-D-glucose 3,5-epimerase/UDP-4-keto-L-rhamnose-reductase selected from enzymes comprising a sequence chosen from SEQ ID NO: 107 and polypeptides comprising a sequence having at least 60, 70, 80, 85, 90 or 95% sequence identity with this sequence and having UDP-glucose 4,6-dehydratase/UDP-4-keto-6-deoxy-D-glucose 3,5-epimerase/UDP-4-keto-L-rhamnose-reductase activity.

Preferably, in this embodiment, the microorganism also comprises a heterologous nucleic acid sequence coding for a phenylalanine ammonia lyase (PAL) and a heterologous nucleic acid sequence coding for a cinnamate 4-hydroxylase (C4H) as described in the preceding embodiment.

Optionally, in these various embodiments, the microorganism also comprises a heterologous or endogenous nucleic acid sequence coding for an S-adenosylmethionine synthetase (SAMT); in particular from Saccharomyces cerevisiae, for example an SAMT comprising a sequence chosen from SEQ ID NO: 81 and a polypeptide comprising a sequence having at least 60, 70, 80, 85, 90 or 95% sequence identity with this sequence and having S-adenosylmethionine synthetase activity.

In another particular embodiment, the microorganism comprises:
a heterologous nucleic acid sequence coding for a flavanone 7-O-beta-D-glucosyltransferase (UGT) from Arabidopsis thaliana, Scutellaria baicalensis or Homo sapiens, preferably from Arabidopsis thaliana or from Scutellaria baicalensis, preferably a flavanone 7-O-beta-D-glucosyltransferase (UGT) selected from enzymes comprising a sequence chosen from SEQ ID NOs: 91, 93, 95, 97, 99 and 101 and polypeptides comprising a sequence having at least 60, 70, 80, 85, 90 or 95% sequence identity with one of these sequences and having flavanone 7-O-beta-D-glucosyltransferase activity; in particular a flavanone 7-O-beta-D-glucosyltransferase (UGT) comprising a sequence chosen from SEQ ID NOs: 91, 93, 95 and 97 and polypeptides comprising a sequence having at least 60, 70, 80, 85, 90 or 95% sequence identity with one of these sequences and having flavanone 7-O-beta-D-glucosyltransferase activity, and preferably a UGT comprising a sequence chosen from SEQ ID NO: 113 and polypeptides comprising a sequence having at least 60, 70, 80, 85, 90 or 95% sequence identity with this sequence and having flavanone 7-O-beta-D-glucosyltransferase activity;
a heterologous nucleic acid sequence coding for a 6″-O-rhamnosyltransferase (RhaT) of the genus Citrus or Petunia hybrida, preferably Citrus sinensis, Citrus maxima, or Citrus clementina, even more preferably Citrus sinensis or Citrus clementina, preferably a 6″-O-rhamnosyltransferase (RhaT) comprising a sequence chosen from SEQ ID NOs: 103 and 105 and polypeptides comprising a sequence having at least 60, 70, 80, 85, 90 or 95% sequence identity with one of these sequences and having 6″-O-rhamnosyltransferase activity, and preferably an RhaT comprising a sequence chosen from SEQ ID NO: 103 and polypeptides comprising a sequence having at least 60, 70, 80, 85, 90 or 95% sequence identity with this sequence and having 6″-O-rhamnosyltransferase activity; and
a heterologous nucleic acid sequence coding for a UDP-glucose 4,6-dehydratase/UDP-4-keto-6-deoxy-D-glucose 3,5-epimerase/UDP-4-keto-L-rhamnose-reductase (RHM) from Citrus sinensis or from Arabidopsis thaliana, preferably an RHM comprising a sequence chosen from SEQ ID NOs: 107, 109 and 111 and polypeptides comprising a sequence having at least 60, 70, 80, 85, 90 or 95% sequence identity with one of these sequences and having UDP-glucose 4,6-dehydratase/UDP-4-keto-6-deoxy-D-glucose 3,5-epimerase/UDP-4-keto-L-rhamnose-reductase activity, in particular an RHM comprising a sequence selected from SEQ ID NOs: 107 and 109 and polypeptides comprising a sequence having at least 60, 70, 80, 85, 90 or 95% sequence identity with this sequence and having UDP-glucose 4,6-dehydratase/UDP-4-keto-6-deoxy-D-glucose 3,5-epimerase/UDP-4-keto-L-rhamnose-reductase activity, and preferably an RHM comprising a sequence selected from SEQ ID NO: 107 and polypeptides comprising a sequence having at least 60, 70, 80, 85, 90 or 95% sequence identity with this sequence and having UDP-glucose 4,6-dehydratase/UDP-4-keto-6-deoxy-D-glucose 3,5-epimerase/UDP-4-keto-L-rhamnose-reductase activity;
a heterologous nucleic acid sequence coding for a flavonoid 3′-monooxygenase (F3′H) which is capable of hydroxylating naringenin and/or apigenin in position 3′; preferably from Perilla frutescens var. crispa, Petunia x hybrida, Callistephus chinensis, Gerbera hybrida, Citrus sinensis, Citrus clementina, Osteospermum hybrid cultivar, Phanerochaete chrysosporium, Streptomyces avermitilis or Pilosella officinarum, in particular from Perilla frutescens var. crispa, Petunia x hybrida, Callistephus chinensis, Gerbera hybrida, Citrus sinensis or Pilosella officinarum, preferably an F3′H comprising a sequence chosen from SEQ ID NOs: 1, 3, 5, 7, 9, 11, 13, 15, 17, 19 and 21 and polypeptides comprising a sequence having at least 60, 70, 80, 85, 90 or 95% sequence identity with one of these sequences and having flavonoid 3′-monooxygenase activity, preferably selected from enzymes comprising a sequence chosen from SEQ ID NOs: 1, 5, 7, 11, 17 and 19 and polypeptides comprising a sequence having at least 60, 70, 80, 85, 90 or 95% sequence identity with one of these sequences and having flavonoid 3′-monooxygenase activity, in particular an F3′H comprising a sequence selected from SEQ ID NOs: 5, 7 and 17 and polypeptides comprising a sequence having at least 60, 70, 80, 85, 90 or 95% sequence identity with this sequence and having flavonoid 3′-monooxygenase activity, more particularly preferably an F3′H comprising a sequence chosen from SEQ ID NO: 7 and polypeptides comprising a sequence having at least 60, 70, 80, 85, 90 or 95% sequence identity with the sequence SEQ ID NO: 7 and having flavonoid 3′-monooxygenase activity;
optionally, a heterologous nucleic acid sequence coding for a cytochrome P450 reductase (CPR); preferably a CPR from Saccharomyces cerevisiae, or from a plant, for example from Catharanthus roseus or Arabidopsis thaliana; preferably a CPR comprising a sequence chosen from SEQ ID NOs: 23, 25, 27, 29 and 31 and polypeptides comprising a sequence having at least 60, 70, 80, 85, 90 or 95% sequence identity with one of these sequences and having cytochrome P450 reductase activity, preferably from enzymes comprising a sequence chosen from SEQ ID NOs: 23, 25 and 29 and polypeptides comprising a sequence having at least 60, 70, 80, 85, 90 or 95% sequence identity with one of these sequences and having cytochrome P450 reductase activity, and particularly a CPR comprising a sequence chosen from SEQ ID NO: 25 and polypeptides comprising a sequence having at least 60, 70, 80, 85, 90 or 95% sequence identity with this sequence and having cytochrome P450 reductase activity;
a heterologous nucleic acid sequence coding for an O-methyltransferase (OMT) which is capable of methylating eriodictyol and/or luteolin in position 4′; preferably an OMT from Arabidopsis thaliana or Homo sapiens, preferably an OMT comprising a sequence chosen from SEQ ID NOs: 87 and 89 and polypeptides comprising a sequence having at least 60, 70, 80, 85, 90 or 95% sequence identity with one of these sequences and having O-methyltransferase activity, notably with eriodictyol and/or luteolin as substrate and with methylation in position 4′, preferably selected from the enzyme comprising a sequence chosen from SEQ ID NO: 89 and polypeptides comprising a sequence having at least 60, 70, 80, 85, 90 or 95% sequence identity with this sequence and having O-methyltransferase activity;
optionally, a heterologous or endogenous nucleic acid sequence coding for an S-adenosylmethionine synthetase (SAMT), in particular from Saccharomyces cerevisiae, for example an SAMT comprising a sequence chosen from SEQ ID NO: 81 and a polypeptide comprising a sequence having at least 60, 70, 80, 85, 90 or 95% sequence identity with this sequence and having S-adenosylmethionine synthetase activity; and
optionally, a heterologous nucleic acid sequence coding for a flavone synthase (FNS) which is capable of transforming naringenin into apigenin, eriodictyol into luteolin, and/or hesperetin into diosmetin, preferably of transforming eriodictyol into luteolin; preferably an FNS from Arabidopsis thaliana, Lonicera japonica, Lonicera macranthoides, Medicago truncatula, Oryza sativa, Petroselinum crispum, Populus deltoides, Zea mays, Callistephus chinensis, Apium graveolens, Medicago truncatula, Cuminum cyminum, Aethusa cynapium, Angelica archangelica, Conium maculatum, Camellia sinensis, Cynara cardunculus var scolymus, Saussurea medusa, Plectranthus barbatus, Scutellaria baicalensis, Dorcoceras hygrometricum, Antirrhinum majus, Perilla frutescens var crispa, Dahlia pinnata or Erythranthe lewisii, in particular from Arabidopsis thaliana, Lonicera japonica, Lonicera macranthoides, Medicago truncatula, Oryza sativa, Petroselinum crispum, Populus deltoides or Zea mays, preferably from Lonicera japonica, Lonicera macranthoides and Petroselinum crispum; preferably an FNS comprising a sequence chosen from SEQ ID NOs: 33, 35, 37, 127, 129, 131, 133, 135, 137, 139, 141, 143, 145, 147, 149, 151, 153, 155, 157 and 159 and polypeptides comprising a sequence having at least 60, 70, 80, 85, 90 or 95% sequence identity with one of these sequences and having flavone synthase activity, preferably from enzymes comprising a sequence chosen from SEQ ID NOs: 33, 35 and 37 and polypeptides comprising a sequence having at least 60, 70, 80, 85, 90 or 95% sequence identity with one of these sequences and having flavone synthase activity, preferably a flavone synthase (FNS) comprising a sequence chosen from SEQ ID NO: 37 and a polypeptide comprising a sequence having at least 60, 70, 80, 85, 90 or 95% sequence identity with this sequence and having flavone synthase activity;
a heterologous nucleic acid sequence coding for a tyrosine ammonia lyase (TAL) which is capable of producing p-coumaric acid from tyrosine; preferably from Rhodotorula glutinis or Flavobacterium johnsoniae; in particular a TAL comprising a sequence chosen from SEQ ID NOs: 39 and 41 and polypeptides comprising a sequence having at least 60, 70, 80, 85, 90 or 95% sequence identity with one of these sequences and having tyrosine ammonia lyase activity; preferably a TAL comprising a sequence chosen from SEQ ID NO: 41 and polypeptides comprising a sequence having at least 60, 70, 80, 85, 90 or 95% sequence identity with this sequence and having tyrosine ammonia lyase activity;
a heterologous nucleic acid sequence coding for a 4-coumarate-CoA ligase (4CL) which is capable of producing coumaryl-CoA from p-coumaric acid and Coenzyme A; preferably from Arabidopsis thaliana, Petroselinum crispum or Streptomyces clavuligerus; a 4CL comprising a sequence chosen from SEQ ID NOs: 43, 45, 47 and 49 and polypeptides comprising a sequence having at least 60, 70, 80, 85, 90 or 95% sequence identity with one of these sequences and having 4-coumarate-CoA ligase activity, preferably a 4CL comprising a sequence selected from SEQ ID NO: 45 and polypeptides comprising a sequence having at least 60, 70, 80, 85, 90 or 95% sequence identity with this sequence and having 4-coumarate-CoA ligase activity;
a heterologous nucleic acid sequence coding for a chalcone synthase (CHS) which is capable of producing naringenin-chalcone from 4-coumaroyl-CoA and malonyl-CoA; preferably from Citrus sinensis, Hordeum vulgare or Streptomyces clavuligerus, in particular a CHS comprising a sequence chosen from SEQ ID NOs: 51, 53, 55 and 57 and polypeptides comprising a sequence having at least 60, 70, 80, 85, 90 or 95% sequence identity with one of these sequences and having chalcone synthase activity, preferably from enzymes comprising a sequence chosen from SEQ ID NOs: 53 and 55 and polypeptides comprising a sequence having at least 60, 70, 80, 85, 90 or 95% sequence identity with one of these sequences and having chalcone synthase activity, and more particularly preferably a CHS comprising a sequence chosen from SEQ ID NO: 53 and polypeptides comprising a sequence having at least 60, 70, 80, 85, 90 or 95% sequence identity with this sequence and having chalcone synthase activity;
a heterologous nucleic acid sequence coding for a chalcone isomerase (CHI) which is capable of producing naringenin from naringenin chalcone; preferably from Arabidopsis thaliana or Streptomyces clavuligerus, in particular a CHI comprising a sequence chosen from SEQ ID NOs: 59 and 61 and polypeptides comprising a sequence having at least 60, 70, 80, 85, 90 or 95% sequence identity with one of these sequences and having chalcone isomerase activity, and more particularly preferably a CHI comprising a sequence chosen from SEQ ID NO: 61 and polypeptides comprising a sequence having at least 60, 70, 80, 85, 90 or 95% sequence identity with this sequence and having chalcone isomerase activity.

In another particular embodiment, the microorganism comprises heterologous nucleic acid sequences coding for the enzymes UGT, RhaT, RHM, F3′H, OMT, 4CL, CHS and CHI, and optionally for the enzymes CPR, FNS and SAMT, as described in the preceding embodiment and also comprises:
a heterologous nucleic acid sequence coding for a phenylalanine ammonia lyase (PAL), in particular a PAL comprising a sequence chosen from SEQ ID NOs: 63, 65 and 77 and polypeptides comprising a sequence having at least 60, 70, 80, 85, 90 or 95% sequence identity with one of these sequences and having phenylalanine ammonia lyase activity, in particular a PAL comprising a sequence chosen from SEQ ID NO: 65 and polypeptides comprising a sequence having at least 60, 70, 80, 85, 90 or 95% sequence identity with this sequence and having phenylalanine ammonia lyase activity, and a heterologous nucleic acid sequence coding for a cinnamate 4-hydroxylase (C4H), in particular a C4H comprising a sequence chosen from SEQ ID NOs: 67, 69 and 79 and polypeptides comprising a sequence having at least 60, 70, 80, 85, 90 or 95% sequence identity with one of these sequences and having cinnamate 4-hydroxylase activity, which are capable of producing p-coumaric acid from phenylalanine, in particular a C4H comprising a sequence chosen from SEQ ID NO: 79 and polypeptides comprising a sequence having at least 60, 70, 80, 85, 90 or 95% sequence identity with this sequence and having cinnamate 4-hydroxylase activity.

In a third particular embodiment, the microorganism comprises heterologous nucleic acid sequences coding for the enzymes UGT, RhaT, RHM, F3′H, OMT, 4CL, CHS and CHI, and optionally for the enzymes CPR, FNS and SAMT, as described in the preceding embodiment and also comprises:
a heterologous nucleic acid sequence coding for a dihydroxyphenylalanine ammonia-lyase (DAL) which is capable of producing caffeic acid from L-DOPA (3,4-dihydroxy-L-phenylalanine);
a heterologous nucleic acid sequence coding for a 4-hydroxyphenylacetate 3-monooxygenase oxygenase subunit (HpaB), preferably comprising a sequence chosen from SEQ ID NO: 83 and polypeptides comprising a sequence having at least 60, 70, 80, 85, 90 or 95% sequence identity therewith and having 4-hydroxyphenylacetate 3-monooxygenase oxygenase activity, and a heterologous nucleic acid sequence coding for a 4-hydroxyphenylacetate 3-monooxygenase reductase subunit (HpaC), preferably comprising a sequence chosen from SEQ ID NO: 85 and polypeptides comprising a sequence having at least 60, 70, 80, 85, 90 or 95% sequence identity therewith and having 4-hydroxyphenylacetate 3-monooxygenase reductase activity; or a heterologous nucleic acid sequence coding for a 4-methoxybenzoate O-demethylase which is capable of converting tyrosine into L-DOPA and also p-coumaric acid into caffeic acid, preferably comprising a sequence chosen from SEQ ID NOs: 73 and 75 and polypeptides comprising a sequence having at least 60, 70, 80, 85, 90 or 95% sequence identity with one of these sequences and having L-tyrosine hydroxylase activity; or a heterologous nucleic acid sequence coding for a p-coumarate 3-hydroxylase which is capable of converting p-coumaric acid into caffeic acid, preferably comprising a sequence chosen from SEQ ID NO: 71 and polypeptides comprising a sequence having at least 60, 70, 80, 85, 90 or 95% sequence identity with one of these sequences and having p-coumarate 3-hydroxylase activity.

In another particular embodiment, the microorganism comprises heterologous nucleic acid sequences coding for the enzymes UGT, RhaT, RHM, F3′H, OMT, 4CL, CHS and CHI, and optionally for the enzymes CPR, FNS and SAMT, as described in the preceding embodiment and also comprises:
a heterologous nucleic acid sequence coding for a tyrosine ammonia lyase (TAL) which is capable of producing p-coumaric acid from tyrosine; preferably from Rhodotorula glutinis or Flavobacterium johnsoniae; in particular a TAL comprising a sequence chosen from SEQ ID NOs: 39 and 41 and polypeptides comprising a sequence having at least 60, 70, 80, 85, 90 or 95% sequence identity with one of these sequences and having tyrosine ammonia lyase activity; preferably a TAL comprising a sequence chosen from SEQ ID NO: 41 and polypeptides comprising a sequence having at least 60, 70, 80, 85, 90 or 95% sequence identity with this sequence and having tyrosine ammonia lyase activity;
optionally, a heterologous nucleic acid sequence coding for a phenylalanine ammonia lyase (PAL), in particular a PAL comprising a sequence chosen from SEQ ID NOs: 63, 65 and 77 and polypeptides comprising a sequence having at least 60, 70, 80, 85, 90 or 95% sequence identity with one of these sequences and having phenylalanine ammonia lyase activity, preferably a PAL comprising a sequence chosen from SEQ ID NO: 65 and polypeptides comprising a sequence having at least 60, 70, 80, 85, 90 or 95% sequence identity with this sequence and having phenylalanine ammonia lyase activity; and a heterologous nucleic acid sequence coding for a cinnamate 4-hydroxylase (C4H), in particular a C4H comprising a sequence chosen from SEQ ID NOs: 67, 69 and 79 and polypeptides comprising a sequence having at least 60, 70, 80, 85, 90 or 95% sequence identity with one of these sequences and having cinnamate 4-hydroxylase activity, which are capable of producing p-coumaric acid from phenylalanine, preferably a C4H comprising a sequence chosen from SEQ ID NO: 79 and polypeptides comprising a sequence having at least 60, 70, 80, 85, 90 or 95% sequence identity with this sequence and having cinnamate 4-hydroxylase activity;
optionally, a heterologous nucleic acid sequence coding for a 4-hydroxyphenylacetate 3-monooxygenase oxygenase subunit (HpaB), preferably comprising a sequence chosen from SEQ ID NO: 83 and polypeptides comprising a sequence having at least 60, 70, 80, 85, 90 or 95% sequence identity therewith and having 4-hydroxyphenylacetate 3-monooxygenase oxygenase activity, and a heterologous nucleic acid sequence coding for a 4-hydroxyphenylacetate 3-monooxygenase reductase subunit (HpaC), preferably comprising a sequence chosen from SEQ ID NO: 85 and polypeptides comprising a sequence having at least 60, 70, 80, 85, 90 or 95% sequence identity therewith and having 4-hydroxyphenylacetate 3-monooxygenase reductase activity; or a heterologous nucleic acid sequence coding for a 4-methoxybenzoate O-demethylase which is capable of converting tyrosine into L-DOPA and also p-coumaric acid into caffeic acid, preferably comprising a sequence chosen from SEQ ID NOs: 73 and 75 and polypeptides comprising a sequence having at least 60, 70, 80, 85, 90 or 95% sequence identity with one of these sequences and having L-tyrosine hydrolase activity; or a heterologous nucleic acid sequence coding for a p-coumarate 3-hydroxylase which is capable of converting p-coumaric acid into caffeic acid, preferably comprising a sequence chosen from SEQ ID NO: 71 and polypeptides comprising a sequence having at least 60, 70, 80, 85, 90 or 95% sequence identity with one of these sequences and having p-coumarate 3-hydroxylase activity;
optionally, a heterologous nucleic acid sequence coding for a dihydroxyphenylalanine ammonia-lyase (DAL) which is capable of producing caffeic acid from L-DOPA (3,4-dihydroxy-L-phenylalanine).

In another particular embodiment, the microorganism comprises:
a heterologous nucleic acid sequence coding for a flavanone 7-O-beta-D-glucosyltransferase (UGT) selected from enzymes comprising a sequence chosen from SEQ ID NO: 113 and polypeptides comprising a sequence having at least 60, 70, 80, 85, 90 or 95% sequence identity with this sequence and having flavanone 7-O-beta-D-glucosyltransferase activity;
a heterologous nucleic acid sequence coding for a 6″-O-rhamnosyltransferase (RhaT) comprising a sequence chosen from SEQ ID NO: 103 and polypeptides comprising a sequence having at least 60, 70, 80, 85, 90 or 95% sequence identity with this sequence and having 6″-O-rhamnosyltransferase activity; and
a heterologous nucleic acid sequence coding for a UDP-glucose 4,6-dehydratase/UDP-4-keto-6-deoxy-D-glucose 3,5-epimerase/UDP-4-keto-L-rhamnose-reductase (RHM) comprising a sequence chosen from SEQ ID NO: 107 and polypeptides comprising a sequence having at least 60, 70, 80, 85, 90 or 95% sequence identity with this sequence and having UDP-glucose 4,6-dehydratase/UDP-4-keto-6-deoxy-D-glucose 3,5-epimerase/UDP-4-keto-L-rhamnose-reductase activity;
a heterologous nucleic acid sequence coding for a phenylalanine ammonia lyase (PAL) comprising a sequence chosen from SEQ ID NO: 65 and polypeptides comprising a sequence having at least 60, 70, 80, 85, 90 or 95% sequence identity with this sequence and having phenylalanine ammonia lyase activity;
a heterologous nucleic acid sequence coding for a cinnamate 4-hydroxylase (C4H), comprising a sequence chosen from SEQ ID NO: 79 and polypeptides comprising a sequence having at least 60, 70, 80, 85, 90 or 95% sequence identity with this sequence and having cinnamate 4-hydroxylase activity;
a heterologous nucleic acid sequence coding for a tyrosine ammonia lyase (TAL) comprising a sequence chosen from SEQ ID NO: 41 and polypeptides comprising a sequence having at least 60, 70, 80, 85, 90 or 95% sequence identity with this sequence and having tyrosine ammonia lyase activity;
a heterologous nucleic acid sequence coding for a 4-coumaroyl-CoA ligase (4CL) comprising a sequence selected from SEQ ID NO: 45 or 123 and polypeptides comprising a sequence having at least 60, 70, 80, 85, 90 or 95% sequence identity with one of these sequences and having 4-coumarate-CoA ligase activity, preferably a sequence selected from SEQ ID NO: 45 and polypeptides comprising a sequence having at least 60, 70, 80, 85, 90 or 95% sequence identity with this sequence and having 4-coumarate-CoA ligase activity;
a heterologous nucleic acid sequence coding for a chalcone synthase (CHS) comprising a sequence chosen from SEQ ID NO: 53 and polypeptides comprising a sequence having at least 60, 70, 80, 85, 90 or 95% sequence identity with this sequence and having chalcone synthase activity;
a heterologous nucleic acid sequence coding for a chalcone isomerase (CHI) comprising a sequence chosen from SEQ ID NO: 61 and polypeptides comprising a sequence having at least 60, 70, 80, 85, 90 or 95% sequence identity with this sequence and having chalcone isomerase activity;
a heterologous nucleic acid sequence coding for a flavonoid 3′-monooxygenase (F3′H) comprising a sequence chosen from SEQ ID NO: 7 and polypeptides comprising a sequence having at least 60, 70, 80, 85, 90 or 95% sequence identity with the sequence SEQ ID NO: 7 and having flavonoid 3′-monooxygenase activity;
a heterologous nucleic acid sequence coding for a flavone synthase (FNS) comprising a sequence chosen from SEQ ID NO: 37 and a polypeptide comprising a sequence having at least 60, 70, 80, 85, 90 or 95% sequence identity with this sequence and having flavone synthase activity; and
a heterologous nucleic acid sequence coding for a cytochrome P450 reductase (CPR) comprising a sequence chosen from SEQ ID NO: 25 and polypeptides comprising a sequence having at least 60, 70, 80, 85, 90 or 95% sequence identity with one of these sequences and having cytochrome P450 reductase activity; and
a heterologous nucleic acid sequence coding for an O-methyltransferase (OMT) which is capable of methylating eriodictyol and/or luteolin in position 4′ and comprising a sequence chosen from SEQ ID NOs: 117 and 119 and polypeptides comprising a sequence having at least 60, 70, 80, 85, 90 or 95% sequence identity with one of these sequences and having O-methyltransferase activity, and preferably an OMT comprising a sequence chosen from SEQ ID NO: 117 and polypeptides comprising a sequence having at least 60, 70, 80, 85, 90 or 95% sequence identity with this sequence and having O-methyltransferase activity.

Preferably, the microorganism also comprises a heterologous or endogenous nucleic acid sequence coding for an S-adenosylmethionine synthetase (SAMT), in particular an SAMT comprising a sequence chosen from SEQ ID NO: 81 and a polypeptide comprising a sequence having at least 60, 70, 80, 85, 90 or 95% sequence identity with this sequence and having S-adenosylmethionine synthetase activity.

The origin of the enzymes or of a set of enzymes may be chosen so that their origin is the same or is similar. For example, the enzymes or the set of enzymes may be obtained from bacteria, for example from bacteria of the same genus or of the same species. In another example, the enzymes or the set of enzymes may be obtained from plants, for example from plants of the same genus or of the same species. The reason for this is that these common origins enable the enzymes to function together optimally.

In one embodiment, the microorganisms comprise a metabolic pathway for the biosynthesis of tyrosine. Notably, the microorganisms may have been modified to have increased production of tyrosine relative to a wild-type strain. Notably, the microorganisms may have been modified so that the carbon flow is redirected toward tyrosine biosynthesis. In addition, the microorganisms may have been modified to reduce or suppress the tyrosine biosynthesis feedback inhibitions.

In another embodiment, the microorganisms comprise a metabolic pathway for the biosynthesis of phenylalanine. Notably, the microorganisms may have been modified to have increased production of phenylalanine relative to a wild-type strain. Notably, the microorganisms may have been modified so that the carbon flow is redirected toward phenylalanine biosynthesis. In addition, the microorganisms may have been modified to reduce or suppress the phenylalanine biosynthesis feedback inhibitions.

In another embodiment, the microorganisms comprise a metabolic pathway for the biosynthesis of phenylalanine and tyrosine. Notably, the microorganisms may have been modified to have increased production of phenylalanine and tyrosine relative to a wild-type strain. Notably, the microorganisms may have been modified so that the carbon flow is redirected toward phenylalanine and tyrosine biosynthesis. In addition, the microorganisms may have been modified to reduce or suppress the phenylalanine and tyrosine biosynthesis feedback inhibitions.

### Recombinant Nucleic Acid and Expression Cassette

Each nucleic acid sequence coding for an enzyme as described previously is included in an expression cassette. Preferably, the coding nucleic acid sequences have been optimized for expression in the host microorganism. The coding nucleic acid sequence is operatively linked to the elements required for the expression of the gene, notably for transcription and translation. These elements are chosen so as to be functional in the host recombinant microorganism. These elements may include, for example, transcription promoters, transcription activators, terminator sequences, and start and stop codons. The methods for selecting these elements as a function of the host cell in which expression is desired are well known to those skilled in the art.

Preferably, the promoter is a strong promoter. The promoter may optionally be inducible.

For example, if the microorganism is prokaryotic, the promoter may be selected from the following promoters: Lacl, LacZ, pLacT, ptac, pARA, pBAD, the RNA polymerase promoters of bacteriophage T3 or T7, the polyhedrin promoter, the PR or PL promoter of lambda phage. In one particular embodiment, the promoter is pLac. If the microorganism is eukaryotic and in particular a yeast, the promoter may be selected from the following promoters: the promoter pTDH3, the promoter pTEF1, the promoter pTEF2, the promoter pCCW12, the promoter pHHF2, the promoter pHTB2 and the promoter pRPL18B. Examples of inducible promoters that may be used in yeast are the promoters tetO-2, GAL10, GAL10-CYC1 and PHO5.

All or part of the expression cassettes comprising the nucleic acid sequences coding for the enzymes as described or a combination of some of them may be included in a common expression vector or in different expression vectors.

The present invention thus relates to a vector comprising two nucleic acid sequences chosen from a heterologous nucleic acid sequence coding for a flavanone 7-O-beta-D-glucosyltransferase (UGT), a heterologous nucleic acid sequence coding for a 6″-O-rhamnosyltransferase (RhaT) and a heterologous nucleic acid sequence coding for a UDP-glucose 4,6-dehydratase/UDP-4-keto-6-deoxy-D-glucose 3,5-epimerase/UDP-4-keto-L-rhamnose-reductase (RHM); preferably, a vector comprises these three sequences.

In particular, the present invention thus relates to a vector comprising two nucleic acid sequences chosen from
(i) a heterologous nucleic acid sequence coding for a flavanone 7-O-beta-D-glucosyltransferase (UGT) from Arabidopsis thaliana, Scutellaria baicalensis or Homo sapiens, preferably from Arabidopsis thaliana or from Scutellaria baicalensis, preferably a flavanone 7-O-beta-D-glucosyltransferase (UGT) selected from enzymes comprising a sequence chosen from SEQ ID NOs: 113, 115, 91, 93, 95, 97, 99 and 101 and polypeptides comprising a sequence having at least 60, 70, 80, 85, 90 or 95% sequence identity with one of these sequences and having flavanone 7-O-beta-D-glucosyltransferase activity; in particular a flavanone 7-O-beta-D-glucosyltransferase (UGT) comprising a sequence chosen from SEQ ID NOs: 113, 115, 91, 93, 95 and 97 or from SEQ ID NOs: 113 and 95 and polypeptides comprising a sequence having at least 60, 70, 80, 85, 90 or 95% sequence identity with one of these sequences and having flavanone 7-O-beta-D-glucosyltransferase activity, preferably selected from enzymes comprising a sequence chosen from SEQ ID NO: 113 and polypeptides comprising a sequence having at least 60, 70, 80, 85, 90 or 95% sequence identity with this sequence and having flavanone 7-O-beta-D-glucosyltransferase activity;
(ii) a heterologous nucleic acid sequence coding for a 6″-O-rhamnosyltransferase (RhaT) of the genus Citrus or Petunia hybrida, preferably Citrus sinensis, Citrus maxima or Citrus clementina, even more preferably Citrus sinensis or Citrus clementina, preferably a 6″-O-rhamnosyltransferase (RhaT) comprising a sequence chosen from SEQ ID NOs: 103 and 105 and polypeptides comprising a sequence having at least 60, 70, 80, 85, 90 or 95% sequence identity with one of these sequences and having 6″-O-rhamnosyltransferase activity, and preferably an RhaT comprising a sequence chosen from SEQ ID NO: 103 and polypeptides comprising a sequence having at least 60, 70, 80, 85, 90 or 95% sequence identity with this sequence and having 6″-O-rhamnosyltransferase activity; and
(iii) a heterologous nucleic acid sequence coding for a UDP-glucose 4,6-dehydratase/UDP-4-keto-6-deoxy-D-glucose 3,5-epimerase/UDP-4-keto-L-rhamnose-reductase (RHM) from Citrus sinensis or from Arabidopsis thaliana, preferably an RHM comprising a sequence chosen from SEQ ID NOs: 107, 109 and 111 and polypeptides comprising a sequence having at least 60, 70, 80, 85, 90 or 95% sequence identity with one of these sequences and having UDP-glucose 4,6-dehydratase/UDP-4-keto-6-deoxy-D-glucose 3,5-epimerase/UDP-4-keto-L-rhamnose-reductase activity, in particular an RHM comprising a sequence selected from SEQ ID NOs: 107 and 109 and polypeptides comprising a sequence having at least 60, 70, 80, 85, 90 or 95% sequence identity with this sequence and having UDP-glucose 4,6-dehydratase/UDP-4-keto-6-deoxy-D-glucose 3,5-epimerase/UDP-4-keto-L-rhamnose-reductase activity, and preferably an RHM comprising a sequence selected from SEQ ID NO: 107 and polypeptides comprising a sequence having at least 60, 70, 80, 85, 90 or 95% sequence identity with this sequence and having UDP-glucose 4,6-dehydratase/UDP-4-keto-6-deoxy-D-glucose 3,5-epimerase/UDP-4-keto-L-rhamnose-reductase activity.

In particular, the vector may comprise two nucleic acid sequences chosen from
(i) a heterologous nucleic acid sequence coding for a flavanone 7-O-beta-D-glucosyltransferase (UGT) from Arabidopsis thaliana, Scutellaria baicalensis or Homo sapiens, preferably from Arabidopsis thaliana or from Scutellaria baicalensis, preferably a flavanone 7-O-beta-D-glucosyltransferase (UGT) selected from enzymes comprising a sequence chosen from SEQ ID NOs: 91, 93, 95, 97, 99 and 101 and polypeptides comprising a sequence having at least 60, 70, 80, 85, 90 or 95% sequence identity with one of these sequences and having flavanone 7-O-beta-D-glucosyltransferase activity; in particular a flavanone 7-O-beta-D-glucosyltransferase (UGT) comprising a sequence chosen from SEQ ID NOs: 91, 93, 95 and 97 and polypeptides comprising a sequence having at least 60, 70, 80, 85, 90 or 95% sequence identity with one of these sequences and having flavanone 7-O-beta-D-glucosyltransferase activity;
(ii) a heterologous nucleic acid sequence coding for a 6″-O-rhamnosyltransferase (RhaT) of the genus Citrus or Petunia hybrida, preferably Citrus sinensis, Citrus maxima or Citrus clementina, even more preferably Citrus sinensis or Citrus clementina, preferably a 6″-O-rhamnosyltransferase (RhaT) comprising a sequence chosen from SEQ ID NOs: 103 and 105 and polypeptides comprising a sequence having at least 60, 70, 80, 85, 90 or 95% sequence identity with one of these sequences and having 6″-O-rhamnosyltransferase activity; and
(iii) a heterologous nucleic acid sequence coding for a UDP-glucose 4,6-dehydratase/UDP-4-keto-6-deoxy-D-glucose 3,5-epimerase/UDP-4-keto-L-rhamnose-reductase (RHM) from Citrus sinensis or from Arabidopsis thaliana, preferably an RHM comprising a sequence chosen from SEQ ID NOs: 107, 109 and 111 and polypeptides comprising a sequence having at least 60, 70, 80, 85, 90 or 95% sequence identity with one of these sequences and having UDP-glucose 4,6-dehydratase/UDP-4-keto-6-deoxy-D-glucose 3,5-epimerase/UDP-4-keto-L-rhamnose-reductase activity, in particular an RHM comprising a sequence selected from SEQ ID NOs: 107 and 109 and polypeptides comprising a sequence having at least 60, 70, 80, 85, 90 or 95% sequence identity with this sequence and having UDP-glucose 4,6-dehydratase/UDP-4-keto-6-deoxy-D-glucose 3,5-epimerase/UDP-4-keto-L-rhamnose-reductase activity.

Preferably, the vector comprises two nucleic acid sequences chosen from
(i) a heterologous nucleic acid sequence coding for a flavanone 7-O-beta-D-glucosyltransferase (UGT) selected from enzymes comprising a sequence chosen from SEQ ID NOs: 113 and 95 and polypeptides comprising a sequence having at least 60, 70, 80, 85, 90 or 95% sequence identity with one of these sequences and having flavanone 7-O-beta-D-glucosyltransferase activity; preferably a flavanone 7-O-beta-D-glucosyltransferase (UGT) comprising a sequence chosen from SEQ ID NO: 113 and polypeptides comprising a sequence having at least 60, 70, 80, 85, 90 or 95% sequence identity with this sequence and having flavanone 7-O-beta-D-glucosyltransferase activity;
(ii) a heterologous nucleic acid sequence coding for a 6″-O-rhamnosyltransferase (RhaT) comprising a sequence chosen from SEQ ID NO: 103 and polypeptides comprising a sequence having at least 60, 70, 80, 85, 90 or 95% sequence identity with this sequence and having 6″-O-rhamnosyltransferase activity; and
(iii) a heterologous nucleic acid sequence coding for a UDP-glucose 4,6-dehydratase/UDP-4-keto-6-deoxy-D-glucose 3,5-epimerase/UDP-4-keto-L-rhamnose-reductase (RHM) comprising a sequence chosen from SEQ ID NO: 107 and polypeptides comprising a sequence having at least 60, 70, 80, 85, 90 or 95% sequence identity with this sequence and having UDP-glucose 4,6-dehydratase/UDP-4-keto-6-deoxy-D-glucose 3,5-epimerase/UDP-4-keto-L-rhamnose-reductase activity.

In a preferred embodiment, the microorganism comprises all three.

The coding nucleic acid sequences and the enzyme sequences are as described above. The term “comprising a nucleic acid sequence” also means comprising an expression cassette comprising the nucleic acid sequence.

Optionally, the vector may also comprise one or more nucleic acid sequences chosen from: a nucleic acid sequence coding for an O-methyltransferase (OMT), a nucleic acid sequence coding for an F3′H, a nucleic acid sequence coding for a CPR, a nucleic acid sequence coding for an FNS, a nucleic acid sequence coding for an SAMT, a nucleic acid sequence coding for a TAL, a nucleic acid sequence coding for a 4CL, a nucleic acid sequence coding for a CHS, a nucleic acid sequence coding for a CHI, a nucleic acid sequence coding for a PAL, a nucleic acid sequence coding for a C4H, a nucleic acid sequence coding for an HpaB, and a nucleic acid sequence coding for a DAL, each of these enzymes being as defined above, and also combinations thereof.

Preferably, the vector also comprises one or more sequences chosen from a nucleic acid sequence coding for an OMT, a nucleic acid sequence coding for an F3′H, a nucleic acid sequence coding for a CPR, a nucleic acid sequence coding for an FNS, a nucleic acid sequence coding for a TAL, a nucleic acid sequence coding for a 4CL, a nucleic acid sequence coding for a CHS, a nucleic acid sequence coding for a CHI, a nucleic acid sequence coding for a PAL and a nucleic acid sequence coding for a C4H.

In particular, the vector may also comprise one or more nucleic acid sequences chosen from:
a heterologous nucleic acid sequence coding for a flavonoid 3′-monooxygenase (F3′H) which is capable of hydroxylating naringenin and/or apigenin in position 3′; preferably from Perilla frutescens var. crispa, Petunia x hybrida, Callistephus chinensis, Gerbera hybrida, Citrus sinensis, Arabidopsis thaliana, Citrus clementina, Osteospermum hybrid cultivar, Phanerochaete chrysosporium, Streptomyces avermitilis or Pilosella officinarum, in particular from Perilla frutescens var. crispa, Petunia x hybrida, Callistephus chinensis, Gerbera hybrida, Citrus sinensis or Pilosella officinarum, preferably an F3′H comprising a sequence chosen from SEQ ID NOs: 1, 3, 5, 7, 9, 11, 13, 15, 17, 19, 21 and 121 and polypeptides comprising a sequence having at least 60, 70, 80, 85, 90 or 95% sequence identity with one of these sequences and having flavonoid 3′-monooxygenase activity, preferably selected from enzymes comprising a sequence chosen from SEQ ID NOs: 1, 5, 7, 11, 17, 19 and 121 and polypeptides comprising a sequence having at least 60, 70, 80, 85, 90 or 95% sequence identity with one of these sequences and having flavonoid 3′-monooxygenase activity, in particular an F3′H comprising a sequence selected from SEQ ID NOs: 5, 7, 17 and 121 and polypeptides comprising a sequence having at least 60, 70, 80, 85, 90 or 95% sequence identity with this sequence and having flavonoid 3′-monooxygenase activity, and preferably an enzyme comprising a sequence selected from SEQ ID NO: 7 and polypeptides comprising a sequence having at least 75%, at least 80%, at least 85%, at least 90% or at least 95% identity with this sequence and having flavonoid 3′-monooxygenase activity;
a heterologous nucleic acid sequence coding for a cytochrome P450 reductase (CPR); preferably a CPR from Saccharomyces cerevisiae, or from a plant, for example from Catharanthus roseus or Arabidopsis thaliana; preferably a CPR comprising a sequence chosen from SEQ ID NOs: 23, 25, 27, 29 and 31 and polypeptides comprising a sequence having at least 60, 70, 80, 85, 90 or 95% sequence identity with one of these sequences and having cytochrome P450 reductase activity, preferably from enzymes comprising a sequence chosen from SEQ ID NOs: 23, 25 and 29 and polypeptides comprising a sequence having at least 60, 70, 80, 85, 90 or 95% sequence identity with one of these sequences and having cytochrome P450 reductase activity, and preferably a CPR comprising a sequence chosen from SEQ ID NO: 25 and polypeptides comprising a sequence having at least 60, 70, 80, 85, 90 or 95% sequence identity with this sequence and having cytochrome P450 reductase activity;
a heterologous nucleic acid sequence coding for an O-methyltransferase (OMT) which is capable of methylating eriodictyol and/or luteolin in position 4′; preferably an OMT from Arabidopsis thaliana or Homo sapiens, preferably an OMT comprising a sequence chosen from SEQ ID NOs: 117, 119, 87 and 89 and polypeptides comprising a sequence having at least 60, 70, 80, 85, 90 or 95% sequence identity with one of these sequences and having O-methyltransferase activity, notably with eriodictyol and/or luteolin as substrate and with methylation in position 4′, preferably selected from the enzyme comprising a sequence chosen from SEQ ID NOs: 117, 119 and 89 and polypeptides comprising a sequence having at least 60, 70, 80, 85, 90 or 95% sequence identity with this sequence and having O-methyltransferase activity;
a heterologous nucleic acid sequence coding for a flavone synthase (FNS) which is capable of transforming naringenin into apigenin, eriodictyol into luteolin, and/or hesperetin into diosmetin, preferably of transforming eriodictyol into luteolin; preferably an FNS from Arabidopsis thaliana, Lonicera japonica, Lonicera macranthoides, Medicago truncatula, Oryza sativa, Petroselinum crispum, Populus deltoides, Zea mays, Callistephus chinensis, Apium graveolens, Medicago truncatula, Cuminum cyminum, Aethusa cynapium, Angelica archangelica, Conium maculatum, Camellia sinensis, Cynara cardunculus var scolymus, Saussurea medusa, Plectranthus barbatus, Scutellaria baicalensis, Dorcoceras hygrometricum, Antirrhinum majus, Perilla frutescens var crispa, Dahlia pinnata or Erythranthe lewisii, in particular from Arabidopsis thaliana, Lonicera japonica, Lonicera macranthoides, Medicago truncatula, Oryza sativa, Petroselinum crispum, Populus deltoides or Zea mays, preferably from Lonicera japonica, Lonicera macranthoides and Petroselinum crispum; preferably an FNS comprising a sequence chosen from SEQ ID NOs: 33, 35, 37, 127, 129, 131, 133, 135, 137, 139, 141, 143, 145, 147, 149, 151, 153, 155, 157 and 159 and polypeptides comprising a sequence having at least 60, 70, 80, 85, 90 or 95% sequence identity with one of these sequences and having flavone synthase activity, preferably from enzymes comprising a sequence chosen from SEQ ID NOs: 33, 35 and 37 and polypeptides comprising a sequence having at least 60, 70, 80, 85, 90 or 95% sequence identity with one of these sequences and having flavone synthase activity, preferably a flavone synthase (FNS) comprising a sequence chosen from SEQ ID NO: 37 and a polypeptide comprising a sequence having at least 60, 70, 80, 85, 90 or 95% sequence identity with this sequence and having flavone synthase activity;
a heterologous or endogenous nucleic acid sequence coding for an S-adenosylmethionine synthetase (SAMT), in particular from Saccharomyces cerevisiae, for example an SAMT comprising a sequence chosen from SEQ ID NO: 81 and a polypeptide comprising a sequence having at least 60, 70, 80, 85, 90 or 95% sequence identity with this sequence and having S-adenosylmethionine synthetase activity;
a heterologous nucleic acid sequence coding for a tyrosine ammonia lyase (TAL) which is capable of producing p-coumaric acid from tyrosine; preferably from Rhodotorula glutinis or Flavobacterium johnsoniae; in particular a TAL comprising a sequence chosen from SEQ ID NOs: 39 and 41 and polypeptides comprising a sequence having at least 60, 70, 80, 85, 90 or 95% sequence identity with one of these sequences and having tyrosine ammonia lyase activity; preferably a TAL comprising a sequence chosen from SEQ ID NO: 41 and polypeptides comprising a sequence having at least 60, 70, 80, 85, 90 or 95% sequence identity with this sequence and having tyrosine ammonia lyase activity;
a heterologous nucleic acid sequence coding for a 4-coumarate-CoA ligase (4CL) which is capable of producing coumaryl-CoA from p-coumaric acid and Coenzyme A; preferably from Arabidopsis thaliana, Citrus clementina, Petroselinum crispum or Streptomyces clavuligerus; a 4CL comprising a sequence chosen from SEQ ID NOs: 43, 45, 47, 49, 123 and 125 and polypeptides comprising a sequence having at least 60, 70, 80, 85, 90 or 95% sequence identity with one of these sequences and having 4-coumarate-CoA ligase activity, preferably a 4CL comprising a sequence selected from SEQ ID NOs: 123, 125 and 45 and polypeptides comprising a sequence having at least 60, 70, 80, 85, 90 or 95% sequence identity with one of these sequences and having 4-coumarate-CoA ligase activity, and in particular a 4CL comprising a sequence selected from SEQ ID NO: 45 and polypeptides comprising a sequence having at least 60, 70, 80, 85, 90 or 95% sequence identity with this sequence and having 4-coumarate-CoA ligase activity;
a heterologous nucleic acid sequence coding for a chalcone synthase (CHS) which is capable of producing naringenin-chalcone from 4-coumaroyl-CoA and malonyl-CoA; preferably from Citrus sinensis, Hordeum vulgare or Streptomyces clavuligerus, in particular a CHS comprising a sequence chosen from SEQ ID NOs: 51, 53, 55 and 57 and polypeptides comprising a sequence having at least 60, 70, 80, 85, 90 or 95% sequence identity with one of these sequences and having chalcone synthase activity, preferably from enzymes comprising a sequence chosen from SEQ ID NOs: 53 and 55 and polypeptides comprising a sequence having at least 60, 70, 80, 85, 90 or 95% sequence identity with one of these sequences and having chalcone synthase activity, and preferably a CHS comprising a sequence chosen from SEQ ID NO: 53 and polypeptides comprising a sequence having at least 60, 70, 80, 85, 90 or 95% sequence identity with this sequence and having chalcone synthase activity;
a heterologous nucleic acid sequence coding for a chalcone isomerase (CHI) which is capable of producing naringenin from naringenin chalcone; preferably from Arabidopsis thaliana or Streptomyces clavuligerus, in particular a CHI comprising a sequence chosen from SEQ ID NOs: 59 and 61 and polypeptides comprising a sequence having at least 60, 70, 80, 85, 90 or 95% sequence identity with one of these sequences and having chalcone isomerase activity, and preferably a CHI comprising a sequence chosen from SEQ ID NO: 61 and polypeptides comprising a sequence having at least 60, 70, 80, 85, 90 or 95% sequence identity with this sequence and having chalcone isomerase activity;
a heterologous nucleic acid sequence coding for a phenylalanine ammonia lyase (PAL), in particular a PAL comprising a sequence chosen from SEQ ID NOs: 63, 65 and 77 and polypeptides comprising a sequence having at least 60, 70, 80, 85, 90 or 95% sequence identity with one of these sequences and having phenylalanine ammonia lyase activity, preferably a PAL comprising a sequence chosen from SEQ ID NO: 65 and polypeptides comprising a sequence having at least 60, 70, 80, 85, 90 or 95% sequence identity with this sequence and having phenylalanine ammonia lyase activity; and
a heterologous nucleic acid sequence coding for a cinnamate 4-hydroxylase (C4H), in particular a C4H comprising a sequence chosen from SEQ ID NOs: 67, 69 and 79 and polypeptides comprising a sequence having at least 60, 70, 80, 85, 90 or 95% sequence identity with one of these sequences and having cinnamate 4-hydroxylase activity, which are capable of producing p-coumaric acid from phenylalanine, and preferably a C4H comprising a sequence chosen from SEQ ID NO: 79 and polypeptides comprising a sequence having at least 60, 70, 80, 85, 90 or 95% sequence identity with this sequence and having cinnamate 4-hydroxylase activity;
a nucleic acid sequence coding for a 4-hydroxyphenylacetate 3-monooxygenase oxygenase subunit (HpaB), preferably comprising a sequence chosen from SEQ ID NO: 83 and polypeptides comprising a sequence having at least 60, 70, 80, 85, 90 or 95% sequence identity therewith and having 4-hydroxyphenylacetate 3-monooxygenase oxygenase activity, and a nucleic acid sequence coding for a 4-hydroxyphenylacetate 3-monooxygenase reductase subunit (HpaC), preferably comprising a sequence chosen from SEQ ID NO: 85 and polypeptides comprising a sequence having at least 60, 70, 80, 85, 90 or 95% sequence identity therewith and having 4-hydroxyphenylacetate 3-monooxygenase reductase activity; or a nucleic acid sequence coding for a 4-methoxybenzoate O-demethylase which is capable of converting tyrosine into L-DOPA and also p-coumaric acid into caffeic acid, preferably comprising a sequence chosen from SEQ ID NOs: 73 and 75 and polypeptides comprising a sequence having at least 60, 70, 80, 85, 90 or 95% sequence identity with one of these sequences and having L-tyrosine hydrolase activity; or a heterologous nucleic acid sequence coding for a p-coumarate 3-hydroxylase which is capable of converting p-coumaric acid into caffeic acid, preferably comprising a sequence SEQ ID NO: 71 and polypeptides comprising a sequence having at least 60, 70, 80, 85, 90 or 95% sequence identity with one of these sequences and having p-coumarate 3-hydroxylase activity, and
a nucleic acid sequence coding for a dihydroxyphenylalanine ammonia-lyase (DAL).

In particular, the vector may also comprise one or more nucleic acid sequences chosen from:
a heterologous nucleic acid sequence coding for a flavonoid 3′-monooxygenase (F3′H) which is capable of hydroxylating naringenin and/or apigenin in position 3′; preferably from Perilla frutescens var. crispa, Petunia x hybrida, Callistephus chinensis, Gerbera hybrida, Citrus sinensis and Pilosella officinarum, preferably an F3′H comprising a sequence chosen from SEQ ID NOs: 1, 3, 5, 7, 9, 11, 13, 15, 17, 19 and 21 and polypeptides comprising a sequence having at least 60, 70, 80, 85, 90 or 95% sequence identity with one of these sequences and having flavonoid 3′-monooxygenase activity, preferably selected from enzymes comprising a sequence chosen from SEQ ID NOs: 1, 5, 7, 11, 17 and 19 and polypeptides comprising a sequence having at least 60, 70, 80, 85, 90 or 95% sequence identity with one of these sequences and having flavonoid 3′-monooxygenase activity, in particular an F3′H comprising a sequence selected from SEQ ID NOs: 5, 7 and 17 and polypeptides comprising a sequence having at least 60, 70, 80, 85, 90 or 95% sequence identity with this sequence and having flavonoid 3′-monooxygenase activity;
a heterologous nucleic acid sequence coding for a cytochrome P450 reductase (CPR); preferably a CPR from Saccharomyces cerevisiae, or from a plant, for example from Catharanthus roseus or Arabidopsis thaliana; preferably a CPR comprising a sequence chosen from SEQ ID NOs: 23, 25, 27, 29 and 31 and polypeptides comprising a sequence having at least 60, 70, 80, 85, 90 or 95% sequence identity with one of these sequences and having cytochrome P450 reductase activity, preferably from enzymes comprising a sequence chosen from SEQ ID NOs: 23, 25 and 29 and polypeptides comprising a sequence having at least 60, 70, 80, 85, 90 or 95% sequence identity with one of these sequences and having cytochrome P450 reductase activity;
a heterologous nucleic acid sequence coding for an O-methyltransferase (OMT) which is capable of methylating eriodictyol and/or luteolin in position 4′; preferably an OMT from Arabidopsis thaliana or Homo sapiens, preferably an OMT comprising a sequence chosen from SEQ ID NOs: 87 and 89 and polypeptides comprising a sequence having at least 60, 70, 80, 85, 90 or 95% sequence identity with one of these sequences and having O-methyltransferase activity, notably with eriodictyol and/or luteolin as substrate and with methylation in position 4′, preferably selected from the enzyme comprising the sequence SEQ ID NO: 89 and polypeptides comprising a sequence having at least 60, 70, 80, 85, 90 or 95% sequence identity with this sequence and having O-methyltransferase activity;
a heterologous nucleic acid sequence coding for a flavone synthase (FNS) which is capable of transforming naringenin into apigenin, eriodictyol into luteolin and/or hesperetin into diosmetin, preferably of transforming eriodictyol into luteolin; preferably an FNS from Arabidopsis thaliana, Lonicera japonica, Lonicera macranthoides, Medicago truncatula, Oryza sativa, Petroselinum crispum, Populus deltoides or Zea mays, preferably from Lonicera japonica, Lonicera macranthoides and Petroselinum crispum; preferably an FNS comprising a sequence chosen from SEQ ID NOs: 33, 35 and 37 and polypeptides comprising a sequence having at least 60, 70, 80, 85, 90 or 95% sequence identity with one of these sequences and having flavone synthase activity, preferably from enzymes comprising a sequence chosen from SEQ ID NOs: 33, 35 and 37 and polypeptides comprising a sequence having at least 60, 70, 80, 85, 90 or 95% sequence identity with one of these sequences and having flavone synthase activity, preferably a flavone synthase (FNS) comprising a sequence chosen from SEQ ID NO: 37 and a polypeptide comprising a sequence having at least 60, 70, 80, 85, 90 or 95% sequence identity with this sequence and having flavone synthase activity;
a heterologous or endogenous nucleic acid sequence coding for an S-adenosylmethionine synthetase (SAMT), in particular from Saccharomyces cerevisiae, for example an SAMT comprising a sequence chosen from SEQ ID NO: 81 and a polypeptide comprising a sequence having at least 60, 70, 80, 85, 90 or 95% sequence identity with this sequence and having S-adenosylmethionine synthetase activity;
a heterologous nucleic acid sequence coding for a tyrosine ammonia lyase (TAL) which is capable of producing p-coumaric acid from tyrosine; preferably from Rhodotorula glutinis or Flavobacterium johnsoniae; in particular a TAL comprising a sequence chosen from SEQ ID NOs: 39 and 41 and polypeptides comprising a sequence having at least 60, 70, 80, 85, 90 or 95% sequence identity with one of these sequences and having tyrosine ammonia lyase activity; preferably a TAL comprising a sequence chosen from SEQ ID NO: 41 and polypeptides comprising a sequence having at least 60, 70, 80, 85, 90 or 95% sequence identity with this sequence and having tyrosine ammonia lyase activity;
a heterologous nucleic acid sequence coding for a 4-coumarate-CoA ligase (4CL) which is capable of producing coumaryl-CoA from p-coumaric acid and Coenzyme A; preferably from Arabidopsis thaliana, Petroselinum crispum or Streptomyces clavuligerus; a 4CL comprising a sequence chosen from SEQ ID NOs: 43, 45, 47 and 49 and polypeptides comprising a sequence having at least 60, 70, 80, 85, 90 or 95% sequence identity with one of these sequences and having 4-coumarate-CoA ligase activity;
a heterologous nucleic acid sequence coding for a chalcone synthase (CHS) which is capable of producing naringenin-chalcone from 4-coumaroyl-CoA and malonyl-CoA; preferably from Citrus sinensis, Hordeum vulgare or Streptomyces clavuligerus, in particular a CHS comprising a sequence chosen from SEQ ID NOs: 51, 53, 55 and 57 and polypeptides comprising a sequence having at least 60, 70, 80, 85, 90 or 95% sequence identity with one of these sequences and having chalcone synthase activity, preferably from enzymes comprising a sequence chosen from SEQ ID NOs: 53 and 55 and polypeptides comprising a sequence having at least 60, 70, 80, 85, 90 or 95% sequence identity with one of these sequences and having chalcone synthase activity;
a heterologous nucleic acid sequence coding for a chalcone isomerase (CHI) which is capable of producing naringenin from naringenin chalcone; preferably from Arabidopsis thaliana or Streptomyces clavuligerus, in particular a CHI comprising a sequence chosen from SEQ ID NOs: 59 and 61 and polypeptides comprising a sequence having at least 60, 70, 80, 85, 90 or 95% sequence identity with one of these sequences and having chalcone isomerase activity;
a heterologous nucleic acid sequence coding for a phenylalanine ammonia lyase (PAL), in particular a PAL comprising a sequence chosen from SEQ ID NOs: 63, 65 and 77 and polypeptides comprising a sequence having at least 60, 70, 80, 85, 90 or 95% sequence identity with one of these sequences and having phenylalanine ammonia lyase activity; and
a heterologous nucleic acid sequence coding for a cinnamate 4-hydroxylase (C4H), in particular a C4H comprising a sequence chosen from SEQ ID NOs: 67, 69 and 79 and polypeptides comprising a sequence having at least 60, 70, 80, 85, 90 or 95% sequence identity with one of these sequences and having cinnamate 4-hydroxylase activity, which are capable of producing p-coumaric acid from phenylalanine;
a nucleic acid sequence coding for a 4-hydroxyphenylacetate 3-monooxygenase oxygenase subunit (HpaB), preferably comprising a sequence chosen from SEQ ID NO: 83 and polypeptides comprising a sequence having at least 60, 70, 80, 85, 90 or 95% sequence identity therewith and having 4-hydroxyphenylacetate 3-monooxygenase oxygenase activity, and a nucleic acid sequence coding for a 4-hydroxyphenylacetate 3-monooxygenase reductase subunit (HpaC), preferably comprising a sequence chosen from SEQ ID NO: 85 and polypeptides comprising a sequence having at least 60, 70, 80, 85, 90 or 95% sequence identity therewith and having 4-hydroxyphenylacetate 3-monooxygenase reductase activity; or a nucleic acid sequence coding for a 4-methoxybenzoate O-demethylase which is capable of converting tyrosine into L-DOPA and also p-coumaric acid into caffeic acid, preferably comprising a sequence chosen from SEQ ID NOs: 73 and 75 and polypeptides comprising a sequence having at least 60, 70, 80, 85, 90 or 95% sequence identity with one of these sequences and having L-tyrosine hydrolase activity; or a heterologous nucleic acid sequence coding for a p-coumarate 3-hydroxylase which is capable of converting p-coumaric acid into caffeic acid, preferably comprising a sequence SEQ ID NO: 71 and polypeptides comprising a sequence having at least 60, 70, 80, 85, 90 or 95% sequence identity with one of these sequences and having p-coumarate 3-hydroxylase activity, and
a nucleic acid sequence coding for a dihydroxyphenylalanine ammonia-lyase (DAL).

Preferably, the vector may also comprise one or more nucleic acid sequences chosen from:
a heterologous nucleic acid sequence coding for a flavonoid 3′-monooxygenase (F3′H) comprising a sequence chosen from SEQ ID NOs: 7, 11, 17 and 121 and polypeptides comprising a sequence having at least 60, 70, 80, 85, 90 or 95% sequence identity with one of these sequences and having flavonoid 3′-monooxygenase activity, preferably selected from enzymes comprising a sequence chosen from SEQ ID NOs: 7, 17 and 121 and polypeptides comprising a sequence having at least 60, 70, 80, 85, 90 or 95% sequence identity with one of these sequences and having flavonoid 3′-monooxygenase activity, and most particularly preferably an F3′H comprising a sequence selected from SEQ ID NO: 7 and polypeptides comprising a sequence having at least 60, 70, 80, 85, 90 or 95% sequence identity with this sequence and having flavonoid 3′-monooxygenase activity;
a heterologous nucleic acid sequence coding for a cytochrome P450 reductase (CPR) comprising a sequence chosen from SEQ ID NOs: 23, 25, 27, 29 and 31 and polypeptides comprising a sequence having at least 60, 70, 80, 85, 90 or 95% sequence identity with one of these sequences and having cytochrome P450 reductase activity, preferably from enzymes comprising a sequence chosen from SEQ ID NOs: 23, 25 and 29 and polypeptides comprising a sequence having at least 60, 70, 80, 85, 90 or 95% sequence identity with one of these sequences and having cytochrome P450 reductase activity, and most particularly preferably from enzymes comprising a sequence chosen from SEQ ID NO: 25 and polypeptides comprising a sequence having at least 60, 70, 80, 85, 90 or 95% sequence identity with this sequence and having cytochrome P450 reductase activity;
a heterologous nucleic acid sequence coding for an O-methyltransferase (OMT) which is capable of methylating eriodictyol and/or luteolin in position 4′ and comprising a sequence chosen from SEQ ID NOs: 117 and 119 and polypeptides comprising a sequence having at least 60, 70, 80, 85, 90 or 95% sequence identity with one of these sequences and having O-methyltransferase activity, notably with eriodictyol and/or luteolin as substrate and with methylation in position 4′, preferably selected from the enzyme comprising a sequence chosen from SEQ ID NO: 117 and polypeptides comprising a sequence having at least 60, 70, 80, 85, 90 or 95% sequence identity with this sequence and having O-methyltransferase activity;
a heterologous nucleic acid sequence coding for a flavone synthase (FNS) comprising a sequence chosen from SEQ ID NO: 37 and a polypeptide comprising a sequence having at least 60, 70, 80, 85, 90 or 95% sequence identity with this sequence and having flavone synthase activity;
a heterologous or endogenous nucleic acid sequence coding for an S-adenosylmethionine synthetase (SAMT) comprising a sequence chosen from SEQ ID NO: 81 and a polypeptide having at least 60, 70, 80, 85, 90 or 95% sequence identity with this sequence and having S-adenosylmethionine synthetase activity;
a heterologous nucleic acid sequence coding for a tyrosine ammonia lyase (TAL) which is capable of producing p-coumaric acid from tyrosine and comprising a sequence chosen from SEQ ID NO: 41 and polypeptides comprising a sequence having at least 60, 70, 80, 85, 90 or 95% sequence identity with this sequence and having tyrosine ammonia lyase activity;
a heterologous nucleic acid sequence coding for a 4-coumarate-CoA ligase (4CL) which is capable of producing coumaryl-CoA from p-coumaric acid and Coenzyme A and comprising a sequence chosen from SEQ ID NOs: 123, 125 and 45 and polypeptides comprising a sequence having at least 60, 70, 80, 85, 90 or 95% sequence identity with one of these sequences and having 4-coumarate-CoA ligase activity, preferably comprising a sequence selected from SEQ ID NO: 45 and polypeptides comprising a sequence having at least 60, 70, 80, 85, 90 or 95% sequence identity with this sequence and having 4-coumarate-CoA ligase activity;
a heterologous nucleic acid sequence coding for a chalcone synthase (CHS) which is capable of producing naringenin-chalcone from 4-coumaroyl-CoA and malonyl-CoA and comprising a sequence chosen from SEQ ID NO: 53 and polypeptides comprising a sequence having at least 60, 70, 80, 85, 90 or 95% sequence identity with this sequence and having chalcone synthase activity;
a heterologous nucleic acid sequence coding for a chalcone isomerase (CHI) which is capable of producing naringenin from naringenin chalcone and comprising a sequence chosen from SEQ ID NO: 61 and polypeptides comprising a sequence having at least 60, 70, 80, 85, 90 or 95% sequence identity with this sequence and having chalcone isomerase activity;
a heterologous nucleic acid sequence coding for a phenylalanine ammonia lyase (PAL) and comprising a sequence chosen from SEQ ID NOs: 65 and 77 and polypeptides comprising a sequence having at least 60, 70, 80, 85, 90 or 95% sequence identity with one of these sequences and having phenylalanine ammonia lyase activity, preferably comprising a sequence chosen from SEQ ID NO: 65 and polypeptides comprising a sequence having at least 60, 70, 80, 85, 90 or 95% sequence identity with this sequence and having phenylalanine ammonia lyase activity; and
a heterologous nucleic acid sequence coding for a cinnamate 4-hydroxylase (C4H) and comprising a sequence chosen from SEQ ID NO: 79 and polypeptides comprising a sequence having at least 60, 70, 80, 85, 90 or 95% sequence identity with this sequence and having cinnamate 4-hydroxylase activity, which are capable of producing p-coumaric acid from phenylalanine.

In a particular embodiment, the vector comprises:
a nucleic acid sequence coding for an O-methyltransferase, in particular which is capable of methylating eriodictyol and/or luteolin in position 4′ and a heterologous nucleic acid sequence coding for a flavonoid 3′-monooxygenase (F3′H), in particular which is capable of adding a hydroxyl in position 3′ of naringenin and/or apigenin; or
a nucleic acid sequence coding for an O-methyltransferase (OMT), in particular which is capable of methylating eriodictyol and/or luteolin in position 4′; a heterologous nucleic acid sequence coding for a flavonoid 3′-monooxygenase (F3′H), in particular which is capable of adding a hydroxyl in position 3′ of naringenin and/or apigenin; and a heterologous nucleic acid sequence coding for a cytochrome P450 reductase; or
a heterologous nucleic acid sequence coding for an O-methyltransferase (OMT), in particular which is capable of methylating eriodictyol and/or luteolin in position 4′; and a heterologous nucleic acid sequence coding for a flavone synthase (FNS), which is capable of transforming naringenin into apigenin, eriodictyol into luteolin and/or hesperetin into diosmetin, preferably of transforming eriodictyol into luteolin; or
a heterologous nucleic acid sequence coding for an O-methyltransferase which is capable of methylating eriodictyol and/or luteolin in position 4′; a heterologous nucleic acid sequence coding for a flavonoid 3′-monooxygenase (F3′H) which is capable of hydroxylating in position 3′ of naringenin and/or apigenin; a heterologous nucleic acid sequence coding for a cytochrome P450 reductase; and a heterologous nucleic acid sequence coding for flavone synthase (FNS) which is capable of transforming naringenin into apigenin, eriodictyol into luteolin and/or hesperetin into diosmetin, preferably of transforming eriodictyol into luteolin.

The vector may thus comprise several nucleic acid sequences chosen therefrom, notably 1, 2, 3, 4, 5, 6, 7, 8, 9 or 10 nucleic acid sequences chosen therefrom.

The vector may notably comprise combinations of particular coding sequences as described above.

The vectors comprise coding sequences that are heterologous insofar as the coding sequences may be optimized for the host microorganism, may be under the control of a heterologous promoter and/or may combine coding sequences which do not originate from the same original organism and/or which are not present in the same arrangement.

The vector may be any DNA sequence in which it is possible to insert foreign nucleic acids, the vectors making it possible to introduce foreign DNA into the host microorganism. For example, the vector may be a plasmid, a phagemid, a cosmid, an artificial chromosome, notably a YAC, or a BAC.

The expression vectors may comprise nucleic acid sequences coding for selection markers. The selection markers may be genes for resistance to one or more antibiotics or auxotrophic genes. The auxotrophic gene may be, for example, URA3, LEU2, HIS3 or TRP1. The antibiotic-resistance gene may preferably be, for example, a gene for resistance to ampicillin, kanamycin, hygromycin, geneticin and/or nourseothricin.

The introduction of vectors into a host microorganism is a process that is widely known to those skilled in the art. Several methods are notably described in “Current Protocols in Molecular Biology”, 13.7.1-13.7.10; or in Ellis T. et al., Integrative Biology, 2011, 3(2), 109-118.

The host microorganism may be transiently or stably transformed/transfected and the nucleic acid, the cassette or the vector may be contained therein in episomal form or in a form incorporated into the genome of the host microorganism.

The expression vector may also comprise one or more sequences allowing the targeted insertion of the vector, of the expression cassette or of the nucleic acid into the genome of the host microorganism.

All or part of the expression cassettes comprising the nucleic acid sequences coding for the enzymes as described above or a combination of some of them may be inserted into the/a chromosome of the recombinant microorganism.

Conversely, all or part of the expression cassettes comprising the nucleic acid sequences coding for the enzymes as described or a combination of some of them may be conserved in episomal form, notably in plasmid form.

Optionally, the microorganism may comprise several copies of nucleic acid sequences coding for an enzyme as described previously. Notably, it may comprise 2 to 10 copies, for example 2, 3, 4, 5, 6, 7, 8, 9 or 10 copies of a nucleic acid sequence coding for an enzyme as described previously.

The present invention relates to a method for preparing a microorganism according to the present invention, comprising the introduction of nucleic acid sequences coding for a flavanone 7-O-beta-D-glucosyltransferase (UGT), in particular which is capable of adding a glucose in position 7 of hesperetin and diosmetin; for a 6″-O-rhamnosyltransferase (RhaT), in particular which is capable of transferring a rhamnose in position 6 of the glucose of hesperetin-7-O-glucoside and/or diosmetin-7-O-glucoside; and for a UDP-glucose 4,6-dehydratase/UDP-4-keto-6-deoxy-D-glucose 3,5-epimerase/UDP-4-keto-L-rhamnose-reductase (RHM), in particular which is capable of producing UDP-rhamnose in the microorganism and the selection of microorganisms comprising said nucleic acid sequences.

The method may also comprise the introduction of one or more nucleic acid sequences chosen from:
a heterologous nucleic acid sequence coding for a flavonoid 3′-monooxygenase (F3′H) which is capable of hydroxylating naringenin and/or apigenin in position 3′; preferably from Perilla frutescens var. crispa, Petunia x hybrida, Callistephus chinensis, Gerbera hybrida, Citrus sinensis, Citrus clementina, Osteospermum hybrid cultivar, Phanerochaete chrysosporium, Streptomyces avermitilis or Pilosella officinarum, in particular from Perilla frutescens var. crispa, Petunia x hybrida, Callistephus chinensis, Gerbera hybrida, Citrus sinensis or Pilosella officinarum, preferably an F3′H comprising a sequence chosen from SEQ ID NOs: 1, 3, 5, 7, 9, 11, 13, 15, 17, 19 and 21 and polypeptides comprising a sequence having at least 60, 70, 80, 85, 90 or 95% sequence identity with one of these sequences and having flavonoid 3′-monooxygenase activity, preferably selected from enzymes comprising a sequence chosen from SEQ ID NOs: 1, 5, 7, 11, 17 and 19 and polypeptides comprising a sequence having at least 60, 70, 80, 85, 90 or 95% sequence identity with one of these sequences and having flavonoid 3′-monooxygenase activity, in particular an F3′H comprising a sequence selected from SEQ ID NOs: 5, 7 and 17 and polypeptides comprising a sequence having at least 60, 70, 80, 85, 90 or 95% sequence identity with this sequence and having flavonoid 3′-monooxygenase activity, preferably an F3′H comprising a sequence chosen from SEQ ID NOs: 7 and polypeptides comprising a sequence having at least 60, 70, 80, 85, 90 or 95% sequence identity with the sequence SEQ ID NO: 7 and having flavonoid 3′-monooxygenase activity;
a heterologous nucleic acid sequence coding for a cytochrome P450 reductase (CPR); preferably a CPR from Saccharomyces cerevisiae, or from a plant, for example from Catharanthus roseus or Arabidopsis thaliana; preferably a CPR comprising a sequence chosen from SEQ ID NOs: 23, 25, 27, 29 and 31 and polypeptides comprising a sequence having at least 60, 70, 80, 85, 90 or 95% sequence identity with one of these sequences and having cytochrome P450 reductase activity, preferably from enzymes comprising a sequence chosen from SEQ ID NOs: 23, 25, 29 and 31 and polypeptides comprising a sequence having at least 60, 70, 80, 85, 90 or 95% sequence identity with one of these sequences and having cytochrome P450 reductase activity, and particularly a CPR comprising a sequence chosen from SEQ ID NO: 25 and polypeptides comprising a sequence having at least 60, 70, 80, 85, 90 or 95% sequence identity with this sequence and having cytochrome P450 reductase activity;
a heterologous nucleic acid sequence coding for an O-methyltransferase (OMT) which is capable of methylating eriodictyol and/or luteolin in position 4′;
preferably an OMT from Arabidopsis thaliana or Homo sapiens, preferably an OMT comprising a sequence chosen from SEQ ID NOs: 87 and 89 and polypeptides comprising a sequence having at least 60, 70, 80, 85, 90 or 95% sequence identity with one of these sequences and having O-methyltransferase activity, notably with eriodictyol and/or luteolin as substrate and with methylation in position 4′, preferably selected from the enzyme comprising a sequence chosen from SEQ ID NO: 89 and polypeptides comprising a sequence having at least 60, 70, 80, 85, 90 or 95% sequence identity with this sequence and having O-methyltransferase activity;
a heterologous nucleic acid sequence coding for a flavone synthase (FNS) which is capable of transforming naringenin into apigenin, eriodictyol into luteolin, and/or hesperetin into diosmetin, preferably of transforming eriodictyol into luteolin; preferably an FNS from Arabidopsis thaliana, Lonicera japonica, Lonicera macranthoides, Medicago truncatula, Oryza sativa, Petroselinum crispum, Populus deltoides, Zea mays, Callistephus chinensis, Apium graveolens, Medicago truncatula, Cuminum cyminum, Aethusa cynapium, Angelica archangelica, Conium maculatum, Camellia sinensis, Cynara cardunculus var scolymus, Saussurea medusa, Plectranthus barbatus, Scutellaria baicalensis, Dorcoceras hygrometricum, Antirrhinum majus, Perilla frutescens var crispa, Dahlia pinnata or Erythranthe lewisii, in particular from Arabidopsis thaliana, Lonicera japonica, Lonicera macranthoides, Medicago truncatula, Oryza sativa, Petroselinum crispum, Populus deltoides or Zea mays, preferably from Lonicera japonica, Lonicera macranthoides and Petroselinum crispum; preferably an FNS comprising a sequence chosen from SEQ ID NOs: 33, 35, 37, 127, 129, 131, 133, 135, 137, 139, 141, 143, 145, 147, 149, 151, 153, 155, 157 and 159 and polypeptides comprising a sequence having at least 60, 70, 80, 85, 90 or 95% sequence identity with one of these sequences and having flavone synthase activity, preferably from enzymes comprising a sequence chosen from SEQ ID NOs: 33, 35 and 37 and polypeptides comprising a sequence having at least 60, 70, 80, 85, 90 or 95% sequence identity with one of these sequences and having flavone synthase activity, preferably a flavone synthase (FNS) comprising a sequence chosen from SEQ ID NO: 37 and a polypeptide comprising a sequence having at least 60, 70, 80, 85, 90 or 95% sequence identity with this sequence and having flavone synthase activity;
a heterologous or endogenous nucleic acid sequence coding for an S-adenosylmethionine synthetase (SAMT), in particular from Saccharomyces cerevisiae, for example an SAMT comprising a sequence chosen from SEQ ID NO: 81 and a polypeptide comprising a sequence having at least 60, 70, 80, 85, 90 or 95% sequence identity with this sequence and having S-adenosylmethionine synthetase activity;
a heterologous nucleic acid sequence coding for a tyrosine ammonia lyase (TAL) which is capable of producing p-coumaric acid from tyrosine; preferably from Rhodotorula glutinis or Flavobacterium johnsoniae; in particular a TAL comprising a sequence chosen from SEQ ID NOs: 39 and 41 and polypeptides comprising a sequence having at least 60, 70, 80, 85, 90 or 95% sequence identity with one of these sequences and having tyrosine ammonia lyase activity; preferably a TAL comprising a sequence chosen from SEQ ID NO: 41 and polypeptides comprising a sequence having at least 60, 70, 80, 85, 90 or 95% sequence identity with this sequence and having tyrosine ammonia lyase activity;
a heterologous nucleic acid sequence coding for a 4-coumarate-CoA ligase (4CL) which is capable of producing coumaryl-CoA from p-coumaric acid and Coenzyme A; preferably from Arabidopsis thaliana, Petroselinum crispum or Streptomyces clavuligerus; a 4CL comprising a sequence chosen from SEQ ID NOs: 43, 45, 47 and 49 and polypeptides comprising a sequence having at least 60, 70, 80, 85, 90 or 95% sequence identity with one of these sequences and having 4-coumarate-CoA ligase activity, and preferably a 4CL comprising a sequence selected from SEQ ID NO: 45 and polypeptides comprising a sequence having at least 60, 70, 80, 85, 90 or 95% sequence identity with this sequence and having 4-coumarate-CoA ligase activity;
a heterologous nucleic acid sequence coding for a chalcone synthase (CHS) which is capable of producing naringenin-chalcone from 4-coumaroyl-CoA and malonyl-CoA; preferably from Citrus sinensis, Hordeum vulgare or Streptomyces clavuligerus, in particular a CHS comprising a sequence chosen from SEQ ID NOs: 51, 53, 55 and 57 and polypeptides comprising a sequence having at least 60, 70, 80, 85, 90 or 95% sequence identity with one of these sequences and having chalcone synthase activity, preferably from enzymes comprising a sequence chosen from SEQ ID NOs: 53 and 55 and polypeptides comprising a sequence having at least 60, 70, 80, 85, 90 or 95% sequence identity with one of these sequences and having chalcone synthase activity, and more particularly preferably a CHS comprising a sequence chosen from SEQ ID NO: 53 and polypeptides comprising a sequence having at least 60, 70, 80, 85, 90 or 95% sequence identity with this sequence and having chalcone synthase activity;
a heterologous nucleic acid sequence coding for a chalcone isomerase (CHI) which is capable of producing naringenin from naringenin chalcone; preferably from Arabidopsis thaliana or Streptomyces clavuligerus, in particular a CHI comprising a sequence chosen from SEQ ID NOs: 59 and 61 and polypeptides comprising a sequence having at least 60, 70, 80, 85, 90 or 95% sequence identity with one of these sequences and having chalcone isomerase activity, and preferably a CHI comprising a sequence chosen from SEQ ID NO: 61 and polypeptides comprising a sequence having at least 60, 70, 80, 85, 90 or 95% sequence identity with this sequence and having chalcone isomerase activity;
a heterologous nucleic acid sequence coding for a phenylalanine ammonia lyase (PAL), in particular a PAL comprising a sequence chosen from SEQ ID NOs: 63, 65 and 77 and polypeptides comprising a sequence having at least 60, 70, 80, 85, 90 or 95% sequence identity with one of these sequences and having phenylalanine ammonia lyase activity, in particular a PAL comprising a sequence chosen from SEQ ID NO: 65 and polypeptides comprising a sequence having at least 60, 70, 80, 85, 90 or 95% sequence identity with this sequence and having phenylalanine ammonia lyase activity; and
a heterologous nucleic acid sequence coding for a cinnamate 4-hydroxylase (C4H), in particular a C4H comprising a sequence chosen from SEQ ID NOs: 67, 69 and 79 and polypeptides comprising a sequence having at least 60, 70, 80, 85, 90 or 95% sequence identity with one of these sequences and having cinnamate 4-hydroxylase activity, which are capable of producing p-coumaric acid from phenylalanine, in particular a C4H comprising a sequence chosen from SEQ ID NO: 79 and polypeptides comprising a sequence having at least 60, 70, 80, 85, 90 or 95% sequence identity with this sequence and having cinnamate 4-hydroxylase activity;
a nucleic acid sequence coding for a 4-hydroxyphenylacetate 3-monooxygenase oxygenase subunit (HpaB), preferably comprising a sequence chosen from SEQ ID NO: 83 and polypeptides comprising a sequence having at least 60, 70, 80, 85, 90 or 95% sequence identity therewith and having 4-hydroxyphenylacetate 3-monooxygenase oxygenase activity, and a nucleic acid sequence coding for a 4-hydroxyphenylacetate 3-monooxygenase reductase subunit (HpaC), preferably comprising a sequence chosen from SEQ ID NO: 85 and polypeptides comprising a sequence having at least 60, 70, 80, 85, 90 or 95% sequence identity therewith and having 4-hydroxyphenylacetate 3-monooxygenase reductase activity; or a nucleic acid sequence coding for a 4-methoxybenzoate O-demethylase which is capable of converting tyrosine into L-DOPA and also p-coumaric acid into caffeic acid, preferably comprising a sequence chosen from SEQ ID NOs: 73 and 75 and polypeptides comprising a sequence having at least 60, 70, 80, 85, 90 or 95% sequence identity with one of these sequences and having L-tyrosine hydrolase activity; or a heterologous nucleic acid sequence coding for a p-coumarate 3-hydroxylase which is capable of converting p-coumaric acid into caffeic acid, preferably comprising a sequence SEQ ID NO: 71 and polypeptides comprising a sequence having at least 60, 70, 80, 85, 90 or 95% sequence identity with one of these sequences and having p-coumarate 3-hydroxylase activity, and
a nucleic acid sequence coding for a dihydroxyphenylalanine ammonia-lyase (DAL).

According to a preferred embodiment, the method comprises the introduction of:
(i) a heterologous nucleic acid sequence coding for a flavanone 7-O-beta-D-glucosyltransferase (UGT) from Arabidopsis thaliana, Scutellaria baicalensis or Homo sapiens, preferably from Arabidopsis thaliana or from Scutellaria baicalensis, preferably a flavanone 7-O-beta-D-glucosyltransferase (UGT) selected from enzymes comprising a sequence chosen from SEQ ID NOs: 113, 115, 91, 93, 95, 97, 99 and 101 and polypeptides comprising a sequence having at least 60, 70, 80, 85, 90 or 95% sequence identity with one of these sequences and having flavanone 7-O-beta-D-glucosyltransferase activity; in particular a flavanone 7-O-beta-D-glucosyltransferase (UGT) comprising a sequence chosen from SEQ ID NOs: 113 and 95 and polypeptides comprising a sequence having at least 60, 70, 80, 85, 90 or 95% sequence identity with one of these sequences and having flavanone 7-O-beta-D-glucosyltransferase activity;
(ii) a heterologous nucleic acid sequence coding for a 6″-O-rhamnosyltransferase (RhaT) of the genus Citrus or Petunia hybrida, preferably Citrus sinensis, Citrus maxima, or Citrus clementina, even more preferably Citrus sinensis or Citrus clementina, preferably an RhaT comprising a sequence chosen from SEQ ID NOs: 103 and 105 and polypeptides comprising a sequence having at least 60, 70, 80, 85, 90 or 95% sequence identity with one of these sequences and having 6″-O-rhamnosyltransferase activity, in particular a 6″-O-rhamnosyltransferase (RhaT) comprising a sequence chosen from SEQ ID NO: 103 and polypeptides comprising a sequence having at least 60, 70, 80, 85, 90 or 95% sequence identity with this sequence and having 6″-O-rhamnosyltransferase activity; and
(iii) a heterologous nucleic acid sequence coding for a UDP-glucose 4,6-dehydratase/UDP-4-keto-6-deoxy-D-glucose 3,5-epimerase/UDP-4-keto-L-rhamnose-reductase (RHM) from Citrus sinensis or from Arabidopsis thaliana, preferably an RHM comprising a sequence chosen from SEQ ID NOs: 107, 109 and 111 and polypeptides comprising a sequence having at least 60, 70, 80, 85, 90 or 95% sequence identity with one of these sequences and having UDP-glucose 4,6-dehydratase/UDP-4-keto-6-deoxy-D-glucose 3,5-epimerase/UDP-4-keto-L-rhamnose-reductase activity, in particular an RHM comprising a sequence selected from SEQ ID NO: 107 and polypeptides comprising a sequence having at least 60, 70, 80, 85, 90 or 95% sequence identity with this sequence and having UDP-glucose 4,6-dehydratase/UDP-4-keto-6-deoxy-D-glucose 3,5-epimerase/UDP-4-keto-L-rhamnose-reductase activity;
   and of at least one nucleic acid sequence chosen from:
a heterologous nucleic acid sequence coding for an O-methyltransferase (OMT) which is capable of methylating eriodictyol and/or luteolin in position 4′; preferably an OMT from Citrus clementina, Citrus sinensis, Arabidopsis thaliana or Homo sapiens, preferably an OMT comprising a sequence chosen from SEQ ID NOs: 117, 119, 87 and 89 and polypeptides comprising a sequence having at least 60, 70, 80, 85, 90 or 95% sequence identity with one of these sequences and having O-methyltransferase activity, notably with eriodictyol and/or luteolin as substrate and methylation in position 4′, preferably an OMT comprising a sequence chosen from SEQ ID NOs: 117 and 119 and polypeptides comprising a sequence having at least 60, 70, 80, 85, 90 or 95% sequence identity with one of these sequences and having O-methyltransferase activity, and most particularly preferably an OMT comprising a sequence chosen from SEQ ID NO: 117 and polypeptides comprising a sequence having at least 60, 70, 80, 85, 90 or 95% sequence identity with this sequence and having O-methyltransferase activity;
a heterologous nucleic acid sequence coding for a flavonoid 3′-monooxygenase (F3′H) which is capable of hydroxylating naringenin and/or apigenin in position 3′ and comprising a sequence selected from SEQ ID NOs: 7, 11, 17 and 121 and polypeptides comprising a sequence having at least 75%, at least 80%, at least 85%, at least 90% or at least 95% identity with one of these sequences and having flavonoid 3′-monooxygenase activity, preferably an enzyme comprising a sequence selected from SEQ ID NOs: 7, 17 and 121 and polypeptides comprising a sequence having at least 75%, at least 80%, at least 85%, at least 90% or at least 95% identity with one of these sequences and having flavonoid 3′-monooxygenase activity, and most particularly preferably an enzyme comprising a sequence selected from SEQ ID NO: 7 and polypeptides comprising a sequence having at least 75%, at least 80%, at least 85%, at least 90% or at least 95% identity with this sequence and having flavonoid 3′-monooxygenase activity; and
a heterologous nucleic acid sequence coding for a cytochrome P450 reductase (CPR) and comprising a sequence chosen from SEQ ID NOs: 23, 25, 27, 29 and 31 and polypeptides comprising a sequence having at least 60, 70, 80, 85, 90 or 95% sequence identity with one of these sequences and having cytochrome P450 reductase activity, preferably from enzymes comprising a sequence chosen from SEQ ID NOs: 23, 25 and 29 and polypeptides comprising a sequence having at least 60, 70, 80, 85, 90 or 95% sequence identity with one of these sequences and having cytochrome P450 reductase activity, and particularly a CPR comprising a sequence chosen from SEQ ID NO: 25 and polypeptides comprising a sequence having at least 60, 70, 80, 85, 90 or 95% sequence identity with this sequence and having cytochrome P450 reductase activity; and
a heterologous nucleic acid sequence coding for an FNS comprising a sequence chosen from SEQ ID NOs: 33, 35, 37, 127, 129, 131, 133, 135, 137, 139, 141, 143, 145, 147, 149, 151, 153, 155, 157 and 159 and polypeptides comprising a sequence having at least 60, 70, 80, 85, 90 or 95% sequence identity with one of these sequences and having flavone synthase activity, in particular a flavone synthase (FNS) and comprising a sequence chosen from SEQ ID NOs: 33, 35 and 37 and polypeptides comprising a sequence having at least 60, 70, 80, 85, 90 or 95% sequence identity with one of these sequences and having flavone synthase activity, preferably a flavone synthase (FNS) comprising a sequence chosen from SEQ ID NO: 37 and a polypeptide comprising a sequence having at least 60, 70, 80, 85, 90 or 95% sequence identity with this sequence and having flavone synthase activity; and
a nucleic acid sequence coding for an S-adenosylmethionine synthetase (SAMT); in particular from Saccharomyces cerevisiae, for example an SAMT comprising a sequence chosen from SEQ ID NO: 81 and a polypeptide comprising a sequence having at least 60, 70, 80, 85, 90 or 95% sequence identity with this sequence and having S-adenosylmethionine synthetase activity, and
a heterologous nucleic acid sequence coding for a tyrosine ammonia lyase (TAL) from Rhodotorula glutinis or Flavobacterium johnsoniae; in particular a TAL comprising a sequence chosen from SEQ ID NOs: 41 and 39 and polypeptides comprising a sequence having at least 60, 70, 80, 85, 90 or 95% sequence identity with one of these sequences and having tyrosine ammonia lyase activity; preferably a TAL comprising a sequence chosen from SEQ ID NO: 41 and polypeptides comprising a sequence having at least 60, 70, 80, 85, 90 or 95% sequence identity with this sequence and having tyrosine ammonia lyase activity;
a heterologous nucleic acid sequence coding for a 4-coumaroyl-CoA ligase (4CL) from Arabidopsis thaliana, Citrus clementina, Petroselinum crispum or Streptomyces clavuligerus; in particular a 4CL comprising a sequence chosen from SEQ ID NOs: 123, 125, 45, 43, 47 and 49 and polypeptides comprising a sequence having at least 60, 70, 80, 85, 90 or 95% sequence identity with one of these sequences and having 4-coumarate-CoA ligase activity; preferably a 4CL comprising a sequence selected from SEQ ID NOs: 123, 125 and 45 and polypeptides comprising a sequence having at least 60, 70, 80, 85, 90 or 95% sequence identity with one of these sequences and having 4-coumarate-CoA ligase activity; most particularly preferably a 4CL comprising a sequence selected from SEQ ID NO: 45 and polypeptides comprising a sequence having at least 60, 70, 80, 85, 90 or 95% sequence identity with this sequence and having 4-coumarate-CoA ligase activity, and
a heterologous nucleic acid sequence coding for a chalcone synthase (CHS) from Citrus sinensis, Hordeum vulgare or Streptomyces clavuligerus, in particular a CHS comprising a sequence chosen from SEQ ID NOs: 53, 51, 55 and 57 and polypeptides comprising a sequence having at least 60, 70, 80, 85, 90 or 95% sequence identity with one of these sequences and having chalcone synthase activity, preferably a CHS comprising a sequence chosen from SEQ ID NO: 53 and polypeptides comprising a sequence having at least 60, 70, 80, 85, 90 or 95% sequence identity with this sequence and having chalcone synthase activity; and
a heterologous nucleic acid sequence coding for a chalcone isomerase (CHI) from Arabidopsis thaliana or Streptomyces clavuligerus, in particular a CHI comprising a sequence chosen from SEQ ID NOs: 61 and 59 and polypeptides comprising a sequence having at least 60, 70, 80, 85, 90 or 95% sequence identity with one of these sequences and having chalcone isomerase activity; preferably a CHI comprising a sequence chosen from SEQ ID NO: 61 and polypeptides comprising a sequence having at least 60, 70, 80, 85, 90 or 95% sequence identity with this sequence and having chalcone isomerase activity.

Preferably, the method comprises the introduction of all these sequences.

Preferably, the method also comprises the introduction of:
a heterologous nucleic acid sequence coding for a phenylalanine ammonia lyase (PAL), in particular a PAL comprising a sequence chosen from SEQ ID NOs: 63, 65 and 77, preferably SEQ ID NOs: 65 and 77 and polypeptides comprising a sequence having at least 60, 70, 80, 85, 90 or 95% sequence identity with one of these sequences and having phenylalanine ammonia lyase activity, and more particularly preferably a phenylalanine ammonia lyase (PAL) comprising a sequence chosen from SEQ ID NO: 65 and polypeptides comprising a sequence having at least 60, 70, 80, 85, 90 or 95% sequence identity with this sequence and having phenylalanine ammonia lyase activity; and
a heterologous nucleic acid sequence coding for a cinnamate 4-hydroxylase (C4H), in particular a C4H comprising a sequence chosen from SEQ ID NOs: 67, 69 and 79 and polypeptides comprising a sequence having at least 60, 70, 80, 85, 90 or 95% sequence identity with one of these sequences and having cinnamate 4-hydroxylase activity, and most particularly preferably a cinnamate 4-hydroxylase (C4H) comprising a sequence chosen from SEQ ID NO: 79 and polypeptides comprising a sequence having at least 60, 70, 80, 85, 90 or 95% sequence identity with this sequence and having cinnamate 4-hydroxylase activity.

Preferably, the method comprises the introduction of combinations of particular coding sequences as described above.

Production of Diosmin and/or Hesperidin

The present invention relates to the use of a microorganism according to the present invention for producing diosmin and/or hesperidin. In a first preferred embodiment, the invention relates to the use of a microorganism according to the present invention for producing diosmin. In a second preferred embodiment, the invention relates to the use of a microorganism according to the present invention for producing hesperidin. In a preferred embodiment, the invention relates to the use of a microorganism according to the present invention for producing diosmin and hesperidin.

The present invention also relates to a method for producing diosmin and/or hesperidin, comprising the cultivation of a microorganism according to the present invention, notably under conditions allowing or that are favourable for the production of diosmin and/or hesperidin and optionally the recovery and/or purification of the diosmin and/or hesperidin produced.

The conditions for cultivating the microorganism according to the invention may be adapted according to the conventional techniques that are well known to those skilled in the art.

The microorganism is cultivated in a suitable culture medium. The term “suitable culture medium” generally denotes a culture medium providing the nutrients that are essential for or beneficial to the maintenance and/or growth of said microorganism, such as carbon sources; nitrogen sources such as ammonium sulfate; phosphorus sources, for example monobasic potassium phosphate; trace elements, for example copper, iodide, iron, magnesium, zinc or molybdate salts; vitamins and other growth factors such as amino acids or other growth promoters. An antifoam may be added if need be. According to the invention, this suitable culture medium may be chemically defined or complex. The culture medium may thus be identical or similar in composition to that of a synthetic medium, as defined by Verduyn et al., (Yeast. 1992. 8: 501-17), adapted by Visser et al., (Biotechnology and Bioengineering. 2002. 79: 674-81), or commercially available such as the YNB medium (Yeast Nitrogen Base, MP Biomedicals or Sigma-Aldrich). Notably, the culture medium may comprise a simple carbon source, such as glucose, fructose, xylose, ethanol, glycerol, galactose, sucrose, cellulose, cellobiose, starch, glucose polymers, molasses, or byproducts of these sugars.

Preferably, the production of diosmin and/or hesperidin by the microorganism according to the invention is obtained without supplying naringenin, apigenin, eriodictyol, luteolin, hesperetin and/or diosmetin to the culture medium, preferably without supplying naringenin, apigenin, eriodictyol, luteolin, hesperetin and diosmetin to the culture medium.

According to the invention, any cultivation method for the industrial-scale production of molecules of interest may be envisioned. Advantageously, the cultivation is performed in bioreactors, notably in batch, fed-batch, chemostat and/or continuous cultivation mode. Controlled feeding with vitamins during the process may also be beneficial to the productivity (Alfenore et al., Appl. Microbiol. Biotechnol. 2002. 60: 67-72).

The cultivation is generally performed in bioreactors, with possible solid and/or liquid preculturing steps in Erlenmeyer flasks, with a suitable culture medium.

In general, the conditions for cultivating the microorganisms according to the invention are readily adaptable by a person skilled in the art, as a function of the microorganism. For example, the cultivation temperature is notably, for yeasts, between 20° C. and 40° C., preferably between 28° C. and 35° C., and more particularly about 30° C. for S. cerevisiae.

The microorganism according to the present invention may be cultivated for 1 to 30 days and preferably for 1 to 10 days.

A microorganism according to the present invention is capable of producing diosmin and/or hesperidin in a minimum amount of 1 mg/l of culture medium, preferably 10, 20, 25, 30, 35, 40, 45, 50, 55, 60, 65, 70, 75, 80, 85, 90, 95 or 100 mg/l of culture medium, optionally 150, 200, 250, 300, 350, 400, 450, 500, 600, 700, 800, 900 or 1000 mg/l of culture medium.

### DESCRIPTION OF THE FIGURES

FIG. 1: Description of the metabolic pathways for producing hesperidin and diosmin.

FIG. 2: Production of eriodictyol from naringenin by the strain FL_405 (F3′H4+CPR2). Control strain: CF235. Observation of disappearance of the naringenin peak and appearance of an eriodictyol peak in the strain FL-405.

FIG. 3: Production of luteolin from apigenin by the strain FL_405 (F3′H4+CPR2). Control strain: CF235. Observation of disappearance of the apigenin peak and appearance of a luteolin peak in the strain FL-405.

FIG. 4: Production of apigenin from naringenin by the strain SC744 (FNSII1+CPR2). Control strain: CF234. Observation of disappearance of the naringenin peak and appearance of an apigenin peak in the strain.

FIG. 5: Production of luteolin from eriodictyol SC744 (FNSII1+CPR2). Control strain: CF234. Observation of disappearance of the eriodictyol peak and appearance of a luteolin peak in the strain.

FIG. 6: Production of eriodictyol and luteolin by the strain SC1500. Control strain: CF237. Observation of the eriodictyol and luteolin peaks.

FIG. 7: Production of hesperetin from eriodictyol by the strains SC 1612 (MET+SAM) and SC 1614 (MET+SAM). Control strain: CF235. Observation of disappearance of the eriodictyol peak and appearance of a hesperetin peak in the strains.

FIG. 8: Production of diosmetin from luteolin by the strains SC 1612 (MET+SAM) and SC 1614 (MET+SAM). Control strain: CF235. Observation of disappearance of the luteolin peak and appearance of a diosmetin peak in the strains.

FIG. 9: Production of diosmetin from hesperetin by the strain SC744 (FNSII+CPR). Control strain: CF234. Observation of disappearance of the hesperetin peak and appearance of a diosmetin peak in the strain.

FIG. 10: Production of hesperetin from eriodictyol by E. coli EC26 (MET+SAM). Control strain: E. coli MH1. Observation of disappearance of the eriodictyol peak and appearance of a hesperetin peak in the strain.

FIG. 11: Production of diosmetin from luteolin by E. coli EC26 (MET+SAM). Control strain: E. coli MH1. Observation of disappearance of the luteolin peak and appearance of a diosmetin peak in the strain.

FIG. 12: Production of diosmetin from hesperetin by E. coli EC30 (FNSII). Control strain: E. coli MH1. Observation of disappearance of the hesperetin peak and appearance of a diosmetin peak in the strain.

FIG. 13: Production of hesperetin and diosmetin by the strain SC1508. Control strain: CF237. Observation of the hesperetin and diosmetin peaks.

FIG. 14: Production of hesperidin from hesperetin by the strain FL 547 (GT+RHM+RHAT). Control strain: CF 233. Observation of disappearance of the hesperetin peak and appearance of the hesperidin peak.

FIG. 15: Production of diosmin from diosmetin by the strain FL 547 (GT+RHM+RHAT). Control strain: CF 233. Observation of disappearance of the diosmetin peak and appearance of the diosmin peak.

FIG. 16: Production of hesperidin from hesperetin by E. coli EC38, EC45 and EC47 (GT+RHM+RHAT). Control strain: E. coli MH1. Observation of disappearance of the hesperetin peak and appearance of the hesperidin peak.

FIG. 17: Production of diosmin from diosmetin by E. coli EC38, EC45 and EC47 (GT+RHM+RHAT). Control strain: E. coli MH1. Observation of disappearance of the diosmetin peak and appearance of the diosmin peak.

FIG. 18: Production of hesperidin and diosmin by the strains SC1509, SC1530, SC1529, SC1568 and SC2410. Control strain: CF237. Observation of the hesperidin and diosmin peaks.

FIG. 19: Production of eriodictyol and luteolin by the strains SC2424, SC2425, SC2426, SC2427, SC2428 and SC1500. Control strain: CF237.

FIG. 20: Production of hesperetin and homoeriodictyol by the strains SC2147, SC2151, SC1612 and SC1614. Control strain: CF235.

FIG. 21: Production of diosmetin and chrysoeriol by the strains SC2147, SC2151, SC1612 and SC1614. Control strain: CF235.

FIG. 22: Production of hesperetin from eriodictyol by E. coli EC41 (MET+SAM). Control strain: E. coli MH1. Observation of disappearance of the eriodictyol peak and appearance of a hesperetin peak in the strain.

FIG. 23: Production of hesperetin from eriodictyol by E. coli EC43 (MET+SAM). Control strain: E. coli MH1. Observation of disappearance of the eriodictyol peak and appearance of a hesperetin peak in the strain.

FIG. 24: Production of diosmetin from luteolin by E. coli EC43 (MET+SAM). Control strain: E. coli MH1. Observation of disappearance of the luteolin peak and appearance of a diosmetin peak in the strain.

FIG. 25: Production of hesperetin and diosmetin by the strain SC2408. Control strain: CF237. Observation of the hesperetin and diosmetin peaks.

FIG. 26: Production of hesperetin and diosmetin by the strain SC2409. Control strain: CF237. Observation of the hesperetin and diosmetin peaks.

FIG. 27: Production of hesperetin and diosmetin by the strains SC2408, SC2409 and SC1508. Control strain: CF237.

FIG. 28: Production of hesperidin and diosmin by the strains SC1579, SC1584, SC1621 and SC1626.

FIG. 29: Production of diosmetin from naringenin by the strains SC2429 to SC2434, SC2436 to SC2444, SC2446 to SC2454, SC2456 to SC2464 and SC2466.

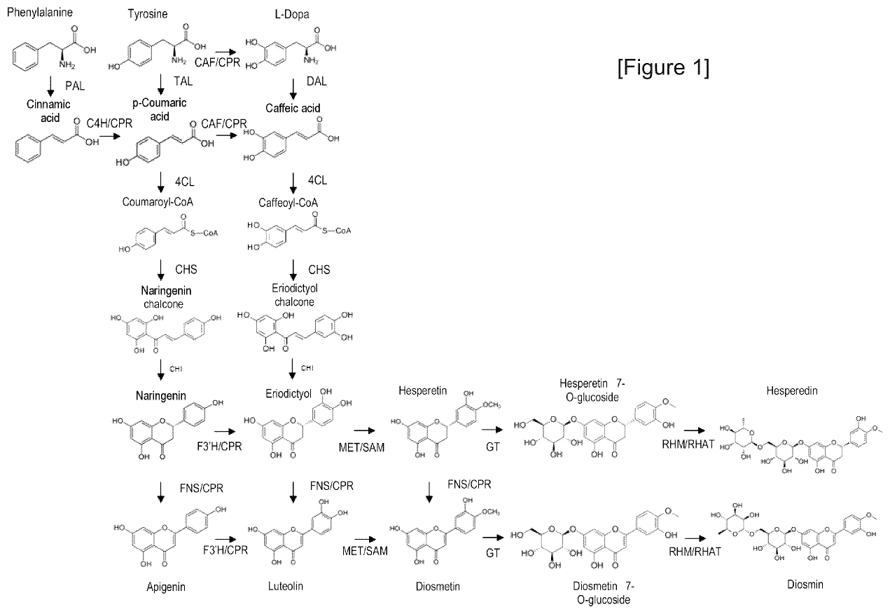

TABLE 1
SEQUENCE DESCRIPTION

| SEQ | |
|---|---|
| ID | |
| NO | Description |
| 39 | Amino acid sequence of tyrosine ammonia lyase from |
| | Flavobacterium johnsoniae |
| 40 | Nucleic acid sequence coding for tyrosine ammonia lyase from |
| | Flavobacterium johnsoniae |
| 41 | Amino acid sequence of tyrosine ammonia lyase from |
| | Rhodotorula glutinis |
| 42 | Nucleic acid sequence coding for tyrosine ammonia lyase from |
| | Rhodotorula glutinis |
| 43 | Amino acid sequence of 4-coumarate-CoA ligase from |
| | Arabidopsis thaliana |
| 44 | Nucleic acid sequence coding for 4-coumarate-CoA ligase |
| | from Arabidopsis thaliana |
| 45 | Amino acid sequence of 4-coumarate-CoA ligase from |
| | Petroselinum crispum |
| 46 | Nucleic acid sequence coding for 4-coumarate-CoA ligase from |
| | Petroselinum crispum |
| 47 | Amino acid sequence of 4-coumarate-CoA ligase from |
| | Petroselinum crispum |
| 48 | Nucleic acid sequence coding for 4-coumarate-CoA ligase from |
| | Petroselinum crispum |
| 49 | Amino acid sequence of 4-coumarate-CoA ligase from |
| | Streptomyces clavuligerus |
| 50 | Nucleic acid sequence coding for 4-coumarate-CoA ligase |
| | from Streptomyces clavuligerus |
| 51 | Amino acid sequence of chalcone synthase from Hordeum |
| | vulgare |
| 52 | Nucleic acid sequence coding for chalcone synthase from |
| | Hordeum vulgare |
| 53 | Amino acid sequence of chalcone synthase from Citrus sinensis |
| 54 | Nucleic acid sequence coding for chalcone synthase from |
| | Citrus sinensis |
| 55 | Amino acid sequence of chalcone synthase from Citrus sinensis |
| 56 | Nucleic acid sequence coding for chalcone synthase from |
| | Citrus sinensis |
| 57 | Amino acid sequence of chalcone synthase from Streptomyces |
| | clavuligerus |
| 58 | Nucleic acid sequence coding for chalcone synthase from |
| | Streptomyces clavuligerus |
| 59 | Amino acid sequence of chalcone isomerase from Streptomyces |
| | clavuligerus |
| 60 | Nucleic acid sequence coding for chalcone isomerase from |
| | Streptomyces clavuligerus |
| 61 | Amino acid sequence of chalcone isomerase from Arabidopsis |
| | thaliana |
| 62 | Nucleic acid sequence coding for chalcone isomerase from |
| | Arabidopsis thaliana |
| 33 | Amino acid sequence of flavone synthase from Lonicera |
| | japonica |
| 34 | Nucleic acid sequence coding for flavone synthase from |
| | Lonicera japonica |
| 35 | Amino acid sequence of flavone synthase from Lonicera |
| | macranthoides |
| 36 | Nucleic acid sequence coding for flavone synthase from |
| | Lonicera macranthoides |
| 37 | Amino acid sequence of flavone synthase from Petroselinum |
| | crispum |
| 38 | Nucleic acid sequence coding for flavone synthase from |
| | Petroselinum crispum |
| 1 | Amino acid sequence of flavonoid 3′-monooxygenase from |
| | Perilla frutescens var. crispa |
| 2 | Nucleic acid sequence coding for flavonoid 3′-monooxygenase |
| | from Perilla frutescens var. crispa |
| 3 | Amino acid sequence of flavonoid 3′-monooxygenase from |
| | Phanerochaete chrysosporium |
| 4 | Nucleic acid sequence coding for flavonoid 3′-monooxygenase |
| | from Phanerochaete chrysosporium |
| 5 | Amino acid sequence of flavonoid 3′-monooxygenase from |
| | Petunia x hybrida |
| 6 | Nucleic acid sequence coding for flavonoid 3′-monooxygenase |
| | from Petunia x hybrida |
| 7 | Amino acid sequence of flavonoid 3′-monooxygenase from |
| | Callistephus chinensis |
| 8 | Nucleic acid sequence coding for flavonoid 3′-monooxygenase |
| | from Callistephus chinensis |
| 9 | Amino acid sequence of flavonoid 3′-monooxygenase from |
| | Callistephus chinensis |
| 10 | Nucleic acid sequence coding for flavonoid 3′-monooxygenase |
| | from Callistephus chinensis |
| 11 | Amino acid sequence of flavonoid 3′-monooxygenase from |
| | Gerbera hybrida |
| 12 | Nucleic acid sequence coding for flavonoid 3′-monooxygenase |
| | from Gerbera hybrida |
| 13 | Amino acid sequence of flavonoid 3′-monooxygenase from |
| | Osteospermum hybrid cultivar |
| 14 | Nucleic acid sequence coding for flavonoid 3′-monooxygenase |
| | from Osteospermum hybrid cultivar |
| 15 | Amino acid sequence of flavonoid 3′-monooxygenase from |
| | Citrus clementina |
| 16 | Nucleic acid sequence coding for flavonoid 3′-monooxygenase |
| | from Citrus clementina |
| 17 | Amino acid sequence of flavonoid 3′-monooxygenase from |
| | Citrus sinensis |
| 18 | Nucleic acid sequence coding for flavonoid 3′-monooxygenase |
| | from Citrus sinensis |
| 19 | Amino acid sequence of flavonoid 3′-monooxygenase from |
| | Pilosella officinarum |
| 20 | Nucleic acid sequence coding for flavonoid 3′-monooxygenase |
| | from Pilosella officinarum |
| 21 | Amino acid sequence of flavonoid 3′-monooxygenase from |
| | Streptomyces avermitilis |
| 22 | Nucleic acid sequence coding for flavonoid 3′-monooxygenase |
| | from Streptomyces avermitilis |
| 23 | Amino acid sequence of cytochrome P450 reductase from |
| | Catharanthus roseus |
| 24 | Nucleic acid sequence coding for cytochrome P450 reductase |
| | from Catharanthus roseus |
| 25 | Amino acid sequence of cytochrome P450 reductase from |
| | Saccharomyces cerevisiae |
| 26 | Nucleic acid sequence coding for cytochrome P450 reductase |
| | from Saccharomyces cerevisiae |
| 27 | Amino acid sequence of chimeric cytochrome P450 reductase |
| 28 | Nucleic acid sequence coding for chimeric cytochrome P450 |
| | reductase |
| 29 | Amino acid sequence of cytochrome P450 reductase from |
| | Arabidopsis thaliana |
| 30 | Nucleic acid sequence coding for cytochrome P450 reductase |
| | from Arabidopsis thaliana |
| 31 | Amino acid sequence of cytochrome P450 reductase from |
| | Arabidopsis thaliana |
| 32 | Nucleic acid sequence coding for cytochrome P450 reductase |
| | from Arabidopsis thaliana |
| 63 | Amino acid sequence of phenylalanine ammonia lyase from |
| | Citrus sinensis |
| 64 | Nucleic acid sequence coding for phenylalanine ammonia lyase |
| | from Citrus sinensis |
| 65 | Amino acid sequence of phenylalanine ammonia lyase from |
| | Citrus sinensis |
| 66 | Nucleic acid sequence coding for phenylalanine ammonia lyase |
| | from Citrus sinensis |
| 67 | Amino acid sequence of cinnamate 4-hydroxylase from Citrus |
| | sinensis |
| 68 | Nucleic acid sequence coding for cinnamate 4-hydroxylase from |
| | Citrus sinensis |
| 69 | Amino acid sequence of cinnamate 4-hydroxylase from Citrus |
| | sinensis |
| 70 | Nucleic acid sequence coding for cinnamate 4-hydroxylase from |
| | Citrus sinensis |
| 71 | Amino acid sequence of coumarate 3-hydroxylase from |
| | Saccharothrix espanaensis |
| 72 | Nucleic acid sequence coding for coumarate 3-hydroxylase from |
| | Saccharothrix espanaensis |
| 73 | Amino acid sequence of 4-methoxybenzoate O-demethylase |
| | from Beta vulgaris |
| 74 | Nucleic acid sequence coding for 4-methoxybenzoate |
| | O-demethylase from Beta vulgaris |
| 75 | Amino acid sequence of 4-methoxybenzoate O-demethylase from |
| | Rhodopseudomonas palustris |
| 76 | Nucleic acid sequence coding for 4-methoxybenzoate |
| | O-demethylase from Rhodopseudomonas palustris |
| 77 | Amino acid sequence of phenylalanine ammonia lyase from |
| | Arabidopsis thaliana |
| 78 | Nucleic acid sequence coding for phenylalanine ammonia lyase |
| | from Arabidopsis thaliana |
| 79 | Amino acid sequence of cinnamate 4-hydroxylase from |
| | Arabidopsis thaliana |
| 80 | Nucleic acid sequence coding for cinnamate 4-hydroxylase from |
| | Arabidopsis thaliana |
| 81 | Amino acid sequence of S-adenosylmethionine synthetase from |
| | Saccharomyces cerevisiae |
| 82 | Nucleic acid sequence coding for S-adenosylmethionine |
| | synthetase from Saccharomyces cerevisiae |
| 83 | Amino acid sequence of 4-hydroxyphenylacetate |
| | 3-monooxygenase oxygenase from Escherichia coli |
| 84 | Nucleic acid sequence coding for 4-hydroxyphenylacetate |
| | 3-monooxygenase oxygenase from Escherichia coli |
| 85 | Amino acid sequence of 4-hydroxyphenylacetate |
| | 3-monooxygenase reductase from Escherichia coli |
| 86 | Nucleic acid sequence coding for 4-hydroxyphenylacetate |
| | 3-monooxygenase reductase from Escherichia coli |
| 87 | Amino acid sequence of O-methyltransferase from Arabidopsis |
| | thaliana |
| 88 | Nucleic acid sequence coding for O-methyltransferase from |
| | Arabidopsis thaliana |
| 89 | Amino acid sequence of O-methyltransferase from Homo |
| | sapiens |
| 90 | Nucleic acid sequence coding for O-methyltransferase from |
| | Homo sapiens |
| 91 | Amino acid sequence of flavanone 7-O-beta-D- |
| | glucosyltransferase from Arabidopsis thaliana |
| 92 | Nucleic acid sequence coding for flavanone 7-O-beta-D- |
| | glucosyltransferase from Arabidopsis thaliana |
| 93 | Amino acid sequence of flavanone 7-O-beta-D- |
| | glucosyltransferase from Scutellaria baicalensis |
| 94 | Nucleic acid sequence coding for flavanone 7-O-beta-D- |
| | glucosyltransferase from Scutellaria baicalensis |
| 95 | Amino acid sequence of flavanone 7-O-beta-D- |
| | glucosyltransferase from Scutellaria baicalensis |
| 96 | Nucleic acid sequence coding for flavanone 7-O-beta-D- |
| | glucosyltransferase from Scutellaria baicalensis |
| 97 | Amino acid sequence of flavanone 7-O-beta-D- |
| | glucosyltransferase from Scutellaria baicalensis |
| 98 | Nucleic acid sequence coding for flavanone 7-O-beta-D- |
| | glucosyltransferase from Scutellaria baicalensis |
| 99 | Amino acid sequence of flavanone 7-O-beta-D- |
| | glucosyltransferase from Homo sapiens |
| 100 | Nucleic acid sequence coding for flavanone 7-O-beta-D- |
| | glucosyltransferase from Homo sapiens |
| 101 | Amino acid sequence of flavanone 7-O-beta-D- |
| | glucosyltransferase from Homo sapiens |
| 102 | Nucleic acid sequence coding for flavanone 7-O-beta-D- |
| | glucosyltransferase from Homo sapiens |
| 103 | Amino acid sequence of 6-O-rhamnosyltransferase from Citrus |
| | clementina |
| 104 | Nucleic acid sequence coding for 6-O-rhamnosyltransferase |
| | from Citrus clementina |
| 105 | Amino acid sequence of 6-O-rhamnosyltransferase from Citrus |
| | sinensis |
| 106 | Nucleic acid sequence coding for 6-O-rhamnosyltransferase |
| | from Citrus sinensis |
| 107 | Amino acid sequence of UDP-glucose 4,6-dehydratase/ |
| | UDP-4-keto-6-deoxy-D-glucose 3,5-epimerase/UDP-4- |
| | keto-L-rhamnose-reductase from Citrus sinensis |
| 108 | Nucleic acid sequence coding for UDP-glucose |
| | 4,6-dehydratase/UDP-4-keto-6-deoxy-D-glucose |
| | 3,5-epimerase/UDP-4-keto-L-rhamnose-reductase from |
| | Citrus sinensis |
| 109 | Amino acid sequence of UDP-glucose 4,6-dehydratase/ |
| | UDP-4-keto-6-deoxy-D-glucose 3,5-epimerase/ |
| | UDP-4-keto-L-rhamnose-reductase from Arabidopsis thaliana |
| 110 | Nucleic acid sequence coding for UDP-glucose |
| | 4,6-dehydratase/UDP-4-keto-6-deoxy-D-glucose |
| | 3,5-epimerase/UDP-4-keto-L-rhamnose-reductase from |
| | Arabidopsis thaliana |
| 111 | Amino acid sequence of UDP-glucose 4,6-dehydratase/ |
| | UDP-4-keto-6-deoxy-D-glucose 3,5-epimerase/ |
| | UDP-4-keto-L-rhamnose-reductase from Arabidopsis thaliana |
| 112 | Nucleic acid sequence coding for UDP-glucose |
| | 4,6-dehydratase/UDP-4-keto-6-deoxy-D-glucose |
| | 3,5-epimerase/UDP-4-keto-L-rhamnose-reductase from |
| | Arabidopsis thaliana |
| 113 | Amino acid sequence of flavanone 7-O-beta-D- |
| | glucosyltransferase from Citrus sinensis |
| 114 | Nucleic acid sequence coding for flavanone 7-O-beta-D- |
| | glucosyltransferase from Citrus sinensis |
| 115 | Amino acid sequence of flavanone 7-O-beta-D- |
| | glucosyltransferase from Citrus clementina |
| 116 | Nucleic acid sequence coding for flavanone 7-O-beta-D- |
| | glucosyltransferase from Citrus clementina |
| 117 | Amino acid sequence of O-methyltransferase from Citrus |
| | clementina |
| 118 | Nucleic acid sequence coding for O-methyltransferase from |
| | Citrus clementina |
| 119 | Amino acid sequence of O-methyltransferase from Citrus |
| | sinensis |
| 120 | Nucleic acid sequence coding for O-methyltransferase from |
| | Citrus sinensis |
| 121 | Amino acid sequence of flavonoid 3′-monooxygenase from |
| | Arabidopsis thaliana |
| 122 | Nucleic acid sequence coding for flavonoid 3′-monooxygenase |
| | from Arabidopsis thaliana |
| 123 | Amino acid sequence of a 4-coumarate-CoA ligase from |
| | Arabidopsis thaliana |
| 124 | Nucleic acid sequence coding for a 4-coumarate-CoA ligase |
| | from Arabidopsis thaliana |
| 125 | Amino acid sequence of a 4-coumarate-CoA ligase from Citrus |
| | clementina |
| 126 | Nucleic acid sequence coding for 4-coumarate-CoA ligase |
| | from Citrus clementina |
| 127 | Amino acid sequence of flavone synthase from Angelica |
| | archangelica |
| 128 | Nucleic acid sequence coding for flavone synthase from |
| | Angelica archangelica |
| 129 | Amino acid sequence of flavone synthase from Cynara |
| | cardunculus var. scolymus |
| 130 | Nucleic acid sequence coding for flavone synthase from Cynara |
| | cardunculus var. scolymus |
| 131 | Amino acid sequence of flavone synthase from Perilla |
| | frutescens var. crispa |
| 132 | Nucleic acid sequence coding for flavone synthase from Perilla |
| | frutescens var. crispa |
| 133 | Amino acid sequence of flavone synthase from Dahlia pinnata |
| 134 | Nucleic acid sequence coding for flavone synthase from Dahlia |
| | pinnata |
| 135 | Amino acid sequence of flavone synthase from Callistephus |
| | chinensis |
| 136 | Nucleic acid sequence coding for flavone synthase from |
| | Callistephus chinensis |
| 137 | Amino acid sequence of flavone synthase from Apium graveolens |
| 138 | Nucleic acid sequence coding for flavone synthase from Apium |
| | graveolens |
| 139 | Amino acid sequence of flavone synthase from Medicago |
| | truncatula |
| 140 | Nucleic acid sequence coding for flavone synthase from |
| | Medicago truncatula |
| 141 | Amino acid sequence of flavone synthase from Cuminum |
| | cyminum |
| 142 | Nucleic acid sequence coding for flavone synthase from |
| | Cuminum cyminum |
| 143 | Amino acid sequence of flavone synthase from Aethusa |
| | cynapium |
| 144 | Nucleic acid sequence coding for flavone synthase from |
| | Aethusa cynapium |
| 145 | Amino acid sequence of flavone synthase from Conium |
| | maculatum |
| 146 | Nucleic acid sequence coding for flavone synthase from |
| | Conium maculatum |
| 147 | Amino acid sequence of flavone synthase from Camellia sinensis |
| 148 | Nucleic acid sequence coding for flavone synthase from |
| | Camellia sinensis |
| 149 | Amino acid sequence of flavone synthase from Saussurea |
| | medusa |
| 150 | Nucleic acid sequence coding for flavone synthase from |
| | Saussurea medusa |
| 151 | Amino acid sequence of flavone synthase from Plectranthus |
| | barbatus |
| 152 | Nucleic acid sequence coding for flavone synthase from |
| | Plectranthus barbatus |
| 153 | Amino acid sequence of flavone synthase from Scutellaria |
| | baicalensis |
| 154 | Nucleic acid sequence coding for flavone synthase from |
| | Scutellaria baicalensis |
| 155 | Amino acid sequence of flavone synthase from Dorcoceras |
| | hygrometricum |
| 156 | Nucleic acid sequence coding for flavone synthase from |
| | Dorcoceras hygrometricum |
| 157 | Amino acid sequence of flavone synthase from Antirrhinum |
| | majus |
| 158 | Nucleic acid sequence coding for flavone synthase from |
| | Antirrhinum majus |
| 159 | Amino acid sequence of flavone synthase from Erythranthe |
| | lewisii |
| 160 | Nucleic acid sequence coding for flavone synthase from |
| | Erythranthe lewisii |

### EXAMPLES

### Materials and Methods

### Strains

The yeasts used in the examples were obtained from Saccharomyces cerevisiae FY1679-28A (Tettelin et al., 1995 https://doi.org/10.1016/S1067-2389(06)80008-7). This yeast is quadruply auxotrophic for uracil, tryptophan, histidine and leucine. The bacterial strains used in the examples were obtained from Escherichia coli MH1.

### Standards

The standards were acquired from the supplier Extrasynthese, France (naringenin, apigenin, eriodictyol, luteolin, hesperetin, hesperidin, diosmetin and diosmin).

### Gene Cloning

The genes optimized to express in the yeast were synthesized by Eurofins Genomics, Ebersberg, Germany or Biomatik, Cambridge, Canada or Twist Biosciences, San Francisco, USA or DC Biosciences, Dundee, UK. By PCR, the gene cpr2 (SEQ ID NO: 26) from S. cerevisiae was amplified from the genomic DNA.

The genes obtained by synthesis or by PCR comprise at the 5′ and 3′ ends a BbsI (GAAGAC) or BsaI (GGTCTC) restriction site.

All the genes, promoters and terminators were restriction-cloned in the vector pSBK for expression in the yeast or in the vector pSB1K3 for expression in E. coli. The promoters and terminators (Wargner et al., 2015 DOI: 10.1016/j.fgb.2015.12.001) were recovered by PCR from the genomic DNA of the yeast S. cerevisiae or of E. coli.

The vector pSBK comprises a URA or LEU or TRP or HIS selection marker for the yeast and the vector pSB1K3 comprises a kanamycin-resistance marker.

### Culture Conditions

The strains were cultivated in 1 ml of minimum nitrogen base medium (Dutscher, Brumath, Fr) supplemented with glucose at 20 g/l for the yeasts and in 1 ml of M9 supplemented with glucose at 4 g·l⁻¹ for E. coli in 24-well plates (Starlab, Orsay, Fr) at 30° C. for 72 hours with continuous stirring at 200 rpm. In certain cases, naringenin or apigenin was added at a concentration of 100 mg·l⁻¹ to determine the activity of the F3′Hs, naringenin or eriodictyol was added at a concentration of 100 mg·l⁻¹ to determine the activity of the FNSIIs, eriodictyol or luteolin was added at a concentration of 100 mg·l⁻¹ to determine the activity of the METs, hesperetin or diosmetin was added at a concentration of 100 mg·l⁻¹ to determine the activity of the GTs, and hesperetin 7-O-glucoside and/or diosmetin 7-O-glucoside was added to determine the activity of the RHMs and RHATs.

Each strain was inoculated at an OD of 0.2 using a 24-hour preculture cultivated under the same conditions.

### Analytical Method

Preparation of the samples: The 1 mL cultures are frozen at −80° C. and then lyophilized for 12 hours at 0.10 mbar. The samples are then taken up in 1 mL of dimethyl sulfoxide (DMSO), stirred for 30 seconds at 1000 rpm and then centrifuged for 5 minutes at 3000 rpm at room temperature. After centrifugation, a known volume of supernatant is added to a known volume of a mixture of internal standards dissolved in methanol.

The final concentrations of the internal standards are:
Diosmin C13 0.5 mg/L
Diosmetin C13 0.015 mg/L

Analysis by UHPLC-TQ: The samples were analysed using a Vanquish-H UHPLC machine (Thermo) coupled to a Quantis triple-quadrupole MS (Thermo). The column is a Waters Acquity UPLC® USST3 column (8 μm 2.1×100 mm) combined with an HSST3 1.8 μm 2.1×5 mm precolumn.

The mobile phase A is a 0.1% solution of formic acid in LC/MS-grade water and the mobile phase B is a 0.1% solution of formic acid in pure LC/MS-grade acetonitrile. The column temperature is 50° C. and the temperature of the sample changer is 10° C.

Two chromatographic conditions were used for detecting the flavonoids of interest:

TABLE 2
Chromatographic conditions method 1

| Time | | | |
|---|---|---|---|
| (min) | Flow rate (ml/min) | Mobile phase A (%) | Mobile phase B (%) |
| 0 | 0.5 | 73 | 27 |
| 8 | 0.5 | 73 | 27 |

TABLE 3
Chromatographic conditions method 2

| Time | | | |
|---|---|---|---|
| (min) | Flow rate (ml/min) | Mobile phase A (%) | Mobile phase B (%) |
| 0 | 0.5 | 83 | 17 |
| 3.75 | 0.5 | 83 | 17 |
| 4 | 0.5 | 73 | 27 |
| 8.5 | 0.5 | 73 | 27 |
| 11.0 | 0.5 | 50 | 50 |
| 13.0 | 0.5 | 0 | 100 |
| 13.5 | 0.5 | 83 | 17 |
| 15.0 | 0.5 | 83 | 17 |

The ions monitored and the fragmentation conditions for the molecules of interest are:

TABLE 4
For method 1

| | | | | | | | Reference |
|---|---|---|---|---|---|---|---|
| | Retention | | Precursor | Daughter | Collision | Lens RF | internal |
| Molecules | time (min) | Polarity | ion | ion | energy | (V) | standard |
| Naringenin | 3.3 | Negative | 271.0 | 119.0 | 27 | 169 | Diosmetin |
| | | | | 150.9 | 18 | 169 | C13 |
| Apigenin | 3.5 | Negative | 269.0 | 117.1 | 35 | 201 | Diosmetin |
| | | | | 150.9 | 24 | 201 | C13 |
| Eriodictyol | 1.9 | Negative | 287.1 | 135.1 | 26 | 147 | Diosmetin |
| | | | | 150.9 | 14 | 147 | C13 |
| Luteolin | 2.1 | Negative | 285.0 | 133.0 | 34 | 213 | Diosmetin |
| | | | | | | | C13 |

TABLE 5
For method 2

| | | | | | | | Reference |
|---|---|---|---|---|---|---|---|
| | Retention | | Precursor | Daughter | Collision | Lens RF | internal |
| Molecules | time (min) | Polarity | ion | ion | energy | (V) | standard |
| Naringenin | 7.8 | Negative | 271.0 | 119.0 | 27 | 169 | Diosmetin |
| | | | | 150.9 | 18 | 169 | C13 |
| Apigenin | 8.2 | Negative | 269.0 | 117.1 | 35 | 201 | Diosmetin |
| | | | | 150.9 | 24 | 201 | C13 |
| Eriodictyol | 6.2 | Negative | 287.1 | 135.1 | 26 | 147 | Diosmetin |
| | | | | 150.9 | 14 | 147 | C13 |
| Luteolin | 6.6 | Negative | 285.0 | 133.0 | 34 | 213 | Diosmetin |
| | | | | 150.9 | 25 | 213 | C13 |
| Hesperetin | 8.7 | Negative | 301.0 | 164.0 | 24 | 169 | Diosmetin |
| | | | | 150.9 | 17 | 169 | C13 |
| Diosmetin | 9.1 | Negative | 299.0 | 256.0 | 30 | 192 | Diosmetin |
| | | | | 284.1 | 21 | 192 | C13 |
| Hesperetin 7- | 4.9 | Negative | 463.2 | 286.0 | 32 | 141 | Diosmin |
| O-glucoside | | | | 301.1 | 15 | 141 | C13 |
| Hesperidin | 3.9 | Negative | 609.2 | 286.0 | 42 | 211 | Diosmin |
| | | | | 301.1 | 25 | 211 | C13 |
| Diosmetin 7- | 5.0 | Negative | 461.1 | 284.0 | 36 | 194 | Diosmin |
| O-glucoside | | | | 299.1 | 10 | 194 | C13 |
| Diosmin | 4.0 | Negative | 607.2 | 284.0 | 50 | 274 | Diosmin |
| | | | | 299.0 | 25 | 274 | C13 |

### F3′ H

Constructs for each of the F3′Hs were made in a vector bearing the URA selection marker (Table 6). Constructs including each SAM2 and only one of the various CPRs were created in a vector bearing the LEU selection marker (Table 7). Two vectors including only the URA or LEU selection marker were also created as controls. The marker genes make it possible to detect and to select the cells that have incorporated the gene of interest.

TABLE 6
List of the various F3′H constructs tested

| Names | Assembled genes | Markers |
|---|---|---|
| FL 23 | F3′H from Perilla frutescens var. crispa | URA |
| | (SEQ ID NO: 2) | |
| FL 24 | F3′H from Phanerochaete chrysosporium | URA |
| | (SEQ ID NO: 4) | |
| FL 25 | F3′H from Petunia x hybrida (SEQ ID NO: 6) | URA |
| FL 26 | F3′H from Callistephus chinensis (SEQ ID NO: 8) | URA |
| FL 27 | F3′H from Callistephus chinensis (SEQ ID NO: | URA |
| | 10) | |
| FL 28 | F3′H from Gerbera hybrida (SEQ ID NO: 12) | URA |
| FL 29 | F3′H from Osteospermum hybrid cultivar | URA |
| | (SEQ ID NO: 14) | |
| FL 30 | F3′H from Citrus clementina (SEQ ID NO: 16) | URA |
| FL 31 | F3′H from Citrus sinensis (SEQ ID NO: 18) | URA |
| FL 32 | F3′H from Pilosella officinarum (SEQ ID NO: 20) | URA |
| FL 1031 | F3′H from Arabidopsis thaliana (SEQ ID NO: | URA |
| | 122) | |
| TT URA | — | URA |

TABLE 7
List of constructs made with the various CPRs

| Names | Assembled genes | Markers |
|---|---|---|
| FL 121 | CPR from Catharanthus roseus (SEQ ID NO: 24), | LEU |
| (CPR + | SAM from Saccharomyces cerevisiae (SEQ ID | |
| SAM) | NO: 82) | |
| FL 274 | chimeric CPR (SEQ ID NO: 28), | LEU |
| (CPR + | SAM from Saccharomyces cerevisiae (SEQ ID | |
| SAM) | NO: 82) | |
| FL 275 | ATR from Arabidopsis thaliana (SEQ ID NO: 30), | LEU |
| (ATR + | SAM from Saccharomyces cerevisiae (SEQ ID | |
| SAM) | NO: 82) | |
| FL 401 | CPR from Saccharomyces cerevisiae (SEQ ID | LEU |
| (CPR + | NO: 26), | |
| SAM) | SAM from Saccharomyces cerevisiae (SEQ ID | |
| | NO: 82) | |
| FL 463 | ATR from Arabidopsis thaliana (SEQ ID NO: 32), | LEU |
| (ATR + | SAM from Saccharomyces cerevisiae (SEQ ID | |
| SAM) | NO: 82) | |
| TT LEU | — | LEU |

Several strains were created with, respectively, all the F3′Hs listed in Table 6 so that they could each be tested with the constructs of Table 7.

These various assemblies make it possible to check the enzymatic activity of the F3′Hs and also make it possible to determine the most efficient F3′H-CPR pairs.

For example, the strain FL 405 contains the constructs FL 26 and FL 401.

The control strain (without the genes) containing the constructs TT URA and TT LEU is called CF235.

### FNSII

For each of the following FNSIIs, constructs in a TRP vector were prepared (Table 8). The same vectors with the LEU selection marker each containing SAM2 and a different CPR were used to test the FNSIIs (Table 9).

TABLE 8
Constructs including the various FNSIIs tested

| Names | Assembled genes | Markers |
|---|---|---|
| FL 620 | TAL from Rhodotorula glutinis (SEQ ID NO: 42), | TRP |
| (TAL + | 4CL from Petroselinum crispum (SEQ ID NO: 46), | |
| 4CL + | CHS from Citrus sinensis (SEQ ID NO: 54), | |
| CHS + | CHI from Arabidopsis thaliana (SEQ ID NO: 62) | |
| CHI + | FNSII from Lonicera japonica (SEQ ID NO: 34) | |
| FNSII) | | |
| FL 621 | TAL from Rhodotorula glutinis (SEQ ID NO: 42), | TRP |
| (TAL + | 4CL from Petroselinum crispum (SEQ ID NO: 46), | |
| 4CL + | CHS from Citrus sinensis (SEQ ID NO: 54), | |
| CHS + | CHI from Arabidopsis thaliana (SEQ ID NO: 62) | |
| CHI + | FNSII from Lonicera macranthoides (SEQ ID NO: | |
| FNSII) | 36) | |
| FL 112 | TAL from Flavobacetrium jonhsoniae (SEQ ID NO: | TRP |
| (TAL + | 40), | |
| 4CL + | 4CL from Petroselinum crispum (SEQ ID NO: 46), | |
| CHS + | CHS from Citrus sinensis (SEQ ID NO: 54), | |
| CHI + | CHI from Arabidopsis thaliana (SEQ ID NO: 62) | |
| FNSII) | FNSII from Petroselinum crispum (SEQ ID NO: 38) | |
| TT TRP | — | TRP |

TABLE 9
List of constructs made with the various CPRs

| Names | Assembled genes | Markers |
|---|---|---|
| FL 121 | CPR from Catharanthus roseus (SEQ ID NO: 24), | LEU |
| (CPR + | SAM from Saccharomyces cerevisiae (SEQ ID | |
| SAM) | NO: 82) | |
| FL 274 | Chimeric CPR (SEQ ID NO: 28), | LEU |
| (CPR + | SAM from Saccharomyces cerevisiae (SEQ ID | |
| SAM) | NO: 82) | |
| FL 275 | ATR from Arabidopsis thaliana (SEQ ID NO: 30), | LEU |
| (ATR + | SAM from Saccharomyces cerevisiae (SEQ ID | |
| SAM) | NO: 82) | |
| FL 401 | CPR from Saccharomyces cerevisiae (SEQ ID | LEU |
| (CPR + | NO: 26), | |
| SAM) | SAM from Saccharomyces cerevisiae (SEQ ID | |
| | NO: 82) | |
| FL 463 | ATR from Arabidopsis thaliana (SEQ ID NO: 32), | LEU |
| (ATR + | SAM from Saccharomyces cerevisiae (SEQ ID | |
| SAM) | NO: 82) | |
| TT LEU | — | LEU |

Several strains were created with, respectively, each of the constructs of the FNSIIs listed in Table 8 and each of the constructs of the CPRs of Table 9.

These various assemblies make it possible to check the enzymatic activity of the FNSIIs and also make it possible to determine the most efficient FNSIIs.

For example, the strain SC 744 contains the constructs FL 620 and FL 401.

The control strain (without the genes) containing the constructs TT TRP and TT LEU is called CF234.

Similar constructs were made to test the FNSIIs of SEQ ID NOs: 127, 129, 131, 133, 135, 137, 139, 141, 143, 145, 147, 149, 151, 153, 155, 157 and 159.

### Yeast Up to Eriodictyol/Luteolin

Strains including the pathway up to eriodictyol and luteolin were also tested:
the strain SC1500 comprises the constructs FL 26, FL 602, FL 808 and FL 822;
the strain SC2424 comprising the constructs FL 1031+FL 602+FL 822+TT HIS;
the strain SC2425 comprising the constructs FL 26+FL 602+FL 822+TT HIS;
the strain SC2426 comprising the constructs FL 31+FL 602+FL 822+TT HIS;
the strain SC2427 comprises the constructs FL 1031, FL 602, FL 808 and FL 822; and
the strain SC2428 comprising the constructs FL 31+FL 602+FL 808+FL 822.

TABLE 10
Lists of constructs used for the strains including the
pathway up to eriodictyol and luteolin

| Names | Assembled genes | Markers |
|---|---|---|
| FL 26 | F3′H from Callistephus chinensis (SEQ ID NO: 8) | URA |
| (F3′H) | | |
| FL 1031 | F3′H from Arabidopsis thaliana (SEQ ID NO: 122) | URA |
| (F3′H) | | |
| FL 31 | F3′H from Citrus sinensis (SEQ ID NO: 18) | URA |
| (F3′H) | | |
| FL 602 | TAL from Rhodotorula glutinis (SEQ ID NO: 42), | TRP |
| (TAL + | 4CL from Petroselinum crispum (SEQ ID NO: 46), | |
| 4CL + | CHS from Citrus sinensis (SEQ ID NO: 54), | |
| CHS + | CHI from Arabidopsis thaliana (SEQ ID NO: 62) | |
| CHI + | FNSII from Petroselinum crispum (SEQ ID NO: 38) | |
| FNS) | | |
| FL 808 | PAL from Arabidopsis thaliana (SEQ ID NO: 78), | HIS |
| (PAL + | C4H from Arabidopsis thaliana (SEQ ID NO: 80), | |
| C4H) | | |
| FL 822 | CPR from Catharanthus roseus (SEQ ID NO: 24), | LEU |
| (CPR + | CAF from Rhodopseudomonas palustris (SEQ ID | |
| CAF) | NO: 76) | |
| TT HIS | | HIS |

The control strain (without the genes) containing the constructs TT URA, TT TRP, TT HIS and TT LEU is called CF237.

### MET:

In order to test each of the METs, constructs were made and are presented in Table 11. The marker genes make it possible to detect and to select the cells that have incorporated the gene of interest.

TABLE 11
List of constructs made to test the various METs

| Names | Assembled genes | Markers |
|---|---|---|
| FL 121 | CPR from Catharanthus roseus | LEU |
| (CPR + SAM) | (SEQ ID NO: 24), | |
| | SAM from Saccharomyces cereyisiae | |
| | (SEQ ID NO: 82) | |
| FL 266 | F3′H from Callistephus chinensis | URA |
| (F3′H + MET) | (SEQ ID NO: 8), | |
| | MET from Arabidopsis thaliana | |
| | (SEQ ID NO: 88) | |
| FL 268 | F3′H from Callistephus chinensis | URA |
| (F3′H + MET) | (SEQ ID NO: 8), | |
| | MET from Homo sapiens | |
| | (SEQ ID NO: 90) | |
| FL 469 | F3′H from Callistephus chinensis | URA |
| (F3′H + MET) | (SEQ ID NO: 8) | |
| | MET from Citrus clementina | |
| | (SEQ ID NO: 118) | |
| FL 475 | F3′H from Callistephus chinensis | URA |
| (F3′H + MET) | (SEQ ID NO: 8) | |
| | MET from Citrus sinensis | |
| | (SEQ ID NO: 120) | |

Four strains SC1612, SC1614, SC2147 and SC2151 were created, with FL 121 and FL 266 for SC1612, FL 121 and FL 268 for SC1614, FL 475 and FL 121 for SC2147 and FL 469 and FL 121 for SC2151 for the conversion of eriodictyol into hesperetin in order to determine which MET is the most efficient.

The control strain (without the genes) containing the constructs TT LEU and TT URA is called CF235.

F3′H, MET, FNS, CPR: Production of Diosmetin from Naringenin

TABLE 12
List of constructs used to test the enzymes
in Saccharomyces cerevisiae (SC)

| Names | Assembled genes | Markers |
|---|---|---|
| FL 121 | CPR from Catharanthus roseus | LEU |
| (CPR + SAM) | (SEQ ID NO: 24), | |
| | SAM from Saccharomyces cerevisiae | |
| | (SEQ ID NO: 82) | |
| TT LEU | — | LEU |
| FL 26 | F3′H from Callistephus chinensis | URA |
| (F3′H) | (SEQ ID NO: 8), | |
| FL 1031 | F3′H from Arabidopsis thaliana | URA |
| (F3′H) | (SEQ ID NO: 122), | |
| FL 1111 | FNSII from Petroselinum crispum | TRP |
| (FNS + MET) | (SEQ ID NO: 33) | |
| | MET from Citrus clementina | |
| | (SEQ ID NO: 118) | |
| FL 1112 | FNSII from Angelica archangelica | TRP |
| (FNS + MET) | (SEQ ID NO: 128) | |
| | MET from Citrus clementina | |
| | (SEQ ID NO: 118) | |
| FL 1113 | FNSII from Cynara cardunculus var. | TRP |
| (FNS + MET) | scolymus | |
| | (SEQ ID NO: 130) | |
| | MET from Citrus clementina | |
| | (SEQ ID NO: 118) | |
| FL 1114 | FNSII from Perilla frutescens var. crispa | TRP |
| (FNS + MET) | (SEQ ID NO: 132) | |
| | MET from Citrus clementina | |
| | (SEQ ID NO: 118) | |
| FL 1115 | FNSII from Dahlia pinnata | TRP |
| (FNS + MET) | (SEQ ID NO: 134) | |
| | MET from Citrus clementina | |
| | (SEQ ID NO: 118) | |
| FL 1116 | FNSII from Petroselinum crispum | TRP |
| (FNS + MET) | (SEQ ID NO: 33) | |
| | MET from Citrus sinensis (SEQ ID NO: 120) | |
| FL 1118 | FNSII from Cynara cardunculus var. | TRP |
| (FNS + MET) | scolymus (SEQ ID NO: 130) | |
| | MET from Citrus sinensis (SEQ ID NO: 120) | |
| FL 1119 | FNSII from Perilla frutescens var. crispa | TRP |
| (FNS + MET) | (SEQ ID NO: 132) | |
| | MET from Citrus sinensis (SEQ ID NO: 120) | |
| FL 1120 | FNSII from Dahlia pinnata | TRP |
| (FNS + MET) | (SEQ ID NO: 134) | |
| | MET from Citrus sinensis (SEQ ID NO: 120) | |

The following strains were constructed:
C2429: FL 1111I+FL 1031+FL 121
SC2430: FL 1112+FL 1031+FL 121
SC2431: FL 1113+FL 1031+FL 121
SC2432: FL 1114+FL 1031+FL 121
SC2433: FL 1115+FL 1031+FL 121
SC2439: FL 1111I+FL 1031+TT LEU
SC2440: FL 1112+FL 1031+TT LEU
SC2441: FL 1113+FL 1031+TT LEU
SC2442: FL 1114+FL 1031+TT LEU
SC2443: FL 1115+FL 1031+TT LEU
SC2434: FL 1116+FL 1031+FL 121
SC2436: FL 1118+FL 1031+FL 121
SC2437: FL 1119+FL 1031+FL 121
SC2438: FL 1120+FL 1031+FL 121
SC2444: FL 1116+FL 1031+TT LEU
SC2446: FL 1118+FL 1031+TT LEU
SC2447: FL 1119+FL 1031+TT LEU
SC2448: FL 1120+FL 1031+TT LEU
SC2449: FL 1111+FL 26+FL 121
SC2450: FL 1112+FL 26+FL 121
SC2451: FL 1113+FL 26+FL 121
SC2452: FL 1114+FL 26+FL 121
SC2453: FL 1115+FL 26+FL 121
SC2459: FL 1111+FL 26+TT LEU
SC2460: FL 1112+FL 26+TT LEU
SC2461: FL 1113+FL 26+TT LEU
SC2462: FL 1114+FL 26+TT LEU
SC2463: FL 1115+FL 26+TT LEU
SC2454: FL 1116+FL 26+FL 121
SC2456: FL 1118+FL 26+FL 121
SC2457: FL 1119+FL 26+FL 121
SC2458: FL 1120+FL 26+FL 121
SC2464: FL 1116+FL 26+TT LEU
SC2466: FL 1118+FL 26+TT LEU
SC2467: FL 1119+FL 26+TT LEU
SC2468: FL 1120+FL 26+TT LEU

The control strain (without the genes) containing the constructs TT URA, TT TRP, TT HIS and TT LEU is called CF237.

E. coli Up to Hesperetin/Diosmetin

TABLE 13
List of constructs used to test the enzymes in E. coli

| Names | Assembled genes |
|---|---|
| EC26 (SAM + | SAM from Saccharomyces cereyisiae (SEQ ID NO: 82) |
| MET) | MET from Homo sapiens (SEQ ID NO: 90) |
| EC41 (SAM + | SAM from Saccharomyces cereyisiae (SEQ ID NO: 82) |
| MET) | MET from Citrus clementina (SEQ ID NO: 118) |
| EC43 (SAM + | SAM from Saccharomyces cereyisiae (SEQ ID NO: 82) |
| MET) | MET from Citrus sinensis (SEQ ID NO: 120) |
| EC30 (FNSII) | FNSII from Petroselinum crispum (SEQ ID NO: 38) |

### Yeast Up to Hesperetin/Diosmetin

Three strains including the pathway up to hesperetin/diosmetin were also tested. The strain SC1508 comprises the constructs FL 121+FL 268+FL 602+FL 808 of Table 14. The strain SC2408 comprises the constructs FL 121+FL 469+FL 602+FL 808 of Table 14. The strain SC2409 comprises the constructs FL 121+FL 475+FL 602+FL 808 of Table 14.

TABLE 14
List of constructs used in the examples

| Names | Assembled genes | Markers |
|---|---|---|
| FL 121 | CPR from Catharanthus roseus | LEU |
| (CPR + SAM) | (SEQ ID NO: 24), | |
| | SAM from Saccharomyces cereyisiae | |
| | (SEQ ID NO: 82) | |
| FL 268 | F3′H from Catllistephus chinensis | URA |
| (F3′H + MET) | (SEQ ID NO: 8), | |
| | MET from Homo sapiens (SEQ ID NO: 90) | |
| FL 469 | F3′H from Callistephus chinensis | URA |
| (F3′H + MET) | (SEQ ID NO: 8) | |
| | MET from Citrus clementina | |
| | (SEQ ID NO: 118) | |
| FL 475 | F3′H from Callistephus chinensis | URA |
| (F3′H + MET) | (SEQ ID NO: 8) | |
| | MET from Citrus sinensis (SEQ ID NO: 120) | |
| FL 602 | TAL from Rhodotorula glutinis | TRP |
| (TAL + 4CL + | (SEQ ID NO: 42), | |
| CHS + | 4CL from Petroselinum crispum | |
| CHI + FNSII) | (SEQ ID NO: 46), | |
| | CHS from Citrus sinensis (SEQ ID NO: 54), | |
| | CHI from Arabidopsis thaliona | |
| | (SEQ ID NO: 62) | |
| | FNSII from Petroselinum crispum | |
| | (SEQ ID NO: 38) | |
| FL 808 | PAL from Arabidopsis thaliona | HIS |
| (PAL + C4H) | (SEQ ID NO: 78), | |
| | C4H from Arabidopsis thaliona | |
| | (SEQ ID NO: 80), | |
| TT LEU | — | LEU |
| TT URA | — | URA |
| TT TRP | — | TRP |
| TT HIS | — | HIS |

The control strain (without the genes) containing the constructs TT LEU, TT URA, TT TRP and TT HIS is called CF237.

### GT

In order to test each of the GTs, constructs were made and are presented in Table 15. The marker genes make it possible to detect and to select the cells that have incorporated the gene of interest.

TABLE 15
List of constructs used to test the various GTs

| Names | Assembled genes | Markers |
|---|---|---|
| FL 545 | F3′H from Callistephus chinensis (SEQ ID NO: 8), | URA |
| (F3′H + | MET from Citrus sinensis (SEQ ID NO: 120), | |
| MET + | GT from Arabidopsis thaliana (SEQ ID NO: 92), | |
| GT + RHM + | RHM from Citrus sinensis (SEQ ID NO: 108) | |
| RHAT) | RHAT from Citrus clementina (SEQ ID NO: 104) | |
| FL 546 | F3′H from Callistephus chinensis (SEQ ID NO: 8), | URA |
| (F3′H + | MET from Citrus sinensis (SEQ ID NO: 120), | |
| MET + | GT from Scutellaria baicalensis (SEQ ID NO: 94), | |
| GT + RHM + | RHM from Citrus sinensis (SEQ ID NO: 108) | |
| RHAT) | RHAT from Citrus clementina (SEQ ID NO: 104) | |
| FL 547 | F3′H from Callistephus chinensis (SEQ ID NO: 8), | URA |
| (F3′H + | MET from Citrus sinensis (SEQ ID NO: 120), | |
| MET + | GT from Scutellaria baicalensis (SEQ ID NO: 96), | |
| GT + RHM + | RHM from Citrus sinensis (SEQ ID NO: 108) | |
| RHAT) | RHAT from Citrus clementina (SEQ ID NO: 104) | |
| FL 548 | F3′H from Callistephus chinensis (SEQ ID NO: 8), | URA |
| (F3′H + | MET from Citrus sinensis (SEQ ID NO: 120), | |
| MET + | GT from Scutellaria baicalensis (SEQ ID NO: 98), | |
| GT + RHM + | RHM from Citrus sinensis (SEQ ID NO: 108 | |
| RHAT) | RHAT from Citrus clementina (SEQ ID NO: 104) | |
| FL 549 | F3′H from Callistephus chinensis (SEQ ID NO: 8), | URA |
| (F3′H + | MET from Citrus sinensis (SEQ ID NO: 120), | |
| MET + | GT from Homo sapiens (SEQ ID NO: 100), | |
| GT + RHM + | RHM from Citrus sinensis (SEQ ID NO: 108) | |
| RHAT) | RHAT from Citrus clementina (SEQ ID NO: 104) | |
| FL 550 | F3′H from Callistephus chinensis (SEQ ID NO: 8), | |
| (F3′H + | MET from Citrus sinensis (SEQ ID NO: 120), | |
| MET + | GT from Homo sapiens (SEQ ID NO: 102), | URA |
| GT + RHM + | RHM from Citrus sinensis (SEQ ID NO: 108) | |
| RHAT) | RHAT from Citrus clementina (SEQ ID NO: 104) | |
| FL 554 | F3′H from Callistephus chinensis (SEQ ID NO: 8), | URA |
| (F3′H + | MET from Citrus sinensis (SEQ ID NO: 120), | |
| MET + GT + | GT from Citrus sinensis (SEQ ID NO: 114), | |
| UDPRHA + | UDPRHA from Citrus sinensis (SEQ ID NO: 108), | |
| RHAT) | RHAT from Citrus clementina (SEQ ID NO: 104) | |
| FL 556 | F3′H from Callistephus chinensis (SEQ ID NO: 8), | URA |
| (F3′H + | MET from Citrus sinensis (SEQ ID NO: 120), | |
| MET + GT + | GT from Citrus clementina (SEQ ID NO: 116), | |
| UDPRHA + | UDPRHA from Citrus sinensis (SEQ ID NO: 108), | |
| RHAT) | RHAT from Citrus clementina (SEQ ID NO: 104) | |
| TT URA | — | URA |

The various constructs with the various GTs make it possible to check the enzymatic activity of the GTs and also make it possible to determine the most efficient GTs.

The control strain (without the genes) containing the construct TT URA is called CF233.

### RHM

In order to test each of the RHMs, constructs were made and are presented in Table 16.

TABLE 16
List of constructs used to test the various RHMs

| Names | Assembled genes | Markers |
|---|---|---|
| FL 545 | F3′H from Callistephus chinensis | URA |
| (F3′H + | (SEQ ID NO: 8), | |
| MET + GT + | MET from Citrus sinensis (SEQ ID NO: 120), | |
| RHM + | GT from Arabidopsis thaliana (SEQ ID NO: 98), | |
| RHAT) | RHM from Citrus sinensis (SEQ ID NO: 108), | |
| | RHAT from Citrus clementina (SEQ ID NO: 104) | |
| FL 512 | F3′H from Callistephus chinensis | URA |
| (F3′H + | (SEQ ID NO: 8), | |
| MET + GT + | MET from Homo sapiens (SEQ ID NO: 90), | |
| RHM + | GT from Arabidopsis thaliana (SEQ ID NO: 98), | |
| RHAT) | RHM from Arabidopsis thaliana | |
| | (SEQ ID NO: 110), | |
| | RHAT from Citrus clementina (SEQ ID NO: 104) | |
| TT URA | — | URA |

The various constructs with the various RHMs make it possible to check the enzymatic activity of the RHMs and also make it possible to determine the most efficient RHMs.

The control strain (without the genes) containing the construct TT URA is called CF233.

### RHAT

In order to test each of the RHATs, constructs were made and are presented in Table 17.

TABLE 17
List of constructs used to test the various RHATs

| Names | Assembled genes | Markers |
|---|---|---|
| FL 512 | F3′H from Callistephus chinensis | URA |
| (F3′H + | (SEQ ID NO: 8), | |
| MET + GT3 + | MET from Homo sapiens (SEQ ID NO: 90), | |
| RHM + | GT from Arabidopsis thaliana (SEQ ID NO: 92), | |
| RHAT) | RHM from Arabidopsis thaliana | |
| | (SEQ ID NO: 110) | |
| | RHAT from Citrus clementina (SEQ ID NO: 104) | |
| FL 165 | F3′H from Callistephus chinensis | URA |
| (F3′H + | (SEQ ID NO: 8), | |
| MET + GT + | MET from Homo sapiens (SEQ ID NO: 90), | |
| RHM + | GT from Arabidopsis thaliana (SEQ ID NO: 92), | |
| RHAT) | RHM from Arabidopsis thaliana | |
| | (SEQ ID NO: 110) | |
| | RHAT from Citrus sinensis (SEQ ID NO: 106) | |
| TT URA2 | — | URA |

The various assemblies made with the various RHATs make it possible to check the enzymatic activity of the RHATs and also make it possible to determine the most efficient RHATs.

The control strain (without the genes) containing the construct TT URA is called CF233.

E. coli Up to Hesperidin/Diosmin

TABLE 18
List of strains created to test the pathway in E. coli

| Names | Assembled genes |
|---|---|
| EC38 | GT from Arabidopsis thaliona (SEQ ID NO: 92), |
| (GT + RHM + | RHM from Citrus sinensis (SEQ ID NO: 108) |
| RHAT) | RHAT from Citrus clementina (SEQ ID NO: 104) |
| EC45 | GT from Citrus sinensis (SEQ ID NO: 114), |
| (GT + UDPRHA + | UDPRHA from Citrus sinensis (SEQ ID NO: 108), |
| RHAT) | RHAT from Citrus clementina (SEQ ID NO: 104) |
| EC47 | GT from Citrus clementina (SEQ ID NO: 116), |
| (GT + UDPRHA + | UDPRHA from Citrus sinensis (SEQ ID NO: 108), |
| RHAT) | RHAT from Citrus clementina (SEQ ID NO: 104) |

### Yeast Up to Hesperidin/Diosmin

Nine strains including the full pathway were also created.

The strain SC1509 comprises the constructs FL 121+FL 511+FL 602+FL 808.

The strain SC1530 comprises the constructs FL 121+FL 603+FL 602+FL 808.

The strain SC1529 comprises the constructs FL 121+FL 554+FL 602+FL 808.

The strain SC1568 comprises the constructs FL 121+FL 556+FL 602+FL 808.

The strain SC2410 comprises the constructs FL 121+FL 1100+FL 602+FL 808.

The strain SC1579 comprises the constructs FL 401+FL 547+FL 602+FL 828.

The strain SC1584 comprises the constructs FL 401+FL 554+FL 602+FL 828.

The strain SC1621 comprises the constructs FL 401+FL 556+FL 602+FL 828.

The strain SC1626 comprises the constructs FL 401+FL 603+FL 602+FL 828.

TABLE 19
Lists of constructs used to create the strains up to hesperidin/diosmin

| Names | Assembled genes | Markers |
|---|---|---|
| FL 121 | CPR from Catharanthus roseus (SEQ ID NO: 24), | LEU |
| (CPR + SAM) | SAM from Saccharomyces cereyisiae | |
| | (SEQ ID NO: 82) | |
| FL 401 | CPR from Saccharomyces cereyisiae | LEU |
| (CPR + SAM) | (SEQ ID NO: 26), | |
| | SAM from Saccharomyces cereyisiae | |
| | (SEQ ID NO: 82) | |
| FL 547 | F3′H from Callistephus chinensis | URA |
| (F3′H + | (SEQ ID NO: 8), | |
| MET + GT + | MET from Citrus sinensis (SEQ ID NO: 120), | |
| RHM + | GT from Scutellaria baicalensis | |
| RHAT) | (SEQ ID NO: 96), | |
| | RHM from Citrus sinensis (SEQ ID NO: 108) | |
| | RHAT from Citrus clementina (SEQ ID NO: 104) | |
| FL 511 | F3′H from Callistephus chinensis | URA |
| (F3′H + | (SEQ ID NO: 8), | |
| MET + GT + | MET from Homo sapiens (SEQ ID NO: 90), | |
| RHM + | GT from Arabidopsis thaliana (SEQ ID NO: 92), | |
| RHAT) | RHM from Citrus sinensis (SEQ ID NO: 108) | |
| | RHAT from Citrus clementina (SEQ ID NO: 104) | |
| FL 603 | F3′H from Callistephus chinensis | |
| (F3′H + | (SEQ ID NO: 8), | |
| MET + GT + | MET from Citrus clementina (SEQ ID NO: 118), | URA |
| UDPRHA + | GT from Citrus sinensis (SEQ ID NO: 114), | |
| RHAT) | UDPRHA from Citrus sinensis | |
| | (SEQ ID NO: 108), | |
| | RHAT from Citrus clementina (SEQ ID NO: 104) | |
| FL 554 | F3′H from Callistephus chinensis | |
| (F3′H + | (SEQ ID NO: 8), | |
| MET + GT + | MET from Citrus sinensis (SEQ ID NO: 120), | URA |
| UDPRHA + | GT from Citrus sinensis (SEQ ID NO: 114), | |
| RHAT) | UDPRHA from Citrus sinensis | |
| | (SEQ ID NO: 108), | |
| | RHAT from Citrus clementina (SEQ ID NO: 104) | |
| FL 556 | F3′H from Callistephus chinensis | |
| (F3′H + | (SEQ ID NO: 8), | |
| MET + GT + | MET from Citrus sinensis (SEQ ID NO: 120), | URA |
| UDPRHA + | GT from Citrus clementina (SEQ ID NO: 116), | |
| RHAT) | UDPRHA from Citrus sinensis | |
| | (SEQ ID NO: 108), | |
| | RHAT from Citrus clementina (SEQ ID NO: 104) | |
| FL 1100 | F3′H from Callistephus chinensis | |
| (F3′H + | (SEQ ID NO: 8), | |
| MET + GT + | MET from Citrus clementina (SEQ ID NO: 118), | URA |
| UDPRHA + | GT from Citrus clementina (SEQ ID NO: 116), | |
| RHAT) | UDPRHA from Citrus sinensis | |
| | (SEQ ID NO: 108), | |
| | RHAT from Citrus clementina (SEQ ID NO: 104) | |
| FL 602 | TAL from Rhodotorula glutinis (SEQ ID NO: 42), | |
| (TAL + | 4CL from Petroselinum crispum | TRP |
| 4CL + CHS + | (SEQ ID NO: 46), | |
| CHI + | CHS from Citrus sinensis (SEQ ID NO: 54), | |
| FNS) | CHI from Arabidopsis thaliona (SEQ ID NO: 62) | |
| | FNSII from Petroselinum crispum | |
| | (SEQ ID NO: 38) | |
| FL 808 | PAL from Arabidopsis thaliona (SEQ ID NO: 78), | HIS |
| (PAL + C4H) | C4H from Arabidopsis thaliona (SEQ ID NO: 80), | |
| FL 828 | PAL from Citrus sinensis (SEQ ID NO: 66), | HIS |
| (PAL + C4H) | C4H from Arabidopsis thaliona (SEQ ID NO: 80), | |
| TT LEU | — | LEU |
| TT URA | — | URA |
| TT TRP | — | TRP |
| TT HIS | — | HIS |

The control strain (without the genes) containing the constructs TT LEU, TT URA, TT TRP and TT HIS is called CF237.

### Results

### F3′H

Tables 20 and 21 below show the production of eriodictyol (Table 20) and of luteolin (Table 21) obtained by cultivating the strains comprising the F3′Hs listed in Table 6 and the constructs of Table 7, in the presence of naringenin and apigenin, respectively.

TABLE 20
Concentration of eriodictyol (in mg.l⁻¹)

| | WITHOUT | CPR | CPR | CPR | ATR | ATR |
|---|---|---|---|---|---|---|
| F3′H | CPR | (SEQ ID No | (SEQ ID No | (SEQ ID No | (SEQ ID No | (SEQ ID No |
| (SEQ ID No) | (TT LEU) | 24; FL121) | 26; FL401) | 28; FL274) | 30; FL275) | 32; FL463) |
| 2 (FL23) | 35.5 ± 2.9 | 42.1 ± 4.3 | 49.9 ± 4.2 | 43.6 ± 4.2 | 38.8 ± 4.1 | 43.3 ± 5.1 |
| 4 (FL24) | 1 ± 0.8 | 6.4 ± 0.5 | 4.0 ± 0.5 | 4.7 ± 0.3 | 5.1 ± 0.4 | 5.3 ± 0.4 |
| 6 (FL25) | 115.2 ± 3.2 | 76.8 ± 4.2 | 42.3 ± 2.6 | 70.2 ± 8.6 | 71.1 ± 8.7 | 71.3 ± 7.4 |
| 8 (FL26) | 108.3 ± 4.0 | 71.1 ± 7.1 | 89.2 ± 9.5 | 87.4 ± 5.0 | 75.8 ± 5.2 | 90.0 ± 6.1 |
| 10 (FL27) | 28.8 ± 1.2 | 57.7 ± 2.6 | 69.3 ± 10.6 | 79.1 ± 4.2 | 52.3 ± 0.5 | 69.7 ± 2.3 |
| 12 (FL28) | 108.0 ± 2.0 | 7.0 ± 1.4 | 9.1 ± 5.9 | 4.6 ± 0.3 | 7.4 ± 2.8 | 9.2 ± 0.6 |
| 14 (FL29) | 119.9 ± 1.1 | 39.9 ± 4.7 | 56.1 ± 16.3 | 64.8 ± 4.1 | 36.8 ± 4.4 | 46.1 ± 5.5 |
| 16 (FL30) | <QL | 76.3 ± 2.6 | 70.9 ± 6.2 | 70.4 ± 4.4 | 58.5 ± 10.9 | 76.9 ± 1.7 |
| 18 (FL31) | 107.3 ± 8.0 | 82.3 ± 17.2 | 102.2 ± 7.1 | 98.8 ± 5.9 | 96.6 ± 4.7 | 101.3 ± 4.0 |
| 20 (FL32) | 33.7 ± 4.0 | 68.9 ± 2.7 | 81.5 ± 3.4 | 63.6 ± 3.7 | 69.5 ± 0.9 | 69.7 ± 1.1 |
| 122 (FL1031) | 4.8 ± 0.3 | 60.5 ± 3.4 | 34.4 ± 2.8 | 25.8 ± 5.8 | 59.0 ± 1.7 | 40.0 ± 9.5 |

QL: below the quantification limit

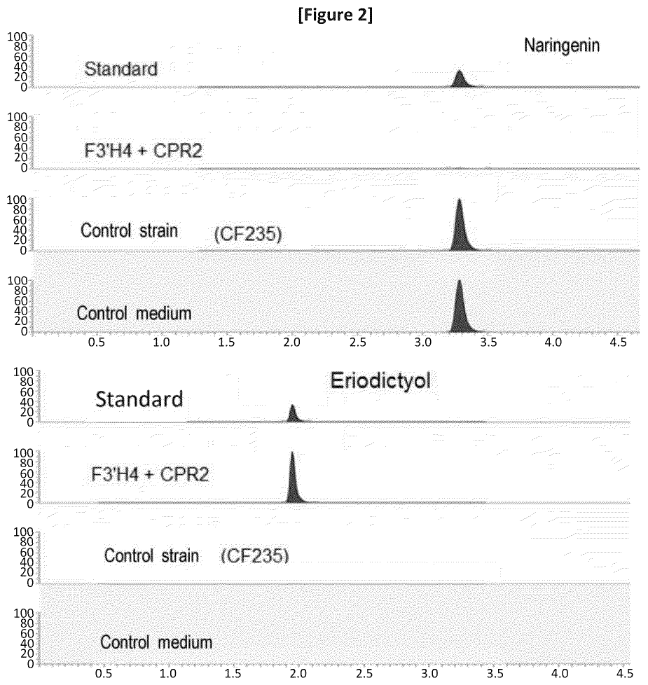

The various strains are indeed capable of producing eriodictyol from naringenin, in different concentrations according to the F3′Hs and the CPR used (see FIG. 2).

TABLE 21
Concentration of luteolin (in mg.l⁻¹)

| | WITHOUT | CPR | CPR | CPR | ATR | ATR |
|---|---|---|---|---|---|---|
| F3′H | CPR | (SEQ ID No | (SEQ ID No | (SEQ ID No | (SEQ ID No | (SEQ ID No |
| (SEQ ID No) | (TT LEU) | 24; FL121) | 26; FL401) | 28; FL274) | 30; FL275) | 32; FL463) |
| 2 (FL23) | 3.5 ± 0.1 | 11.7 ± 0.7 | 9.1 ± 2.1 | 11.01 ± 0.4 | 10.8 ± 1.7 | 10.2 ± 1.4 |
| 4 (FL24) | <QL | <QL | <QL | <QL | <QL | <QL |
| 6 (FL25) | 10.2 ± 0.9 | 12.8 ± 0.7 | 7.8 ± 1.4 | 11.9 ± 0.8 | 10.1 ± 1.2 | 12.9 ± 1.4 |
| 8 (FL26) | 9.5 ± 0.4 | 13.2 ± 1.1 | 8.2 ± 0.7 | 10.9 ± 0.7 | 12.2 ± 0.4 | 12.1 ± 0.7 |
| 10 (FL27) | <QL | 2.5 ± 0.3 | <QL | 0.5 ± 0 | 2.7 ± 0.1 | 2.77 ± 0.4 |
| 12 (FL28) | 12.1 ± 0.4 | 13.3 ± 1.2 | 14.7 ± 1.8 | 14.1 ± 1.7 | 12.5 ± 3.8 | 15.3 ± 0.9 |
| 14 (FL29) | 1.5 ± 0.1 | 0.6 ± 0.04 | 1.1 ± 0.2 | 0.8 ± 0.03 | 0.7 ± 0.06 | 1.0 ± 0.08 |
| 16 (FL30) | 0.5 ± 0.02 | 1.3 ± 0.1 | 2.5 ± 1.5 | 1.6 ± 0.1 | 1.5 ± 0.5 | 2.0 ± 0.1 |
| 18 (FL31) | 12.2 ± 0.7 | 13.2 ± 0.8 | 13.7 ± 1.2 | 12.7 ± 0.4 | 14.0 ± 1.8 | 12.7 ± 0.6 |
| 20 (FL32) | 1.2 ± 0.2 | 9.9 ± 1.4 | 2.8 ± 0.4 | 4.3 ± 0.1 | 11.0 ± 0.9 | 9.3 ± 1.8 |
| 122 (FL1031) | 0.4 ± 0.1 | 10.9 ± 0.1 | 3.0 ± 0.6 | 3.0 ± 0.9 | 11.4 ± 0.4 | 9.5 ± 1.6 |

QL: below the quantification limit

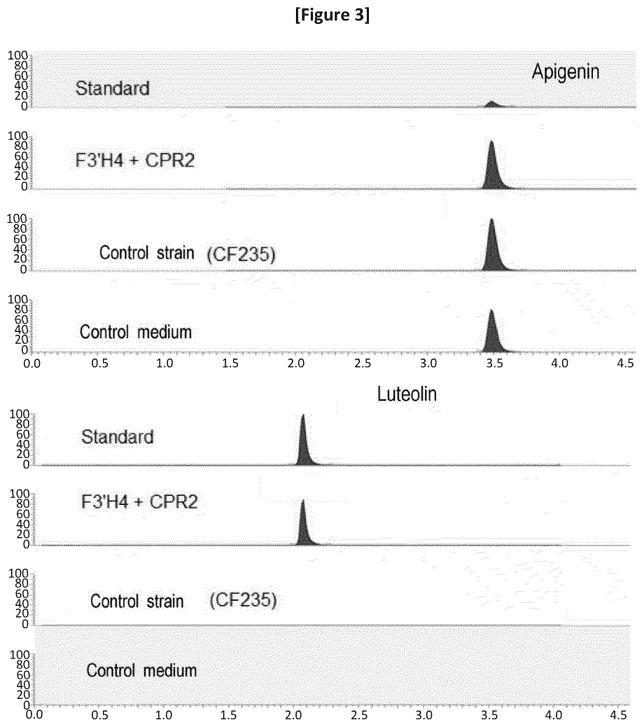

The various strains are indeed capable of producing luteolin from apigenin, in different concentrations according to the F3′Hs and the CPR used (see FIG. 3).

### FNS

Tables 22 and 23 below show the production of apigenin (Table 22) and of luteolin (Table 23) obtained by cultivating the strains comprising the FNSIIs listed in Table 8 and the constructs of Table 9, in the presence of naringenin and eriodictyol, respectively.

TABLE 22
Concentration of apigenin (in mg.l⁻¹)

| | WITHOUT | CPR | CPR | CPR | ATR | ATR |
|---|---|---|---|---|---|---|
| FNSII | CPR | (SEQ ID No | (SEQ ID No | (SEQ ID No | (SEQ ID No | (SEQ ID No |
| (SEQ ID No) | (TT LEU) | 24; FL121) | 26; FL401) | 28; FL274) | 30; FL275) | 32; FL463) |
| 34 (FL620) | 11.6 ± 0.3 | 34.7 ± 1.0 | 47.6 ± 5.7 | 37.7 ± 1.6 | 50.5 ± 1.5 | 51.3 ± 3.4 |
| 36 (FL621) | 3.5 ± 0.1 | 35.6 ± 0.2 | 14.9 ± 1.3 | 16.4 ± 1.4 | 29.8 ± 3.9 | 33.2 ± 1.5 |
| 38 (FL112) | 2.9 ± 0.1 | 40.7 ± 1.2 | 41.4 ± 1.5 | 34.2 ± 1.7 | 38.0 ± 0.9 | 43.5 ± 0.0 |

TABLE 23
Concentration of luteolin (in mg.l⁻¹)

| FNSII | WITHOUT | CPR | CPR | CPR | ATR | ATR |
|---|---|---|---|---|---|---|
| (SEQ ID | CPR | (SEQ ID No | (SEQ ID No | (SEQ ID No | (SEQ ID No | (SEQ ID No |
| No) | (TT LEU) | 24; FL121) | 26; FL401) | 28; FL274) | 30; FL275) | 32; FL463) |
| 34 (FL620) | 2.5 ± 0.2 | 1.4 ± 0.3 | 7.8 ± 1.4 | 4.5 ± 0.9 | 4.5 ± 2.9 | 8.5 ± 0.8 |
| 36 (FL621) | 0.2 ± 0.2 | 1.5 ± 0.1 | 1.3 ± 0.2 | 0.9 ± 0.1 | 1.3 ± 0.3 | 1.2 ± 0.2 |
| 38 (FL112) | 0.2 ± 0.0 | 4.5 ± 1.9 | 2.3 ± 0.5 | 2.6 ± 1.2 | 1.4 ± 0.0 | 1.6 ± 0.0 |

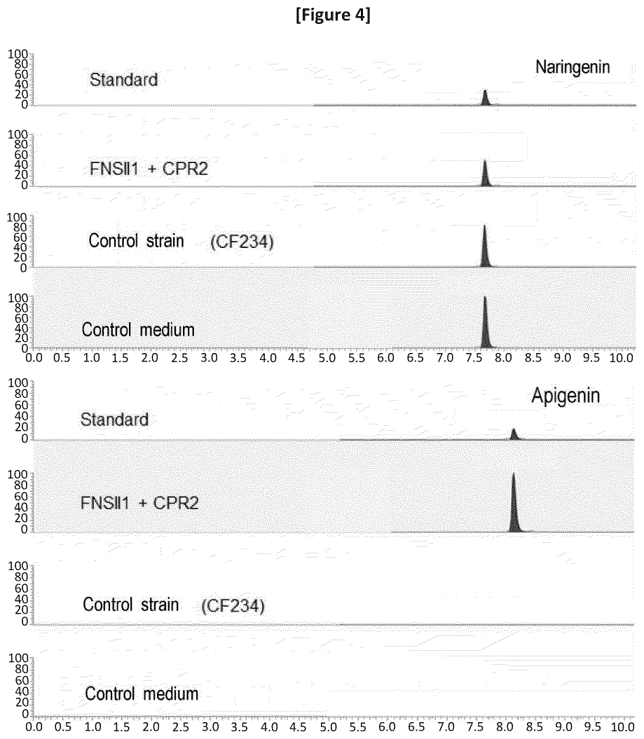

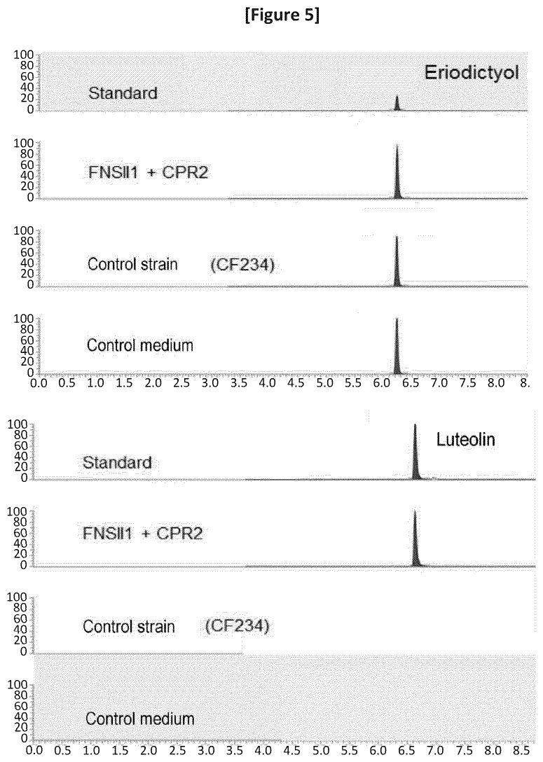

The various strains are indeed capable of producing apigenin and luteolin from naringenin and eriodictyol, in different concentrations according to the FNS used (FIGS. 4 and 5).

Similar results were obtained with the FNSIIs of SEQ ID NOs: 127, 129, 131, 133, 135, 137, 139, 141, 143, 145, 147, 149, 151, 153, 155, 157 and 159.

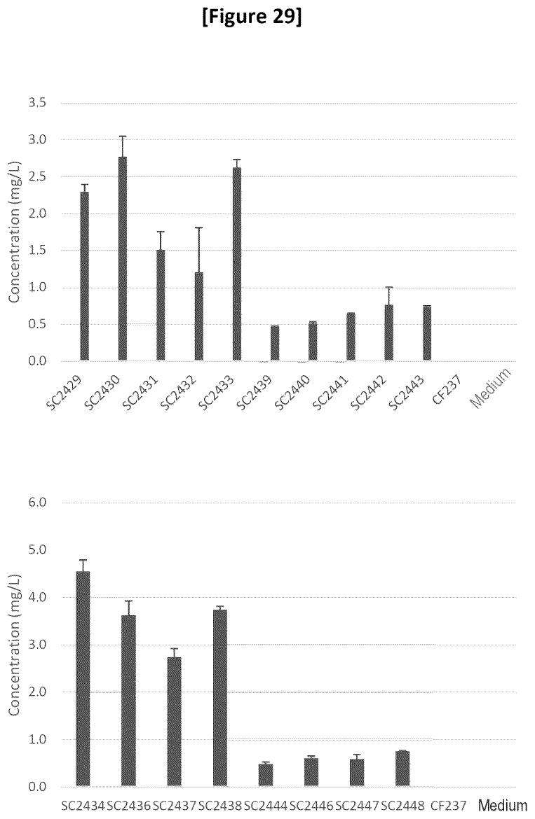

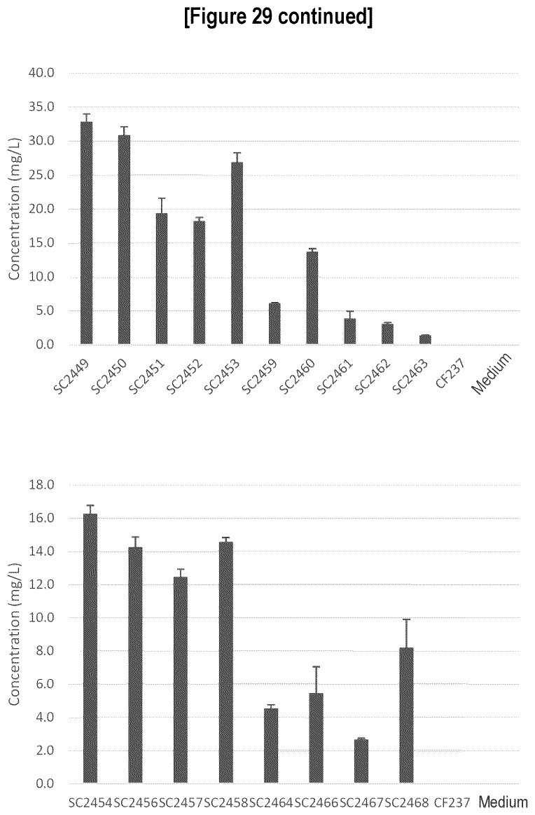

F3′H, MET, FNS, CPR: PRODUCTION OF DIOSMETIN FROM NARINGENIN The results for the production of diosmetin from naringenin by the strains SC2429 to SC2434, SC2436 to SC2444, SC2446 to SC2454, SC2456 to SC2464 and SC2466 to SC2468 are presented in FIG. 29.

All the strains are capable of producing diosmetin from naringenin. The production of diosmetin is largely increased by adding a CPR.

### Yeast Up to Eriodictyol/Luteolin

The strains SC2424, SC2425, SC2426, SC2427, SC1500 and SC2428 contain all the enzymes of the pathway up to eriodictyol and luteolin and are capable of producing luteolin and eriodictyol from glucose.

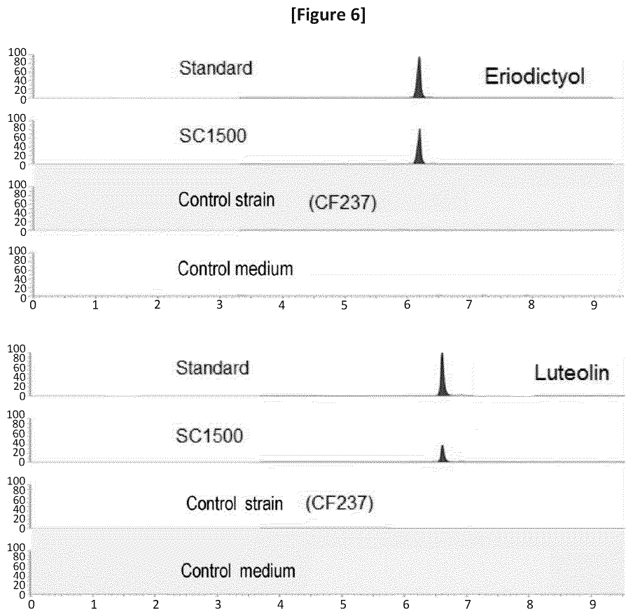

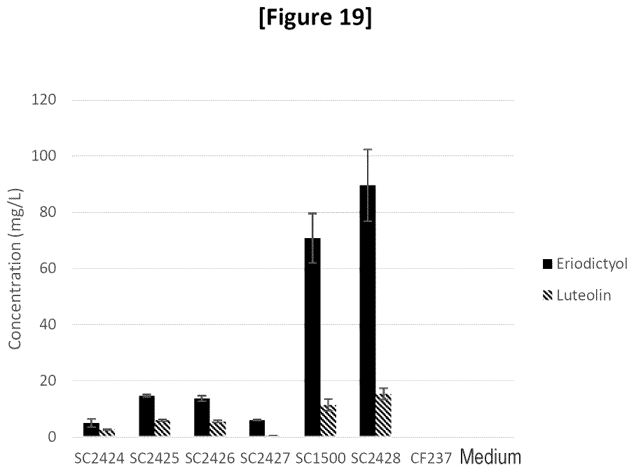

The results for the strain SC1500 correspond to FIG. 6, in which the eriodictyol and luteolin peaks are observed. Similar results are obtained for the strains SC2424, SC2425, SC2426, SC2427 and SC2428. The production of eriodictyol and of luteolin for each of the strains SC2424, SC2425, SC2426, SC2427, SC1500 and SC2428 is presented in FIG. 19.

It should be noted that the addition of the enzymes PAL and C4H to the biosynthetic pathway makes it possible to obtain markedly higher eriodictyol and luteolin concentrations. These concentrations may be up to six times higher than the concentrations obtained with the strains containing the same enzymes with the exception of PAL and C4H (cf. FIG. 19, for example by comparing the strain SC2425 without PAL/C4H and the strain SC1500 with PAL/C4H or the strain SC2426 without PAL/C4H and the strain SC2428 with PAL/C4H).

### MET

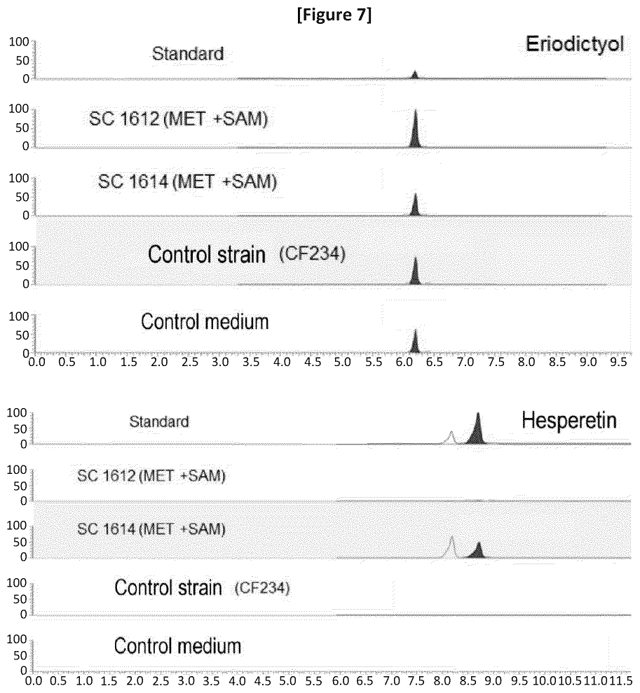

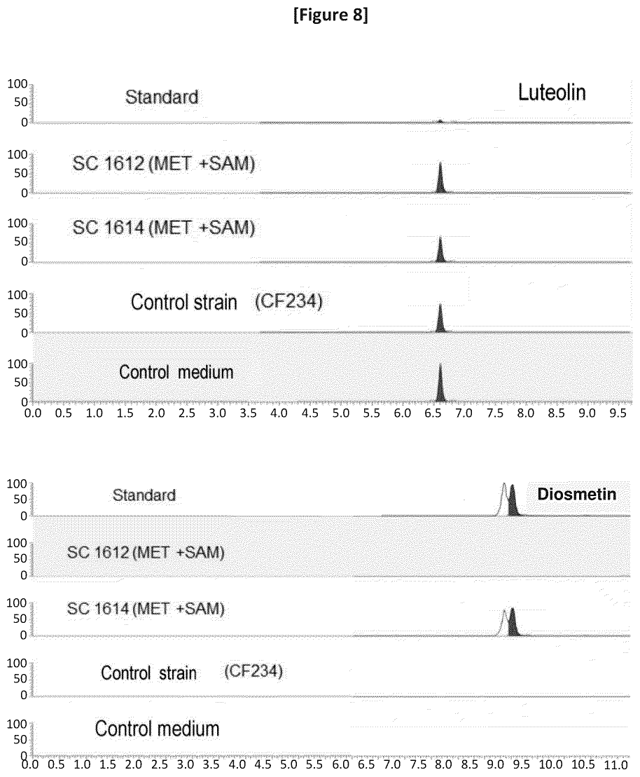

The results for the production of hesperetin and diosmetin from eriodictyol and luteolin by the strains SC1612, SC1614, SC2147 and SC2151 are presented, respectively, in FIGS. 7, 8, 20 and 21.

The yeast strains SC1612, SC1614, SC2147 and SC2151 are indeed capable of producing hesperetin and/or diosmetin.

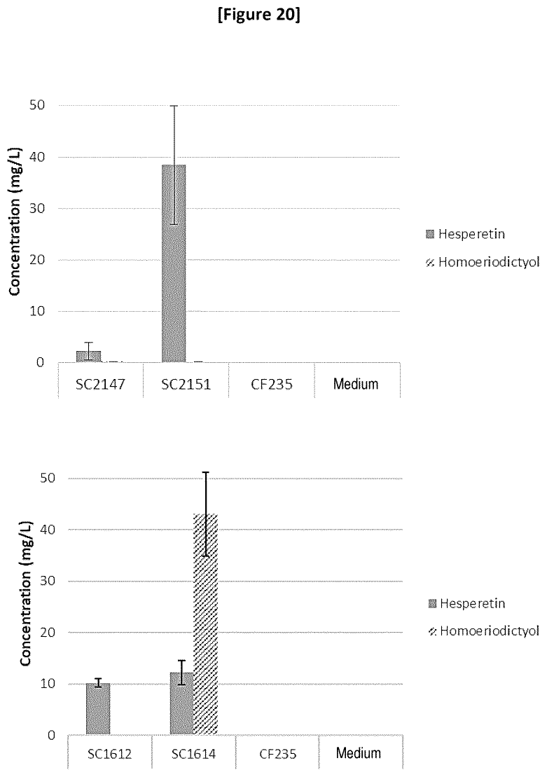

Starting with eriodictyol, the strains SC2147, SC2151 and SC1612 are capable of specifically producing hesperetin, i.e. of specifically methylating the hydroxyl in position 4′ of eriodictyol (FIG. 20). The strain SC1614 produces, for its part, a mixture of hesperetin and of homoeriodictyol.

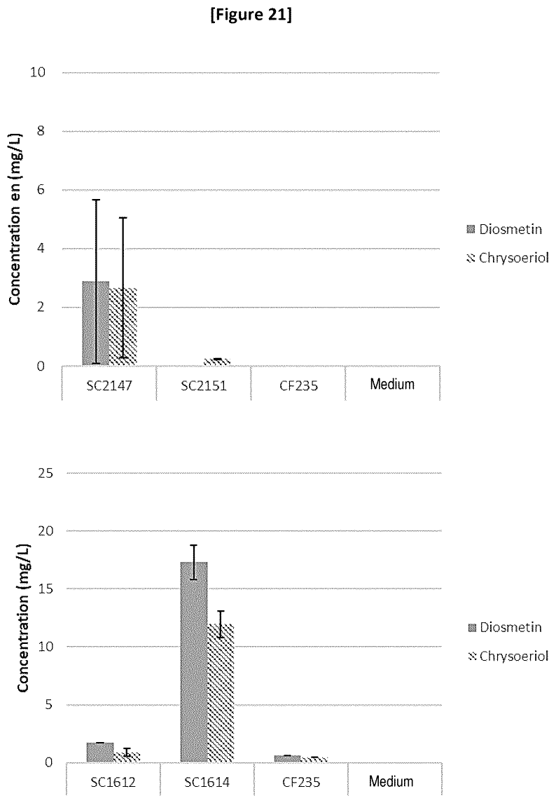

In a noteworthy manner, the strain SC2151 is moreover capable of producing about 40 mg/L of hesperetin (FIG. 20). The strains SC2147, SC1612 and SC1614, for their part, are capable of producing diosmetin from luteolin (FIG. 21).

### FNSII

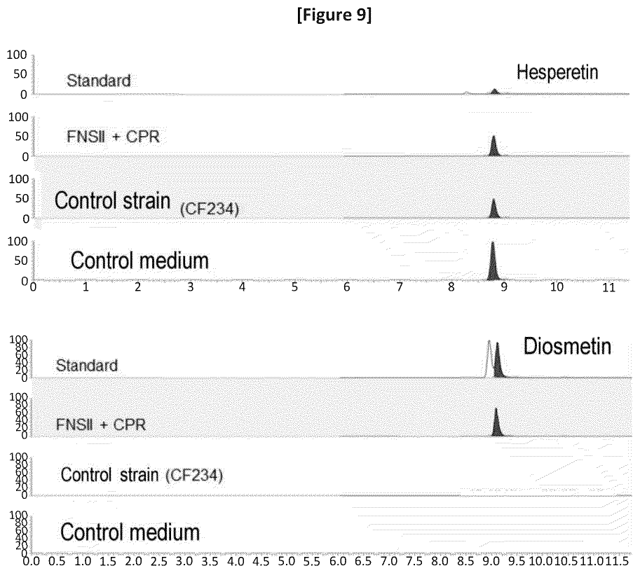

The results for the production of diosmetin from hesperetin by the strain SC744 are presented in FIG. 9.

The yeast strain SC744 is indeed capable of producing diosmetin from hesperetin.

E. coli UP TO HESPERETIN/DIOSMETIN

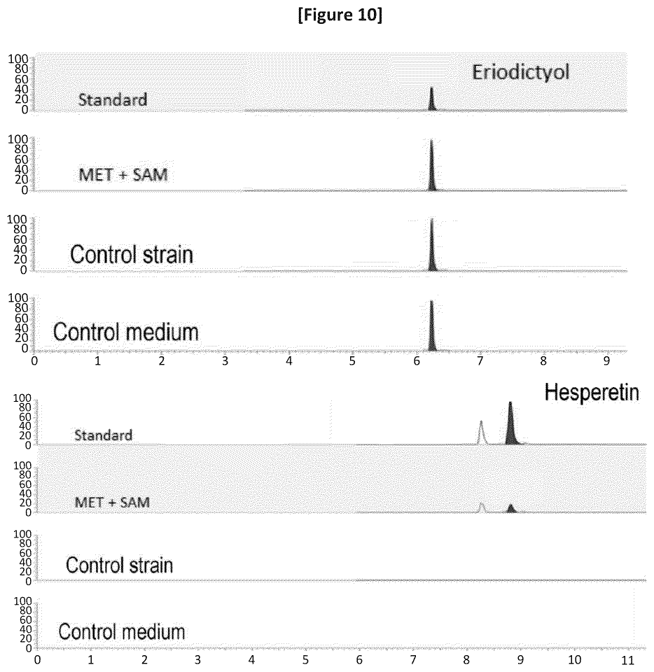

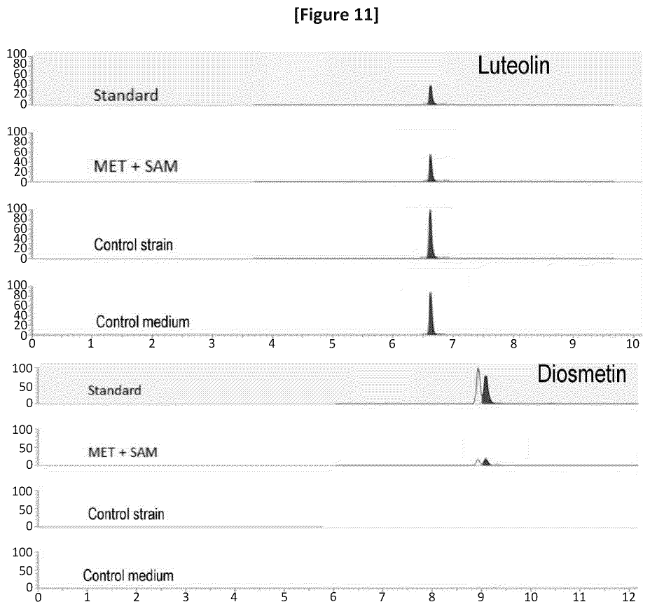

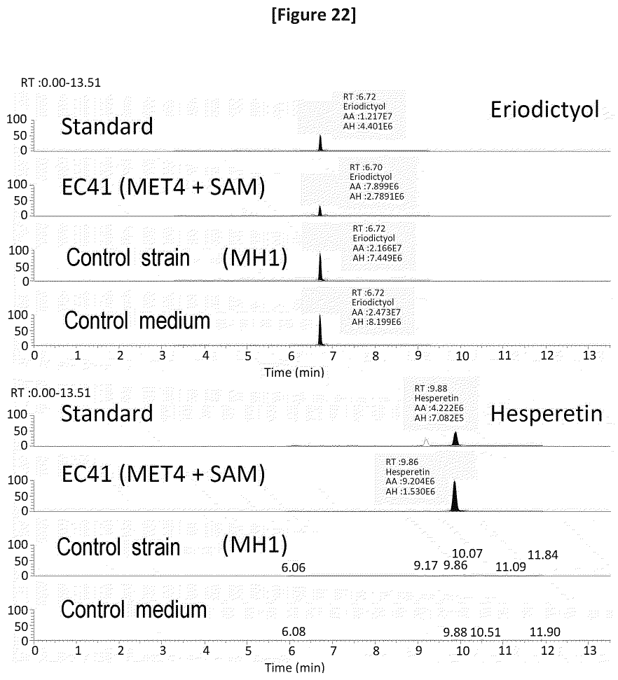

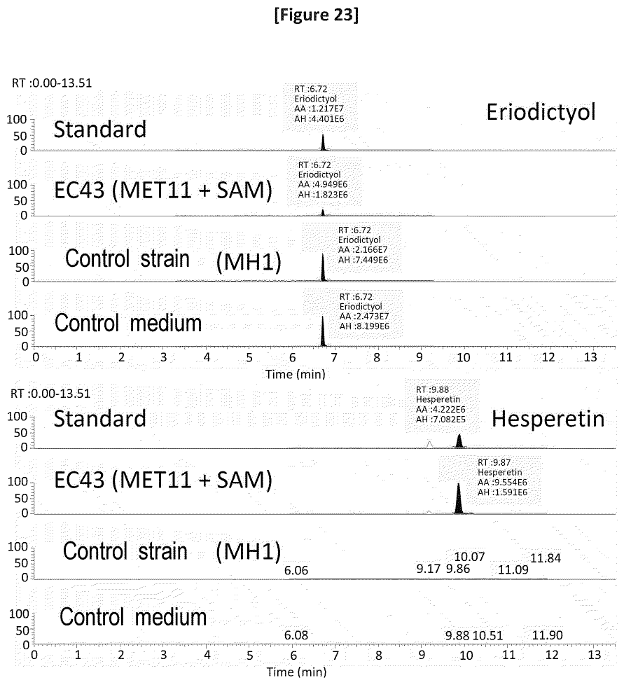

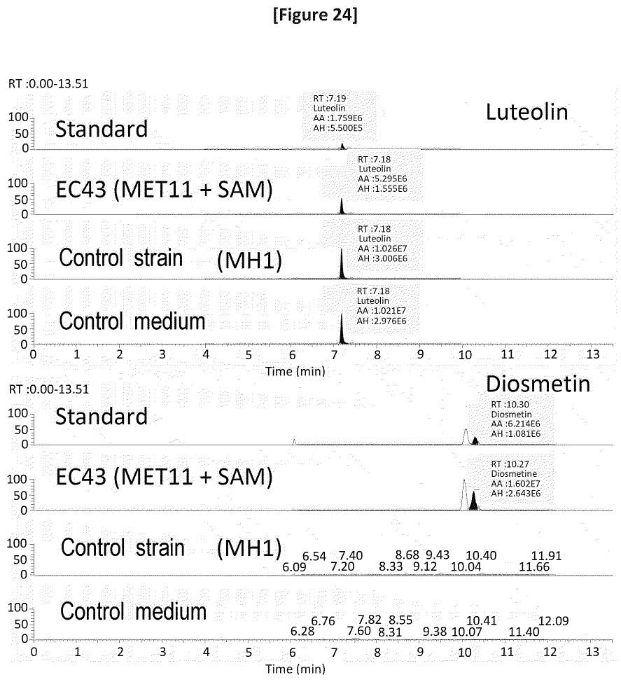

The results for the production of hesperetin from eriodictyol by the strains EC26, EC41 and EC43 are presented in FIGS. 10, 22 and 23 and the production of diosmetin from luteolin by the strains EC26 and EC43 are presented in FIGS. 11 and 24.

The E. coli strains EC26, EC41 and EC43 are indeed capable of producing hesperetin and/or diosmetin.

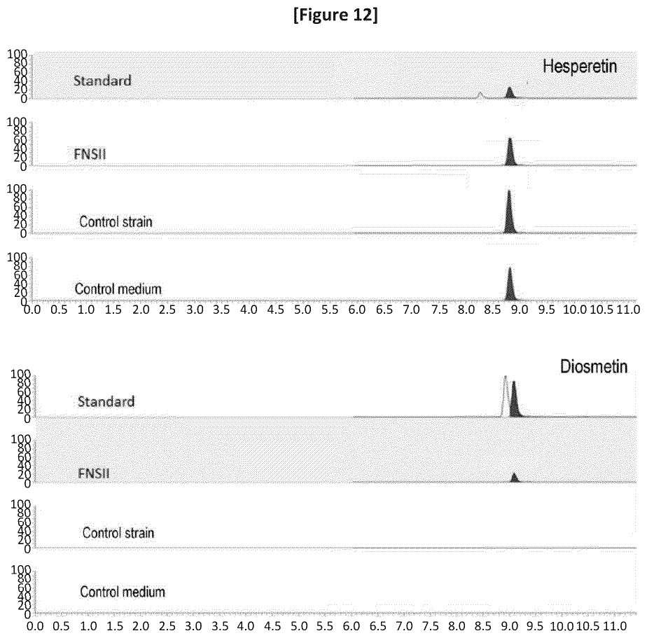

The results for the production of diosmetin from hesperetin by the strain EC30 are presented in FIG. 12.

The E. coli strain EC30 is indeed capable of producing diosmetin from hesperetin.

### Yeast Up to Hesperetin/Diosmetin

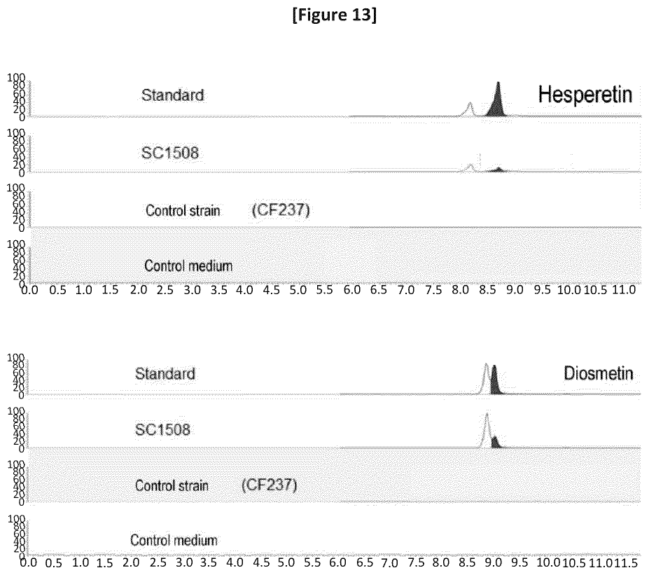

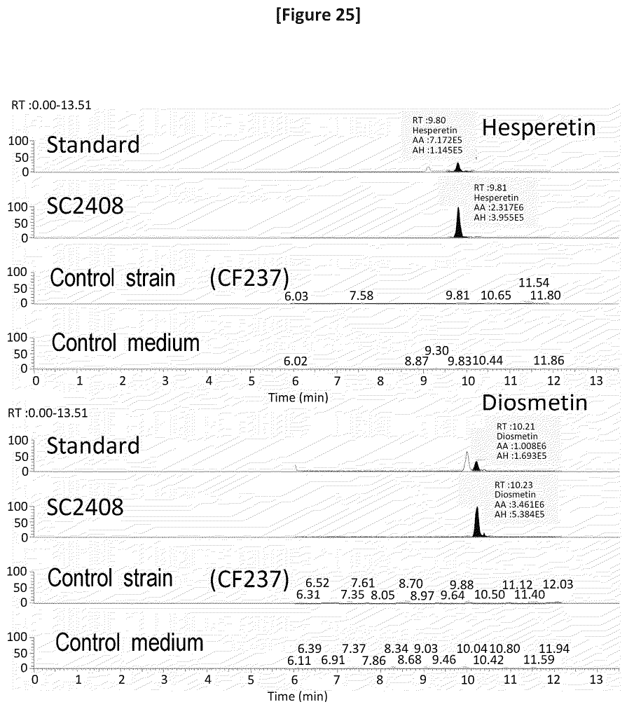

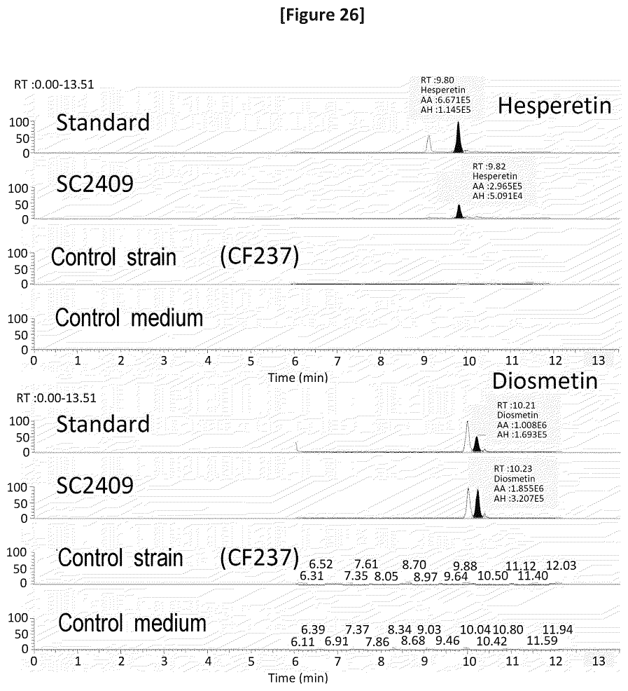

The results for the production of hesperetin and diosmetin from glucose by the strains SC1508, SC2408 and SC2409 are presented in FIGS. 13, 25 and 26.

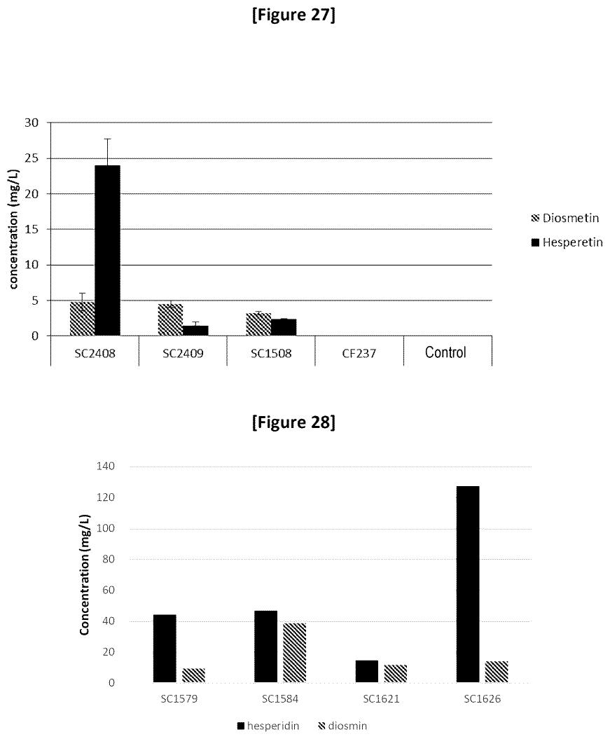

The yeast strains SC1508, SC2408 and SC2409 containing all the enzymes of the pathway up to hesperetin and diosmetin are capable of producing hesperetin and/or diosmetin from glucose (FIG. 27). In a noteworthy manner, the strain SC2408 produces about 25 mg/L of hesperetin and about 5 mg/L of diosmetin.

### GT

TABLE 24
Concentration of hesperetin, hesperetin 7-O-glucoside and hesperidin
according to the GTs used (in mg.l⁻¹)

| | | Hesperetin 7- | |
|---|---|---|---|
| | Hesperetin | O-glucoside | Hesperidin |
| medium | 90 ± 8.1 | N/D | N/D |
| Control - | 84.2 ± 4.6 | N/D | N/D |
| FL 545 - GT SEQ ID No 92 | 59.2 ± 1.1 | 7.1 ± 0.2 | 70.4 ± 2.2 |
| FL 546 - GT SEQ ID No 94 | 85.1 ± 1.4 | N/D | 22.2 ± 1.2 |
| FL 547 - GT SEQ ID No 96 | N/D | 6 ± 0.5 | 109.9 ± 4.8 |
| FL 548 - GT SEQ ID No 98 | 46.7 ± 2.4 | N/D | 71.9 ± 2.1 |
| FL 554 - GT SEQ ID No | | | |
| 114 | 46.7 ± 1.7 | // | 70.4 ± 5.0 |
| FL 556 - GT SEQ ID No | | | |
| 116 | 107.0 ± 3.0 | // | 11.3 ± 1.1 |

// amount not determined

TABLE 25
Concentration of diosmetin, diosmetin 7-O-glucoside and
diosmin according to the GTs used (in mg.l⁻¹)

| | | Diosmetin 7- | |
|---|---|---|---|
| | Diosmetin | O-glucoside | Diosmin |
| medium | 82.1 ± 3.9 | N/D | N/D |
| Control - | 75.3 ± 6.1 | N/D | N/D |
| FL 545 - GT SEQ ID No 92 | 67.1 ± 2.6 | N/D | 21.8 ± 0.4 |
| FL 546 - GT SEQ ID No 94 | 61.9 ± 3.3 | N/D | 23.2 ± 3.2 |
| FL 547 - GT SEQ ID No 96 | 2.4 ± 0.4 | 7.1 ± 0.5 | 122.8 ± 8.5 |
| FL 548 - GT SEQ ID No 98 | 17.7 ± 0.7 | N/D | 76 ± 4.1 |
| FL 554 - GT SEQ ID No | | | |
| 114 | 61.4 ± 4.2 | 7.1 ± 1.1 | 108.5 ± 8.3 |
| FL 556 - GT SEQ ID No | | | |
| 116 | 138.6 ± 3.5 | N/D | N/D |

The various strains are indeed capable of producing hesperidin and/or diosmin from hesperetin and diosmetin, in different concentrations according to the GT used.

### RHM

TABLE 26
Concentration of hesperetin, hesperetin 7-O-glucoside and
hesperidin according to the RHMs used (in mg.l⁻¹)

| | | Hesperetin 7- | |
|---|---|---|---|
| | Hesperetin | O-glucoside | Hesperidin |
| medium | 90 ± 8.1 | N/D | N/D |
| Control - | 84.2 ± 4.6 | N/D | N/D |
| FL 545 - RHM SEQ ID No 108 | 59.2 ± 1.1 | 7.1 ± 0.3 | 70.4 ± 2.2 |
| FL 512 - RHM SEQ ID No 110 | 45.6 ± 2.4 | N/D | 82.2 ± 5.2 |

TABLE 27
Concentration of diosmetin, diosmetin 7-O-glucoside and
diosmin according to the RHMs used (in mg.l⁻¹)

| | | Diosmetin 7- | |
|---|---|---|---|
| | Diosmetin | O-glucoside | Diosmin |
| medium | 82.1 ± 3.9 | N/D | N/D |
| Control - | 75.3 ± 6.1 | N/D | N/D |
| FL 545 - RHM SEQ ID No 108 | 67.1 ± 2.6 | N/D | 21.8 ± 0.4 |
| FL 512 - RHM SEQ ID No 110 | 61.4 ± 3.6 | N/D | 23.7 ± 1 |

The various strains are indeed capable of producing hesperidin and diosmin from hesperetin and diosmetin, in different concentrations according to the RHMs used.

### RHAT

TABLE 28
Concentration of hesperetin, hesperetin 7-O-glucoside and
hesperidin according to the RHATs used (in mg.l⁻¹)

| | | Hesperetin 7- | |
|---|---|---|---|
| | Hesperetin | O-glucoside | Hesperidin |
| medium | 90 ± 8.1 | N/D | N/D |
| Control - | 84.2 ± 4.6 | N/D | N/D |
| FL 512 - RHAT SEQ ID No 104 | 45.6 ± 2.4 | N/D | 82.2 ± 5.2 |
| FL 165 - RHAT SEQ ID No 106 | 46 ± 1.5 | 8.1 ± 0.2 | 69.8 ± 1.4 |

TABLE 29
Concentration of diosmetin, diosmetin 7-O-glucoside and
diosmin according to the RHATs used (in mg.l⁻¹)

| | | Diosmetin 7- | |
|---|---|---|---|
| | Diosmetin | O-glucoside | Diosmin |
| medium | 82.1 ± 3.9 | N/D | N/D |
| Control - | 75.3 ± 6.1 | N/D | N/D |
| FL 512 - RHAT SEQ ID No 104 | 61.4 ± 3.6 | N/D | 23.7 ± 1 |
| FL 165 - RHAT SEQ ID No 106 | 66.7 ± 2.5 | N/D | 23 ± 1.5 |

The various strains are indeed capable of producing hesperidin and diosmin from hesperetin and diosmetin, in different concentrations according to the GTs, the RHMs and the RHATs used.

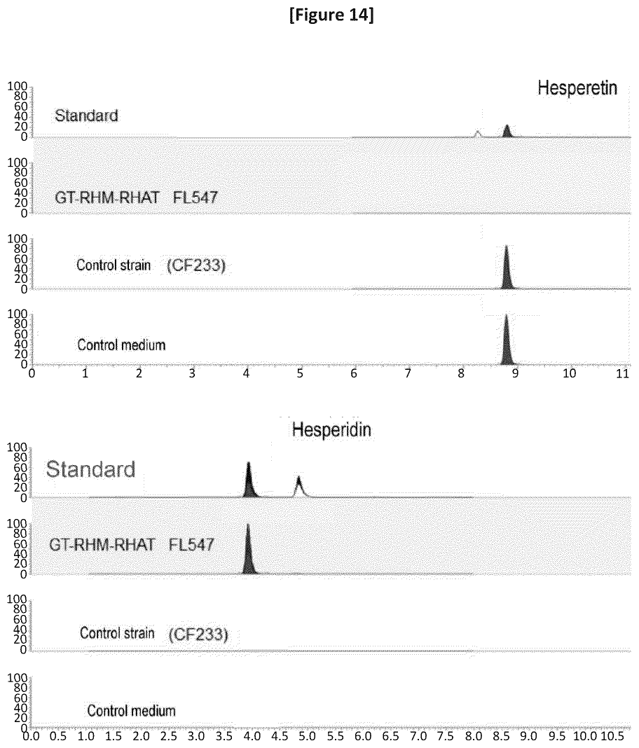

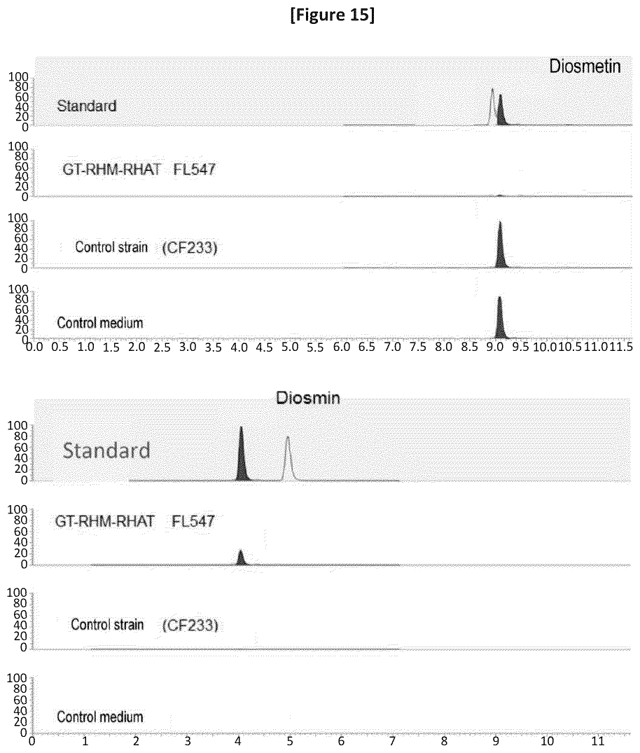

The results for the production of hesperidin and diosmin from hesperetin and diosmetin by the strain FL 547 are presented, respectively, in FIGS. 14 and 15.

The yeasts tested with the various constructs are indeed capable of producing hesperidin and diosmin.

E. coli Up to Hesperidin/Diosmin

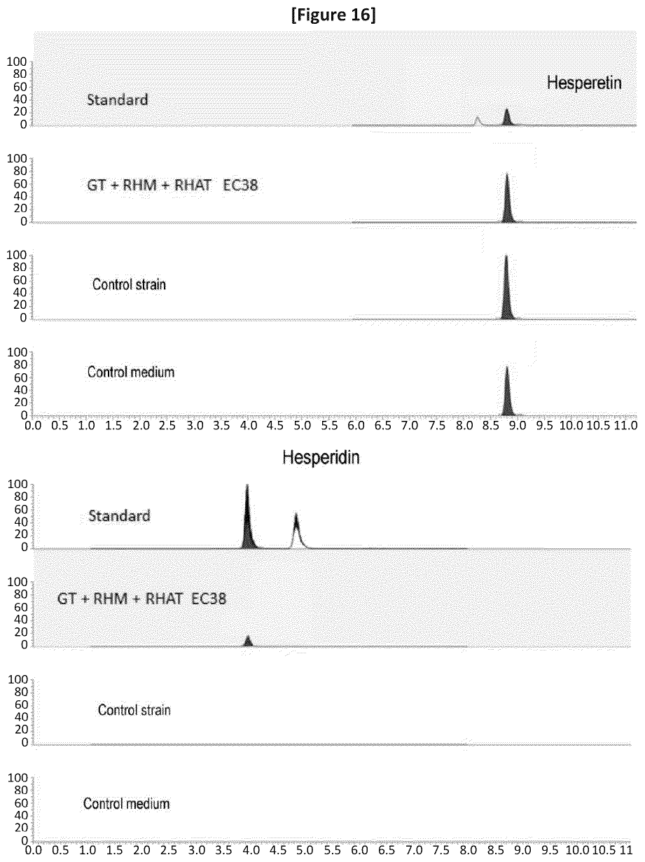

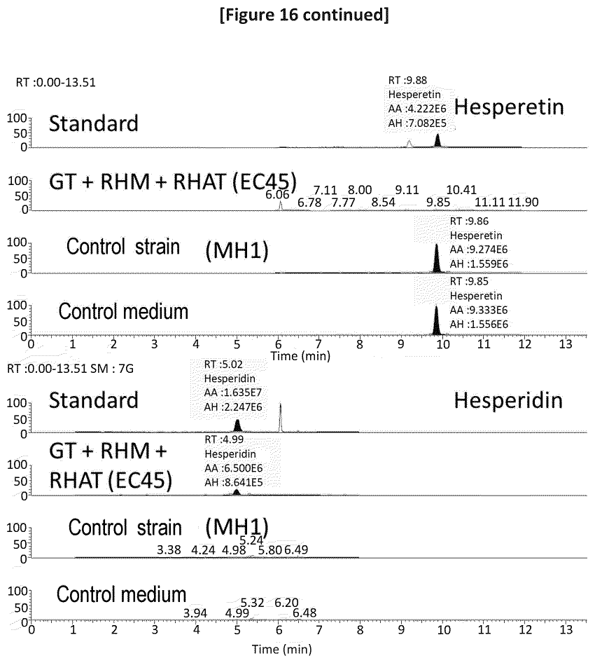

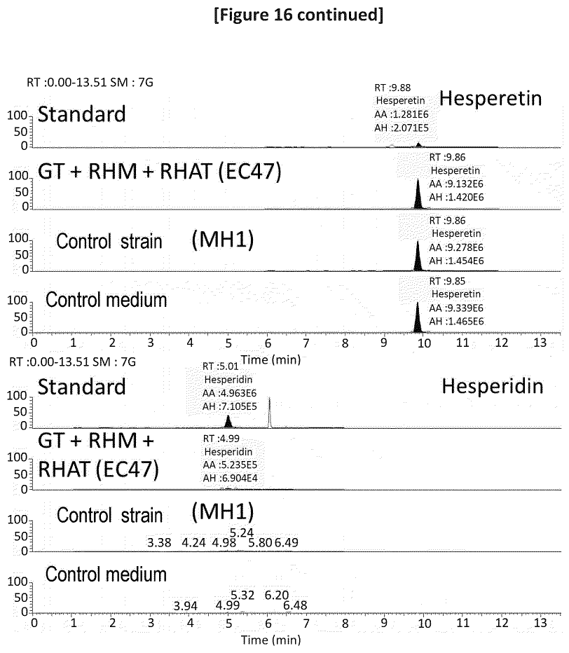

The results for the production of hesperidin from hesperetin by the strains EC38, EC45 and EC47 are presented in FIG. 16.

These strains are indeed capable of producing hesperidin from hesperetin.

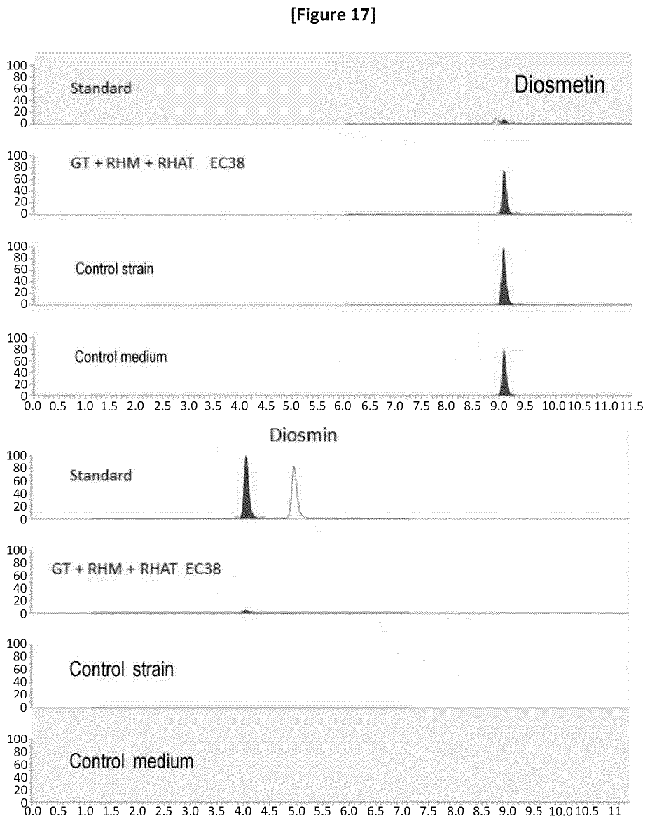

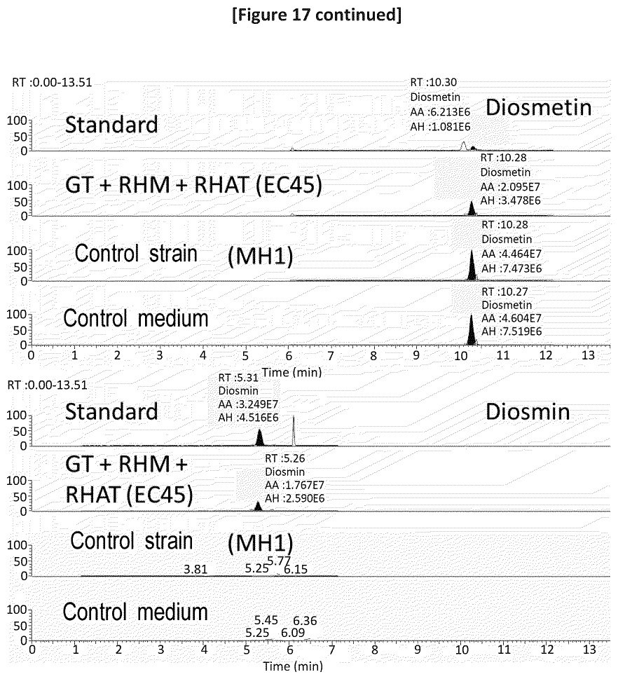

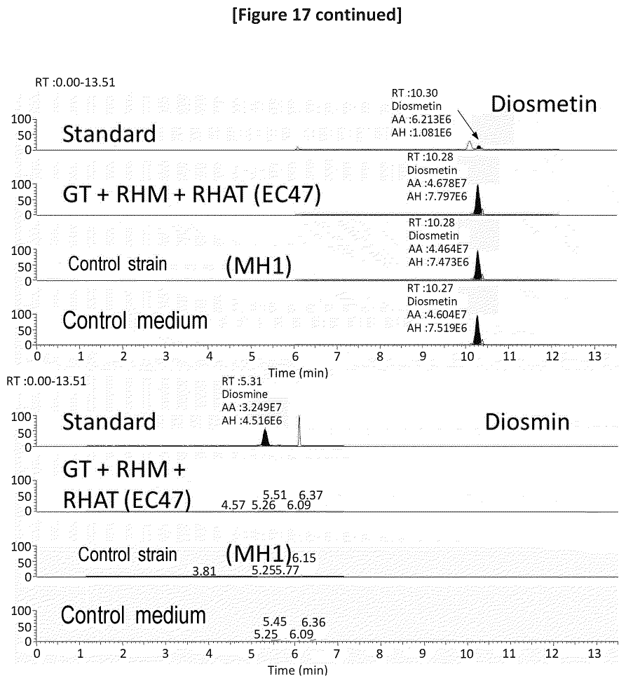

The results for the production of diosmin from diosmetin by the strains EC38, EC45 and EC47 are presented in FIG. 17.

These strains are indeed capable of producing diosmin from diosmetin.

### Yeast Up to Hesperidin/Diosmin

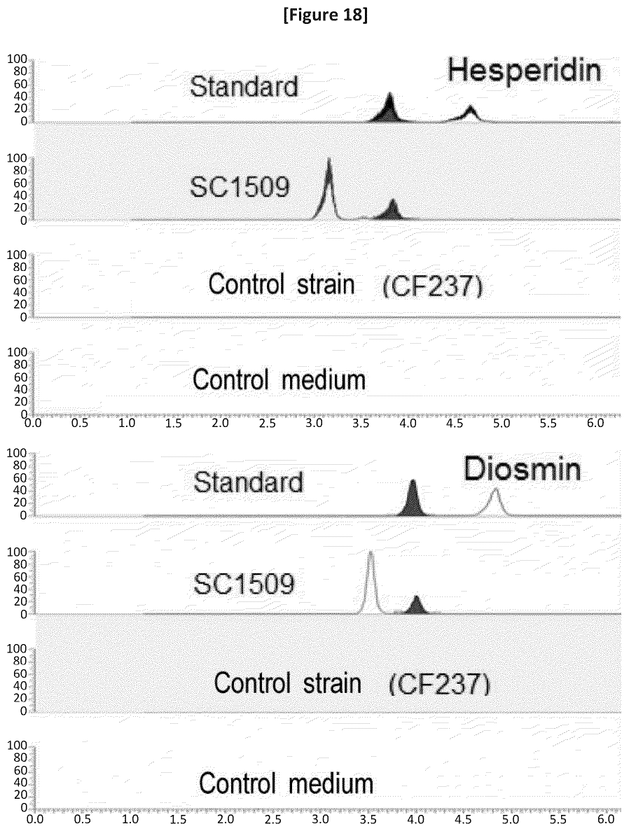

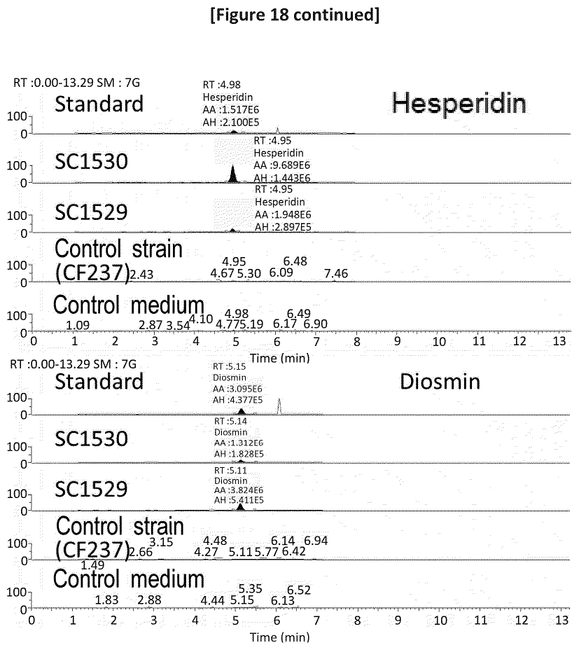

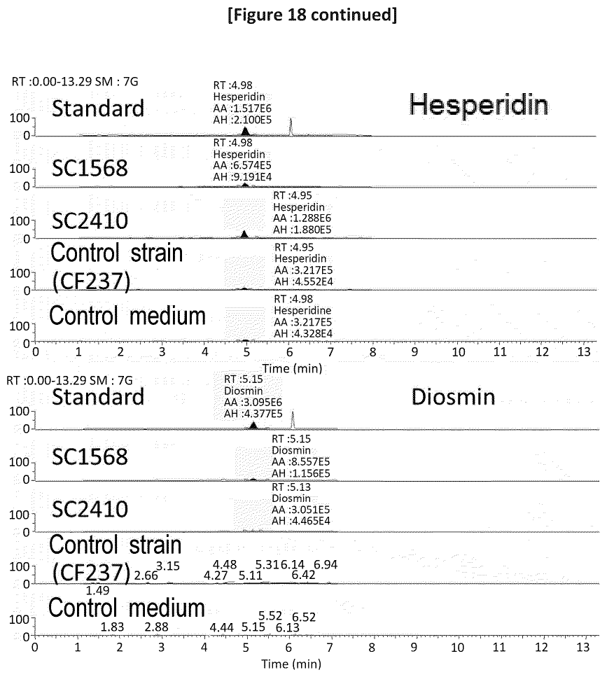

The results for the production of hesperidin and diosmin from glucose by the strains SC1509, SC1530, SC1529, SC1568 and SC2410 are presented in FIG. 18. The results for the production of hesperidin from diosmin by the strains SC1579, SC1584, SC1621 and SC1626 are presented in FIG. 28.

All the strains containing all the enzymes of the pathway are capable of producing hesperidin and/or diosmin.

1. A method for producing diosmin and/or hesperidin, comprising culturing a recombinant microorganism in a culture medium and harvesting diosmin and/or hesperidin produced by said recombinant microorganism, wherein the recombinant microorganism comprises
a heterologous nucleic acid sequence coding for a flavanone 7-O-beta-D-glucosyltransferase (UGT) belonging to EC 2.4.1.185, which is capable of adding a glucose in position 7 of hesperetin and/or diosmetin; and
a heterologous nucleic acid sequence coding for a 6″-O-rhamnosyltransferase (RhaT) belonging to EC 2.4.1.B53, which is capable of transferring a rhamnose into position 6 of the glucose of hesperetin-7-O-glucoside and/or diosmetin-7-O-glucoside; and
a heterologous nucleic acid sequence coding for a UDP-glucose 4,6-dehydratase/UDP-4-keto-6-deoxy-D-glucose 3,5-epimerase/UDP-4-keto-L-rhamnose-reductase (RHM) belonging to EC 4.2.1.76, which is capable of producing UDP-rhamnose.

2. The method of claim 1, wherein the culture medium does not comprise naringenin, apigenin, eriodictyol, luteolin, hesperetin and/or diosmetin.

3. The method of claim 1, wherein the culture is performed in a bioreactor in batch, fed-batch, chemostat or continuous cultivation mode.

4. The method of claim 1, wherein the flavanone 7-O-beta-D-glucosyltransferase is selected from the group consisting of SEQ ID NOs: 113, 115, 91, 93, 95, 97, 99 and 101 and polypeptides comprising a sequence having at least 60% sequence identity with one of these sequences and having flavanone 7-O-beta-D-glucosyltransferase activity.

5. The method of claim 1, wherein the 6″-O-rhamnosyltransferase is selected from the group consisting of SEQ ID NOs: 103 and 105 and polypeptides comprising a sequence having at least 60% sequence identity with one of these sequences and having 6″-O-rhamnosyltransferase activity.

6. The method of claim 1, wherein the UDP-glucose 4,6-dehydratase/UDP-4-keto-6-deoxy-D-glucose 3,5-epimerase/UDP-4-keto-L-rhamnose-reductase is selected from the group consisting of SEQ ID NOs: 107, 109 and 111 and polypeptides comprising a sequence having at least 60% sequence identity with one of these sequences and having UDP-glucose 4,6-dehydratase/UDP-4-keto-6-deoxy-D-glucose 3,5-epimerase/UDP-4-keto-L-rhamnose-reductase activity.

7. The method of claim 1, wherein
the flavanone 7-O-beta-D-glucosyltransferase is selected from the group consisting of SEQ ID NOs: 113 and 95 and polypeptides comprising a sequence having at least 60% sequence identity with one of these sequences and having flavanone 7-O-beta-D-glucosyltransferase activity; and
the 6″-O-rhamnosyltransferase is selected from the group consisting of SEQ ID NO: 103 and polypeptides comprising a sequence having at least 60% sequence identity with this sequence and having 6″-O-rhamnosyltransferase activity; and
the UDP-glucose 4,6-dehydratase/UDP-4-keto-6-deoxy-D-glucose 3,5-epimerase/UDP-4-keto-L-rhamnose-reductase is selected from the group consisting of SEQ ID NO: 107 and polypeptides comprising a sequence having at least 60% sequence identity with this sequence and having UDP-glucose 4,6-dehydratase/UDP-4-keto-6-deoxy-D-glucose 3,5-epimerase/UDP-4-keto-L-rhamnose-reductase activity.

8. The method of claim 1, wherein the recombinant microorganism also comprises:
a heterologous nucleic acid sequence coding for a tyrosine ammonia lyase (TAL);
a heterologous nucleic acid sequence coding for a 4-coumarin-CoA ligase (4CL);
a heterologous nucleic acid sequence coding for a naringenin-chalcone synthase (CHS); and
a heterologous nucleic acid sequence coding for a chalcone isomerase (CHI).

9. The method of claim 8, wherein the recombinant microorganism comprises:
a heterologous nucleic acid sequence coding for a tyrosine ammonia lyase (TAL) comprising a sequence selected from the group consisting of SEQ ID NOs: 41 and 39 and polypeptides comprising a sequence having at least 60% sequence identity with one of these sequences and having tyrosine ammonia lyase activity;
a heterologous nucleic acid sequence coding for a 4-coumaroyl-CoA ligase (4CL) comprising a sequence selected from the group consisting of SEQ ID NOs: 123, 125, 43, 45, 47 and 49 and polypeptides comprising a sequence having at least 60% sequence identity with one of these sequences and having 4-coumarate-CoA ligase activity;
a heterologous nucleic acid sequence coding for a chalcone synthase (CHS) comprising a sequence selected from the group consisting of SEQ ID NOs: 53, 51, 55 and 57 and polypeptides comprising a sequence having at least 60% sequence identity with one of these sequences and having chalcone synthase activity; and
a heterologous nucleic acid sequence coding for a chalcone isomerase (CHI) comprising a sequence selected from the group consisting of SEQ ID NOs: 61 and 59 and polypeptides comprising a sequence having at least 60% sequence identity with one of these sequences and having chalcone isomerase activity.

10. The method of claim 1, wherein the recombinant microorganism also comprises a heterologous nucleic acid sequence coding for a flavonoid 3′-monooxygenase (F3′H).

11. The method of claim 10, wherein the recombinant microorganism comprises a heterologous nucleic acid sequence coding for a flavonoid 3′-monooxygenase (F3′H) comprising a sequence selected from the group consisting of SEQ ID NOs: 7, 1, 3, 5, 9, 11, 13, 15, 17, 19, 21 and 121 and polypeptides comprising a sequence having at least 60% sequence identity with one of these sequences and having flavonoid 3′-monooxygenase activity.

12. The method of claim 1, wherein the recombinant microorganism also comprises a heterologous nucleic acid sequence coding for an O-methyltransferase (OMT).

13. The method of claim 12, wherein the recombinant microorganism comprises a heterologous nucleic acid sequence coding for an O-methyltransferase (OMT) comprising a sequence selected from the group consisting of SEQ ID NOs: 119, 117, 87 and 89 and polypeptides comprising a sequence having at least 60% sequence identity with one of these sequences and having O-methyltransferase activity.

14. The method of claim 1, wherein the recombinant microorganism also comprises:
a heterologous nucleic acid sequence coding for a phenylalanine ammonia lyase (PAL) comprising a sequence selected from the group consisting of SEQ ID NOs: 63, 65 and 77 and polypeptides comprising a sequence having at least 60% sequence identity with one of these sequences and having phenylalanine ammonia lyase activity; and
a heterologous nucleic acid sequence coding for a cinnamate 4-hydroxylase (C4H) comprising a sequence selected from the group consisting of SEQ ID NOs: 67, 69 and 79 and polypeptides comprising a sequence having at least 60% sequence identity with one of these sequences and having cinnamate 4-hydroxylase activity.

15. The method of claim 1, wherein the recombinant microorganism also comprises a heterologous or endogenous nucleic acid sequence coding for a flavone synthase (FNS).

16. The method of claim 15, wherein the recombinant microorganism comprises a heterologous nucleic acid sequence coding for a flavone synthase (FNS) comprising a sequence selected from the group consisting of SEQ ID NOs: 33, 35, 37, 127, 129, 131, 133, 135, 137, 139, 141, 143, 145, 147, 149, 151, 153, 155, 157 and 159 and polypeptides comprising a sequence having at least 60% sequence identity with one of these sequences and having flavone synthase activity.

17. The method of claim 1, wherein the recombinant microorganism also comprises:
a heterologous nucleic acid sequence coding for a cytochrome P450 reductase (CPR); and/or
a heterologous or endogenous nucleic acid sequence coding for an S-adenosylmethionine synthetase (SAMT).

18. The method of claim 17, wherein the recombinant microorganism comprises a heterologous nucleic acid sequence coding for a cytochrome P450 reductase (CPR) comprising a sequence selected from the group consisting of SEQ ID NOs: 25, 23, 27, 29 and 31 and polypeptides comprising a sequence having at least 60% sequence identity with one of these sequences and having cytochrome P450 reductase activity.

19. The method of claim 1, wherein the recombinant microorganism comprises:
a heterologous nucleic acid sequence coding for a phenylalanine ammonia lyase (PAL) comprising SEQ ID NO: 65 or a polypeptide comprising a sequence having at least 60% sequence identity with this sequence and having phenylalanine ammonia lyase activity;
a heterologous nucleic acid sequence coding for a cinnamate 4-hydroxylase (C4H) comprising SEQ ID NO: 79 or a polypeptide comprising a sequence having at least 60% sequence identity with this sequence and having cinnamate 4-hydroxylase activity;
a heterologous nucleic acid sequence coding for a tyrosine ammonia lyase (TAL) comprising SEQ ID NO: 41 or a polypeptide comprising a sequence having at least 60% sequence identity with this sequence and having tyrosine ammonia lyase activity;
a heterologous nucleic acid sequence coding for a 4-coumaroyl-CoA ligase (4CL) comprising SEQ ID NO: 45, 123 or a polypeptide comprising a sequence having at least 60% sequence identity with one of these sequences and having 4-coumarate-CoA ligase activity;
a heterologous nucleic acid sequence coding for a chalcone synthase (CHS) comprising SEQ ID NO: 53 or a polypeptide comprising a sequence having at least 60% sequence identity with this sequence and having chalcone synthase activity;
a heterologous nucleic acid sequence coding for a chalcone isomerase (CHI) comprising SEQ ID NO: 61 or a polypeptide comprising a sequence having at least 60% sequence identity with this sequence and having chalcone isomerase activity;
a heterologous nucleic acid sequence coding for a flavonoid 3′-monooxygenase (F3′H) comprising SEQ ID NOs: 7, 17, 121 or a polypeptide comprising a sequence having at least 60% sequence identity with one of these sequences and having flavonoid 3′-monooxygenase activity;
a heterologous nucleic acid sequence coding for a flavone synthase (FNS) comprising SEQ ID NO: 37 or a polypeptide comprising a sequence having at least 60% sequence identity with this sequence and having flavone synthase activity;
a heterologous nucleic acid sequence coding for a cytochrome P450 reductase (CPR) comprising SEQ ID NO: 25 or a polypeptide comprising a sequence having at least 60% sequence identity with this sequence and having cytochrome P450 reductase activity;
a heterologous nucleic acid sequence coding for an O-methyltransferase (OMT) comprising SEQ ID NOs: 117, 119 or a polypeptide comprising a sequence having at least 60% identity with one of these sequences and having O-methyltransferase activity;
a heterologous nucleic acid sequence coding for a flavanone 7-O-beta-D-glucosyltransferase comprising SEQ ID NOs: 113, 95 or a polypeptide comprising a sequence having at least 60% sequence identity with one of these sequences and having flavanone 7-O-beta-D-glucosyltransferase activity;
a heterologous nucleic acid sequence coding for a 6″-O-rhamnosyltransferase comprising SEQ ID NO: 103 or a polypeptide comprising a sequence having at least 60% sequence identity with this sequence and having 6″-O-rhamnosyltransferase activity; and
a heterologous nucleic acid sequence coding for a UDP-glucose 4,6-dehydratase/UDP-4-keto-6-deoxy-D-glucose 3,5-epimerase/UDP-4-keto-L-rhamnose-reductase comprising SEQ ID NO: 107 or a polypeptide comprising a sequence having at least 60% sequence identity with this sequence and having UDP-glucose 4,6-dehydratase/UDP-4-keto-6-deoxy-D-glucose 3,5-epimerase/UDP-4-keto-L-rhamnose-reductase activity.

20. The method of claim 1, wherein the recombinant microorganism is a yeast or a bacterium.


SEQUENCE LISTING (160 sequences)

SEQ ID NO: 1 - protein, 524 aa, Perilla frutescens
MSISAAVSLIICTSILGVLVYFLFLRRGGGSNGRPLPPGPRPWPIVGNLPQLGPKPHQSMAALARVHGPLMHLKMGFVHVVVAASATVAEKFLKVHDTNFLSRPPNSGAEHIAYNYNDLVFAPHGPRWRLLRKICALHLFSSKALDDFRHVREEEVGILIRNLASVGEMPASIGQMMYVCATNAISRVMLGRHVLGDEHRGAAGGGDTTAEEFKAMVVELMALAGVFNVGDFIPPLKGLDLQGVVAKMKKLHQRFDAFFSGILHDHKINGSNAAEGHVDLLTTLISLKDVDNNGEGGKLTDTEIKALLLNLFTAGTDTTSSTVEWAITELIRNPNILARVRKELDLIVGKDKLVKESDLGQLTYLQAVIKENFRLHPSTPLSLPRVAQESCEINGYYIPKDSTLLVNVWAIGRDPNVWPDPLEFRPERFLMGGEKPNVDVRGNDFELIPFGSGRRICAGMNLGIRMVQLLIATMVHAFDFELANGQLAKDLNMEEAYGITLQRADPLVVHPRPRLARHVYQAQV

SEQ ID NO: 2 - DNA, 1575 bp, synthetic construct
  misc_feature 1..1575: note = coding sequence for SEQ ID NO: 1
  source 1..1575: mol_type = other DNA
atgtccatctctgccgccgtttctttgatcatctgtacttccattttgggtgttttggtttacttcttgttcttgagaagaggtggtggttctaacggtagaccattgccaccaggtccaagaccatggccaattgtcggtaacttgccacaattgggtccaaagccacaccaatctatggctgccttggccagagttcacggtccattgatgcacttgaagatgggtttcgttcacgttgttgttgccgcctccgccaccgttgctgaaaagttcttgaaggttcacgacaccaacttcttgtctagaccaccaaactccggtgccgaacacattgcttacaactacaacgacttggttttcgctccacacggtccaagatggagattgttgagaaagatttgtgccttgcacttgttctcctccaaggccttggatgacttcagacacgttagagaagaagaagttggtatcttgattagaaacttggcttctgttggtgaaatgccagcttctatcggtcaaatgatgtacgtttgtgccactaacgctatctctagagtcatgttgggtagacacgttttgggtgacgaacacagaggtgccgccggtggtggtgataccactgctgaagaattcaaggctatggttgttgaattgatggctttggccggtgttttcaacgttggtgatttcattccaccattgaagggtttggacttgcaaggtgttgttgctaagatgaagaagttgcaccaaagattcgacgctttcttctctggtatcttgcacgatcacaagatcaacggttctaacgccgctgaaggtcacgttgacttgttgactactttgatttctttgaaggacgttgacaacaacggtgaaggtggtaagttgaccgatactgaaattaaggctttgttgttgaacttgttcactgctggtactgacactacttcttctactgttgaatgggccatcactgaattgatcagaaacccaaacattttggctagagttagaaaggaattggacttgatcgttggtaaggataagttggttaaggaatccgatttgggtcaattgacctacttgcaagccgttatcaaggaaaacttcagattgcacccatctactccattgtctttgccaagagtcgctcaagaatcttgtgaaatcaacggttactacatcccaaaggattctaccttgttggtcaacgtttgggccatcggtagagatccaaacgtttggccagatccattggaattcagaccagaaagattcttgatgggtggtgaaaagccaaacgttgatgttagaggtaacgatttcgaattgattccattcggttctggtagaagaatttgtgctggtatgaacttgggtattagaatggttcaattgttgattgctactatggttcacgctttcgatttcgaattggctaacggtcaattggccaaggacttgaacatggaagaagcttacggtattactttgcaaagagccgacccattggttgtccacccaagaccaagattggccagacacgtttaccaagctcaagtttaa

SEQ ID NO: 3 - protein, 627 aa, Phanerochaete chrysosporium
MSPLLSAVPAAALPLLAAAMYVLWTFLALLVRQARSPLRHLRGPPSPSFLVGNLREMHDQENTALFARWEHRYGSTFVYHGFLGGARLLTTDPVAVAHILAHGYDFPKPEFIRDALASMAAGHEGLLVVEGDGHRRQRKILSPAFATPHIKSLSPIIWSKATQLRDVWIDLASSPSLTPAATPSDPLAHAEEQPARASGFLPNPFSSFLSSKAHPRSALPRAEAHPEDRMSSPGTKVDVLAWLARATLDVIGEAGFGYAFNSVRAAACPGDAAEDELARAFAVIFSTARKFRLITVLQVWFPFLRRFRRNSAAEDHARATMRRIGLALIAERRQEVLDDKAHASQEAMDGKDLLTVMIKSSLSSDPSQQLSTNEMLCQIATFLAAGHETSASALSWALYALARAPACQHTLRRELRALTLPADPSAADLQAVLALPYLDAVVRETLRVHAPVTSTMRVAAHDAAVPVGTPFRDAHGAQHAAIRLRAGDVVTLPLQAMNKARALWGADAACFRPERWLAHGDAPREPRGLWGGVMTFGTGVVANGNRSCIGYRFAVNEIKLFLYALVRDIEFTIDPWIEIEKRVNVVTRPCVKSEPHLGNQMPLRLRRVAVEETVGDSSGDGAPRTVS

SEQ ID NO: 4 - DNA, 1884 bp, synthetic construct
  misc_feature 1..1884: note = coding sequence for SEQ ID NO: 3
  source 1..1884: mol_type = other DNA
atgtccccattgttgtctgccgttccagccgccgctttgccattgttggccgccgctatgtacgttttgtggactttcttggctttgttggttagacaagccagatccccattgagacacttgagaggtccaccatctccatctttcttggtcggtaacttgagagaaatgcacgaccaagaaaacaccgctttgttcgctagatgggaacacagatacggttctaccttcgtctaccacggtttcttgggtggtgccagattgttgaccaccgacccagtcgccgtcgcccacatcttggcccacggttacgacttcccaaagccagaattcatcagagatgccttggcttctatggccgccggtcacgaaggtttgttggtcgtcgaaggtgacggtcacaggagacaaagaaagatcttgtccccagctttcgccactccacacatcaagtctttgtctccaatcatctggtctaaggctactcaattgagagatgtttggattgacttggcctcttctccatccttgactccagctgctaccccatccgacccattggctcacgctgaagaacaaccagctagagcttctggtttcttgccaaacccattctcttctttcttgtcatctaaggctcacccaagatccgctttgccaagagctgaagctcacccagaagatagaatgtcctctccaggtaccaaggtcgacgttttggcttggttggctagagccactttggacgtcatcggtgaagccggtttcggttacgctttcaactctgtcagagccgctgcttgtccaggtgacgccgctgaagatgaattggctagagctttcgctgtcatcttctctactgctagaaagttcagattgatcactgtcttgcaagtttggttcccattcttgagaagattcagaagaaactctgctgctgaagatcacgctagagctactatgagaagaatcggtttggctttgatcgctgaaaggagacaagaagtcttggacgacaaggctcacgcctctcaagaagctatggatggtaaggacttgttgactgtcatgatcaagtcatctttgtcatccgatccatctcaacaattgtctactaacgaaatgttgtgtcaaatcgctactttcttggctgctggtcacgaaacctctgcttctgctttgtcttgggccttgtacgccttggctagagctccagcttgtcaacacactttgagaagagaattgagagctttgactttgccagctgacccatctgccgctgacttgcaagctgttttggctttgccatacttggacgccgtcgttagagaaactttgagagttcacgctccagttacttctactatgagagtcgctgctcacgacgccgctgttccagtcggtactccattcagagatgctcacggtgctcaacacgccgctatcagattgagagctggtgacgtcgtcactttgccattgcaagctatgaacaaggccagagctttgtggggtgccgacgccgcttgtttcagaccagaaagatggttggctcacggtgacgccccaagagaaccaagaggtttgtggggtggtgtcatgactttcggtaccggtgtcgtcgctaacggtaacagatcttgtattggttacagattcgctgttaacgaaatcaagttgttcttgtacgccttggttagagacatcgaattcactatcgacccatggatcgaaatcgaaaagagagttaacgtcgtcaccagaccatgtgtcaagtccgaaccacacttgggtaaccaaatgccattgagattgagaagagtcgctgttgaagaaactgttggtgattcttctggtgacggtgctccaagaactgtttcttaa

SEQ ID NO: 5 - protein, 513 aa, Petunia x hybrida
MSEILSLILYTVIFSFLLQFILRSFFRKRYPLPLPPGPKPWPIIGNLVHLGPKPHQSTAAMAQTYGPLMYLKMGFVDVVVAASASVAAQFLKTHDANFSSRPPNSGAEHMAYNYQDLVFAPYGPRWRMLRKICSVHLFSTKALDDFRHVRQDEVKTLTRALASAGQKPVKLGQLLNVCTTNALARVMLGKRVFADGSGDVDPQAAEFKSMVVEMMVVAGVFNIGDFIPQLNWLDIQGVAAKMKKLHARFDAFLTDILEEHKGKIFGEMKDLLSTLISLKNDDADNDGGKLTDTEIKALLLNLFVAGTDTSSSTVEWAIAELIRNPKILAQAQQEIDKVVGRDRLVGELDLAQLTYLEAIVKETFRLHPSTPLSLPRIASESCEINGYFIPKGSTLLLNVWAIARDPNAWADPLEFRPERFLPGGEKPKVDVRGNDFEVIPFGAGRRICAGMNLGIRMVQLMIATLIHAFNWDLVSGQLPEMLNMEEAYGLTLQRADPLVVHPRPRLEAQAYIG

SEQ ID NO: 6 - DNA, 1542 bp, synthetic construct
  misc_feature 1..1542: note = coding sequence for SEQ ID NO: 5
  source 1..1542: mol_type = other DNA
atgtccgaaatcttgtctttgattttgtacaccgtcattttctctttcttgttgcaattcattttgagatctttcttcagaaagagatacccattgccattgccaccaggtccaaagccatggccaattatcggtaacttggtccacttgggtccaaagccacaccaatctactgctgccatggctcaaacttacggtccattgatgtacttgaagatgggtttcgttgacgttgttgttgctgcctctgcttctgttgctgctcaattcttaaagactcacgatgctaacttctcttctagaccaccaaactctggtgctgaacacatggcttacaactaccaagatttggttttcgctccatacggtccaagatggagaatgttgagaaagatttgttctgttcacttgttctctaccaaggctttggatgacttcagacacgtcagacaagatgaagttaagactttgactagagctttggcttctgctggtcaaaagccagtcaagttgggtcaattgttgaacgtttgtactactaacgctttggctagagttatgttgggtaagagagttttcgccgacggttctggtgatgttgatccacaagctgctgaattcaagtctatggttgttgaaatgatggttgtcgccggtgttttcaacattggtgatttcattccacaattgaactggttggatattcaaggtgttgccgctaagatgaagaagttgcacgctagattcgacgctttcttgactgatatcttggaagaacacaagggtaagattttcggtgaaatgaaggatttgttgtctactttgatctctttgaagaacgatgatgctgataacgatggtggtaagttgactgatactgaaattaaggctttgttgttgaacttgttcgttgctggtactgacacttcttcttctactgttgaatgggccattgctgaattgattagaaacccaaagatcttggcccaagcccaacaagaaatcgacaaggtcgttggtagagacagattggttggtgaattggacttggcccaattgacttacttggaagctatcgtcaaggaaaccttcagattgcacccatctaccccattgtctttgccaagaattgcttctgaatcttgtgaaatcaacggttacttcattccaaagggttctactttgttgttgaacgtttgggccattgctagagatccaaacgcttgggctgatccattggaattcagaccagaaagattcttgccaggtggtgaaaagccaaaggttgatgtcagaggtaacgacttcgaagtcatcccattcggtgctggtagaagaatttgtgctggtatgaacttgggtatcagaatggtccaattgatgattgctactttgatccacgctttcaactgggatttggtatctggtcaattgccagaaatgttgaacatggaagaagcttacggtttgaccttgcaaagagctgatccattggttgttcacccaagaccaagattggaagcccaagcttacattggttaa

SEQ ID NO: 7 - protein, 519 aa, Callistephus chinensis
MSTILPFIFYTCITALVLYVLLNLLTRNPNRLPPGPTPWPIVGNLPHLGMIPHHSLAALAQKYGPLMHLRLGFVDVVVAASASVAAQFLKTHDANFASRPPNSGAKHIAYNYQDLVFAPYGPRWRMLRKICSVHLFSTKALDDFRHVREEEVAILTRVLVHAGESAVKLGQLLNVCTTNALARVMLGRRVFADGSEGRGVDPKADEFKDMVVELMELAGEFNIGDFIPPLDCLDLQGITKKMKKLHARFDKFLNIILDDHKIEKGAAGRRHSDLLTTLISLKDVDAADDDEEGKLSDIEIKALLLNLFAAGTDTSSSTVEWAVAELIRHPELLKQAREEMDIVVGRDRLVTELDLSRLTFLQAIVKETFRLHPSTPLSLPRMASESCEVDGYYIPKGSTLLVNVWAIARDPKMWTNPLEFRPSRFLPGGEKPDADIKGNDFEVIPFGAGRRICAGMSLGMRMVQLLIATLVQTFDWELANGLDPEKLNMEEAYGLTLQRAEPLMVHPRPRLSPHVYESR

SEQ ID NO: 8 - DNA, 1560 bp, synthetic construct
  misc_feature 1..1560: note = coding sequence for SEQ ID NO: 7
  source 1..1560: mol_type = other DNA
atgtccaccattttgccattcattttctacacttgtatcactgccttggttttgtacgttttgttgaacttgttgaccagaaacccaaacagattgccaccaggtccaaccccatggccaatcgttggtaacttgccacacttgggtatgatcccacaccactctttggctgccttggcccaaaagtacggtccattgatgcacttgagattgggtttcgttgacgttgtcgttgccgcttctgcttccgttgctgctcaattcttaaagactcacgacgctaacttcgcttctagaccaccaaactctggtgccaagcacattgcctacaactaccaagatttggttttcgctccatacggtccaagatggagaatgttgagaaagatttgttctgttcacttgttctccactaaggctttggacgacttcagacacgttagagaagaagaagttgctatcttgactagagttttggtccacgctggtgaatctgctgttaagttgggtcaattgttgaacgtttgtaccactaacgctttggctagagttatgttgggtagaagagttttcgctgacggttctgaaggtagaggtgtcgacccaaaggctgatgaattcaaggacatggttgttgaattgatggaattggccggtgaattcaacatcggtgacttcatcccaccattggactgtttggatttgcaaggtatcaccaagaagatgaagaagttgcacgctagattcgacaagttcttgaacatcatcttggacgaccacaagatcgaaaagggtgctgccggtagaaggcactctgacttgttgaccactttgatttctttgaaggatgttgatgctgctgatgatgatgaagaaggtaagttgtctgacattgaaatcaaggctttgttgttgaacttgttcgctgctggtactgacacttcttcttctaccgttgaatgggctgttgccgaattgattagacacccagaattgttgaagcaagctagagaagaaatggatatcgttgttggtagagacagattggttaccgaattggacttgtctagattgactttcttgcaagccattgttaaggaaaccttcagattgcacccatctactccattgtccttgccaagaatggcttctgaatcttgtgaagttgatggttactacattccaaagggttccactttgttggttaacgtttgggccatcgccagagatccaaagatgtggactaacccattggaattcagaccatctagattcttgccaggtggtgaaaagccagatgctgatatcaagggtaacgatttcgaagtcatcccattcggtgccggtagaagaatctgtgctggtatgtctttgggtatgagaatggtccaattgttgattgctactttggtccaaaccttcgattgggaattggctaacggtttggacccagaaaagttgaacatggaagaagcttacggtttgaccttgcaaagagctgaaccattgatggttcacccaagaccaagattgtctccacacgtttacgaatctagataa

SEQ ID NO: 9 - protein, 511 aa, Callistephus chinensis
MSSILSLLVYFCISLLVIIALVNMFITRHTNRLPPGPAPWPVVGNLPHLGAIPHHTLAALATKYGPLVYLRLGFVHVVVASSPSVAAQFLKVHDLKFASRPPNSGAKHIAYNYQDMVFAPYGPQWTMFRKICKDHLFSSKALDDFRHVRQEEVAILARGLAGAGRSKVNLGQQLNMCTANTLARMMLDKRVFGNESGGDDPKANEFKEMATELMFLAGQFNIGDYIPVLDWLDLQGIVKKMKKLHTRFDKFLDVILDEHKVIASGHIDMLSTLISLKDDTSVDGRKPSDIEIKALLLELFVAGTDTSSNTVEWAIAELIRQPHLLKRAQEEMDSVVGQNRLVTEMDLSQLTFLQAIVKEAFRLHPSTPLSLPRIASESCEVDGYYIPKGSTLLVNIWAIGRHPEVWTDPLEFRPTRFLPGGEKPGIVVKVNDFEVLPFGAGRRICAGMSLALRTVQLLMGTLVQAFDWELANGIKPEKLNMDEAFGLSVQRAEPLVVHPRPRLPPHVYKSG

SEQ ID NO: 10 - DNA, 1536 bp, synthetic construct
  misc_feature 1..1536: note = coding sequence for SEQ ID NO: 9
  source 1..1536: mol_type = other DNA
atgtcctctattttgtctttgttggtctacttctgtatctctttgttggttatcattgctttggttaacatgttcatcaccagacacaccaacagattgccaccaggtccagccccatggccagtcgtcggtaacttgccacacttgggtgctattccacaccacactttggctgctttggctactaagtacggtccattggtttacttgagattgggtttcgttcacgttgttgttgcctcttctccatccgttgctgctcaattcttgaaggttcacgacttgaagttcgcctctagaccaccaaactctggtgctaagcacatcgcttacaactaccaagatatggttttcgctccatacggtccacaatggactatgttcagaaagatttgtaaggatcacttgttctcttctaaggctttggatgatttcagacacgttagacaagaagaagttgctatcttggctagaggtttggctggtgctggtagatctaaggttaacttgggtcaacaattgaacatgtgtaccgctaacactttggctagaatgatgttggacaagagagttttcggtaacgaatctggtggtgatgatccaaaggctaacgaattcaaggaaatggctactgaattgatgttcttggctggtcaattcaacattggtgactacatcccagttttggactggttggacttgcaaggtattgttaagaagatgaagaagttgcacactagattcgataagttcttggacgttatcttggatgaacacaaggttatcgcttctggtcacatcgacatgttgtctactttgatttctttgaaggatgatacctctgttgacggtagaaagccatccgacatcgaaatcaaggctttgttgttggaattgttcgttgctggtactgacacttcttctaacaccgttgaatgggctatcgctgaattgattagacaaccacacttgttgaagagagcccaagaagaaatggactctgttgttggtcaaaacagattggttaccgaaatggacttgtctcaattgactttcttgcaagccattgttaaggaagccttcagattgcacccatctactccattgtccttgccaagaattgcttccgaatcttgtgaagttgatggttactacatcccaaagggttccactttgttggttaacatctgggccattggtagacacccagaagtttggaccgacccattggaattcagaccaactagattcttgccaggtggtgaaaagccaggtattgtcgtcaaggttaacgatttcgaagtcttgccattcggtgccggtagaagaatctgtgctggtatgtctttggccttgagaactgtccaattgttgatgggtactttggtccaagccttcgattgggaattggctaacggtatcaagccagaaaagttgaacatggacgaagccttcggtttgtctgttcaaagagctgaaccattggttgttcacccaagaccaagattgccaccacacgtttacaagtctggttaa

SEQ ID NO: 11 - protein, 513 aa, Gerbera hybrida
MSTPLTLLIGTCVTGLFLYVLLNRCTRNPNRLPPGPTPWPVVGNLPHLGTIPHHSLAAMAKKYGPLMHLRLGFVDVVVAASASVAAQFLKTHDANFADRPPNSGAKHIAYNYQDLVFAPYGPRWRMLRKICSVHLFSTKALDDFRHVRQEEVAILARALVGAGKSPVKLGQLLNVCTTNALARVMLGRRVFDSGDAQADEFKDMVVELMVLAGEFNIGDFIPVLDWLDLQGVTKKMKKLHAKFDSFLNTILEEHKTGAGDGVASGKVDLLSTLISLKDDADGEGGKLSDIEIKALLLNLFTAGTDTSSSTIEWAIAELIRNPQLLNQARKEMDTIVGQDRLVTESDLGQLTFLQAIIKETFRLHPSTPLSLPRMALESCEVGGYYIPKGSTLLVNVWAISRDPKIWADPLEFQPTRFLPGGEKPNTDIKGNDFEVIPFGAGRRICVGMSLGLRMVQLLTATLIHAFDWELADGLNPKKLNMEEAYGLTLQRAAPLVVHPRPRLAPHVYETTKV

SEQ ID NO: 12 - DNA, 1542 bp, synthetic construct
  misc_feature 1..1542: note = coding sequence for SEQ ID NO:11
  source 1..1542: mol_type = other DNA
atgtccactccattgactttgttgatcggtacctgtgtcactggtttgttcttgtacgttttgttgaacagatgtaccagaaacccaaacagattgccaccaggtccaactccatggccagtcgtcggtaacttgccacacttgggtactatcccacaccactctttggctgctatggctaagaagtacggtccattgatgcacttgagattgggtttcgtcgacgtcgttgttgccgcctccgcctccgtcgccgctcaattcttaaagactcacgacgctaacttcgccgatagaccaccaaactccggtgccaagcacatcgcttacaactaccaagatttggttttcgctccatacggtccaagatggagaatgttgagaaagatttgttctgttcacttgttctccaccaaggctttggatgatttcagacacgtcagacaagaagaagttgctatcttggctagagctttggtcggtgccggtaagtctccagttaagttgggtcaattgttgaacgtttgtaccactaacgctttggctagagttatgttgggtagaagagttttcgactccggtgatgctcaagctgatgaattcaaggacatggttgttgaattgatggttttggccggtgaattcaacatcggtgacttcatcccagttttggactggttggacttgcaaggtgttactaagaagatgaagaagttgcacgctaagttcgactctttcttgaacactatcttggaagaacacaagaccggtgccggtgacggtgtcgcttctggtaaggttgacttgttgtctactttgatttctttgaaggatgacgctgatggtgaaggtggtaagttgtctgacattgaaatcaaggctttgttgttgaacttgttcactgctggtactgacacttcttcttctactattgaatgggctatcgctgaattgattagaaacccacaattgttgaaccaagccagaaaggaaatggacaccatcgttggtcaagacagattggttaccgaatctgacttgggtcaattgactttcttgcaagccattatcaaggaaactttcagattgcacccatctaccccattgtctttgccaagaatggctttggaatcttgtgaagttggtggttactacatcccaaagggttccactttgttggttaacgtttgggccatttctagagatccaaagatttgggccgatccattggaattccaaccaactagattcttgccaggtggtgaaaagccaaacactgatatcaagggtaacgatttcgaagtcatcccattcggtgccggtagaagaatttgtgtcggtatgtctttgggtttgagaatggtccaattgttgactgctaccttgatccacgccttcgattgggaattggctgatggtttgaacccaaagaagttgaacatggaagaagcttacggtttgaccttgcaaagagccgctccattggttgttcacccaagaccaagattggccccacacgtttacgaaactactaaggtctaa

SEQ ID NO: 13 - protein, 515 aa, Osteospermum hybrid cultivar
MSTILPLVLYSCITGLVIYVLLNLRTRHSNRLPPGPTPWPIVGNLPHLGVVPHHSLAAMAEKYGPLMHLRLGFVDVVVAASAAVAAQFLKVHDANFASRPPNSGAKHIAYNYQDLVFAPYYGPRWRMLRKICSVHLFSSKALDDFRHVRQEEVAILTRALIGAGDSPVKLGQLLNVCTTNALARVMLGKRVFGDRSGGGDPKADEFKDMVVEVMELAGEFNIGDFIPVLDSLDLQGIAKKMKELHVRFDSFLGKILEEHKTGNGGASSQHTDLLTTLISLKDDTDEEGGKLSDIEIKALLLNLFTAGTDTSSSTVEWAIAELIRHPQLLKQAQEEIDNVVGRDHLVTELDLTQLPFLQAIVKETFRLHPSTPLSLPRIASESCEVNGYHIPKGSTLLVNVWAIARDPKMWSEPLEFRPARFLPGGEKPDADVKGNDFEVIPFGAGRRSCAGMSLGLRMVQLLVATLVQTFDWELANGLKPEKLNMEEAYGLTLQRAAPLLVHPKPRLAPHVYGSN

SEQ ID NO: 14 - DNA, 1548 bp, synthetic construct
  misc_feature 1..1548: note = coding sequence for SEQ ID NO: 13
  source 1..1548: mol_type = other DNA
atgtccaccattttgccattggttttgtactcttgtatcactggtttggttatctacgttttgttgaacttgagaaccagacactctaacagattgccaccaggtccaactccatggccaatcgtcggtaacttgccacacttgggtgttgttccacaccactctttggctgctatggctgaaaagtacggtccattgatgcacttgagattgggtttcgttgacgttgttgttgctgcttctgctgccgttgctgctcaattcttgaaggttcacgatgctaacttcgcttctagaccaccaaactccggtgctaagcacatcgcttacaactaccaagacttggttttcgctccatactacggtccaagatggagaatgttgagaaagatttgttccgttcacttgttctcttctaaggctttggatgatttcagacacgtcagacaagaagaagttgctatcttgactagagctttgatcggtgccggtgactctccagttaagttgggtcaattgttgaacgtttgtactactaacgctttggctagagttatgttgggtaagagagttttcggtgacagatctggtggtggtgatccaaaggctgatgaattcaaggatatggttgttgaagttatggaattggccggtgaattcaacatcggtgatttcatcccagttttggattctttggatttgcaaggtatcgctaagaagatgaaggaattgcacgttagattcgattctttcttgggtaagatcttggaagaacacaagaccggtaacggtggtgcttcttctcaacacactgacttgttgactaccttgatttctttgaaggatgatactgatgaagaaggtggtaagttgtctgacattgaaatcaaggctttgttgttgaacttgttcactgctggtactgacacttcttcttctaccgttgaatgggctatcgccgaattgattagacacccacaattgttgaagcaagcccaagaagaaatcgacaacgttgttggtagagatcacttggttaccgaattggacttgacccaattgccattcttgcaagccattgttaaggaaaccttcagattgcacccatctactccattgtctttgccaagaattgcttccgaatcttgtgaagtcaacggttaccacatcccaaagggttccactttgttggttaacgtttgggccatcgccagagatccaaagatgtggtccgaaccattggaattcagaccagccagattcttgccaggtggtgaaaagccagatgctgatgttaagggtaacgatttcgaagtcatcccattcggtgccggtagaagatcttgtgctggtatgtctttgggtttgagaatggttcaattgttggttgctactttggttcaaaccttcgactgggaattggctaacggtttgaagccagaaaagttgaacatggaagaagcttacggtttgactttgcaaagagctgctccattgttggttcacccaaagccaagattggctccacacgtttacggttctaactaa

SEQ ID NO: 15 - protein, 539 aa, Citrus clementina
MSQPLVRLLVPFLRFLWETKQRAMSTLPLLILYTSLLAIVISFLFSLLRNRRRHSSHRLPPGPKPWPIVGNLPHLGPMPHQSIAGLARTHGPLMYLRLGFVDVVVAASASVAAQFLKIHDSNFSNRPPNSGAKHIAYNYQDIVFRPYGPRWRMLRKISSVHLFSGKALDDYRHVRQEEMAVLTRALASAGTEPVNLAQRLNLCVVNALGRVMLGFRVFGDGTGGSDPRADEFKSMVVELMVLAGVFNVGDFVPALERLDLQGVARKMKKLHKRFDVFLSDILEERKMNGRDGGNKLTDLLGTLISLMDDANGEEKLTETEIKALLLNMFTAGTDTSSSTIEWAIAELIRHPKVWAQVQQELDSVVGRDRLVTELDLPQLTYLQAVIKEIFRLHPSTPLSLPRAASESCKINGYDIPKGSTLLVNIWAIARDPNEWADPLEFRPERFLPGGEKYNVDVKGNDYELIPFGAGRRICAGLSWGLRMVQLGTATLAHAFNWELPGGLKPEKLNMDEAYGLTLQRAAPLVVHPRPRLSPNAYQA

SEQ ID NO: 16 - DNA, 1620 bp, synthetic construct
  misc_feature 1..1620: note = coding sequence for SEQ ID NO: 15
  source 1..1620: mol_type = other DNA
atgtcccaaccattggttagattgttggttccattcttgagattcttgtgggaaactaagcaaagagctatgtctactttgccattgttgatcttgtacacttctttgttggctattgtcatttctttcttgttctctttgttgagaaacagaaggagacactcctcccacagattgccaccaggtccaaagccatggccaatcgtcggtaacttgccacacttgggtccaatgccacaccaatccatcgctggtttggccagaacccacggtccattgatgtacttgagattgggtttcgttgacgtcgtcgttgccgcttccgcttccgttgctgcccaattcttgaagatccacgattccaacttctccaacagaccaccaaactccggtgccaagcacatcgcttacaactaccaagacatcgttttcagaccatacggtccaagatggagaatgttgagaaagatctcttccgttcacttgttctccggtaaggccttggatgactacagacacgttagacaagaagaaatggctgttttgactagagctttggcttctgctggtactgaaccagttaacttggctcaaagattgaacttgtgtgttgttaacgccttgggtagagttatgttgggtttcagagttttcggtgacggtaccggtggttctgacccaagagctgacgaattcaagtctatggttgttgaattgatggttttggctggtgttttcaacgtcggtgatttcgtcccagccttggaaagattggacttgcaaggtgttgctagaaagatgaagaagttgcacaagagattcgatgttttcttgtctgatatcttggaagaaagaaagatgaacggtagagatggtggtaacaagttgactgacttgttgggtaccttgatttctttgatggatgatgctaacggtgaagaaaagttgactgaaactgaaatcaaggctttgttgttgaacatgttcactgctggtaccgacacttcttcttctactatcgaatgggccattgctgaattgatcagacacccaaaggtctgggcccaagtccaacaagaattggattctgttgttggtagagatagattggtcaccgaattggacttgccacaattgacttacttgcaagccgttatcaaggaaatcttcagattgcacccatctactccattgtctttgccaagagctgcttccgaatcttgtaagatcaacggttacgatattccaaagggttccactttgttggtcaacatctgggccattgctagagatccaaacgaatgggccgacccattggaattcagaccagaaagattcttgccaggtggtgaaaagtacaacgttgatgttaagggtaacgattacgaattgatcccattcggtgccggtagaagaatttgtgctggtttgtcttggggtttgagaatggttcaattgggtactgctactttggcccacgctttcaactgggaattgccaggtggtttgaagccagaaaagttgaacatggacgaagcttacggtttgaccttgcaaagagctgctccattggttgttcacccaagaccaagattgtctccaaacgcttaccaagcttaa

SEQ ID NO: 17 - protein, 517 aa, Citrus sinensis
MSSTLPLLILYTSLLAIVISFLFSLLRNRRRHSSHRLPPGPKPWPIVGNLPHLGPMPHQSIAGLARTHGPLMYLRLGFVDVVVAASASVAAQFLKIHDSNFSNRPPNSGAKHIAYNYQDIVFRPYGPRWRMLRKISSVHLFSGKALDDYRHVRQEEMAVLARALASAGTEPVNLAQRLNLCVVNALGRVMLGFRVFGDGTGGSDPRADEFKSMVVELMVLAGVFNVGDFVPALERLDLQGVARKMKKLHKRFDVFLSDILEERKMNGRDGGNKHTDLLGTLISLMDDANGEEKLTETEIKALLLNMFTAGTDTSSSTIEWAIAELIRHPKVRAQVQQELDSVVGRDRLVTELDLPQLTYLQAVIKEIFRLHPSTPLSLPRAASESCKINGYDIPKGSTLLVNIWAIARDPNEWADPLEFRPERFLPGGEKYNVDVKGNDYELIPFGAGRRICAGLSWGLRMVQLGTATLAHAFNWELPGGLKPEKLSMDEAYGLTLQRAAPLVVHPRPRLSPNAYQA

SEQ ID NO: 18 - DNA, 1554 bp, synthetic construct
  misc_feature 1..1554: note = coding sequence for SEQ ID NO: 17
  source 1..1554: mol_type = other DNA
atgtcctctactttgccattgttgatcttgtacacttctttgttggctattgtcatttctttcttgttctctttgttgagaaacagaaggagacactcctcccacagattgccaccaggtccaaagccatggccaatcgtcggtaacttgccacacttgggtccaatgccacaccaatccatcgctggtttggccagaacccacggtccattgatgtacttgagattgggtttcgttgacgtcgtcgttgccgcttctgcttccgttgctgcccaattcttgaagatccacgattccaacttctccaacagaccaccaaactccggtgccaagcacatcgcttacaactaccaagacatcgttttcagaccatacggtccaagatggagaatgttgagaaagatctcttccgttcacttgttctccggtaaggccttggatgactacagacacgttagacaagaagaaatggctgttttggctagagctttggcttctgctggtactgaaccagttaacttggctcaaagattgaacttgtgtgttgttaacgccttgggtagagttatgttgggtttcagagttttcggtgacggtaccggtggttctgacccaagagctgacgaattcaagtctatggttgttgaattgatggttttggctggtgttttcaacgtcggtgacttcgtcccagccttggaaagattggacttgcaaggtgttgctagaaagatgaagaagttgcacaagagattcgatgttttcttgtctgatatcttggaagaaagaaagatgaacggtagagatggtggtaacaagcacactgacttgttgggtaccttgatttctttgatggatgatgctaacggtgaagaaaagttgactgaaactgaaatcaaggctttgttgttgaacatgttcactgctggtaccgacacttcttcttctactatcgaatgggccattgctgaattgatcagacacccaaaggtcagagcccaagtccaacaagaattggattctgttgttggtagagatagattggtcaccgaattggacttgccacaattgacttacttgcaagccgttatcaaggaaatcttcagattgcacccatctactccattgtctttgccaagagctgcttccgaatcttgtaagatcaacggttacgatattccaaagggttccactttgttggtcaacatctgggccattgctagagatccaaacgaatgggccgacccattggaattcagaccagaaagattcttgccaggtggtgaaaagtacaacgttgatgttaagggtaacgattacgaattgatcccattcggtgccggtagaagaatttgtgctggtttgtcttggggtttgagaatggttcaattgggtactgctactttggcccacgctttcaactgggaattgccaggtggtttgaagccagaaaagttgtctatggacgaagcttacggtttgaccttgcaaagagctgctccattggttgttcacccaagaccaagattgtctccaaacgcttaccaagcttaa

SEQ ID NO: 19 - protein, 513 aa, Pilosella officinarum
MSTLLSLIIYLCITGVTAYVLVNLRTRRANRLPPGPTPWPIVGNLPHLGTIPHHSLADLATRYGPLMHLRLGFVDVVVAASASVAAQFLKTHDANFASRPPNSGAKHMAYNYQDLVFAPYGPRWRMLRKICSVHLFSAKSLDDFRHVRQEEVAILTRALVGAGKSTVKLGQLLHVCTTNALVRVMLGRRVFGDGSGGGDPKADEFKNMVIEMMVLAGEFNLGDFIPVLDLLDLQGVTKKMKKLHTRFDSFLNSILEEHRTSSGGASGHVDLLSTLISLKDEADGEGGKLTDTEIKALLLNLFVAGTDTSSSTVEWAIAELIRNPQLLKQAQQELDTVVGQGRLVNESDLSQLTFLQAIVKETFRLHPSTPLSLPRIASESCEINGYNIPKGSTLLVNVWAIARDPKMWTEPLEFRPSRFLPDGEKPNADVKGNDFEVIPFGAGRRICAGMSLGLRMVQLLTATLIQAFDWELANGLEPRNLNMEEAYGLTLQRAQPLMVHPRPRLAPHVYGTG

SEQ ID NO: 20 - DNA, 1542 bp, synthetic construct
  misc_feature 1..1542: note = coding sequence for SEQ ID NO: 19
  source 1..1542: mol_type = other DNA
atgtccaccttgttgtccttgatcatctacttgtgtatcactggtgttactgcctacgttttggttaacttgagaactagaagagctaacagattgccaccaggtccaaccccatggccaatcgtcggtaacttgccacacttgggtactattccacaccactctttggctgatttggctactagatacggtccattgatgcacttgagattgggtttcgttgacgttgttgttgccgcctctgcttccgtcgctgctcaattcttaaagactcacgacgctaacttcgcttctagaccaccaaactctggtgctaagcacatggcttacaactaccaagatttggttttcgctccatacggtccaagatggagaatgttgagaaagatttgttccgttcacttgttctctgccaagtctttggatgatttcagacacgttagacaagaagaagttgctatcttgactagagctttggtcggtgccggtaagtctactgttaagttgggtcaattgttgcacgtttgtaccactaacgctttggttagagttatgttgggtagaagagttttcggtgatggttctggtggtggtgatccaaaggctgatgaattcaagaacatggttattgaaatgatggttttggccggtgaattcaacttgggtgatttcatcccagttttggatttgttggacttgcaaggtgttactaagaagatgaagaagttgcacactagattcgattctttcttgaactctatcttggaagaacacagaacttcttctggtggtgcttctggtcacgtcgatttgttgtctactttgatttctttgaaggatgaagccgatggtgaaggtggtaagttgactgacactgaaattaaggctttgttgttgaacttgttcgttgctggtactgacacttcttcttctaccgttgaatgggctatcgctgaattgattagaaacccacaattgttgaagcaagcccaacaagaattggacactgttgttggtcaaggtagattggttaacgaatctgacttgtctcaattgaccttcttgcaagccattgttaaggaaactttcagattgcacccatctactccattgtccttgccaagaatcgcttccgaatcttgtgaaatcaacggttacaacattccaaagggttccactttgttggttaacgtttgggccatcgctagagatccaaagatgtggaccgaaccattggaattcagaccatccagattcttgccagacggtgaaaagccaaacgctgatgttaagggtaacgatttcgaagtcatcccattcggtgctggtagaagaatttgtgctggtatgtctttgggtttgagaatggtccaattgttgaccgctactttgattcaagccttcgattgggaattggctaacggtttggaaccaagaaacttgaacatggaagaagcctacggtttgaccttgcaaagagctcaaccattgatggttcacccaagaccaagattggccccacacgtttacggtactggttaa

SEQ ID NO: 21 - protein, 406 aa, Streptomyces avermitilis
MSAAAFDLAFDPWDPAFLADPYPAYADLRAKGRVHYYEPTNQWLVPHHADVSALLRDRRLGRAYQHRYTHEDFGRTAPPAEHEPFHTLNDHGMLDLEPPDHTRIRRLVSKAFTPRTVEQLKPYVAKLAGELVDRLVAAGGGDLLADVAEPLPVAVIAEMLGIPESDRAPLRPWSADICGMYELNPPKDVAAKAVRASVEFSDYLRELIAERRKEPGDDLISGLIAAHDEGDRLTEQEMISTCVLLLNAGHEATVNATVNGWYALFRNPDQLAALRADHSLVPAAVEELMRYDTPLQLFERWVLDEIEIDGTTVPRGAEIAMLFGSANHDPEVFRNPEKLDLTREDNPHISFSAGIHYCIGAPLARIELAASMTALLEKAPTLGLVAEPKRKPNFVIRGLEGLSVAV

SEQ ID NO: 22 - DNA, 1221 bp, synthetic construct
  misc_feature 1..1221: note = coding sequence for SEQ ID NO: 21
  source 1..1221: mol_type = other DNA
atgtccgctgctgctttcgacttggccttcgacccatgggacccagctttcttggccgacccatacccagcctacgccgatttgagagccaagggtagagttcactactacgaaccaactaaccaatggttggttccacaccacgctgacgtttctgctttgttgagagacagaagattgggtagagcttaccaacacagatacactcacgaagatttcggtagaaccgctccaccagctgaacacgaaccattccacaccttgaacgaccacggtatgttggacttggaaccaccagaccacaccagaatcagaagattggtttctaaggctttcactccaagaaccgttgaacaattgaagccatacgttgccaagttggccggtgaattggttgacagattggtcgctgctggtggtggtgatttgttggctgatgtcgccgaaccattgccagttgccgtcatcgccgaaatgttgggtatcccagaatccgacagagccccattaagaccatggtctgctgacatctgtggtatgtacgaattgaacccaccaaaggacgttgctgctaaggctgttagagcttctgttgaattctccgactacttgagagaattgatcgccgaaagaagaaaggaaccaggtgacgatttgatctctggtttgatcgccgcccacgacgaaggtgacagattgaccgaacaagaaatgatctccacctgtgtcttgttgttgaacgctggtcacgaagccaccgtcaacgccactgtcaacggttggtacgccttgttcagaaacccagaccaattggccgccttgagagccgaccactctttggttccagccgccgttgaagaattgatgagatacgacactccattgcaattgttcgaaagatgggtcttggacgaaatcgaaatcgacggtactactgtcccaagaggtgctgaaatcgccatgttgttcggttccgccaaccacgacccagaagttttcagaaacccagaaaagttggacttgaccagagaagataacccacacatttccttctctgctggtatccactactgtatcggtgctccattggctagaatcgaattggctgcttctatgactgccttgttggaaaaggccccaactttgggtttggttgctgaaccaaagagaaagccaaacttcgttatcagaggtttggaaggtttgtctgtcgctgtctaa

SEQ ID NO: 23 - protein, 715 aa, Catharanthus roseus
MSDSSSEKLSPFELMSAILKGAKLDGSNSSDSGVAVSPAVMAMLLENKELVMILTTSVAVLIGCVVVLIWRRSSGSGKKVVEPPKLIVPKSVVEPEEIDEGKKKFTIFFGTQTGTAEGFAKALAEEAKARYEKAVIKVIDIDDYAADDEEYEEKFRKETLAFFILATYGDGEPTDNAARFYKWFVEGNDRGDWLKNLQYGVFGLGNRQYEHFNKIAKVVDEKVAEQGGKRIVPLVLGDDDQCIEDDFAAWRENVWPELDNLLRDEDDTTVSTTYTAAIPEYRVVFPDKSDSLISEANGHANGYANGNTVYDAQHPCRSNVAVRKELHTPASDRSCTHLDFDIAGTGLSYGTGDHVGVYCDNLSETVEEAERLLNLPPETYFSLHADKEDGTPLAGSSLPPPFPPCTLRTALTRYADLLNTPKKSALLALAAYASDPNEADRLKYLASPAGKDEYAQSLVANQRSLLEVMAEFPSAKPPLGVFFAAIAPRLQPRFYSISSSPRMAPSRIHVTCALVYEKTPGGRIHKGVCSTWMKNAIPLEESRDCSWAPIFVRQSNFKLPADPKVPVIMIGPGTGLAPFRGFLQERLALKEEGAELGTAVFFFGCRNRKMDYIYEDELNHFLEIGALSELLVAFSREGPTKQYVQHKMAEKASDIWRMISDGAYVYVCGDAKGMARDVHRTLHTIAQEQGSMDSTQAEGFVKNLQMTGRYLRDVW

SEQ ID NO: 24 - DNA, 2148 bp, synthetic construct
  misc_feature 1..2148: note = coding sequence for SEQ ID NO: 23
  source 1..2148: mol_type = other DNA
atgtccgattcttcttctgaaaagttgtctccattcgaattgatgtctgctatcttgaagggtgctaagttggatggttctaactcttctgattctggtgttgctgtttctccagctgttatggctatgttgttggaaaacaaggaattggttatgattttgactacttctgttgctgttttgatcggttgtgtcgttgttttgatctggagaagatcttccggttctggtaagaaggtcgttgaaccaccaaagttgatcgttccaaagtctgttgttgaaccagaagaaattgatgaaggtaagaagaagttcaccatcttcttcggtactcaaactggtactgctgaaggtttcgctaaggctttggctgaagaagccaaggctagatacgaaaaggctgttatcaaggttattgatatcgatgattacgctgctgatgatgaagaatacgaagaaaagttcagaaaggaaaccttggctttcttcatcttggccacttacggtgatggtgaaccaaccgacaacgctgctagattctacaagtggttcgttgaaggtaacgatagaggtgactggttgaagaacttgcaatacggtgttttcggtttgggtaacagacaatacgaacacttcaacaagattgctaaggttgttgatgaaaaggttgctgaacaaggtggtaagagaattgttccattggttttgggtgacgatgaccaatgtattgaagatgacttcgctgcttggagagaaaacgtttggccagaattggataacttgttgagagatgaagatgatactactgtttctactacctacactgctgctattccagaatacagagttgttttcccagacaagtctgattctttgatttctgaagctaacggtcacgccaacggttacgctaacggtaacaccgtttacgatgcccaacacccatgtagatctaacgttgctgttagaaaggaattgcacactccagcttctgatagatcttgtacccacttggatttcgacattgctggtactggtttgtcttacggtactggtgatcacgttggtgtttactgtgataacttgtctgaaaccgttgaagaagctgaaagattgttgaacttgccaccagaaacttacttctctttgcacgctgataaggaagatggtaccccattggctggttcttctttgccaccaccattcccaccatgtactttgagaaccgccttgactagatacgctgatttgttgaacactccaaagaagtctgctttgttggctttggctgcttacgcttctgatccaaacgaagccgatagattgaagtacttggcttctccagccggtaaggatgaatacgctcaatctttggttgctaaccaaagatctttgttggaagtcatggctgaattcccatctgctaagccaccattgggtgttttcttcgctgctattgctccaagattgcaaccaagattctactctatctcttcttctccaagaatggctccatctagaattcacgtcacttgtgctttggtttacgaaaagactccaggtggtagaattcacaagggtgtttgttctacttggatgaagaacgccattccattggaagaatctagagactgttcttgggctccaatcttcgtcagacaatctaacttcaagttgccagccgatccaaaggttccagttatcatgatcggtccaggtactggtttggctccattcagaggtttcttgcaagaaagattggctttgaaggaagaaggtgctgaattgggtactgctgttttcttcttcggttgtagaaacagaaagatggattacatctacgaagatgaattgaaccacttcttggaaattggtgctttgtccgaattgttggttgctttctctagagaaggtccaactaagcaatacgttcaacacaagatggctgaaaaggcttctgatatttggagaatgatttctgatggtgcttacgtttacgtctgtggtgatgccaagggtatggccagagatgtccacagaactttgcacaccattgctcaagaacaaggttctatggattctactcaagctgaaggtttcgttaagaacttgcaaatgaccggtagatacttgagagatgtctggtaa

SEQ ID NO: 25 - protein, 692 aa, Saccharomyces cerevisiae
MSPFGIDNTDFTVLAGLVLAVLLYVKRNSIKELLMSDDGDITAVSSGNRDIAQVVTENNKNYLVLYASQTGTAEDYAKKFSKELVAKFNLNVMCADVENYDFESLNDVPVIVSIFISTYGEGDFPDGAVNFEDFICNAEAGALSNLRYNMFGLGNSTYEFFNGAAKKAEKHLSAAGAIRLGKLGEADDGAGTTDEDYMAWKDSILEVLKDELHLDEQEAKFTSQFQYTVLNEITDSMSLGEPSAHYLPSHQLNRNADGIQLGPFDLSQPYIAPIVKSRELFSSNDRNCIHSEFDLSGSNIKYSTGDHLAVWPSNPLEKVEQFLSIFNLDPETIFDLKPLDPTVKVPFPTPTTIGAAIKHYLEITGPVSRQLFSSLIQFAPNADVKEKLTLLSKDKDQFAVEITSKYFNIADALKYLSDGAKWDTVPMQFLVESVPQMTPRYYSISSSSLSEKQTVHVTSIVENFPNPELPDAPPVVGVTTNLLRNIQLAQNNVNIAETNLPVHYDLNGPRKLFANYKLPVHVRRSNFRLPSNPSTPVIMIGPGTGVAPFRGFIRERVAFLESQKKGGNNVSLGKHILFYGSRNTDDFLYQDEWPEYAKKLDGSFEMVVAHSRLPNTKKVYVQDKLKDYEDQVFEMINNGAFIYVCGDAKGMAKGVSTALVGILSRGKSITTDEATELIKMLKTSGRYQEDVW

SEQ ID NO: 26 - DNA, 2079 bp, synthetic construct
  misc_feature 1..2079: note = coding sequence for SEQ ID NO: 25
  source 1..2079: mol_type = other DNA
atgtccccgtttggaatagacaacaccgacttcactgtcctggcggggctagtgcttgccgtgctactgtacgtaaagagaaactccatcaaggaactgctgatgtccgatgacggagatatcacagctgtcagctcgggcaacagagacattgctcaggtggtgaccgaaaacaacaagaactacttggtgttgtatgcgtcgcagactgggactgccgaggattacgccaaaaagttttccaaggagctggtggccaagttcaacctaaacgtgatgtgcgcagatgttgagaactacgactttgagtcgctaaacgatgtgcccgtcatagtctcgatttttatctctacatatggtgaaggagacttccccgacggggcggtcaactttgaggactttatttgtaatgcggaagcgggtgcactatcgaacctgaggtataatatgtttggtctgggaaattctacttatgaattctttaatggtgccgccaagaaggccgagaagcatctctccgccgcgggcgctatcagactaggcaagctcggtgaagctgatgatggtgcaggaactacagacgaagattacatggcctggaaggactccatcctggaggttttgaaagacgaactgcatttggacgaacaggaagccaagttcacctctcaattccagtacactgtgttgaacgaaatcactgactccatgtcgcttggtgaaccctctgctcactatttgccctcgcatcagttgaaccgcaacgcagacggcatccaattgggtcccttcgatttgtctcaaccgtatattgcacccatcgtgaaatctcgcgaactgttctcttccaatgaccgtaattgcatccactctgaatttgacttgtccggctctaacatcaagtactccactggtgaccatcttgctgtttggccttccaacccattggaaaaggtcgaacagttcttatccatattcaacctggaccctgaaaccatttttgacttgaagcccctggatcccaccgtcaaagtgcccttcccaacgccaactactattggcgctgctattaaacactatttggaaattacaggacctgtctccagacaattgttttcatctttgattcagttcgcccccaacgctgacgtcaaggaaaaattgactctgctttcgaaagacaaggaccaattcgccgtcgagataacctccaaatatttcaacatcgcagatgctctgaaatatttgtctgatggcgccaaatgggacaccgtacccatgcaattcttggtcgaatcagttccccaaatgactcctcgttactactctatctcttcctcttctctgtctgaaaagcaaaccgtccatgtcacctccattgtggaaaactttcctaacccagaattgcctgatgctcctccagttgttggtgttacgactaacttgttaagaaacattcaattggctcaaaacaatgttaacattgccgaaactaacctacctgttcactacgatttaaatggcccacgtaaacttttcgccaattacaaattgcccgtccacgttcgtcgttctaacttcagattgccttccaacccttccaccccagttatcatgatcggtccaggtaccggtgttgccccattccgtgggtttatcagagagcgtgtcgcgttcctcgaatcacaaaagaagggcggtaacaacgtttcgctaggtaagcatatactgttttatggatcccgtaacactgatgatttcttgtaccaggacgaatggccagaatacgccaaaaaattggatggttcgttcgaaatggtcgtggcccattccaggttgccaaacaccaaaaaagtttatgttcaagataaattaaaggattacgaggaccaagtatttgaaatgattaacaacggtgcatttatctacgtctgtggtgatgcaaagggtatggccaagggtgtgtcaaccgcattggttggcatcttatcccgtggtaaatccattaccactgatgaagcaacagagctaatcaagatgctcaagacttcaggtagataccaagaagatgtctggtaa

SEQ ID NO: 27 - protein, 658 aa, synthetic construct
  REGION 1..658: note = chimeric cytochrome P450 reductase
MSPFGIDNTDFTVLAGLVLAVLLYVKRNSIKELLMSDDGDITAVSSGNRDIAQVVTENNKNYLVLYASQTGTAEDYAKKFSKELVAKFNLNVMCADVENYDFESLNDVPVIVSIFISTYGEGDFPDGAVNFEDFICNAEAGALSNLRYNMFGLGNSTYEFFNGAAKKAEKHLSAAGAIRLGKLGEADDGAGTTDEDYMAWKDSILEVLKDELGVEATGEESSIRQYELVVHTDIDAAKVYMGEMGRLKSYENQKPPFDAKNPFLAAVTTNRKLNQGTERHLMHLELDISDSKIRYESGDHVAVYPANDSALVNQLGKILGADLDVVMSLNNLDEESNKKHPFPCPTSYRTALTYYLDITNPPRTNVLYELAQYASEPSEQELLRKMASSSGEGKELYLSWVVEARRHILAILQDCPSLRPPIDHLCELLPRLQARYYSIASSSKVHPNSVHICAVVVEYETKAGRINKGVATNWLRAKEPAGENGGRALVPMFVRKSQFRLPFKATTPVIMVGPGTGVAPFIGFIQERAWLRQQGKEVGETLLYYGCRRSDEDYLYREELAQFHRDGALTQLNVAFSREQSHKVYVQHLLKQDREHLWKLIEGGAHIYVCGDARNMARDVQNTFYDIVAELGAMEHAQAVDYIKKLMTKGRYSLDVWS

SEQ ID NO: 28 - DNA, 1977 bp, synthetic construct
  misc_feature 1..1977: note = coding sequence for SEQ ID NO: 27
  source 1..1977: mol_type = other DNA
atgtccccattcggtatcgacaacaccgacttcactgtcttggctggtttggttttggccgttttgttgtacgttaagagaaactccatcaaggaattgttgatgtccgatgacggtgatatcactgctgtctcttctggtaacagagacattgctcaagttgttaccgaaaacaacaagaactacttggttttgtacgcttctcaaactggtactgccgaagattacgccaagaagttctccaaggaattggttgccaagttcaacttgaacgttatgtgtgctgatgttgaaaactacgacttcgaatctttgaacgatgttccagtcatcgtatctattttcatctctacttacggtgaaggtgacttcccagacggtgctgtcaacttcgaagatttcatttgtaacgctgaagctggtgctttgtctaacttgagatacaacatgttcggtttgggtaactctacttacgaattcttcaacggtgccgccaagaaggccgaaaagcacttgtccgccgctggtgctatcagattgggtaagttgggtgaagctgatgatggtgctggtactactgacgaagattacatggcctggaaggactccatcttggaagttttgaaggacgaattgggtgttgaagccactggtgaagaatcctctattagacaatacgaattggttgtccacaccgacatcgatgctgccaaggtttacatgggtgaaatgggtagattgaagtcttacgaaaaccaaaagccaccattcgatgccaagaacccattcttggctgctgtcaccaccaacagaaagttgaaccaaggtaccgaaagacacttgatgcacttggaattggacatctctgactccaagatcagatacgaatctggtgaccacgttgctgtttacccagccaacgactctgctttggtcaaccaattgggtaagatcttgggtgccgacttggacgtcgtcatgtccttgaacaacttggatgaagaatccaacaagaagcacccattcccatgtccaacttcctacagaactgccttgacctactacttggacatcaccaacccaccaagaaccaacgttttgtacgaattggctcaatacgcctctgaaccatctgaacaagaattgttgagaaagatggcctcctcctccggtgaaggtaaggaattgtacttgtcttgggttgttgaagccaggagacacatcttggccatcttgcaagactgtccatccttaagaccaccaatcgaccacttgtgtgaattgttgccaagattgcaagccagatactactccatcgcctcttcctccaaggtccacccaaactctgttcacatctgtgctgttgttgttgaatacgaaaccaaggctggtagaatcaacaagggtgttgccaccaactggttgagagccaaggaaccagccggtgaaaacggtggtagagctttggttccaatgttcgttagaaagtcccaattcagattgccattcaaggccaccactccagtcatcatggttggtccaggtaccggtgttgctccattcatcggtttcatccaagaaagagcctggttgagacaacaaggtaaggaagttggtgaaactttgttgtactacggttgtagaagatctgatgaagattacttgtacagagaagaattggctcaattccacagagatggtgctttgacccaattgaacgttgccttctccagagaacaatcccacaaggtctacgtccaacacttgttgaagcaagacagagaacacttgtggaagttgatcgaaggtggtgcccacatctacgtctgtggtgatgctagaaacatggccagagatgttcaaaacaccttctacgacatcgttgctgaattgggtgccatggaacacgctcaagctgttgactacatcaagaagttgatgaccaagggtagatactccttggacgtttggtcttaa

SEQ ID NO: 29 - protein, 693 aa, Arabidopsis thaliana
MSTSALYASDLFKQLKSIMGTDSLSDDVVLVIATTSLALVAGFVVLLWKKTTADRSGELKPLMIPKSLMAKDEDDDLDLGSGKTRVSIFFGTQTGTAEGFAKALSEEIKARYEKAAVKVIDLDDYAADDDQYEEKLKKETLAFFCVATYGDGEPTDNAARFYKWFTEENERDIKLQQLAYGVFALGNRQYEHFNKIGIVLDEELCKKGAKRLIEVGLGDDDQSIEDDFNAWKESLWSELDKLLKDEDDKSVATPYTAVIPEYRVVTHDPRFTTQKSMESNVANGNTTIDIHHPCRVDVAVQKELHTHESDRSCIHLEFDISRTGITYETGDHVGVYAENHVEIVEEAGKLLGHSLDLVFSIHADKEDGSPLESAVPPPFPGPCTLGTGLARYADLLNPPRKSALVALAAYATEPSEAEKLKHLTSPDGKDEYSQWIVASQRSLLEVMAAFPSAKPPLGVFFAAIAPRLQPRYYSISSSPRLAPSRVHVTSALVYGPTPTGRIHKGVCSTWMKNAVPAEKSHECSGAPIFIRASNFKLPSNPSTPIVMVGPGTGLAPFRGFLQERMALKEDGEELGSSLLFFGCRNRQMDFIYEDELNNFVDQGVISELIMAFSREGAQKEYVQHKMMEKAAQVWDLIKEEGYLYVCGDAKGMARDVHRTLHTIVQEQEGVSSSEAEAIVKKLQTEGRYLRDVW

SEQ ID NO: 30 - DNA, 2082 bp, synthetic construct
  misc_feature 1..2082: note = coding sequence for SEQ ID NO: 29
  source 1..2082: mol_type = other DNA
atgtccacttctgctttgtacgcttccgatttgttcaagcaattgaagtctattatgggtactgattctttgtccgacgatgttgttttggttattgctactacttctttggctttggttgctggtttcgttgttttgttgtggaagaaaactactgctgatagatctggtgaattgaagccattgatgatcccaaagtctttgatggctaaggacgaagatgatgatttggatttgggttccggtaagactagagtctctatcttcttcggtactcaaactggtactgctgaaggtttcgctaaggctttgtccgaagaaatcaaggctagatacgaaaaggctgctgtcaaggtcattgacttggatgactacgctgccgatgatgaccaatacgaagaaaagttgaagaaggaaactttggctttcttctgtgttgctacttacggtgatggtgaaccaactgacaacgctgccagattctacaagtggttcactgaagaaaacgaaagagatatcaagttgcaacaattggcttacggtgttttcgctttgggtaacagacaatacgaacacttcaacaagatcggtatcgttttggatgaagaattgtgtaagaagggtgctaagagattgattgaagtcggtttgggtgatgatgatcaatctattgaagatgatttcaacgcctggaaggaatctttgtggtctgaattggacaagttgttgaaggacgaagatgataagtctgttgctactccatacactgctgttattccagaatacagagttgttactcacgatccaagattcactactcaaaagtctatggaatctaacgttgccaacggtaacactactattgacattcaccacccatgtagagttgatgttgctgttcaaaaggaattgcacactcacgaatctgatagatcttgtattcacttggaattcgacatctccagaactggtattacttacgaaactggtgaccacgttggtgtttacgctgaaaaccacgttgaaatcgttgaagaagctggtaagttgttgggtcactctttggatttggttttctccatccacgctgacaaggaagatggttccccattggaatctgctgttccaccaccattcccaggtccatgtactttgggtactggtttggctagatacgctgacttgttgaacccaccaagaaagtctgctttggttgccttggctgcctacgccactgaaccatctgaagccgaaaagttgaagcacttgacttctccagatggtaaggatgaatactctcaatggattgttgcttctcaaagatctttgttggaagttatggctgctttcccatctgctaagccaccattgggtgttttcttcgctgctatcgctccaagattgcaaccaagatactactccatctcttcctctccaagattggctccatctagagttcacgttacttccgctttggtttacggtccaactccaactggtagaatccacaagggtgtttgttctacttggatgaagaacgctgttccagctgaaaagtctcacgaatgttctggtgccccaatcttcattagagcttctaacttcaagttgccatccaacccatctactccaatcgttatggttggtccaggtactggtttggctccattcagaggtttcttgcaagaaagaatggctttgaaggaagatggtgaagaattgggttcttctttgttgttcttcggttgtagaaacagacaaatggacttcatctacgaagatgaattgaacaacttcgttgatcaaggtgttatctctgaattgatcatggctttctccagagaaggtgctcaaaaggaatacgttcaacacaagatgatggaaaaggctgctcaagtttgggatttgatcaaggaagaaggttacttgtacgtttgtggtgatgctaagggtatggctagagatgtccacagaactttgcacaccattgttcaagaacaagaaggtgtttcttcttctgaagctgaagctatcgttaagaagttgcaaaccgaaggtagatacttgagagatgtctggtaa

SEQ ID NO: 31 - protein, 712 aa, Arabidopsis thaliana
MSSSSSSSTSMIDLMAAIIKGEPVIVSDPANASAYESVAAELSSMLIENRQFAMIVTTSIAVLIGCIVMLVWRRSGSGNSKRVEPLKPLVIKPREEEIDDGRKKVTIFFGTQTGTAEGFAKALGEEAKARYEKTRFKIVDLDDYAADDDEYEEKLKKEDVAFFFLATYGDGEPTDNAARFYKWFTEGNDRGEWLKNLKYGVFGLGNRQYEHFNKVAKVVDDILVEQGAQRLVQVGLGDDDQCIEDDFTAWREALWPELDTILREEGDTAVATPYTAAVLEYRVSIHDSEDAKFNDINMANGNGYTVFDAQHPYKANVAVKRELHTPESDRSCIHLEFDIAGSGLTYETGDHVGVLCDNLSETVDEALRLLDMSPDTYFSLHAEKEDGTPISSSLPPPFPPCNLRTALTRYACLLSSPKKSALVALAAHASDPTEAERLKHLASPAGKVDEYSKWVVESQRSLLEVMAEFPSAKPPLGVFFAGVAPRLQPRFYSISSSPKIAETRIHVTCALVYEKMPTGRIHKGVCSTWMKNAVPYEKSENCSSAPIFVRQSNFKLPSDSKVPIIMIGPGTGLAPFRGFLQERLALVESGVELGPSVLFFGCRNRRMDFIYEEELQRFVESGALAELSVAFSREGPTKEYVQHKMMDKASDIWNMISQGAYLYVCGDAKGMARDVHRSLHTIAQEQGSMDSTKAEGFVKNLQTSGRYLRDVW

SEQ ID NO: 32 - DNA, 2139 bp, synthetic construct
  misc_feature 1..2139: note = coding sequence for SEQ ID NO: 31
  source 1..2139: mol_type = other DNA
atgtcctcttcttcttcttcttctacctccatgatcgatttgatggctgctatcatcaagggtgaaccagttattgtctccgacccagctaacgcctccgcttacgaatccgttgctgctgaattgtcctctatgttgatcgaaaacagacaattcgccatgattgttaccacttccattgctgttttgattggttgtatcgttatgttggtttggagaagatccggttctggtaactctaagagagtcgaaccattgaagccattggttattaagccaagagaagaagaaattgatgatggtagaaagaaggttaccatcttcttcggtactcaaactggtactgctgaaggtttcgctaaggctttgggtgaagaagctaaggctagatacgaaaagaccagattcaagatcgttgatttggatgattacgctgctgatgatgatgaatacgaagaaaagttgaagaaggaagatgttgctttcttcttcttggccacttacggtgatggtgaaccaaccgacaacgctgctagattctacaagtggttcaccgaaggtaacgacagaggtgaatggttgaagaacttgaagtacggtgttttcggtttgggtaacagacaatacgaacacttcaacaaggttgccaaggttgttgatgacattttggtcgaacaaggtgctcaaagattggttcaagttggtttgggtgatgatgaccaatgtattgaagatgacttcaccgcttggagagaagctttgtggccagaattggatactatcttgagagaagaaggtgatactgctgttgccactccatacactgctgctgttttggaatacagagtttctattcacgactctgaagatgccaagttcaacgatatcaacatggctaacggtaacggttacactgttttcgatgctcaacacccatacaaggctaacgtcgctgttaagagagaattgcacactccagaatctgatagatcttgtatccacttggaattcgacattgctggttctggtttgacttacgaaactggtgatcacgttggtgttttgtgtgataacttgtctgaaactgttgatgaagctttgagattgttggatatgtctccagatacttacttctctttgcacgctgaaaaggaagatggtactccaatctcttcttctttgccaccaccattcccaccatgtaacttgagaactgctttgactagatacgcttgtttgttgtcatctccaaagaagtctgctttggttgctttggctgctcacgcttctgatccaaccgaagctgaaagattgaagcacttggcttctccagctggtaaggttgatgaatactctaagtgggttgttgaatctcaaagatctttgttggaagttatggccgaattcccatctgccaagccaccattgggtgttttcttcgctggtgttgctccaagattgcaaccaagattctactctatctcttcttctccaaagattgctgaaactagaattcacgtcacttgtgctttggtttacgaaaagatgccaactggtagaattcacaagggtgtttgttccacttggatgaagaacgctgttccatacgaaaagtctgaaaactgttcctctgctccaatcttcgttagacaatccaacttcaagttgccatctgattctaaggttccaatcatcatgatcggtccaggtactggtttggctccattcagaggtttcttgcaagaaagattggctttggttgaatctggtgttgaattgggtccatctgttttgttcttcggttgtagaaacagaagaatggatttcatctacgaagaagaattgcaaagattcgttgaatctggtgctttggctgaattgtctgtcgccttctctagagaaggtccaaccaaggaatacgttcaacacaagatgatggacaaggcttctgatatctggaacatgatctctcaaggtgcttacttgtacgtttgtggtgacgccaagggtatggctagagatgttcacagatctttgcacactatcgctcaagaacaaggttctatggattctactaaggctgaaggtttcgttaagaacttgcaaacttctggtagatacttgagagatgtttggtaa

SEQ ID NO: 33 - protein, 522 aa, Lonicera japonica
MSWIFDLTISFTTLLFLIFTTALLLLLKVFKKNHKLRPPPSPFTLPIIGHLHLLGPLIHQSFHRLSTLYGPLIQLKIGYIPCVVASTPELAKEFLKTHELAFSSRKHSAAIKLLTYDVSFAFSPYGPYWKFIKKTCTFELLGTRNMNHFLPIRTNEIRRFLQVMLEKAKASEGVNVTEELIKLTNNVISQMMFSTRSSGTEGEAEEMRTLVREVTQIFGEFNVSDFIKLCKNIDIGGFKKRSKDIQKRYDALLEKIISERESERARRGKNRETLGEEGGKDFLDMMLDTMEDGKCEVEITRDHIKALVLDFLTAATDTTAIAVEWTLAELISNPEVFDKAREEIDKVVGKHRLVTELDTPNLPYIHAIIKESFRLHPPIPLLIRKSVQDCTVGGYHISANTILFVNIWAIGRNPKYWESPMKFWPERFLESNGPGPVGSMDIKGHHYELLPFGSGRRGCPGMALAMQELPVVLAAMIQCFNWKPVTLDGEELDMSERPGLTAPRAHDLVCVPSARINSFDNF

SEQ ID NO: 34 - DNA, 1569 bp, synthetic construct
  misc_feature 1..1569: note = coding sequence for SEQ ID NO: 33
  source 1..1569: mol_type = other DNA
atgtcctggatcttcgacttgactatctctttcaccactttgttgttcttgatcttcaccaccgccttgttgttgttgttgaaggttttcaagaagaaccacaagttaagaccaccaccatctccattcaccttgccaatcatcggtcacttgcacttgttgggtccattgatccaccaatccttccacagattgtccaccttgtacggtccattgatccaattgaagatcggttacatcccatgtgttgttgcctctactccagaattggctaaggaattcttaaagactcacgaattggctttctcctctagaaagcactccgctgccattaagttgttgacctacgatgtttctttcgctttctctccatacggtccatactggaagttcatcaaaaagacttgtaccttcgaattgttgggtactagaaacatgaaccacttcttgccaattagaaccaacgaaattagaagattcttgcaagttatgttggaaaaggccaaggcttctgaaggtgttaacgttactgaagaattgatcaagttgactaacaacgttatctctcaaatgatgttctctactagatcttctggtaccgaaggtgaagctgaagaaatgagaactttggttagagaagttactcaaatcttcggtgaattcaacgtttctgatttcatcaagttgtgtaagaacattgatattggtggtttcaagaagagatctaaggatatccaaaagagatacgatgctttgttggaaaagatcatctctgaaagagaatctgaaagagctagaagaggtaagaacagagaaactttgggtgaagaaggtggtaaggatttcttggatatgatgttggatactatggaagatggtaagtgtgaagttgaaatcactagagatcacattaaggccttggttttggatttcttgactgctgccactgatactactgctattgctgttgaatggactttggccgaattgatctctaacccagaagttttcgataaggctagagaagaaatcgataaggtcgttggtaagcacagattggtcactgaattggacactccaaacttgccatacatccacgctatcatcaaggaatctttcagattgcacccaccaattccattgttgatcagaaagtctgtccaagattgtactgttggtggttaccacatctctgctaacaccatcttgttcgtcaacatttgggccatcggtagaaacccaaagtactgggaatctccaatgaagttctggccagaaagattcttggaatccaacggtccaggtccagttggttctatggatattaagggtcaccactacgaattgttgccattcggttctggtagaagaggttgtccaggtatggctttggccatgcaagaattgccagttgttttggccgccatgatccaatgtttcaactggaagccagttactttggacggtgaagaattggatatgtctgaaagaccaggtttgactgctccaagagcccacgatttggtttgtgttccatccgctagaattaactctttcgataacttctaa

SEQ ID NO: 35 - protein, 520 aa, Lonicera macranthoides
MSLIFDLTISFTTLLFLIFTTALLLKVFKKNHKLQPPPSPFTLPIIGHLHLLGPLIHQSFHRLSTLYGPLIQLKIGYIPCVVASTPELAKEFLKTHELAFSSRKHSAAIKLLTYDVSFAFAPYGPYWKFIKKTCTFELLGTRNMNHFLPIRTNEIRRFLQVMLEKAKASEGVNVTEELIKLTNNVISQMMFSTRSSGTEGEAEEVRTLVREVTQIFGEFNVSDFIKLCKNIDIGGFKKRSEDIQKRYDALLEKIISERESERARRGKNRETLGEEGGKDFLDMMLDTMEDGKCEVEITRDHIKALVLDFLTAATDTTAIAVEWTLAELISNPEVFDKAREEIDKVVGKHRLVTELDTPNLPYIHAIIKESFRLHPPIPLVIRKSVQDCTVGGYHISANTILFINIWAIGRNPKYWESPMKFWPERFLESNEPGSVGSTDIKGHHYELLPFGSGRRGCPGMALAMQELPVVLAAMIQCFNWKPVTLDGEELDMSERPGLTAPRAHDLVCVPSARINSFDNF

SEQ ID NO: 36 - DNA, 1563 bp, synthetic construct
  misc_feature 1..1563: note = coding sequence for SEQ ID NO: 35
  source 1..1563: mol_type = other DNA
atgtccttgatcttcgacttgactatctctttcaccactttgttgttcttgatcttcaccaccgccttgttgttgaaggttttcaagaagaaccacaagttgcaaccaccaccatctccattcaccttgccaatcatcggtcacttgcacttgttgggtccattgatccaccaatccttccacagattgtccaccttgtacggtccattgatccaattgaagatcggttacatcccatgtgttgttgcttctactccagaattggctaaggaattcttaaagactcacgaattggctttctcctctagaaagcactccgctgccattaagttgttgacctacgatgtttctttcgctttcgctccatacggtccatactggaagttcatcaaaaagacttgtaccttcgaattgttgggtactagaaacatgaaccacttcttgccaattagaaccaacgaaattagaagattcttgcaagttatgttggaaaaggccaaggcttctgaaggtgttaacgttactgaagaattgatcaagttgactaacaacgttatctctcaaatgatgttctctactagatcttctggtaccgaaggtgaagctgaagaagttagaactttggttagagaagttactcaaatcttcggtgaattcaacgtttctgatttcatcaagttgtgtaagaacattgatattggtggtttcaagaagagatctgaagatatccaaaagagatacgatgctttgttggaaaagatcatctctgaaagagaatctgaaagagctagaagaggtaagaacagagaaactttgggtgaagaaggtggtaaggatttcttggacatgatgttggatactatggaagatggtaagtgtgaagttgaaatcactagagatcacattaaggccttggttttggatttcttgactgctgccactgatactactgctattgctgttgaatggactttggctgaattgatctctaacccagaagttttcgataaggctagagaagaaatcgataaggtcgttggtaagcacagattggtcactgaattggacactccaaacttgccatacatccacgctatcatcaaggaatctttcagattgcacccaccaattccattggtcatcagaaagtctgtccaagattgtactgttggtggttaccacatctctgctaacactatcttgttcatcaacatttgggccatcggtagaaacccaaagtactgggaatctccaatgaagttctggccagaaagattcttggaatccaacgaaccaggttctgttggttctactgatattaagggtcaccactacgaattgttgccattcggttctggtagaagaggttgtccaggtatggctttggccatgcaagaattgccagttgttttggccgccatgatccaatgtttcaactggaagccagttactttggacggtgaagaattggatatgtctgaaagaccaggtttgactgctccaagagcccacgatttggtttgtgttccatccgctagaattaactctttcgataacttctaa

SEQ ID NO: 37 - protein, 366 aa, Petroselinum crispum
MSAPTTITALAKEKTLNLDFVRDEDERPKVAYNQFSNEIPIISLAGLDDDSDGRRPEICRKIVKACEDWGIFQVVDHGIDSGLISEMTRLSREFFALPAEEKLEYDTTGGKRGGFTISTVLQGDDAMDWREFVTYFSYPINARDYSRWPKKPEGWRSTTEVYSEKLMVLGAKLLEVLSEAMGLEKGDLTKACVDMEQKVLINYYPTCPQPDLTLGVRRHTDPGTITILLQDMVGGLQATRDGGKTWITVQPVEGAFVVNLGDHGHYLSNGRFRNADHQAVVNSTSSRLSIATFQNPAQNAIVYPLKIREGEKAILDEAITYAEMYKKCMTKHIEVATRKKLAKEKRLQDEKAKLEMKSKSADENLA

SEQ ID NO: 38 - DNA, 1101 bp, synthetic construct
  misc_feature 1..1101: note = coding sequence for SEQ ID NO: 37
  source 1..1101: mol_type = other DNA
atgtccgctccaactactatcaccgctttggccaaggaaaagactttgaacttggacttcgttagagatgaagatgaaagaccaaaggttgcttacaaccaattctctaacgaaattccaattatttctttggccggtttggatgacgattctgatggtagaaggccagaaatctgtagaaagatcgttaaggcttgtgaagattggggtattttccaagttgttgatcacggtattgactctggtttgatttccgaaatgactagattgtctagagaattcttcgctttgccagctgaagaaaagttggaatacgatactactggtggtaagagaggtggtttcactatctccactgttttgcaaggtgacgacgctatggattggagagaattcgttacttacttctcttacccaatcaacgctagagactactctagatggccaaagaagccagaaggttggagatctaccactgaagtttactctgaaaagttgatggttttgggtgccaagttgttggaagttttgtctgaagccatgggtttggaaaagggtgatttgactaaggcttgtgttgatatggaacaaaaggttttgattaactactacccaacttgtccacaaccagacttgactttgggtgtcaggagacacactgatccaggtactattaccattttgttgcaagacatggttggtggtttgcaagccaccagagatggtggtaagacttggattactgttcaaccagttgaaggtgctttcgttgtcaacttgggtgatcacggtcactacttgtctaacggtagattcagaaacgctgaccaccaagctgttgttaactctacctcttctagattgtctattgctactttccaaaacccagctcaaaacgctatcgtttacccattgaagatcagagaaggtgaaaaggctattttggatgaagccatcacctacgctgaaatgtacaagaagtgtatgactaagcacattgaagttgctactagaaagaagttggccaaggaaaagagattgcaagacgaaaaggccaagttggaaatgaagtccaagtctgctgatgaaaacttggcttaa

SEQ ID NO: 39 - protein, 507 aa, Flavobacterium johnsoniae
MSNTINEYLSLEEFEAIIFGNQKVTISDVVVNRVNESFNFLKEFSGNKVIYGVNTGFGPMAQYRIKESDQIQLQYNLIRSHSSGTGKPLSPVCAKAAILARLNTLSLGNSGVHPSVINLMSELINKDITPLIFEHGGVGASGDLVQLSHLALVLIGEGEVFYKGERRPTPEVFEIEGLKPIQVEIREGLALINGTSVMTGIGVVNVYHAKKLLDWSLKSSCAINELVQAYDDHFSAELNQTKRHKGQQEIALKMRQNLSDSTLIRKREDHLYSGENTEEIFKEKVQEYYSLRCVPQILGPVLETINNVASILEDEFNSANDNPIIDVKNQHVYHGGNFHGDYISLEMDKLKIVITKLTMLAERQLNYLLNSKINELLPPFVNLGTLGFNFGMQGVQFTATSTTAESQMLSNPMYVHSIPNNNDNQDIVSMGTNSAVITSKVIENAFEVLAIEMITIVQAIDYLGQKDKISSVSKKWYDEIRNIIPTFKEDQVMYPFVQKVKDHLINN

SEQ ID NO: 40 - DNA, 1524 bp, synthetic construct
  misc_feature 1..1524: note = coding sequence for SEQ ID NO: 39
  source 1..1524: mol_type = other DNA
atgtccaacactattaacgaatacttgtctttggaagaattcgaagccatcatcttcggtaaccaaaaggttactatttctgacgttgtcgttaacagagttaacgaatctttcaacttcttgaaggaattctctggtaacaaggttatttacggtgttaacactggtttcggtccaatggctcaatacagaattaaggaatctgatcaaattcaattgcaatacaacttgattagatctcactcttctggtaccggtaagccattgtctccagtttgtgctaaggctgctattttggctagattgaacactttgtctttgggtaactctggtgttcacccatctgttatcaacttgatgtctgaattgatcaacaaggatattaccccattgatcttcgaacacggtggtgttggtgcctctggtgacttggttcaattgtctcacttggctttggttttgattggtgaaggtgaagttttctacaagggtgaaagaaggccaactccagaagttttcgaaattgaaggtttgaagccaattcaagttgaaattagagaaggtttggctttgattaacggtacttctgttatgactggtattggtgttgttaacgtttaccacgctaagaagttgttggactggtctttgaaatcttcttgtgctattaacgaattggttcaagcttacgacgatcacttctctgctgaattgaaccaaactaagagacacaagggtcaacaagaaattgctttgaagatgagacaaaacttgtctgactctactttgatcagaaagagagaagatcacttgtactctggtgaaaacaccgaagaaatcttcaaggaaaaggttcaagaatactactctttgagatgtgttccacaaattttgggtccagttttggaaactattaacaacgttgcttctattttggaagatgaattcaactctgctaacgataacccaattatcgatgttaagaaccaacacgtttaccacggtggtaacttccacggtgattacatttctttggaaatggataagttgaagattgttattaccaagttgactatgttggctgaaagacaattgaactacttgttgaactctaagatcaacgaattgttgccaccattcgttaacttgggtactttgggtttcaacttcggtatgcaaggtgttcaattcactgctacttctactactgctgaatctcaaatgttgtctaacccaatgtacgttcactctattccaaacaacaacgacaaccaagatatcgtttctatgggtactaactctgctgttattacctctaaggttatcgaaaacgctttcgaagttttggctatcgaaatgattactatcgttcaagctattgattacttgggtcaaaaggacaagatttcttctgtttctaagaagtggtacgatgaaattagaaacatcattccaactttcaaggaagatcaagttatgtacccattcgttcaaaaggttaaggatcacttgatcaacaactaa

SEQ ID NO: 41 - protein, 694 aa, Rhodotorula glutinis
MSAPRPTSQSQARTCPTTQVTQVDIVEKMLAAPTDSTLELDGYSLNLGDVVSAARKGRPVRVKDSDEIRSKIDKSVEFLRSQLSMSVYGVTTGFGGSADTRTEDAISLQKALLEHQLCGVLPSSFDSFRLGRGLENSLPLEVVRGAMTIRVNSLTRGHSAVRLVVLEALTNFLNHGITPIVPLRGTISASGDLSPLSYIAAAISGHPDSKVHVVHEGKEKILYAREAMALFNLEPVVLGPKEGLGLVNGTAVSASMATLALHDAHMLSLLSQSLTAMTVEAMVGHAGSFHPFLHDVTRPHPTQIEVAGNIRKLLEGSRFAVHHEEEVKVKDDEGILRQDRYPLRTSPQWLGPLVSDLIHAHAVLTIEAGQSTTDNPLIDVENKTSHHGGNFQAAAVANTMEKTRLGLAQIGKLNFTQLTEMLNAGMNRGLPSCLAAEDPSLSYHCKGLDIAAAAYTSELGHLANPVTTHVQPAEMANQAVNSLALISARRTTESNDVLSLLLATHLYCVLQAIDLRAIEFEFKKQFGPAIVSLIDQHFGSAMTGSNLRDELVEKVNKTLAKRLEQTNSYDLVPRWHDAFSFAAGTVVEVLSSTSLSLAAVNAWKVAAAESAISLTRQVRETFWSAASTSSPALSYLSPRTQILYAFVREELGVKARRGDVFLGKQEVTIGSNVSKIYEAIKSGRINNVLLKMLA

SEQ ID NO: 42 - DNA, 2085 bp, synthetic construct
  misc_feature 1..2085: note = coding sequence for SEQ ID NO: 41
  source 1..2085: mol_type = other DNA
atgtccgctccaagaccaacttctcaatctcaagccagaacttgtccaaccacccaagttacccaagttgatatcgttgaaaagatgttggctgctccaactgattctaccttggaattggacggttactctttgaacttgggtgatgttgtttctgctgctagaaagggtagaccagttagagttaaggattctgatgaaatcagatctaagatcgacaagtctgttgaattcttgagatctcaattgtctatgtctgtttacggtgttaccaccggtttcggtggttccgctgacaccagaaccgaagatgctatttctttgcaaaaggctttgttggaacaccaattgtgtggtgttttgccatcttctttcgactctttcagattgggtagaggtttggaaaactctttgccattggaagttgttagaggtgctatgaccattagagttaactctttgaccagaggtcactctgctgttagattggttgttttggaagctttgaccaacttcttgaaccacggtattaccccaattgttccattgagaggtaccatctccgcttctggtgatttgtctccattgtcttacattgctgctgctatttctggtcacccagattctaaggttcacgttgttcacgaaggtaaggaaaagatcttgtacgctagagaagctatggctttgttcaacttggaaccagttgttttgggtccaaaggaaggtttgggtttggttaacggtaccgctgtttccgcttctatggctaccttggctttgcacgacgctcacatgttgtctttgttgtctcaatctttgaccgctatgaccgttgaagctatggttggtcacgctggttctttccacccattcttgcacgatgttaccagaccacacccaacccaaatcgaagttgctggtaacattagaaagttgttggaaggttctagattcgctgttcaccacgaagaagaagttaaggttaaggatgatgaaggtattttgagacaagatagatacccattgagaacctctccacaatggttgggtccattggtttccgacttgattcacgctcacgccgttttgaccatcgaagctggtcaatctaccaccgataacccattgatcgatgttgaaaacaagacctctcaccacggtggtaacttccaagctgctgctgttgccaacactatggaaaagaccagattgggtttggcccaaatcggtaagttgaacttcacccaattgaccgaaatgttgaacgctggtatgaacagaggtttgccatcttgtttggctgctgaagatccatccttgtcttaccactgtaagggtttggacattgctgctgctgcttacacctctgaattgggtcacttggctaacccagttaccacccacgttcaaccagctgaaatggctaaccaagctgttaactctttggctttgatttctgctagaagaaccaccgaatctaacgacgttttgtccttgttgttggctacccacttgtactgtgttttgcaagctatcgacttgagagctattgaattcgaattcaagaagcaattcggtccagccattgtttctttgatcgaccaacacttcggttctgctatgaccggttctaacttgagagatgaattggttgaaaaggttaacaagactttggccaagagattggaacaaaccaactcttacgatttggttccaagatggcacgacgctttctctttcgctgctggtactgttgttgaagttttgtcctctacctctttgtctttggctgccgttaacgcttggaaggttgctgctgccgaatctgctatctccttgaccagacaagttagagaaaccttctggtccgctgcttctacctcctctccagctttgtcttacttgtctccaagaacccaaatcttgtacgctttcgttagagaagaattgggtgttaaggccagaagaggtgacgttttcttgggtaagcaagaagttaccatcggttctaacgtttctaagatttacgaagccatcaagtctggtagaatcaacaacgttttgttgaagatgttggcttaa

SEQ ID NO: 43 - protein, 562 aa, Arabidopsis thaliana
MSAPQEQAVSQVMEKQSNNNNSDVIFRSKLPDIYIPNHLSLHDYIFQNISEFATKPCLINGPTGHVYTYSDVHVISRQIAANFHKLGVNQNDVVMLLLPNCPEFVLSFLAASFRGATATAANPFFTPAEIAKQAKASNTKLIITEARYVDKIKPLQNDDGVVIVCIDDNESVPIPEGCLRFTELTQSTTEASEVIDSVEISPDDVVALPYSSGTTGLPKGVMLTHKGLVTSVAQQVDGENPNLYFHSDDVILCVLPMFHIYALNSIMLCGLRVGAAILIMPKFEINLLLELIQRCKVTVAPMVPPIVLAIAKSSETEKYDLSSIRVVKSGAAPLGKELEDAVNAKFPNAKLGQGYGMTEAGPVLAMSLGFAKEPFPVKSGACGTVVRNAEMKIVDPDTGDSLSRNQPGEICIRGHQIMKGYLNNPAATAETIDKDGWLHTGDIGLIDDDDELFIVDRLKELIKYKGFQVAPAELEALLIGHPDITDVAVVAMKEEAAGEVPVAFVVKSKDSELSEDDVKQFVSKQVVFYKRINKVFFTESIPKAPSGKILRKDLRAKLANGL

SEQ ID NO: 44 - DNA, 1689 bp, synthetic construct
  misc_feature 1..1689: note = coding sequence for SEQ ID NO: 43
  source 1..1689: mol_type = other DNA
atgtccgctccacaagaacaagctgtttctcaagttatggaaaagcaatctaacaacaacaactctgacgtcattttcagatctaagttgccagatatttacatcccaaaccacttgtctttgcacgactacatcttccaaaacatctccgaattcgccactaagccatgtttgatcaacggtccaaccggtcacgtttacacttactccgacgtccacgtcatctccagacaaatcgccgccaacttccacaagttgggtgttaaccaaaacgacgtcgtcatgttgttgttgccaaactgtccagaattcgtcttgtctttcttggccgcctccttcagaggtgctaccgccaccgccgctaacccattcttcactccagctgaaatcgctaagcaagccaaggcctccaacaccaagttgatcatcaccgaagctagatacgtcgacaagatcaagccattgcaaaacgacgacggtgttgtcatcgtctgtatcgacgacaacgaatccgttccaatcccagaaggttgtttgagattcaccgaattgactcaatctactaccgaagcttctgaagtcatcgactctgttgaaatttctccagacgacgttgttgctttgccatactcctctggtactactggtttgccaaagggtgttatgttgactcacaagggtttggtcacttctgttgctcaacaagtcgacggtgaaaacccaaacttgtacttccactctgatgacgtcatcttgtgtgttttgccaatgttccacatctacgctttgaactctatcatgttgtgtggtttgagagttggtgctgctattttgatcatgccaaagttcgaaatcaacttgttgttggaattgatccaaagatgtaaggttactgttgctccaatggttccaccaattgttttggccattgctaaatcttctgaaactgaaaagtacgatttatcttctatcagagttgttaagtctggtgctgctccattgggtaaggaattggaagatgccgttaacgccaagttcccaaacgccaagttgggtcaaggttacggtatgactgaagctggtccagttttggctatgtctttgggtttcgctaaggaaccattcccagttaagtctggtgcttgtggtactgttgttagaaacgctgaaatgaagatcgttgatccagacaccggtgattctttgtctagaaaccaaccaggtgaaatttgtattagaggtcaccaaatcatgaagggttacttgaacaacccagctgctactgctgaaaccattgataaggacggttggttgcacactggtgatattggtttgatcgatgacgatgacgaattgttcatcgttgatagattgaaggaattgatcaagtacaagggtttccaagttgctccagctgaattggaagctttgttgatcggtcacccagacattactgatgttgctgttgtcgctatgaaggaagaagctgctggtgaagttccagttgctttcgttgttaagtctaaggattctgaattgtctgaagatgatgttaagcaattcgtttctaagcaagttgttttctacaagagaatcaacaaggttttcttcactgaatccattccaaaggctccatctggtaagatcttgagaaaggatttgagagctaagttggctaacggtttgtaa

SEQ ID NO: 45 - protein, 545 aa, Petroselinum crispum
MSGDCVAPKEDLIFRSKLPDIYIPKHLPLHTYCFENISKVGDKSCLINGATGETFTYSQVELLSRKVASGLNKLGIQQGDTIMLLLPNSPEYFFAFLGASYRGAISTMANPFFTSAEVIKQLKASQAKLIITQACYVDKVKDYAAEKNIQIICIDDAPQDCLHFSKLMEADESEMPEVVINSDDVVALPYSSGTTGLPKGVMLTHKGLVTSVAQQVDGDNPNLYMHSEDVMICILPLFHIYSLNAVLCCGLRAGVTILIMQKFDIVPFLELIQKYKVTIGPFVPPIVLAIAKSPVVDKYDLSSVRTVMSGAAPLGKELEDAVRAKFPNAKLGQGYGMTEAGPVLAMCLAFAKEPYEIKSGACGTVVRNAEMKIVDPETNASLPRNQRGEICIRGDQIMKGYLNDPESTRTTIDEEGWLHTGDIGFIDDDDELFIVDRLKEIIKYKGFQVAPAELEALLLTHPTISDAAVVPMIDEKAGEVPVAFVVRTNGFTTTEEEIKQFVSKQVVFYKRIFRVFFVDAIPKSPSGKILRKDLRARIASGDLPK

SEQ ID NO: 46 - DNA, 1638 bp, synthetic construct
  misc_feature 1..1638: note = coding sequence for SEQ ID NO: 45
  source 1..1638: mol_type = other DNA
atgtccggtgattgtgttgctccaaaggaagatttgattttcagatctaagttgccagatatttacatcccaaagcacttgccattgcacacttactgtttcgaaaacatctctaaggttggtgacaagtcctgtttgatcaacggtgctactggtgaaactttcacttactctcaagttgaattgttgtccagaaaggttgcttctggtttgaacaagttgggtattcaacaaggtgataccatcatgttgttgttgccaaactccccagaatacttcttcgctttcttgggtgcttcttacagaggtgctatttctactatggccaacccattcttcacttctgctgaagttatcaagcaattgaaggcttcccaagctaagttgatcattactcaagcttgttacgttgacaaggttaaggactacgctgctgaaaagaacatccaaatcatttgtatcgatgatgctccacaagattgtttgcacttctccaagttgatggaagctgatgaatctgaaatgccagaagttgttatcaactctgacgatgtcgtcgctttgccatactcttctggtactactggtttgccaaagggtgttatgttgactcacaagggtttggttacttctgttgctcaacaagttgatggtgacaacccaaacttgtacatgcactctgaagatgttatgatctgtatcttgccattgttccacatttactctttgaacgctgttttgtgttgtggtttgagagctggtgttactatcttgattatgcaaaagttcgatattgttccattcttggaattgatccaaaagtacaaggttactattggtccattcgttccaccaattgttttggctattgctaagtctccagttgttgataagtacgacttatcttctgttagaactgttatgtctggtgctgctccattgggtaaggaattggaagatgctgttagagctaagttcccaaacgccaagttgggtcaaggttacggtatgactgaagctggtccagttttggctatgtgtttggctttcgctaaggaaccatacgaaatcaagtctggtgcctgtggtactgttgttagaaacgctgaaatgaagattgttgatccagaaaccaacgcctctttgccaagaaaccaaagaggtgaaatttgtattagaggtgaccaaattatgaagggttacttgaacgatccagaatctactagaactactatcgacgaagaaggttggttgcacactggtgatatcggtttcattgacgacgatgatgaattgttcattgttgatagattgaaggaaatcatcaagtacaagggtttccaagttgccccagctgaattggaagctttgttgttgactcacccaaccatttccgatgctgctgttgttccaatgatcgatgaaaaggctggtgaagttccagttgctttcgttgttagaactaacggtttcaccaccactgaagaagaaatcaagcaattcgtttctaagcaagttgttttctacaagagaatcttcagagttttcttcgttgatgctattccaaagtctccatctggtaagattttgagaaaggacttgagagctagaatcgcttccggtgatttgccaaagtaa

SEQ ID NO: 47 - protein, 545 aa, Petroselinum crispum
MSGDCVAPKEDLIFRSKLPDIYIPKHLPLHTYCFENISKVGDKSCLINGATGETFTYSQVELLSRKVASGLNKLGIQQGDTIMLLLPNSPEYFFAFLGASYRGAISTMANPFFTSAEVIKQLKASLAKLIITQACYVDKVKDYAAEKNIQIICIDDAPQDCLHFSKLMEADESEMPEVVIDSDDVVALPYSSGTTGLPKGVMLTHKGLVTSVAQQVDGDNPNLYMHSEDVMICILPLFHIYSLNAVLCCGLRAGVTILIMQKFDIVPFLELIQKYKVTIGPFVPPIVLAIAKSPVVDKYDLSSVRTVMSGAAPLGKELEDAVRAKFPNAKLGQGYGMTEAGPVLAMCLAFAKEPYEIKSGACGTVVRNAEMKIVDPETNASLPRNQRGEICIRGDQIMKGYLNDPESTRTTIDEEGWLHTGDIGFIDDDDELFIVDRLKEIIKYKGFQVAPAELEALLLTHPTISDAAVVPMIDEKAGEVPVAFVVRTNGFTTTEEEIKQFVSKQVVFYKRIFRVFFVDAIPKSPSGKILRKDLRAKIASGDLPK

SEQ ID NO: 48 - DNA, 1638 bp, synthetic construct
  misc_feature 1..1638: note = coding sequence for SEQ ID NO: 47
  source 1..1638: mol_type = other DNA
atgtccggtgactgtgttgctccaaaggaagatttgattttcagatctaagttgccagatatttacatcccaaagcacttgccattgcacacttactgtttcgaaaacatctctaaggttggtgacaagtcctgtttgatcaacggtgctactggtgaaactttcacttactctcaagttgaattgttgtccagaaaggttgcttctggtttgaacaagttgggtattcaacaaggtgataccatcatgttgttgttgccaaactccccagaatacttcttcgctttcttgggtgcttcttacagaggtgctatttctactatggccaacccattcttcacttctgctgaagttatcaagcaattgaaggcttccttggctaagttgatcattactcaagcttgttacgttgacaaggttaaggactacgctgctgaaaagaacatccaaatcatttgtatcgatgatgctccacaagattgtttgcacttctccaagttgatggaagctgatgaatctgaaatgccagaagttgttatcgattctgacgatgtcgtcgctttgccatactcttctggtactactggtttgccaaagggtgttatgttgacccacaagggtttggttacttctgttgctcaacaagttgatggtgacaacccaaacttgtacatgcactctgaagatgttatgatctgtatcttgccattgttccacatttactctttgaacgctgttttgtgttgtggtttgagagctggtgttactatcttgattatgcaaaagttcgatattgttccattcttggaattgatccaaaagtacaaggttactattggtccattcgttccaccaattgttttggctattgctaagtctccagttgttgataagtacgacttatcttctgttagaactgttatgtctggtgctgctccattgggtaaggaattggaagatgctgttagagctaagttcccaaacgccaagttgggtcaaggttacggtatgactgaagctggtccagttttggctatgtgtttggctttcgctaaggaaccatacgaaatcaagtctggtgcctgtggtactgttgttagaaacgctgaaatgaagattgttgatccagaaaccaacgcctctttgccaagaaaccaaagaggtgaaatttgtattagaggtgaccaaattatgaagggttacttgaacgatccagaatctactagaactactatcgacgaagaaggttggttgcacactggtgatatcggtttcattgacgacgatgatgaattgttcattgttgatagattgaaggaaatcatcaagtacaagggtttccaagttgccccagctgaattggaagctttgttgttgactcacccaaccatttccgatgctgctgttgttccaatgatcgatgaaaaggctggtgaagttccagttgctttcgttgttagaactaacggtttcaccaccactgaagaagaaatcaagcaattcgtttctaagcaagttgttttctacaagagaatcttcagagttttcttcgttgatgctattccaaagtctccatctggtaagattttgagaaaggacttgagagctaagatcgcttccggtgatttgccaaagtaa

SEQ ID NO: 49 - protein, 579 aa, Streptomyces clavuligerus
MSASTPSPGPSGTPSGTPPSGPSGTPSPGPAGAGTAPVFRSRYPDIEPVSEPLHEAVLGRAAGYGSEPALVDGLTGAVVSYARLDRDHRRIAAALAAAGVRKGDVVALHSPNSTGYPAVLYGALRAGATVTTAHPLATAEELARQLRDSAARWIVTAAPCLETARRAAELTPGIGEIFVFDRAEGHTGVAAMLDSTAPEPAVPVDPDQDVALLPYSSGTTGTPKGVMLTHRSLVTNLVQAHRLIPLRPGDRVLAVLPFFHIYGLVGLMSAPLRNGATVVVLPRFDLEGFLAAVEKHRVTTLYVAPPIVLALAKHPAVARYDLSSVRHVFSAAAPLDAEIAAACAARVGVPLVRQAYGMTELSPGCYAVPLDEPAPPPGTVGLLFPSTEMRLLRLDDPGRCVGPGEDGEIAIRGPQVMKGYLGRPEATAEMIDADGWLRTGDVGRVDADGWLHVVDRVKELIKYKGFQVAPAELEALLLTHGGIADAAVIGVYDEDEGTEIPHAFVVRRPGGAGDSLTAADVAAHVAARVSPYKKVRRVSFVSGVPRAASGKILRRELRAARRSPRGTDPGDEDREGATP

SEQ ID NO: 50 - DNA, 1740 bp, synthetic construct
  misc_feature 1..1740: note = coding sequence for SEQ ID NO: 49
  source 1..1740: mol_type = other DNA
atgtccgcttctactccatctccaggtccatctggtaccccatccggtactccaccatccggtccatctggtactccatccccaggtccagctggtgccggtaccgctccagtattcagatctagatacccagacatcgaaccagtttctgaaccattgcacgaagctgttttgggtagagccgccggttacggttccgaaccagccttggtcgacggtttgaccggtgccgtcgtatcttacgctagattggacagagatcacagaagaatcgccgccgccttggccgccgctggtgtcagaaagggtgacgtcgtcgccttgcactctccaaactctaccggttacccagccgtcttgtacggtgccttgagagccggtgccaccgttaccactgctcacccattggctactgctgaagaattggccagacaattgagagactctgccgccagatggatcgttaccgccgccccatgtttggaaaccgccagaagagccgccgaattgaccccaggtatcggtgaaatcttcgtattcgacagagccgaaggtcacaccggtgttgccgctatgttggactccaccgctccagaaccagccgtcccagtcgacccagaccaagacgtcgctttgttgccatactcctctggtaccaccggtaccccaaagggtgttatgttgactcacagatccttggtcaccaacttggtccaagcccacagattgatcccattgaggccaggtgacagagtcttggccgttttgccattcttccacatctacggtttggtcggtttgatgtctgccccattgagaaacggtgctaccgtcgtcgtcttgccaagattcgacttggaaggtttcttggctgccgtcgaaaagcacagagtcaccactttgtacgttgccccaccaatcgttttggctttggccaagcacccagctgttgccagatacgacttgtcctctgtcagacacgtattctctgccgccgccccattggacgctgaaatcgctgctgcctgtgccgccagagtcggtgttccattggtcagacaagcttacggtatgaccgaattgtctccaggttgttacgccgtcccattggacgaaccagccccaccaccaggtactgtcggtttgttgttcccatccaccgaaatgagattgttgagattggacgacccaggtagatgtgtcggtccaggtgaggacggtgaaatcgccatcagaggtccacaagtcatgaagggttacttgggtagaccagaagccaccgccgaaatgatcgacgctgatggttggttgagaaccggtgacgtcggtagagtcgacgctgacggttggttgcacgtcgttgacagagttaaggaattgatcaagtacaagggtttccaagtcgctccagccgaattggaagctttgttgttgacccacggtggtatcgctgacgccgccgttatcggtgtttacgacgaggacgaaggtactgaaatcccacacgctttcgttgtcagaaggccaggtggtgctggtgactccttgaccgccgccgacgtcgccgcccacgtcgccgctagagtctccccatacaagaaggtcagaagagtctctttcgtatccggtgttccaagagccgcttctggtaagatcttgagaagagaattgagagccgctagaagatccccaagaggtactgacccaggtgacgaggacagagaaggtgccactccataa

SEQ ID NO: 51 - protein, 400 aa, Hordeum vulgare
MSAAVRLKEVRMAQRAEGLATVLAIGTAVPANCVYQATYPDYYFRVTKSEHLADLKEKFQRMCDKSMIRKRHMHLTEEILIKNPKICAHMETSLDARHAIALVEVPKLGQGAAEKAIKEWGQPLSKITHLVFCTTSGVDMPGADYQLTKLLGLSPTVKRLMMYQQGCFGGATVLRLAKDIAENNRGARVLVVCSEITAMAFRGPCKSHLDSLVGHALFGDGAAAAIIGADPDQLDEQPVFQLVSASQTILPESEGAIDGHLTEAGLTIHLLKDVPGLISENIEQALEDAFEPLGIHNWNSIFWIAHPGGPAILDRVEDRVGLDKKRMRASREVLSEYGNMSSASVLFVLDVMRKSSAKDGLATTGEGKDWGVLFGFGPGLTVETLVLHSVPVPVPTAASA

SEQ ID NO: 52 - DNA, 1203 bp, synthetic construct
  misc_feature 1..1203: note = coding sequence for SEQ ID NO: 51
  source 1..1203: mol_type = other DNA
atgtccgctgctgttagattgaaggaagttagaatggctcaaagagctgaaggtttggctactgttttggccattggtaccgccgttccagccaactgtgtttaccaagctacctacccagactactacttcagagtcactaagtctgaacacttggctgacttgaaggaaaagttccaaagaatgtgtgacaagtccatgatcagaaagagacacatgcacttgaccgaagaaatcttgattaagaacccaaagatctgtgctcacatggaaacctctttggacgctagacacgccattgctttggtcgaagtcccaaagttgggtcaaggtgctgctgaaaaggctatcaaggaatggggtcaaccattgtccaagatcacccacttggttttctgtactacctctggtgtcgacatgccaggtgccgactaccaattgaccaagttgttgggtttgtccccaactgttaagagattgatgatgtaccaacaaggttgtttcggtggtgccactgttttgagattggccaaggacatcgctgaaaacaacagaggtgctagagttttggttgtctgttctgaaatcaccgccatggccttcagaggtccatgtaagtcccacttggactctttggtcggtcacgctttgttcggtgatggtgctgctgctgccatcatcggtgctgacccagaccaattggacgaacaaccagttttccaattggtttctgcttctcaaaccatcttgccagaatctgaaggtgccatcgacggtcacttgactgaagctggtttgaccatccacttgttgaaggacgttccaggtttgatctctgaaaacatcgaacaagctttggaagatgctttcgaaccattgggtatccacaactggaactccatcttctggattgctcacccaggtggtccagccatcttggacagagtcgaagatagagttggtttggacaagaagagaatgagagcttctagagaagttttgtctgaatacggtaacatgtcctctgcttctgttttgttcgtcttggacgttatgagaaagtcatccgccaaggatggtttggccactactggtgaaggtaaggactggggtgtcttgttcggtttcggtccaggtttgaccgtcgaaactttggtcttgcactctgtcccagtcccagtcccaaccgccgcttctgcttaa

SEQ ID NO: 53 - protein, 392 aa, Citrus sinensis
MSATVQEIRNAQRADGPATVLAIGTATPAHSVNQADYPDYYFRITKSEHMTELKEKFKRMCDKSMIKKRYMYLTEEILKENPNMCAYMAPSLDARQDIVVVEVPKLGKEAATKAIKEWGQPKSKITHLIFCTTSGVDMPGADYQLTKLIGLRPSVKRFMMYQQGCFAGGTVLRLAKDLAENNKGARVLVVCSEITAVTFRGPADTHLDSLVGQALFGDGAAAVIVGADPDTSVERPLYQLVSTSQTILPDSDGAIDGHLREVGLTFHLLKDVPGLISKNIEKSLSEAFAPLGISDWNSIFWIAHPGGPAILDQVESKLGLKGEKLKATRQVLSEYGNMSSACVLFILDEMRKKSVEEAKATTGEGLDWGVLFGFGPGLTVETVVLHSVPIKA

SEQ ID NO: 54 - DNA, 1179 bp, synthetic construct
  misc_feature 1..1179: note = coding sequence for SEQ ID NO: 53
  source 1..1179: mol_type = other DNA
atgtccgctaccgttcaagaaatcagaaacgctcaaagagccgacggtccagccaccgtcttggccatcggtactgccactccagcccactctgtcaaccaagctgattacccagactactacttcagaatcactaagtctgaacacatgactgaattgaaggaaaagttcaagagaatgtgtgacaagtctatgattaagaagagatacatgtacttgactgaagaaattttgaaggaaaacccaaacatgtgtgcttacatggctccatctttggacgctagacaagacattgttgttgtcgaagttccaaagttgggtaaggaagctgctactaaggccatcaaggaatggggtcaaccaaagtctaagatcacccacttgatcttctgtaccacctccggtgtcgacatgccaggtgccgactaccaattgaccaagttgatcggtttaagaccatccgtcaagagattcatgatgtaccaacaaggttgtttcgccggtggtactgttttgagattggctaaggacttggctgaaaacaacaagggtgctagagttttggttgtctgttctgaaatcactgctgtcactttcagaggtccagccgatactcacttggattctttggttggtcaagctttgttcggtgatggtgctgctgctgttatcgttggtgccgatccagacacttctgtcgaaagaccattgtaccaattggtttctacttctcaaactatcttgccagactctgacggtgctattgacggtcacttgagagaagtcggtttgactttccacttgttgaaggacgtcccaggtttgatctctaagaacatcgaaaagtctttgtctgaagctttcgccccattgggtatctctgactggaactctatcttctggatcgctcacccaggtggtccagctattttggaccaagttgaatctaagttgggtttgaagggtgaaaagttgaaggccactagacaagttttgtctgaatacggtaacatgtcatctgcttgtgtcttgttcatcttggacgaaatgagaaagaagtctgttgaagaagctaaggccaccaccggtgaaggtttggattggggtgttttgttcggtttcggtccaggtttgaccgtcgaaaccgttgttttgcactctgtcccaatcaaggcttaa

SEQ ID NO: 55 - protein, 391 aa, Citrus sinensis
MSLTVDEVRKAQRAEGPATIMAIGTATPPNCVDQSTYPDYYFRITNSEHMTDLKEKFKRMCDKSMIKKRYMYLTEEILKENPNVCAYMAPSLDTRQDMVVVEVPRLGKEAATKAIKEWGQPKSKITHLVFCTTSGVDMPGADYRLTKLLGLRPSVKRLMMYQQGCFAGGTVLRLAKDLAENNKGARVLVVCSEITAVTFRGPSDTHLDSLVGQALFGDGAAAIIIGADPIPEIEKPMFELVSTAQTILPDSDGSIDGHLREVGLTFHLLKDVPGLISKNIQKSLTEAFKPLGISDWNSIFWIAHPGGPTILDQVEEKLGLKPEKLRATRHVLSEYGNMSSACVLFILDEMRKKSAEDGLETAGEGLEWGVLFGFGPGLTVETVVLHSVAAA

SEQ ID NO: 56 - DNA, 1176 bp, synthetic construct
  misc_feature 1..1176: note = coding sequence for SEQ ID NO: 55
  source 1..1176: mol_type = other DNA
atgtccttgaccgtcgatgaagttagaaaggctcaaagagccgaaggtccagccactatcatggccattggtactgctaccccaccaaactgtgttgatcaatctacttacccagattactacttcagaatcactaactctgaacacatgactgatttgaaggaaaagttcaagagaatgtgtgacaagtccatgatcaagaagagatacatgtacttgactgaagaaatcttgaaggaaaacccaaacgtttgtgcttacatggctccatctttggatactagacaagatatggttgttgttgaagttccaagattgggtaaggaagctgccaccaaggccattaaggaatggggtcaaccaaagtccaagatcacccacttggtattctgtaccacttctggtgttgacatgccaggtgccgattacagattgactaagttgttgggtttaagaccatccgtcaagagattgatgatgtaccaacaaggttgtttcgccggtggtactgttttgagattggccaaggacttggccgaaaacaacaagggtgctagagtcttggttgtttgttccgaaatcaccgctgttactttcagaggtccatctgacacccacttggattctttggttggtcaagccttgttcggtgatggtgctgctgctattatcattggtgccgacccaatcccagaaatcgaaaagccaatgttcgaattggtatctactgcccaaactatcttgccagattctgatggttctatcgacggtcacttgagagaagttggtttgaccttccacttgttgaaggatgttccaggtttgatttctaagaacatccaaaagtctttgaccgaagctttcaagccattgggtatctctgactggaactctattttctggatcgctcacccaggtggtccaactattttggaccaagtcgaagaaaagttgggtttgaagccagaaaagttgagagccactagacacgttttgtctgaatacggtaacatgtcatctgcttgtgttttgttcattttggacgaaatgagaaagaagtctgctgaagatggtttggaaaccgctggtgaaggtttggaatggggtgtcttgttcggtttcggtccaggtttgactgttgaaaccgttgtcttgcactctgttgccgctgcttaa

SEQ ID NO: 57 - protein, 352 aa, Streptomyces clavuligerus
MSAVLCKPAIAVPDHIITNEETLELARRLHSDHPQLALACRLIEHTGVRKRHLIQPIDEVLKHPGLDARSATYETESKARVPSVVRRALDQAELEPDQIDLIIYVSCTGFMMPSLASWLVNTMGFRADTRQLPIAQLGCAAGGAAVNRAHDFCTAYPGTNVLIVACEFCSLCYQPTDLGIGSLLSNGLFGDGIAAAVVRGEEGTGMRLERNGTYLIPHTEEWISYAVRSTGFHFQLDKRVPGTMEPLSPALRALAEQHQWNAGKLDFYIIHAGGPRILDDLSRFLDVPPGAFRHSRATLTEYGNIASAVVLDALGRLFDEQSALDGHHGMLAGFGPGIIAEMSLGTWVSPES

SEQ ID NO: 58 - DNA, 1059 bp, synthetic construct
  misc_feature 1..1059: note = coding sequence for SEQ ID NO: 57
  source 1..1059: mol_type = other DNA
atgtccgctgttttgtgtaagccagccatcgccgttccagaccacatcatcaccaacgaagaaaccttggaattggctagaagattgcactctgaccacccacaattggctttggcttgtagattgatcgaacacactggtgttagaaagagacacttgatccaaccaatcgacgaagttttgaagcacccaggtttggacgctagatctgccacctacgaaaccgaatccaaggctagagttccatctgttgttagaagagctttggaccaagctgaattggaaccagatcaaatcgacttgatcatctacgtatcctgtaccggtttcatgatgccatccttggcctcctggttggtcaacaccatgggtttcagagctgacaccagacaattgccaatcgcccaattgggttgtgctgctggtggtgctgccgtcaacagagcccacgacttctgtaccgcctacccaggtaccaacgtcttgatcgttgcctgtgaattctgttctttgtgttaccaaccaaccgatttgggtatcggttctttgttgtccaacggtttgttcggtgacggtatcgccgccgctgttgtcagaggtgaagaaggtaccggtatgagattggaaagaaacggtacctacttgatcccacacaccgaagaatggatctcctacgctgttagatccaccggtttccacttccaattggacaagagagtcccaggtaccatggaaccattgtctccagctttgagagccttggccgaacaacaccaatggaacgccggtaagttggacttctacatcatccacgctggtggtccaagaatcttggacgatttgtctagattcttggacgttccaccaggtgccttcagacactctagagccactttgaccgaatacggtaacatcgcctctgccgtcgttttggacgccttgggtagattgttcgacgaacaatccgccttggacggtcaccacggtatgttggctggtttcggtccaggtatcatcgccgaaatgtccttgggtacctgggtttctccagaatcctaa

SEQ ID NO: 59 - protein, 401 aa, Streptomyces clavuligerus
MSSTGSSAHYCPFDYAEALEFDPTLRRFMREEPVARIRLPHGAGEAWLVTGYDDVRTVTTDRRFSRHAVVGRDFPRMTPEPIVQDEAINVMDPPASSRLRSLVSKGFAPEQIERMRPYIQRAVDDLLDRMAEDSSADLMRHLAGPLPLITICEVLEIPPADQETLRGHARTMMNISVDNKAAAVRAKADLRAYFADLTARRRADPGEDLISVLATARDGDELLDDQELTVMAMVLLITGQDTTTYELGNLSYTLLTRPDVRDLLRDRPERLAQTINELLRFIPFRKGVGIPRVATEDVELSGVTIPAGDIVHVSYLTANRDGRKFDRPDELDFDRTAPSHMTFGWGAHHCLGAPLAQAEMETAFRTLLERFPGIALAKPAEDVEWNTTSIWRYPLALPVTW

SEQ ID NO: 60 - DNA, 1206 bp, synthetic construct
  misc_feature 1..1206: note = coding sequence for SEQ ID NO: 59
  source 1..1206: mol_type = other DNA
atgtcctccaccggttcctctgctcactactgtccattcgactacgccgaagccttggaattcgacccaactttgagaagattcatgagagaagaaccagtcgctagaatcagattgccacacggtgctggtgaagcttggttggtcaccggttacgacgatgttagaaccgttaccaccgacagaagattctctagacacgccgttgtcggtagagacttcccaagaatgactccagaaccaatcgtccaagacgaagccatcaacgtcatggacccaccagcttcttctagattgagatctttggtttccaagggtttcgccccagaacaaatcgaaagaatgaggccatacatccaaagagccgttgacgacttgttggacagaatggctgaggactcctccgccgacttgatgagacacttggctggtccattgccattgatcaccatctgtgaagtcttggaaatcccaccagccgaccaagaaaccttgagaggtcacgccagaactatgatgaacatctctgttgacaacaaggccgccgccgttagagccaaggccgatttgagagcctacttcgctgacttgactgccagaagaagagctgacccaggtgaggacttgatctctgttttggccactgccagggacggtgacgaattgttggacgaccaagaattgaccgtcatggctatggtcttgttgatcaccggtcaagacaccaccacctacgaattgggtaacttgtcctacaccttgttgaccagaccagacgtcagagatttgttgagagacagaccagaaagattggctcaaactatcaacgaattgttgagattcatcccattcagaaagggtgtcggtatcccaagagttgccaccgaggacgttgaattgtctggtgttactatcccagccggtgacatcgtccacgtatcctacttgaccgccaacagagatggtagaaagttcgacagaccagacgaattggacttcgacagaaccgccccatcccacatgaccttcggttggggtgctcaccactgtttgggtgccccattggctcaagccgaaatggaaaccgccttcagaactttgttggaaagattcccaggtatcgctttggctaagccagccgaggacgttgaatggaacaccacctctatctggagatacccattggctttgccagtcacctggtaa

SEQ ID NO: 61 - protein, 246 aa, Arabidopsis thaliana
MSSSNACASPSPFPAVTKLHVDSVTFVPSVKSPASSNPLFLGGAGVRGLDIQGKFVIFTVIGVYLEGNAVPSLSVKWKGKTTEELTESIPFFREIVTGAFEKFIKVTMKLPLTGQQYSEKVTENCVAIWKQLGLYTDCEAKAVEKFLEIFKEETFPPGSSILFALSPTGSLTVAFSKDDSIPETGIAVIENKLLAEAVLESIIGKNGVSPGTRLSVAERLSQLMMKNKDEKEVSDHSVEEKLAKEN

SEQ ID NO: 62 - DNA, 741 bp, synthetic construct
  misc_feature 1..741: note = coding sequence for SEQ ID NO: 61
  source 1..741: mol_type = other DNA
atgtcctctagcaatgcgtgtgcctccccgtccccgttcccggctgttacgaagctgcatgtcgattcagttacctttgtcccgtccgtgaagtccccggcgagcagcaacccgctgtttctgggcggtgcaggtgtccgtggtctggatattcagggcaaatttgtgattttcaccgtgatcggcgtttatctggaaggcaatgcggtcccgtcactgtcggtgaaatggaagggtaaaaccacggaagaactgaccgaatctattccgtttttccgcgaaatcgttacgggcgcgttcgaaaagttcatcaaggtcaccatgaaactgccgctgacgggtcagcaatattcagaaaaggttaccgaaaactgcgtcgccatctggaaacaactgggcctgtacacggactgtgaagcgaaggccgtcgaaaagtttctggaaattttcaaagaagaaacctttccgccgggcagttccatcctgttcgcactgagcccgaccggttctctgacggttgctttcagtaaagatgactccatcccggaaaccggcattgcagtgatcgaaaacaagctgctggcagaagctgttctggaaagcattatcggcaaaaatggtgtgtcaccgggtacgcgtctgtcggttgcggaacgcctgagccagctgatgatgaaaaataaagatgaaaaggaagtgtcggaccacagcgttgaagaaaaactggcaaaggaaaactaa

SEQ ID NO: 63 - protein, 719 aa, Citrus sinensis
MSEIGATTENGHQNGGLEGLCKNNNYNYSSGDALNWGVMAETLKGSHLEEVKRMVAEYRKPVVNLGGETLTVAQVAAIATSSTNVELSESAREGVKASSDWVMESMNKGTDSYGVTTGFGATSHRRTKNGGALQKELIRFLNAGIFGNGTESSHTLPHSATRAAMLVRVNTLLQGYSGIRFEILEAITKLLNHNITPCLPLRGTITASGDLVPLSYIAGLLTGRPNSKATGPNGEIIDAQEASKQAGFGFFELQPKEGLALVNGTAVGSGLASMVLFEANNLALLSEILSAIFAEVMQGKPEFTDHLTHKLKHHPGQIEAAAIMEHILDGSSYVNVAKKLHEIDPLQKPKQDRYALRTSPQWLGPQIEVIRFATKSIEREINSVNDNPLIDVSRNKALHGGNFQGTPIGVSMDNTRLAIAAIGKLMFAQFSELVNDFYNNGLPSNLSGGRNPSLDYGFKGAEIAMASYCSELQFLANPVTNHVQSAEQHNQDVNSLGLISSRKTAEAVDILKLMSSTFLVALCQAIDLRHLEENLKHTVKNTVSQVAKKVLTVGASGELHPSRFCEKDLLKAADREHVFAYIDDPCSATYPLMQKLRQVLVEHALNNGENEKTANSSIFQKIAAFEEELKTVLPKEVENARQTVENGSPTIPNRIKECRSYPLYRFVREGLGSNFLTGEKVTSPGEEFDKVFTAMCQGKIIDPMLECLREWNGAPLPIC

SEQ ID NO: 64 - DNA, 2160 bp, synthetic construct
  misc_feature 1..2160: note = coding sequence for SEQ ID NO: 63
  source 1..2160: mol_type = other DNA
atgtccgaaatcggtgccactactgaaaacggtcaccaaaacggtggcctcgaaggcttgtgtaagaacaataactacaactactcttctggtgatgctttgaactggggtgttatggctgaaactttgaagggttctcacttggaagaagttaagagaatggttgctgaatacagaaagccagttgtcaacttgggtggtgaaactttgaccgttgctcaagttgctgccattgccacttcctctactaacgttgaattgtctgaatctgccagagaaggtgtcaaggcctcttctgattgggttatggaatctatgaacaagggtaccgactcttacggtgttactactggcttcggtgccacttcgcacagaagaaccaagaacggtggtgctttgcaaaaggaattgattagattcttgaacgctggtatcttcggtaacggtactgaatcttctcacactttgccacactctgctactagagctgccatgttggttcgtgttaacacattgttgcaaggttactccggtatcagattcgaaatcttggaagctatcactaagttgttgaaccacaacatcactccatgtttgccattgagaggtactatcactgcttctggtgatttggttccattgtcctacatagccggcttgttgaccggtcgtccaaactctaaggccaccggtccaaacggtgaaatcattgatgcccaagaagcctctaagcaagctggtttcggtttcttcgaattgcaaccaaaggagggcttggctctcgtcaacggtactgctgttggttctggtttggcttctatggttttgttcgaagctaacaatctcgcgttgttgtcggaaattttgtctgctattttcgctgaagtcatgcaaggtaagccagaattcaccgatcacttgactcacaagttgaagcaccacccaggtcaaattgaagctgctgctatcatggaacacatcttggatggttcttcttacgttaacgttgctaagaagttgcacgaaattgatccattgcaaaagccaaagcaagatagatacgccttgagaacttctccacaatggttgggtccacaaatcgaagttattagattcgctaccaagtctattgaaagagaaatcaactctgttaacgacaacccattgatcgacgtttctagaaacaaggccttgcacggtggtaacttccaaggtaccccaattggtgtctctatggacaacaccagattggctattgctgctatcggtaagttgatgttcgcccaattctctgaattggtcaacgatttctacaacaacggtttgccatctaacttgtctggtggtagaaacccatctttggattacggtttcaagggtgctgaaattgctatggcttcctactgttctgaattgcaattcttggccaacccagttactaaccacgtccaatctgctgaacaacacaaccaagatgttaactccttgggtttgatctcttccagaaagactgctgaagctgtcgacatcttgaagttgatgtcatccactttcttggttgctttgtgtcaagctattgatttgagacacttggaagaaaacttgaagcacactgtcaagaacactgtctctcaagttgctaagaaggtcttgaccgtcggtgcttctggtgaattgcacccatctagattctgtgaaaaggatttgttgaaggctgctgatagagaacacgttttcgcttacattgatgacccatgttctgctacctacccattgatgcaaaagttgagacaagttttggttgaacacgctttgaacaacggtgaaaacgaaaagactgccaactcttctatcttccaaaagattgctgccttcgaagaagaattaaagaccgttttgccaaaggaagttgaaaacgccagacaaactgttgaaaacggttctccaactattccaaacagaatcaaggaatgtagatcttacccattgtacagattcgttagagaaggtttgggttctaacttcttgactggtgaaaaggttacttctccaggtgaagaattcgacaaggttttcactgctatgtgtcaaggtaagatcattgatccaatgttggaatgtttgagagaatggaacggtgccccattgccaatttgttaa

SEQ ID NO: 65 - protein, 715 aa, Citrus sinensis
MSEASHENQSGGNIPSGKLCTNIDPLNWVSASESLKGSHLDEVKRMVSEYRKQVVRLGGETLTIAQVAAVASRDGGVTVELNEEARAGVKASSDWVMESMNKGTDSYGITTGFGATSHRRTKQGAALQKELIRFLNAGIFGKGTESCQMLPHTATRAAMLVRINTLLQGYSGIRFEILEAITKFLNRNITPCLPLRASITASGDLIPFSYIAGLLTGRLNSVAVGPNGESLNAAEAFSQAGIDGGFFELQPKEGLALVNGTGVGAGLASIVLFEANILTVLSEVLSAIFAEAMLGKPEFTDHLTHKLKHHPGQIEAAAIMEHILDGSSYVKAAQKLHEIDPLQKPKQDRYALRTSPQWLGPQAEVIRASTKSIEREINSVNDNPLIDVSRNKALHGGNFQGTPIGVSMDNSRLAIASIGKLMFAQFSELVNDFYSNGLPSNLSGGRNPSLDYGFKGAEIAMAAYCSELQFLANPVTNHVQSAEQHNQDVNSLGLISARKTAEAVDILKLMSSTYLIALCQAIDLRHLEENLKSTVKNTISQVVKKVLTMGVNGELHPSRFCEKDLLKVVDREYVFSYADDPCSATYPLMQKLRQVLVDHALTNNEDLKNANASIFLKIGAFEEELKTLLPKEVESARSAFESGNLEIPNRIKECRSYPLYRFVREELGARYLTGEKAISPGEECDKVFTAICQGKIIDPLLECLKEWDGSPLPIC

SEQ ID NO: 66 - DNA, 2148 bp, synthetic construct
  misc_feature 1..2148: note = coding sequence for SEQ ID NO: 65
  source 1..2148: mol_type = other DNA
atgtccgaagcttctcacgaaaaccaatctggtggtaacattccatctggtaagttgtgtactaacattgatccattgaactgggtttctgcttctgaatctttgaagggttctcacttggatgaagttaagagaatggtttctgaatacagaaagcaagttgttagattgggtggtgaaaccttgactatcgctcaagttgctgctgttgcttctagagatggtggtgtcactgttgaattgaacgaagaagccagagctggtgtcaaggcttcttctgattgggttatggaatctatgaacaagggtactgattcttacggcatcactactggcttcggtgctacttcgcacagaagaaccaagcaaggtgctgccttgcaaaaggaattgattagattcttgaacgctggtatcttcggtaagggtactgaatcctgtcaaatgttgccacacactgctactagagctgctatgctcgttcgtatcaacacattgttgcaaggttactctggtattagattcgaaattttggaagctatcactaagttcttgaacagaaacattactccatgtttgccattgagagcctccatcactgcgtcgggtgatctcatcccattctcgtacatcgccggtttgttgaccggtagattgaactctgttgctgttggtccaaacggtgaatctttgaacgctgctgaagctttctctcaagctggtattgatggtggtttcttcgaattgcaaccaaaggagggcttggcgttggttaacggtactggtgttggtgctggtttggcttctattgttttgttcgaagctaacattttgactgttttgtccgaagttttgtctgctattttcgctgaagctatgttgggtaagccagaattcactgatcacttgactcacaagttgaagcaccacccaggtcaaattgaagctgctgctatcatggaacacatcttggatggttcttcttacgttaaggctgctcaaaagttgcacgaaattgacccattgcaaaagccaaagcaagatagatacgctttgagaacttctccacaatggttgggtccacaagctgaagttattagagcttctactaagtctattgaaagagaaattaactctgttaacgataacccattgattgatgtttctagaaacaaggctttgcacggtggtaacttccaaggtaccccaattggtgtctctatggataactctagattggctattgcttctattggtaagttgatgttcgctcaattctctgaattggttaacgatttctactctaacggtttgccatctaacttgtctggtggtagaaacccatctttggattacggtttcaagggtgccgaaatcgctatggctgcttactgttccgaattgcaattcttggccaacccagttactaaccacgtccaatctgctgaacaacacaaccaagatgttaactctttgggtttgatctctgccagaaagactgctgaagctgttgatatcttgaagttgatgtcctctacttacttgattgctttgtgtcaagctatcgacttgagacacttggaagaaaacttgaagtctactgttaagaacactatctctcaagttgttaagaaggtcttgactatgggtgtcaacggtgaattgcacccatctagattctgtgaaaaggatttgttgaaggttgttgatagagaatacgttttctcttacgctgatgatccatgttctgctacttacccattgatgcaaaagttgagacaagtcttggttgatcacgctttgactaacaacgaagatttgaagaacgctaacgcttctatcttcttgaaaataggtgcgttcgaagaagaactcaagaccttgttgccaaaggaagttgaatctgctagatctgctttcgaatctggtaacttggaaatcccaaacagaattaaggaatgtagatcctacccattgtacagattcgttagagaagaattgggtgctagatacttgactggtgaaaaggctatctctccaggtgaagaatgtgataaggttttcactgctatttgtcaaggtaagatcattgatccattgttggaatgtttgaaggaatgggatggttctccattgccaatttgttaa

SEQ ID NO: 67 - protein, 537 aa, Citrus sinensis
MSANLVTISFFSILLTISLLSFNKSLNLISITLPLVPLIAYVLKSFLKSSKAFYPPTPISIPIFGNWLQVGNDLNHRLLASMAQIYGPVFRLKLGSKNLIVVSEPDLATQVLHTQGVEFGSRPRNVVFDIFTGNGQDMVFTVYGEHWRKMRRIMTLPFFTNKVVHNYSDMWEQEMDLVVHDLKNDYESVSTKGIVIRKRLQLMLYNIMYRMMFDAKFESQEDPLFIEATRFNSERSRLAQSFEYNYGDFIPLLRPFLRGYLNKCRDLQCRRLAFFNNNFVEKRRKIMAANGEKHKISCAIDHIIDAQMKGEITEENVIYIVENINVAAIETTLWSMEWAIAELVNHPEVQQKIRREISTVLKGNPVTESNLHELPYLQAAVKEVLRLHTPIPLLVPHMNLEEAKLGGFTIPKESKIVVNAWWLANNPKWWEKPEEFRPERFLEEECNIDAVAGGGKVDFRYLPFGVGRRSCPGIILALPILGLVIAKLVTSFEMKAPQGIDKIDVSEKGGQFSLHIANHSTVVFDPIMESLSQPMPQ

SEQ ID NO: 68 - DNA, 1614 bp, synthetic construct
  misc_feature 1..1614: note = coding sequence for SEQ ID NO: 67
  source 1..1614: mol_type = other DNA
atgtccgctaacttggttactatttctttcttctctatcttgttgactatctctttgttgtctttcaacaagtctttgaacttgatctctatcactttgccattggttccattgattgcttacgttttgaagtccttcttgaagtcatctaaggccttctacccaccaactccaatctctatcccaatcttcggtaactggttgcaagttggtaacgacttgaaccacagattgttggcttctatggctcaaatttacggtccagttttcagattgaagttgggttctaagaacttgatcgttgtttctgaaccagacctcgctactcaagtgctacacactcaaggtgttgaattcggttccagaccaagaaacgttgttttcgatattttcactggtaacggtcaagacatggtatttactgtctacggcgaacactggagaaagatgagaagaattatgactttgccattcttcaccaacaaggttgttcacaactactctgacatgtgggaacaagaaatggacttggttgttcacgacttgaagaacgattacgagtctgtaagcactaagggtattgttattagaaagagattgcaattgatgttgtacaacattatgtacagaatgatgttcgatgctaagttcgaatctcaagaagatccattgttcattgaagctactagattcaactctgaaagatctagattggcgcagagcttcgaatacaactacggtgatttcattccattgttaagaccattcttgagaggttacttgaacaagtgtagagacttgcaatgtagaagattggctttcttcaacaacaacttcgttgaaaagagaagaaagatcatggctgccaacggtgaaaagcacaagatctcttgtgccattgatcacatcattgatgctcaaatgaagggtgaaatcactgaagaaaacgttatttacattgttgaaaacatcaacgttgctgctatcgaaactactttgtggtccatggaatgggctatcgctgaattggtcaaccacccagaagttcaacaaaagatcagaagagaaatctctactgtcttgaagggtaacccagtcactgaatctaacttgcacgaattgccatacttgcaagccgctgttaaggaagttttgagattgcacactccaattccattgttggttccacacatgaacttggaagaagctaagttgggtggtttcactattccaaaggaatccaagattgttgttaacgcttggtggttggctaacaacccaaagtggtgggaaaagccagaagaattcagaccagaaagattcttggaagaagaatgtaacattgatgctgttgctggtggtggtaaggttgacttcagatacttgccattcggtgttggtagaagatcttgtccaggtataatattggctctcccaatcttgggcttggttattgctaagttggttacttctttcgaaatgaaggctccacaaggtatcgataagattgacgtttctgaaaagggtggtcaattctctttgcacattgctaaccactctactgttgtctttgatccaatcatggaatctttgtcccaaccaatgccacaataa

SEQ ID NO: 69 - protein, 520 aa, Citrus sinensis
MSDLNGWCNSGNQNMCCCQSYVKRGYDRVLSFNGLITVSKLRGKRFKLPPGPLPVPVFGNWLQVGDDLNHRNLSDLAKKYGDVLLLRMGQRNLVVVSSPDHAKEVLHTQGVEFGSRTRNVVFDIFTGKGQDMVFTVYGEHWRKMRRIMTVPFFTNKVVQQQRFNWEDEAARVVEDVKKDPEAATNGIVLRRRLQLMMYNNMYRIMFDRRFESQDDPLFNRLKALNGERSRLAQSFEYNYGDFIPILRPFLRGYLKICKEVKERRLQLFKDYFVEERKKLASTKSMSNESLKCAIDHILDAQTKGEINEDNVLYIVENINVAAIETTLWSIEWGIAELVNHPEIQKKLRNELDTVLGPGHQITEPDTHKLPYLQAVIKETLRLRMAIPLLVPHMNLHDAKLGGYDVPAESKILVNAWWLANNPAQWKKPEEFRPERFLEEESKVEANGNDFRYLPFGVGRRSCPGIILALPILGITIGRLVQNFELLPPPGQSKIDTAEKGGQFSLHILKHSTIVAKPRSF

SEQ ID NO: 70 - DNA, 1563 bp, synthetic construct
  misc_feature 1..1563: note = coding sequence for SEQ ID NO: 69
  source 1..1563: mol_type = other DNA
atgtccgacttgaacggttggtgtaactctggtaaccaaaacatgtgttgttgtcaatcttacgttaagagaggttacgatagagttttgtctttcaacggtttgattaccgtttctaagttgagaggtaagagattcaagttgccaccaggtccattgccagtcccagttttcggtaactggttgcaagtcggtgatgacttgaaccacagaaacttgtccgacttggccaagaagtacggtgatgtcttgttgttgagaatgggtcaaagaaacttggtcgtcgtttcttctccagaccacgccaaggaagtcttgcacactcaaggtgttgaattcggttctagaactcgtaatgtcgtgttcgacatcttcactggtaagggtcaagatatggttttcactgtctacggcgaacactggagaaagatgagaagaatcatgaccgtcccattcttcactaacaaggttgttcaacaacaaagattcaactgggaagatgaagccgctagagtcgttgaagatgttaagaaggacccagaagctgctaccaacggtatcgtcttgagaagaagattgcaattgatgatgtacaacaacatgtacagaattatgttcgatagaagattcgaatctcaagatgatccattgttcaacagattgaaggctttgaacggtgaaagatctagattggcgcagagcttcgaatacaactacggtgatttcattccaattttaagaccattcttgagaggttacttgaagatttgtaaggaagttaaggaaagaagattgcaattgttcaaggactacttcgttgaagaaagaaagaagttggcttctactaagtctatgtctaacgaatctttgaagtgtgctatcgatcacattttggacgctcaaactaagggtgaaatcaacgaagataacgttttgtacatcgttgaaaacatcaacgttgccgctatcgaaactactttgtggtctatcgaatggggtattgctgaattggttaaccacccagaaatccaaaagaagttgagaaacgaattggacaccgttttgggtccaggtcaccaaatcactgaaccagacacccacaagttgccatacttgcaagccgttatcaaggaaactttgagattgagaatggctattccattgttggttccacacatgaacttgcacgacgccaagttgggtggttacgacgtcccagctgaatctaagatcttggttaacgcttggtggttggccaacaacccagcccaatggaagaagccagaagaattcagaccagaaagattcttggaagaagaatccaaggttgaagctaacggtaacgacttcagatacttgccattcggtgttggtagaagatcttgtccaggtataatattggctctcccaattttgggcatcactattggtagattggttcaaaacttcgaattgttgccaccaccaggtcaatctaagattgacactgctgaaaagggtggtcaattctctttgcacattttgaagcactctaccattgttgccaagccaagatctttctaa

SEQ ID NO: 71 - protein, 513 aa, Saccharothrix espanaensis
MSTITSPAPAGRLNNVRPMTGEEYLESLRDGREVYIYGERVDDVTTHLAFRNSVRSIARLYDVLHDPASEGVLRVPTDTGNGGFTHPFFKTARSSEDLVAAREAIVGWQRLVYGWMGRTPDYKAAFFGTLDANAEFYGPFEANARRWYRDAQERVLYFNHAIVHPPVDRDRPADRTADICVHVEEETDSGLIVSGAKVVATGSAMTNANLIAHYGLPVRDKKFGLVFTVPMNSPGLKLICRTSYELMVATQGSPFDYPLSSRLDENDSIMIFDRVLVPWENVFMYDAGAANSFATGSGFLERFTFHGCTRLAVKLDFIAGCVMKAVEVTGTTHFRGVQAQVGEVLNWRDVFWGLSDAMAKSPNSWVGGSVQPNLNYGLAYRTFMGVGYPRIKEIIQQTLGSGLIYLNSSAADWKNPDVRPYLDRYLRGSRGIQAIDRVKLLKLLWDAVGTEFAGRHELYERNYGGDHEGIRVQTLQAYQANGQAAALKGFAEQCMSEYDLDGWTRPDLINPGT

SEQ ID NO: 72 - DNA, 1542 bp, synthetic construct
  misc_feature 1..1542: note = coding sequence for SEQ ID NO: 71
  source 1..1542: mol_type = other DNA
atgtccaccatcacttctccagctccagctggtagattgaacaacgtcagaccaatgactggtgaagaatacttggaatctttgagagatggtagagaagtctacatctacggtgaaagagtcgacgacgtcaccactcacttggccttcagaaactctgttagatccatcgctagattgtacgacgttttgcacgacccagcttcggagggtgtactaagagttccaaccgacactggtaatggtggcttcactcatccattcttcaagaccgcccgttcttctgaagatttggtcgccgctagagaagccatcgtcggttggcaaagattggtttacggttggatgggtagaaccccagattacaaggctgcgttcttcggtactctcgacgccaacgccgaattctacggtccattcgaagccaacgccagaagatggtacagagatgcccaagaaagagttttgtacttcaaccacgctatcgttcacccaccagtcgacagagacagaccagccgacagaaccgccgacatctgtgttcacgttgaagaagaaaccgactctggtttgatcgtttccggtgccaaggttgtcgctaccggttccgctatgaccaacgctaacttgatcgctcactacggtttgccagttagagacaagaagttcggtttggttttcactgtcccaatgaactctccaggtttgaagttgatctgtagaacctcctacgaattgatggtcgctactcaaggttctccattcgactacccattatcttctagattggacgaaaacgactctatcatgatcttcgacagagttttggttccatgggaaaacgttttcatgtacgacgctggtgctgccaactccttcgccaccggttctggtttcttggaaagattcaccttccacggttgtaccagattggctgtcaagttggacttcatcgccggttgtgtcatgaaggctgttgaagtcaccggtaccactcacttcagaggtgttcaagctcaagtcggtgaagttttgaactggagagatgttttctggggtttgtccgacgctatggccaagtctccaaactcttgggtcggtggttctgttcaaccaaacttgaactacggtttggcttacagaaccttcatgggtgttggttacccaagaatcaaggaaatcatccaacaaaccttgggttctggtttgatctacttgaactcctctgccgccgactggaagaacccagacgtcagaccatacttggacagatacttgagaggttctagaggtatccaagctatcgacagagtcaagttgttgaagttgttgtgggacgctgtcggtaccgaattcgccggtagacacgaattgtacgaaagaaactacggtggtgaccacgaaggtatcagagttcaaaccttgcaagcttaccaagctaacggtcaggcggccgctctcaagggtttcgcggaacaatgtatgtccgaatacgacttggacggttggactagaccagacttgatcaacccaggtacctaa

SEQ ID NO: 73 - protein, 498 aa, Beta vulgaris
MSDHATLAMILAILFISFHFIKLLFSQQTTKLLPPGPKPLPIIGNILEVGKKPHRSFANLAKIHGPLISLRLGSVTTIVVSSADVAKEMFLKKDHPLSNRTIPNSVTAGDHHKLTMSWLPVSPKWRNFRKITAVHLLSPQRLDACQTFRHAKVQQLYEYVQECAQKGQAVDIGKAAFTTSLNLLSKLFFSVELAHHKSHTSQEFKELIWNIMEDIGKPNYADYFPILGCVDPSGIRRRLACSFDKLIAVFQGIICERLAPDSSTTTTTTTDDVLDVLLQLFKQNELTMGEINHLLVDIFDAGTDTTSSTLEWVMTELIRNPEMMEKAQEEIKQVLGKDKQIQESDIINLPYLQAIIKETLRLHPPTVFLLPRKADTDVELYGYIVPKDAQILVNLWAIGRDPNAWQNADIFSPERFIGCEIDVKGRDFGLLPFGAGRRICPGMNLAIRMLTLMLATLLQFFNWKLEGDISPKDLDMDEKFGIALQKTKPLKLIPIPRY

SEQ ID NO: 74 - DNA, 1497 bp, synthetic construct
  misc_feature 1..1497: note = coding sequence for SEQ ID NO: 73
  source 1..1497: mol_type = other DNA
atgtccgatcacgctactttggcgatgatattggccatcctattcatttctttccacttcatcaagttgttgttctctcaacaaactaccaagttgttgccaccaggtccaaagccattgccaatcattggtaacatcttggaagttggtaagaagccacacagatctttcgctaacttggctaagattcacggtccattgatctctttgagattgggttctgttactactattgttgtttcttctgctgatgttgctaaggaaatgttcttgaagaaggaccacccattgtctaacagaactattccaaactctgtcactgccggtgaccaccacaagttgaccatgtcttggttgccagtttctccaaagtggagaaacttcagaaagattactgccgtccacttgttgtctccacaaagattggatgcttgtcaaaccttcagacacgccaaggttcaacaattgtacgaatacgttcaagaatgtgctcaaaagggtcaagctgttgatattggtaaggctgctttcactacctccttgaacttgttgtctaagttgttcttctctgttgaattggcccaccacaagtctcacacttctcaagaattcaaggaattgatctggaacattatggaagatattggtaagccaaactacgctgattacttcccaattttgggttgtgttgatccatctggtattagaagaagattggcttgttctttcgacaagttgattgctgttttccaaggtatcatctgtgaaagattggctccagattcttctactacaaccactactaccactgatgatgttttggacgttttgttgcaattgttcaagcaaaacgaattgactatgggtgaaattaaccacttgttggtcgacattttcgatgctggtactgacactacttcttctactttggaatgggtcatgactgaattgattagaaacccagaaatgatggaaaaggctcaagaagaaattaagcaagttttgggtaaggataagcaaattcaagaatctgacattattaacttgccatacttgcaagccattatcaaggaaactttgagattgcacccaccaactgttttcttgttgccaagaaaggccgacactgatgttgaattgtacggttacattgttccaaaggatgctcaaatcttggttaacttgtgggctattggtagagatccaaacgcttggcaaaacgctgatattttctctccagaaagattcatcggttgtgaaattgatgtcaagggtagagatttcggtttgttgccattcggtgccggtagaagaatctgtccaggtatgaacttggccattagaatgttgactttgatgttggctactttgttgcaattcttcaactggaagttggaaggtgacatctctccaaaggacttggacatggatgaaaagttcggtattgctttgcaaaagactaagccattgaagttgattccaatcccaagatactaa

SEQ ID NO: 75 - protein, 413 aa, Rhodopseudomonas palustris
MSTTAPSLVPVTTPSQHGAGVPHLGIDPFALDYFADPYPEQETLREAGPVVYLDKWNVYGVARYAEVYAVLNDPLTFCSSRGVGLSDFKKEKPWRPPSLILEADPPAHTRTRAVLSKVLSPATMKRLRDGFAAAADAKIDELLARGGNIDAIADLAEAYPLSVFPDAMGLKQEGRENLLPYAGLVLNAFGPPNELRQSAIERSAPHQAYVAEQCQRPNLAPGGFGACIHAFSDTGEITPEEAPLLVRSLLSAGLDTTVNGIAAAVYCLARFPDEFARLRADPSLARNAFEEAVRFESPVQTFFRTTTRDVELAGATIGEGEKVLMFLGSANRDPRRWDDPDRYDITRKTSGHVGFGSGVHMCVGQLVARLEGEVVLAALARKVAAIEIAGPLKRRFNNTLRGLESLPIQLTPA

SEQ ID NO: 76 - DNA, 1242 bp, synthetic construct
  misc_feature 1..1242: note = coding sequence for SEQ ID NO: 75
  source 1..1242: mol_type = other DNA
atgtccaccactgctccatctttggttccagttaccactccatcccaacacggtgctggtgttccacacttgggtatcgatccattcgctttggactacttcgccgatccatacccagaacaagaaactttgagagaagctggtccagttgtctacttggacaagtggaacgtctacggtgtcgccagatacgctgaagtatacgccgtactcaacgatccattgaccttctgttcctctagaggtgttggtttgtctgatttcaagaaggaaaagccatggaggccaccatctttgatcttggaagccgacccaccagctcacaccagaaccagagctgttttgtctaaggttttgtctccagctactatgaagagattgagagatggtttcgctgctgctgctgacgccaagatcgatgaattgttggccagaggtggtaacatcgatgctatcgccgacttggccgaagcctacccattgtctgttttcccagatgcgatgggtttgaagcaagaaggcagagaaaacttgttgccatacgcgggcttggttttgaacgcattcggtccaccaaacgaattgagacaatctgctatcgaaagatctgctccacaccaagcctacgttgccgaacaatgtcaaagaccaaacttggctccaggtggtttcggtgcctgtatccacgccttctctgacaccggtgaaatcactccagaagaagccccattgttggttagatctttgttgtctgctggtttggacactaccgtcaacggtattgctgctgctgtttactgtttggctagattcccagacgaattcgctagattgagagccgatccatctttggccagaaacgccttcgaagaagctgttagattcgaatctccagttcaaaccttcttcagaaccaccactagagatgtcgaattggctggtgccactatcggtgaaggtgaaaaggttttgatgttcttgggttccgccaacagagatccaagaagatgggacgatccagacagatacgacatcaccagaaagacctctgggcatgtcggcttcggttctggtgttcacatgtgtgtcggtcaattggtcgccagattggaaggtgaagttgttttggccgctttggctagaaaggttgctgctatcgaaatcgccggtccattgaagagaagattcaacaacactttgagaggtttggaatctttgccaatccaattgactccagcttaa

SEQ ID NO: 77 - protein, 718 aa, Arabidopsis thaliana
MSDQIEAMLCGGGEKTKVAVTTKTLADPLNWGLAADQMKGSHLDEVKKMVEEYRRPVVNLGGETLTIGQVAAISTVGGSVKVELAETSRAGVKASSDWVMESMNKGTDSYGVTTGFGATSHRRTKNGTALQTELIRFLNAGIFGNTKETCHTLPQSATRAAMLVRVNTLLQGYSGIRFEILEAITSLLNHNISPSLPLRGTITASGDLVPLSYIAGLLTGRPNSKATGPDGESLTAKEAFEKAGISTGFFDLQPKEGLALVNGTAVGSGMASMVLFEANVQAVLAEVLSAIFAEVMSGKPEFTDHLTHRLKHHPGQIEAAAIMEHILDGSSYMKLAQKVHEMDPLQKPKQDRYALRTSPQWLGPQIEVIRQATKSIEREINSVNDNPLIDVSRNKAIHGGNFQGTPIGVSMDNTRLAIAAIGKLMFAQFSELVNDFYNNGLPSNLTASSNPSLDYGFKGAEIAMASYCSELQYLANPVTSHVQSAEQHNQDVNSLGLISSRKTSEAVDILKLMSTTFLVGICQAVDLRHLEENLRQTVKNTVSQVAKKVLTTGINGELHPSRFCEKDLLKVVDREQVFTYVDDPCSATYPLMQRLRQVIVDHALSNGETEKNAVTSIFQKIGAFEEELKAVLPKEVEAARAAYGNGTAPIPNRIKECRSYPLYRFVREELGTKLLTGEKVVSPGEEFDKVFTAMCEGKLIDPLMDCLKEWNGAPIPIC

SEQ ID NO: 78 - DNA, 2157 bp, synthetic construct
  misc_feature 1..2157: note = coding sequence for SEQ ID NO: 77
  source 1..2157: mol_type = other DNA
atgtccgatcaaatcgaagctatgttgtgtggtggtggtgaaaaaacaaaagttgctgttactactaagaccttggccgatccattgaattggggtttggctgctgatcaaatgaagggttctcatttggatgaagtcaagaagatggtcgaagaatacagaaggccagttgttaatttgggtggtgaaactttgactattggtcaagttgctgctatttctactgttggtggttctgttaaggttgaattggctgaaacttctagagctggtgttaaggcttcttctgattgggttatggaatctatgaacaagggtactgattcttacggtgttactacaggttttggtgctacttctcatagaagaactaagaatggtactgccttgcaaaccgaattgatcagatttttgaacgccggtattttcggtaacaccaaagaaacttgtcataccttgccacaatctgctactagagctgctatgttggttagagttaacactttgttgcaaggttactccggtatcagattcgaaattttggaagctatcacctccttgttgaaccataacatttctccatctttgccattgagaggtactattactgcttctggtgatttggttccattgtcttatattgctggtttgttgactggtagaccaaactctaaagctactggtccagatggtgaatcattgactgctaaagaagcttttgaaaaggctggtatctctactggttttttcgacttgcaacctaaagaaggtttggctttggttaatggtacagctgttggttctggtatggcttctatggttttgtttgaagctaacgttcaagctgttttggccgaagttttgtctgctatttttgctgaagttatgtccggtaagccagaattcactgatcatttgacccatagattgaaacatcacccaggtcaaattgaagctgctgcaattatggaacatatcttggatggttcctcttacatgaagttggctcaaaaagttcacgaaatggacccattgcaaaagccaaaacaagatagatacgctttgagaacttctccacaatggttgggtccacaaatagaagttattagacaagccaccaagtccatcgaaagagaaatcaattctgttaacgacaacccattgatcgacgtcagtagaaacaaagctattcatggtggtaacttccaaggtactccaattggtgtttctatggacaacactagattggctattgctgccattggtaaattgatgttcgctcaattctccgaattggtcaacgatttttacaacaacggtttgccttctaacttgaccgcttcttctaatccatcattggattacggttttaagggtgctgaaattgctatggcttcatactgttctgaattgcaatacttggctaacccagttacctctcatgttcaatctgctgaacaacacaatcaagacgttaactccttgggtttgatctcttctagaaagacttctgaagccgttgacatcttgaagttgatgtctactacattcttggtcggtatttgccaagctgttgatttgagacatttggaagaaaacttgagacaaaccgtcaagaacaccgtttcacaagttgctaagaaagttttgaccaccggtattaacggtgaattgcatccatctagattctgcgaaaaggatttgttgaaggtcgttgatagagaacaagttttcacctacgttgatgatccatgttctgctacttatccattgatgcaaagattgagacaagtcatcgttgatcatgctttgtctaatggtgaaaccgaaaagaacgctgttacctccattttccaaaagattggtgctttcgaagaagaattgaaggccgttttgccaaaagaagttgaagcagctagagcagcttacggtaacggtactgctccaattccaaatagaatcaaagaatgcagatcctacccattatacagattcgttagagaagaattaggtactaagttgttgaccggtgaaaaggttgtttctccaggtgaagaattcgataaggttttcactgctatgtgcgaaggtaaattgatcgatccattgatggactgcttgaaagaatggaatggtgctcctattcctatctgctaa

SEQ ID NO: 79 - protein, 506 aa, Arabidopsis thaliana
MSDLLLLEKSLIAVFVAVILATVISKLRGKKLKLPPGPIPIPIFGNWLQVGDDLNHRNLVDYAKKFGDLFLLRMGQRNLVVVSSPDLTKEVLLTQGVEFGSRTRNVVFDIFTGKGQDMVFTVYGEHWRKMRRIMTVPFFTNKVVQQNREGWEFEAASVVEDVKKNPDSATKGIVLRKRLQLMMYNNMFRIMFDRRFESEDDPLFLRLKALNGERSRLAQSFEYNYGDFIPILRPFLRGYLKICQDVKDRRIALFKKYFVDERKQIASSKPTGSEGLKCAIDHILEAEQKGEINEDNVLYIVENINVAAIETTLWSIEWGIAELVNHPEIQSKLRNELDTVLGPGVQVTEPDLHKLPYLQAVVKETLRLRMAIPLLVPHMNLHDAKLAGYDIPAESKILVNAWWLANNPNSWKKPEEFRPERFFEEESHVEANGNDFRYVPFGVGRRSCPGIILALPILGITIGRMVQNFELLPPPGQSKVDTSEKGGQFSLHILNHSIIVMKPRNC

SEQ ID NO: 80 - DNA, 1521 bp, synthetic construct
  misc_feature 1..1521: note = coding sequence for SEQ ID NO: 79
  source 1..1521: mol_type = other DNA
atgtccgacttgttgttgttggaaaagtccttgattgctgttttcgttgctgttattttggccaccgttatctctaaattgagaggtaagaaattgaagttgccaccaggtccaattccaatcccaatttttggtaattggttgcaagttggtgatgacttgaaccacagaaacttggttgattacgctaaaaagttcggtgatttgttcttgttgagaatgggtcaaagaaatttggtcgttgtttcctcaccagacttgaccaaagaagttttgttgactcaaggtgtcgaattcggttccagaactagaaatgttgttttcgatatcttcaccggtaagggtcaagatatggtttttactgtttacggtgaacattggagaaagatgagaagaattatgaccgttccattcttcaccaacaaggttgtccaacaaaacagagaaggttgggaatttgaagctgcttctgttgttgaagatgtcaagaagaatccagattctgctactaagggtatcgttttgagaaaaagattgcaattgatgatgtacaacaacatgttcagaatcatgttcgacagaagatttgaatccgaagatgaccctttgtttttgagattgaaggctttgaacggtgaaagatctagattggctcaatccttcgaatacaactacggtgatttcatcccaatcttaagaccattcttgagaggttacttgaagatctgccaagatgttaaggatagaagaatcgccttgttcaaaaagtacttcgttgacgaaagaaagcaaatcgcttcttctaaacctactggttctgaaggtttgaagtgcgccattgatcatattttggaagctgaacaaaagggtgaaatcaacgaagataacgtcttgtacatcgtcgaaaacattaacgttgctgctattgaaactaccttgtggtctattgaatggggtattgctgaattggttaatcacccagaaatccaatccaagttgagaaacgaattggatactgttttgggtccaggtgttcaagttactgaacctgacttgcataagttgccatacttgcaagctgttgtaaaagaaaccttgagattaagaatggccatccctttgttggttccacatatgaacttgcatgatgctaaattggccggttatgatattccagccgaatccaagattttggttaatgcttggtggttggctaacaatccaaattcttggaaaaagccagaagaattcagaccagaaagatttttcgaagaagaaagtcacgttgaagccaacggtaatgattttagatacgttccatttggtgttggtagaagatcttgtccaggtattatcttggctttgccaattttgggtattaccatcggtagaatggtccaaaacttcgaattattgccaccacctggtcaatctaaggttgatacttctgaaaagggtggtcaattctccttgcatattttgaaccactccatcatcgttatgaagccaagaaactgttaa

SEQ ID NO: 81 - protein, 384 aa, Saccharomyces cerevisiae
MSKSKTFLFTSESVGEGHPDKICDQVSDAILDACLEQDPFSKVACETAAKTGMIMVFGEITTKARLDYQQIVRDTIKKIGYDDSAKGFDYKTCNVLVAIEQQSPDIAQGLHYEKSLEDLGAGDQGIMFGYATDETPEGLPLTILLAHKLNMAMADARRDGSLPWLRPDTKTQVTVEYEDDNGRWVPKRIDTVVISAQHADEISTADLRTQLQKDIVEKVIPKDMLDENTKYFIQPSGRFVIGGPQGDAGLTGRKIIVDAYGGASSVGGGAFSGKDYSKVDRSAAYAARWVAKSLVAAGLCKRVQVQFSYAIGIAEPLSLHVDTYGTATKSDDEIIEIIKKNFDLRPGVLVKELDLARPIYLPTASYGHFTNQEYSWEKPKKLEF

SEQ ID NO: 82 - DNA, 1155 bp, synthetic construct
  misc_feature 1..1155: note = coding sequence for SEQ ID NO: 81
  source 1..1155: mol_type = other DNA
atgtccaagagcaaaactttcttatttacctctgaatccgtcggtgaaggtcacccagacaagatttgtgaccaagtttctgatgctattttggacgcttgtttagaacaagatccattctccaaggttgcctgtgaaacagctgccaaaactggtatgattatggttttcggtgaaattaccaccaaagctagacttgactaccaacaaatagtaagagataccatcaagaagattggttatgacgattctgccaagggtttcgactacaagacatgtaatgttttagtagctatcgaacaacaatctccagatatcgctcaaggtctgcactatgaaaagagcttagaagatttaggtgctggtgaccaaggtataatgtttggttacgctacagacgaaactccagaagggttaccattgaccattcttttggctcacaaattgaacatggctatggcagatgctagaagagatggttctctcccatggttgaggccagacacaaagactcaagtcactgtcgaatacgaggacgacaatggtagatgggttccaaagaggatagataccgttgttatttctgctcaacatgctgatgaaatttccaccgctgacttgagaactcaacttcaaaaagatattgttgaaaaggtcataccaaaggatatgttagacgaaaataccaaatatttcatccaaccatccggtagattcgtcatcggtggtcctcaaggtgacgctggtttgaccggtagaaagattattgtcgacgcttacggtggtgcctcatccgtcggtggtggtgccttctccggtaaggactattccaaggtcgatcgttccgctgcttacgctgctagatgggttgccaagtctctagttgccgctggtttgtgtaagagagtccaagtccaattttcatatgctattggtattgctgaaccattgtctttacatgtggacacctatggtacagctacaaaatcagatgacgaaatcattgaaattattaagaagaacttcgacttgaggccaggtgtgttagtaaaggaattagatttggctagaccaatttacttaccaaccgcttcttatggtcacttcactaatcaagagtactcatgggaaaaaccaaagaaattggaattttaa

SEQ ID NO: 83 - protein, 521 aa, Escherichia coli
MSKPEDFRASTQRPFTGEEYLKSLQDGREIYIYGERVKDVTTHPAFRNAAASVAQLYDALHKPEMQDSLCWNTDTGSGGYTHKFFRVAKSADDLRQQRDAIAEWSRLSYGWMGRTPDYKAAFGCALGANPGFYGQFEQNARNWYTRIQETGLYFNHAIVNPPIDRHLPTDKVKDVYIKLEKETDAGIIVSGAKVVATNSALTHYNMIGFGSAQVMGENPDFALMFVAPMDADGVKLISRASYEMVAGATGSPYDYPLSSRFDENDAILVMDNVLIPWENVLIYRDFDRCRRWTMEGGFARMYPLQACVRLAVKLDFITALLKKSLECTGTLEFRGVQADLGEVVAWRNTFWALSDSMCSEATPWVNGAYLPDHAALQTYRVLAPMAYAKIKNIIERNVTSGLIYLPSSARDLNNPQIDQYLAKYVRGSNGMDHVQRIKILKLMWDAIGSEFGGRHELYEINYSGSQDEIRLQCLRQAQNSGNMDKMMAMVDRCLSEYDQDGWTVPHLHNNDDINMLDKLLK

SEQ ID NO: 84 - DNA, 1566 bp, synthetic construct
  misc_feature 1..1566: note = coding sequence for SEQ ID NO: 83
  source 1..1566: mol_type = other DNA
atgtccaaaccagaagatttccgcgccagtacccaacgtcctttcaccggggaagagtatctgaaaagcctgcaggatggtcgcgagatctatatctatggcgagcgagtgaaagacgtcaccactcatccggcatttcgtaatgcggcagcgtctgttgcccagctgtacgacgcactgcacaaaccggagatgcaggactctctgtgttggaacaccgacaccggcagcggcggctatacccataaattcttccgcgtggcgaaaagtgccgacgacctgcgccagcaacgcgacgccatcgctgagtggtcacgcctgagctatggctggatgggccgtaccccagactacaaagccgctttcggttgcgcactgggcgcgaatccgggcttttacggtcagttcgagcagaacgcccgtaactggtacacccgtattcaggaaactggcctctactttaaccacgcgattgttaacccaccgatcgatcgtcatttgccgaccgataaagtgaaagacgtttacatcaagctggaaaaagagactgacgccgggattatcgtcagcggtgcgaaagtggttgccaccaactcggcgctgactcactacaacatgattggcttcggctcggcacaagtgatgggcgaaaacccggacttcgcactgatgttcgttgcgccaatggatgccgatggcgtgaaattaatctcccgcgcctcttatgagatggtcgcgggtgctaccggctcgccatacgactacccgctctccagccgcttcgatgagaacgatgcgattctggtgatggataacgtgctgattccatgggaaaacgtgctgatctaccgcgattttgatcgctgccgtcgctggacgatggaaggcggttttgcccgtatgtatccgctgcaagcctgtgtgcgcctggcagtgaaattagacttcattacggcactgctgaaaaaatcactcgaatgtaccggcaccctggagttccgtggtgtgcaggccgatctcggtgaagtggtagcgtggcgcaacaccttctgggcattgagtgactcgatgtgttcagaagcaacgccgtgggtcaacggggcttatttaccggatcatgccgcactgcaaacctatcgcgtactggcaccaatggcctacgcgaagatcaaaaacattatcgaacgcaacgttaccagtggcctgatctatctcccttccagtgcccgtgacctgaataatccgcagatcgaccagtatctggcgaagtatgtgcgcggttcgaacggtatggatcacgtccagcgcatcaagatcctcaaactgatgtgggatgctattggcagcgaatttggtggtcgtcacgaactgtatgaaatcaactactccggtagccaggatgagattcgcctgcagtgtctgcgccaggcacaaaactccggcaatatggacaagatgatggcgatggttgatcgctgcctgtcggaatacgaccaggacggctggactgtgccgcacctgcacaacaacgacgatatcaacatgctggataagctgctgaaataa

SEQ ID NO: 85 - protein, 171 aa, Escherichia coli
MSQLDEQRLRFRDAMASLSAAVNIITTEGDAGQCGITATAVCSVTDTPPSLMVCINANSAMNPVFQGNGKLCVNVLNHEQELMARHFAGMTGMAMEERFSLSCWQKGPLAQPVLKGSLASLEGEIRDVQAIGTHLVYLVEIKNIILSAEGHGLIYFKRRFHPVMLEMEAAI

SEQ ID NO: 86 - DNA, 516 bp, synthetic construct
  misc_feature 1..516: note = coding sequence for SEQ ID NO: 85
  source 1..516: mol_type = other DNA
atgtcccaattagatgaacaacgcctgcgctttcgtgacgcgatggccagcctgtcggcagcggtaaatattatcaccaccgagggcgacgccggacaatgcgggattacggcaacggccgtctgctcggtcacggatacaccaccgtcgctgatggtgtgcattaacgccaacagtgcgatgaacccggtttttcagggcaacggcaagttgtgcgtcaacgtcctcaaccatgagcaggaactgatggcacgccacttcgcgggcatgacaggcatggcgatggaagagcgttttagcctctcatgctggcaaaaaggtccgctggcgcagccggtgctaaaaggttcgctggccagtcttgaaggtgagatccgcgatgtgcaggcaattggcacacatctggtgtatctggtggagattaaaaacatcatcctcagtgcagaaggtcatggacttatctactttaaacgccgtttccatccggtgatgctggaaatggaagctgcgatttaa

SEQ ID NO: 87 - protein, 233 aa, Arabidopsis thaliana
MSAKDEAKGLLKSEELYKYILETSVYPREPEVLRELRNITHNHPQAGMATAPDAGQLMGMLLNLVNARKTIEVGVFTGYSLLLTALTLPEDGKVIAIDMNRDSYEIGLPVIKKAGVEHKIDFKESEALPALDELLNNKVNEGGFDFAFVDADKLNYWNYHERLIRLIKVGGIIVYDNTLWGGSVAEPDSSTPEWRIEVKKATLELNKKLSADQRVQISQAALGDGITICRRLY

SEQ ID NO: 88 - DNA, 702 bp, synthetic construct
  misc_feature 1..702: note = coding sequence for SEQ ID NO: 87
  source 1..702: mol_type = other DNA
atgtccgctaaggatgaagccaagggtttgttgaagtctgaagaattgtacaagtacatcttggaaacttctgtttacccaagagaaccagaagttttgagagaattgagaaacattactcacaaccacccacaagctggtatggctactgctccagacgctggtcaattgatgggtatgttgttgaacttggttaacgctagaaagactatcgaagttggtgttttcactggttactctttgttgttgactgctttgactttgccagaagatggtaaggttatcgccattgacatgaacagagactcttacgaaatcggtttgccagttatcaagaaggctggtgttgaacacaagattgatttcaaggaatctgaagctttgccagccttggacgaattgttgaacaacaaggttaacgaaggtggtttcgacttcgctttcgttgatgctgacaagttgaactactggaactaccacgaaagattgattagattgatcaaggttggtggtatcatcgtttacgataacactttgtggggtggttctgttgctgaaccagactcttccactccagaatggagaatcgaagtcaagaaggctaccttggaattgaacaagaagttgtctgctgatcaaagagttcaaatctctcaagctgctttgggtgatggtatcactatttgtagaagattgtactaa

SEQ ID NO: 89 - protein, 222 aa, Homo sapiens
MSGDTKEQRILNHVLQHAEPGNAQSVLEAIDTYCEQKEWAMNVGDKKGKIVDAVIQEHQPSVLLELGAYCGYSAVRMARLLSPGARLITIEINPDCAAITQRMVDFAGVKDKVTLVVGASQDIIPQLKKKYDVDTLDMVFLDHWKDRYLPDTLLLEECGLLRKGTVLLADNVICPGAPDFLAHVRGSSCFECTHYQSFLEYREVVDGLEKAIYKGPGSEAGP

SEQ ID NO: 90 - DNA, 669 bp, synthetic construct
  misc_feature 1..669: note = coding sequence for SEQ ID NO: 89
  source 1..669: mol_type = other DNA
atgtccggtgacaccaaggaacaaagaatcttgaaccacgttttgcaacacgctgaaccaggtaacgctcaatctgttttggaagccattgacacctactgtgaacaaaaggaatgggccatgaacgttggtgacaagaagggtaagatcgttgacgccgttattcaagaacaccaaccatccgttttgttggaattgggtgcctactgtggttactctgctgttagaatggccagattgttgtctccaggtgctagattgatcaccatcgaaatcaacccagactgtgccgccatcacccaaagaatggttgatttcgctggtgttaaggacaaggtcaccttggttgttggtgcttcccaagacatcatcccacaattgaagaagaagtacgatgttgacactttggacatggttttcttggaccactggaaggacagatacttgccagacactttgttgttggaagaatgtggtttgttgagaaagggtactgttttgttggctgacaacgttatctgtccaggtgctccagacttcttggctcacgttagaggttcttcttgtttcgaatgtactcactaccaatctttcttggaatacagagaagttgttgacggtttggaaaaggccatctacaagggtccaggttctgaagctggtccataa

SEQ ID NO: 91 - protein, 489 aa, Arabidopsis thaliana
MSGTPVEVSKLHFLLFPFMAHGHMIPTLDMAKLFATKGAKSTILTTPLNAKLFFEKPIKSFNQDNPGLEDITIQILNFPCTELGLPDGCENTDFIFSTPDLNVGDLSQKFLLAMKYFEEPLEELLVTMRPDCLVGNMFFPWSTKVAEKFGVPRLVFHGTGYFSLCASHCIRLPKNVATSSEPFVIPDLPGDILITEEQVMETEEESVMGRFMKAIRDSERDSFGVLVNSFYELEQAYSDYFKSFVAKRAWHIGPLSLGNRKFEEKAERGKKASIDEHECLKWLDSKKCDSVIYMAFGTMSSFKNEQLIEIAAGLDMSGHDFVWVVNRKGSQVEKEDWLPEGFEEKTKGKGLIIRGWAPQVLILEHKAIGGFLTHCGWNSLLEGVAAGLPMVTWPVGAEQFYNEKLVTQVLKTGVSVGVKKMMQVVGDFISREKVEGAVREVMVGEERRKRAKELAEMAKNAVKEGGSSDLEVDRLMEELTLVKLQKEKV

SEQ ID NO: 92 - DNA, 1470 bp, synthetic construct
  misc_feature 1..1470: note = coding sequence for SEQ ID NO: 91
  source 1..1470: mol_type = other DNA
atgtccggtactccagtcgaagtctctaagttgcacttcttgttgttcccattcatggctcacggtcacatgatcccaactttggacatggctaagttgttcgccaccaagggtgctaagtccactatcttgactactccattgaacgccaagttgttcttcgaaaagccaatcaagtctttcaaccaagacaacccaggtttggaagatatcaccatccaaatcttgaacttcccatgtactgaattgggtttgccagatggttgtgaaaacactgatttcatcttctccactccagacttgaacgttggtgacttgtctcaaaagttcttgttggctatgaagtacttcgaagaaccattggaagaattgttggttactatgaggccagactgtttggtcggtaacatgttcttcccatggtccactaaggttgctgaaaagttcggtgttccaagattggttttccacggtactggttacttctctttgtgtgcttctcactgtatcagattgccaaagaacgttgctacttcttctgaaccattcgttattccagatttgccaggtgacattttgattactgaagaacaagtcatggaaactgaagaagaatctgttatgggtagattcatgaaggctatcagagactctgaaagagattctttcggtgttttggttaactctttctacgaattggaacaagcttactctgattacttcaagtctttcgttgctaagagagcttggcacatcggtccattgtccttgggtaacagaaagttcgaagaaaaggctgaaagaggtaagaaggcttctattgatgaacacgaatgtttgaagtggttggactccaagaagtgtgattctgttatttacatggccttcggtaccatgtcctctttcaagaacgaacaattgatcgaaattgctgctggtttggatatgtctggtcacgatttcgtctgggttgttaacagaaagggttctcaagttgaaaaggaagattggttgccagaaggtttcgaagaaaagaccaagggtaagggtttgatcatcagaggttgggctccacaagttttgatcttggaacacaaggctattggtggtttcttgactcactgtggttggaactctttgttggaaggtgttgctgctggtttgccaatggttacttggccagttggtgccgaacaattctacaacgaaaagttggttactcaagttttaaagactggtgtttctgttggtgttaagaagatgatgcaagttgttggtgacttcatttctagagaaaaggttgaaggtgctgttagagaagttatggttggtgaagaaagaagaaagagagccaaggaattggctgaaatggctaagaacgctgttaaggaaggtggttcttctgatttggaagttgatagattgatggaagaattgactttggttaagttgcaaaaggaaaaggtttaa

SEQ ID NO: 93 - protein, 470 aa, Scutellaria baicalensis
MSGQLHIVLVPMIAHGHMIPMLDMAKLFSSRGVRTTIIATPAFAEPIRKARESGHDIGLTTTKFPPKGSSLPDNILSLDQVTHDLLPHFFRALELLQEPVEEIMEDLKPNCLVSDMFLPWTTDSAAKFGIPRLLFHGTSLFARCFAEQMSLQKPYKNVSSDSEPFVLRGLPHEVSFVRTQIPGYELQEGGDDAFSKMAKQMRDADKKSYGDVINSFEELESEYVDHYKNVFGKKAWHIGPLSLCNNRAEQKSPIDDHECLAWLNSKKPNSVVYMCFGSMATFSPAQLHETAVGLESSGQDFIWVVRNGGENEDWLPQGFEERIKGKGLMIRGWAPQVVILDHPSTGAFVTHCGWNSTLEGICAGLPMVTWPVFAEQFYNEKLVTEVLKTGVSVGNKKWQRVGEWVGSEAVKEAVEWVMVGDGAAEMRSRANYYKEMARKAVEDGGSSYNNLNALIQELSAYVPPMKQGLN

SEQ ID NO: 94 - DNA, 1413 bp, synthetic construct
  misc_feature 1..1413: note = coding sequence for SEQ ID NO: 93
  source 1..1413: mol_type = other DNA
atgtccggtcaattgcacatcgtcttggttccaatgatcgctcacggtcacatgatcccaatgttggacatggccaagttgttctcttccagaggtgtcagaaccaccatcatcgccactccagccttcgctgaaccaatcagaaaggccagagaatctggtcacgacatcggtttgaccaccaccaagttcccaccaaagggttcctctttgccagacaacatcttgtctttggatcaagttactcacgatttgttgccacacttcttcagagccttggaattgttgcaagaaccagttgaagaaatcatggaagatttgaagccaaactgtttggtttctgacatgttcttgccatggactaccgactctgctgccaagttcggtattccaagattgttgttccacggtacctctttgttcgctagatgtttcgctgaacaaatgtctttgcaaaagccatacaagaacgtttcttctgattccgaaccattcgttttgagaggtttgccacacgaagtttctttcgttagaactcaaatcccaggttacgaattgcaagaaggtggtgatgatgccttctctaagatggctaagcaaatgagagatgctgataagaagtcttacggtgacgttatcaactctttcgaagaattggaatctgaatacgttgatcactacaagaacgttttcggtaagaaggcttggcacatcggtccattgtctttgtgtaacaacagagctgaacaaaagtctccaatcgacgatcacgaatgtttggcttggttgaactctaagaagccaaactctgttgtttacatgtgtttcggttctatggccactttctctccagcccaattgcacgaaactgctgtcggtttggaatcctctggtcaagatttcatctgggttgttagaaacggtggtgaaaacgaagattggttgccacaaggtttcgaagaaagaatcaagggtaagggtttgatgatcagaggttgggccccacaagttgttattttggatcacccatctactggtgctttcgttactcactgtggttggaactctactttggaaggtatctgtgctggtttgccaatggttacttggccagttttcgctgaacaattctacaacgaaaagttggttactgaagttttaaagactggtgtttctgttggtaacaagaagtggcaaagagttggtgaatgggttggttctgaagccgttaaggaagctgttgaatgggtcatggtcggtgacggtgctgctgaaatgagatctagagctaactactacaaggaaatggctagaaaggctgttgaagatggtggttcttcttacaacaacttgaacgctttgatccaagaattgtctgcctacgtcccaccaatgaagcaaggtttgaactaa

SEQ ID NO: 95 - protein, 470 aa, Scutellaria baicalensis
MSGQLHIVLVPMIAHGHMIPMLDMAKLFSSRGVKTTIIATPAFAEPIRKARESGHDIGLTTTKFPPKGSSLPDNIWSLDQVTDDLLPHFFRALELLQEPVEEIMEDLKPNCLVSDMFLPWTTDSAAKFGIPRLLFHGTSLFARCFAEQMSLQKPYKNVSSDSEPFVLRGLPHEVSFVRTQIPGYELQEGGDDAFSKMAKQMRDADKKSYGDVINNFEELESEYVDHYKNVFGKKAWHIGPLSLCNNRAEQKSPIDDHECLAWLNSKKPNSVVYMCFGSMATFTPAQLHETAVGLESSGQDFIWVVRNGGENEDWLPQGFEERIKGKGLMIRGWAPQVMILDHPSTGAFVTHCGWNSTLEGICAGLPMVTWPVFAEQFYNEKLVTEVLKTGVSVGNKKWQRVGEGVGSEAVKEAVERVMVGDGAAEMRSRAIYYKEMARKAVEEGGSSYNNLNALIEELSAYVPPMKQGLN

SEQ ID NO: 96 - DNA, 1413 bp, synthetic construct
  misc_feature 1..1413: note = coding sequence for SEQ ID NO: 95
  source 1..1413: mol_type = other DNA
atgtccggtcaattgcacatcgtcttggttccaatgatcgctcacggtcacatgatcccaatgttggacatggccaagttgttctcttccagaggtgtcaagaccaccatcatcgccactccagccttcgctgaaccaatcagaaaggccagagaatctggtcacgacatcggtttgaccaccaccaagttcccaccaaagggttcctctttgccagacaacatctggtctttggatcaagttactgatgatttgttgccacacttcttcagagccttggaattgttgcaagaaccagttgaagaaatcatggaagatttgaagccaaactgtttggtttctgacatgttcttgccatggactaccgactctgctgccaagttcggtattccaagattgttgttccacggtacctctttgttcgctagatgtttcgctgaacaaatgtctttgcaaaagccatacaagaacgtttcttctgattccgaaccattcgttttgagaggtttgccacacgaagtttctttcgttagaactcaaatcccaggttacgaattgcaagaaggtggtgatgatgccttctctaagatggctaagcaaatgagagatgctgataagaagtcttacggtgacgttatcaacaacttcgaagaattggaatctgaatacgttgatcactacaagaacgttttcggtaagaaggcttggcacatcggtccattgtctttgtgtaacaacagagctgaacaaaagtctccaatcgacgatcacgaatgtttggcttggttgaactctaagaagccaaactctgttgtttacatgtgtttcggttctatggccactttcactccagcccaattgcacgaaactgctgtcggtttggaatcctctggtcaagatttcatctgggttgttagaaacggtggtgaaaacgaagattggttgccacaaggtttcgaagaaagaatcaagggtaagggtttgatgatcagaggttgggccccacaagttatgattttggatcacccatctactggtgctttcgttactcactgtggttggaactctactttggaaggtatctgtgctggtttgccaatggttacttggccagttttcgctgaacaattctacaacgaaaagttggttactgaagttttaaagactggtgtttctgttggtaacaagaagtggcaaagagttggtgaaggtgttggttctgaagctgttaaggaagctgttgaaagagtcatggtcggtgacggtgctgctgaaatgagatctagagctatctactacaaggaaatggctagaaaggctgttgaagaaggtggttcttcttacaacaacttgaacgctttgatcgaagaattgtctgcctacgtcccaccaatgaagcaaggtttgaactaa

SEQ ID NO: 97 - protein, 477 aa, Scutellaria baicalensis
MSGQLHIVLVPMIAHGHMIPMLDMAKLFSSRGVKTTIIATPAFAEPIRKARESGHDIGLTTTKFPPKGSSLPDNIRSLDQVTGDLLPHFFRALELLQEPVEEIMEDLKPNCLVSDMFLPWTTDSAAKFGIPRLVFHGTSLFARCFSEQMSIQKPYKNVSSDSEPFVLRGLPHEVSFVRTQIPDYELQEGGDDAFSKMAKQMRDADKKSYGDVINSFEELESEYADYNKNVFGKKAWHIGPLKLFNNRAEQKSSQRGKESAIDDHECLAWLNSKKPNSVVYMCFGSMATFTPAQLHETAVGLESSGQDFIWVVRNGGKNEDWLPQGFEERIKGKGLMIRGWAPQVMILDHPSTGAFVTHCGWNSTLEGICAGLPMVTWPVFAEQFYNEKLVTEVLKTGVSVGNKKWQRVGEGVGSEAVKEAVERVMVGDGAAEMRSRAVYYKEMARKAVEEGGSSYNNLNALIQELSAYVPPMKQGLN

SEQ ID NO: 98 - DNA, 1434 bp, synthetic construct
  misc_feature 1..1434: note = coding sequence for SEQ ID NO:97
  source 1..1434: mol_type = other DNA
atgtccggtcaattgcacatcgtcttggttccaatgatcgctcacggtcacatgatcccaatgttggacatggccaagttgttctcttccagaggtgtcaagaccaccatcatcgccactccagccttcgctgaaccaatcagaaaggccagagaatctggtcacgacatcggtttgaccaccaccaagttcccaccaaagggttcctctttgccagacaacatcagatctttggatcaagttactggtgatttgttgccacacttcttcagagccttggaattgttgcaagaaccagttgaagaaatcatggaagatttgaagccaaactgtttggtttctgacatgttcttgccatggactaccgactctgctgccaagttcggtattccaagattggttttccacggtacctctttgttcgctagatgtttctctgaacaaatgtctatccaaaagccatacaagaacgtttcttctgattccgaaccattcgttttgagaggtttgccacacgaagtttctttcgttagaactcaaatcccagattacgaattgcaagaaggtggtgatgatgccttctctaagatggctaagcaaatgagagatgctgataagaagtcttacggtgacgttatcaactctttcgaagaattggaatctgaatacgctgattacaacaagaacgttttcggtaagaaggcttggcacatcggtccattgaagttgttcaacaacagagctgaacaaaagtcatctcaaagaggtaaggaatctgccatcgacgatcacgaatgtttggcttggttgaactctaagaagccaaactctgttgtttacatgtgtttcggttctatggccactttcactccagcccaattgcacgaaactgctgtcggtttggaatcctctggtcaagatttcatctgggttgttagaaacggtggtaagaacgaagattggttgccacaaggtttcgaagaaagaatcaagggtaagggtttgatgatcagaggttgggccccacaagttatgattttggatcacccatctactggtgctttcgttactcactgtggttggaactctactttggaaggtatctgtgccggtttgccaatggttacttggccagttttcgctgaacaattctacaacgaaaagttggttactgaagttttaaagactggtgtttctgttggtaacaagaagtggcaaagagttggtgaaggtgttggttctgaagctgttaaggaagctgttgaaagagtcatggtcggtgacggtgctgctgaaatgagatctagagctgtctactacaaggaaatggctagaaaggctgttgaagaaggtggttcttcttacaacaacttgaacgctttgatccaagaattgtctgcctacgttccaccaatgaagcaaggtttgaactaa

SEQ ID NO: 99 - protein, 533 aa, Homo sapiens
MSACLLRSFQRISAGVFFLALWGMVVGDKLLVVPQDGSHWLSMKDIVEVLSDRGHEIVVVVPEVNLLLKESKYYTRKIYPVPYDQEELKNRYQSFGNNHFAERSFLTAPQTEYRNNMIVIGLYFINCQSLLQDRDTLNFFKESKFDALFTDPALPCGVILAEYLGLPSVYLFRGFPCSLEHTFSRSPDPVSYIPRCYTKFSDHMTFSQRVANFLVNLLEPYLFYCLFSKYEELASAVLKRDVDIITLYQKVSVWLLRYDFVLEYPRPVMPNMVFIGGINCKKRKDLSQEFEAYINASGEHGIVVFSLGSMVSEIPEKKAMAIADALGKIPQTVLWRYTGTRPSNLANNTILVKWLPQNDLLGHPMTRAFITHAGSHGVYESICNGVPMVMMPLFGDQMDNAKRMETKGAGVTLNVLEMTSEDLENALKAVINDKSYKENIMRLSSLHKDRPVEPLDLAVFWVEFVMRHKGAPHLRPAAHDLTWYQYHSLDVIGFLLAVVLTVAFITFKCCAYGYRKCLGKKGRVKKAHKSKTH

SEQ ID NO: 100 - DNA, 1602 bp, synthetic construct
  misc_feature 1..1602: note = coding sequence for SEQ ID NO: 99
  source 1..1602: mol_type = other DNA
atgtccgcctgtttgttgagatctttccaaagaatttctgctggtgttttcttcttggctttgtggggtatggttgttggtgacaagttgttggttgtcccacaagacggttctcactggttgtctatgaaggatatcgttgaagttttgtctgacagaggtcacgaaattgttgttgttgttccagaagttaacttgttgttgaaggaatccaagtactacactagaaagatctacccagttccatacgaccaagaagaattgaagaacagataccaatctttcggtaacaaccacttcgctgaaagatctttcttgactgctccacaaactgaatacagaaacaacatgattgttattggtttgtacttcatcaactgtcaatctttgttgcaagacagagacaccttgaacttcttcaaggaatctaagttcgatgctttgttcactgacccagccttgccatgtggtgttatcttggctgaatacttgggtttgccatctgtttacttgttcagaggtttcccatgttccttggaacacactttctctagatctccagacccagtttcctacattccaagatgttacactaagttctctgaccacatgactttctcccaaagagttgccaacttcttggttaacttgttggaaccatacttgttctactgtttgttctctaagtacgaagaattggcttctgctgtcttgaagagagatgttgatatcatcaccttgtaccaaaaggtatctgtttggttgttgagatacgacttcgttttggaatacccaagaccagtcatgccaaacatggtattcattggtggtatcaactgtaagaagagaaaggacttgtctcaagaattcgaagcctacattaacgcttctggtgaacacggtattgttgttttctctttgggttctatggtatctgaaattccagaaaagaaggctatggctattgctgatgctttgggtaagatcccacaaactgtcttgtggagatacactggtaccagaccatctaacttggctaacaacactatcttggttaagtggttgccacaaaacgatttgttgggtcacccaatgaccagagccttcatcacccacgctggttcccacggtgtttacgaatctatctgtaacggtgttccaatggttatgatgccattgttcggtgatcaaatggacaacgctaagagaatggaaactaagggtgctggtgttaccttgaacgttttggaaatgacttctgaagatttggaaaacgctttgaaggctgtcatcaacgacaagtcttacaaggaaaacatcatgagattgtcctctttgcacaaggacagaccagttgaaccattggacttggccgttttctgggttgaattcgttatgagacacaagggtgctccacacttaagaccagctgcccacgacttgacctggtaccaataccactccttggacgttattggtttcttgttggccgtcgttttgactgttgccttcatcaccttcaagtgttgtgcttacggttacagaaagtgtttgggtaagaagggtagagttaagaaggcccacaagtccaagacccactaa

SEQ ID NO: 101 - protein, 531 aa, Homo sapiens
MVARAGWTGLLPLYVCLLLTCGFAKAGKLLVVPMDGSHWFTMQSVVEKLILRGHEVVVVMPEVSWQLGRSLNCTVKTYSTSYTLEDQDREFMVFADARWTAPLRSAFSLLTSSSNGIFDLFFSNCRSLFNDRKLVEYLKESCFDAVFLDPFDACGLIVAKYFSLPSVVFARGIFCHYLEEGAQCPAPLSYVPRLLLGFSDAMTFKERVWNHIMHLEEHLFCPYFFKNVLEIASEILQTPVTAYDLYSHTSIWLLRTDFVLEYPKPVMPNMIFIGGINCHQGKPVPMEFEAYINASGEHGIVVFSLGSMVSEIPEKKAMAIADALGKIPQTVLWRYTGTRPSNLANNTILVKWLPQNDLLGHPMTRAFITHAGSHGVYESICNGVPMVMMPLFGDQMDNAKRMETKGAGVTLNVLEMTSEDLENALKAVINDKSYKENIMRLSSLHKDRPVEPLDLAVFWVEFVMRHKGAPHLRPAAHDLTWYQYHSLDVIGFLLAVVLTVAFITFKCCAYGYRKCLGKKGRVKKAHKSKTH

SEQ ID NO: 102 - DNA, 1596 bp, synthetic construct
  misc_feature 1..1596: note = coding sequence for SEQ ID NO: 101
  source 1..1596: mol_type = other DNA
atgtccgctagagctggttggactggtttgttgccattgtacgtttgtttgttgttgacctgtggtttcgccaaggctggtaagttgttggttgttccaatggatggttctcactggttcaccatgcaatctgttgttgaaaagttgatcttgagaggtcacgaagttgtcgttgtcatgccagaagtttcttggcaattgggtagatctttgaactgtactgttaagacttactctacctcttacactttggaagatcaagacagagaattcatggttttcgccgatgctagatggactgctccattgagatctgctttctctttgttgacttcttcttccaacggtattttcgacttgttcttctctaactgtagatctttgttcaacgacagaaagttggttgaatacttgaaggaatcttgtttcgatgctgttttcttggatccattcgatgcctgtggtttgattgttgccaagtacttctccttgccatctgttgtattcgccagaggtatcttctgtcactacttggaagaaggtgctcaatgtccagctccattgtcctacgtcccaagattgttgttgggtttctctgacgccatgactttcaaggaaagagtttggaaccacatcatgcacttggaagaacacttgttctgtccatacttcttcaagaacgtcttggaaatcgcctctgaaattttgcaaaccccagtcactgcttacgatttgtactctcacacttctatttggttgttgagaactgacttcgttttggaatacccaaagccagttatgccaaacatgatcttcattggtggtatcaactgtcaccaaggtaagccagttccaatggaattcgaagcctacattaacgcttctggtgaacacggtattgttgttttctctttgggttctatggtatctgaaattccagaaaagaaggctatggctattgctgatgctttgggtaagatcccacaaactgtcttgtggagatacactggtaccagaccatctaacttggctaacaacactatcttggttaagtggttgccacaaaacgatttgttgggtcacccaatgaccagagccttcatcacccacgctggttcccacggtgtttacgaatctatctgtaacggtgttccaatggttatgatgccattgttcggtgatcaaatggacaacgctaagagaatggaaactaagggtgctggtgttaccttgaacgttttggaaatgacttctgaagatttggaaaacgctttgaaggctgtcatcaacgacaagtcttacaaggaaaacatcatgagattgtcctctttgcacaaggacagaccagttgaaccattggacttggccgttttctgggttgaattcgttatgagacacaagggtgctccacacttaagaccagctgcccacgacttgacctggtaccaataccactccttggacgttattggtttcttgttggccgtcgttttgactgttgccttcatcaccttcaagtgttgtgcttacggttacagaaagtgtttgggtaagaagggtagagttaagaaggcccacaagtccaagacccactaa

SEQ ID NO: 103 - protein, 482 aa, Citrus clementina
MSHASSNMHAPSNQHHKMGTESAEADQLHVVMFPWFAFGHISPFVQLSNKLSLHGVKVSFFSAPGNIPRIKSSLNLTPMADIIPLQIPHVDGLPPGLDSTSEMTPHMAELLKQALDLMQPQIKTLLSQLKPHFVFFDFTHYWLPGLVGSQLGIKTVNFSVFSAISQAYLVVPARKLNNSLADLMKSPDGFPATSITSLDEFVARDYLYVYTKFNGGPSVYERGIQGVDGCDVLAIKTCNEMEGPYLDFVRTQFKKPVLLTGPLVNPEPPSGELEERWAKWLCKYPPKSVIYCSFGSETFLTVDQIKELAIGLEITGLPFFLVLNFPPNVDAQSELVRTLPPGFMDRVKDRGVVHTGWVQQQLILRHESVGCYVCHSGFSSVTEAVISDCQLVLLPLKGDQFLNSKLVAGDLKAGVEVNRRDHDGHFGKEDIFKAVKTVMVDVNKEPGASIRANQKWWREFLLNGQIQDKFIADFVKDLKTLA

SEQ ID NO: 104 - DNA, 1449 bp, synthetic construct
  misc_feature 1..1449: note = coding sequence for SEQ ID NO: 103
  source 1..1449: mol_type = other DNA
atgtcccacgcctcttctaacatgcacgccccatctaaccaacaccacaagatgggtactgaatctgctgaagctgatcaattgcacgttgttatgttcccatggttcgctttcggtcacatctctccattcgttcaattgtccaacaagttgtctttgcacggtgtcaaggtatctttcttctctgctccaggtaacattccaagaatcaagtcatctttgaacttgactccaatggccgacattatcccattgcaaatcccacacgtcgatggtttgccaccaggtttggattctacctctgaaatgactccacacatggctgaattgttgaagcaagccttggacttgatgcaaccacaaatcaagactttgttgtctcaattgaagccacacttcgttttcttcgatttcacccactactggttgccaggtttggttggttctcaattgggtatcaagactgttaacttctctgttttctctgccatttctcaagcctacttggttgttccagccagaaagttgaacaactctttggctgatttgatgaagtctccagatggtttcccagctacttccattacctctttggatgaattcgttgctagagattacttgtacgtttacactaagttcaacggtggtccatctgtttacgaaagaggtattcaaggtgttgatggttgtgatgttttggccatcaagacttgtaacgaaatggaaggtccatacttggatttcgttagaacccaattcaagaagccagttttgttgaccggtccattggttaacccagaaccaccatccggtgaattggaagaaagatgggctaagtggttgtgtaagtacccaccaaagtctgttatttactgttccttcggttctgaaactttcttgaccgttgatcaaatcaaggaattggccattggtttggaaattactggtttgccattcttcttggtcttgaacttcccaccaaacgtcgatgctcaatctgaattggtcagaactttgccaccaggtttcatggacagagttaaggacagaggtgttgttcacactggttgggttcaacaacaattgattttgagacacgaatctgttggttgttacgtttgtcactctggtttctcttctgttactgaagctgttatttctgattgtcaattggttttgttgccattgaagggtgaccaattcttgaactccaagttggttgctggtgacttgaaggctggtgttgaagttaacagaagagatcacgatggtcacttcggtaaggaagatattttcaaggctgttaagactgttatggttgatgttaacaaggaaccaggtgcttctatcagagctaaccaaaagtggtggagagaattcttgttgaacggtcaaattcaagataagttcattgctgatttcgtcaaggatttaaagaccttggcttaa

SEQ ID NO: 105 - protein, 476 aa, Citrus sinensis
MSHAPSNQHHKMGTESAEADQLHVVMFPWFASGHISPFVQLSNKLSLHGVKVSFFSAPGNIPRIKSSLNLTPMADIIPLQIPHVDGLPPGLDSTSEMTPHMAELLKQALDLMQPQIKTLLSQLKPHFVFFDFTHYWLPGLVGSQLGIKTVNFSVFSAISQAYLVVPARKLNNSLADLMKSPDGFPATSITSLDEFVARDYLYVYTKFNGGPSVYERGIQGVDGCDVLAIKTCNEMEGPYLDFVRTQFKKPVLLTGPLVNPEPPSGELEERWANWLGKFPPKSVIYCSFGSETFLTVDQIKELAIGLEITGLPFFLVLNFPPNVDGQSELVRTLPPGFMDRVKDRGVVHTGWVQQQLILRHESVGCYVCHSGFSSVTEAVISDCQLVLLPLKGDQFLNSKLVAGDLKAGVEVNRRDHDGHFGKEDIFKAVKTVMVDVNKEPGASIRANQKWWREFLLNGQIQDKFIADFVKDLKALA

SEQ ID NO: 106 - DNA, 1431 bp, synthetic construct
  misc_feature 1..1431: note = coding sequence for SEQ ID NO: 105
  source 1..1431: mol_type = other DNA
atgtcccacgccccatctaaccaacaccacaagatgggtactgaatctgctgaagctgatcaattgcacgttgttatgttcccatggttcgcttctggtcacatctctccattcgttcaattgtccaacaagttgtctttgcacggtgtcaaggtatctttcttctctgctccaggtaacattccaagaatcaagtcatctttgaacttgactccaatggccgacattatcccattgcaaatcccacacgtcgatggtttgccaccaggtttggattctacctctgaaatgactccacacatggctgaattgttgaagcaagccttggacttgatgcaaccacaaatcaagactttgttgtctcaattgaagccacacttcgttttcttcgatttcacccactactggttgccaggtttggttggttctcaattgggtatcaagaccgttaacttctctgttttctctgccatttctcaagcctacttggttgtcccagccagaaagttgaacaactctttggctgatttgatgaagtctccagatggtttcccagctacttccattacctctttggatgaattcgttgctagagattacttgtacgtttacactaagttcaacggtggtccatctgtttacgaaagaggtattcaaggtgttgatggttgtgatgttttggccatcaagacttgtaacgaaatggaaggtccatacttggatttcgttagaacccaattcaagaagccagttttgttgaccggtccattggttaacccagaaccaccatccggtgaattggaagaaagatgggctaactggttgggtaagttcccaccaaagtctgtcatttactgttctttcggttctgaaactttcttgaccgttgatcaaatcaaggaattggccattggtttggaaattactggtttgccattcttcttggtcttgaacttcccaccaaacgttgatggtcaatctgaattggtcagaactttgccaccaggtttcatggacagagttaaggacagaggtgttgttcacactggttgggttcaacaacaattgattttgagacacgaatctgttggttgttacgtttgtcactctggtttctcttctgttactgaagctgttatttctgattgtcaattggttttgttgccattgaagggtgaccaattcttgaactccaagttggttgctggtgacttgaaggctggtgttgaagttaacagaagagatcacgatggtcacttcggtaaggaagatattttcaaggctgttaagactgttatggttgatgttaacaaggaaccaggtgcttctatcagagctaaccaaaagtggtggagagaattcttgttgaacggtcaaattcaagataagttcattgctgatttcgtcaaggatttgaaggccttggcttaa

SEQ ID NO: 107 - protein, 669 aa, Citrus sinensis
MSATYTPKNILITGAAGFIASHVCNRLIRNYPEYKIVVLDKLDYCSNLKNLIPSKASSNFKFVKGDIASADLVNFLLITESIDTIMHFAAQTHVDNSFGNSFEFTKNNIYGTHVLLEACKVTGQIRRFIHVSTDEVYGETDEDAVVGNHEASQLLPTNPYSATKAGAEMLVMAYGRSYGLPVITTRGNNVYGPNQFPEKLIPKFILLAMRGLPLPIHGDGSNVRSYLYCEDVAEAFECILHKGEVGHVYNVGTKKERRVIDVAKDICKLFSMDPETSIKFVENRPFNDQRYFLDDQKLTSLGWSERTIWEEGLRKTIEWYTQNPDWWGDVSGALLPHPRMLMMPGGRHFDGSEENKAVSSVSTNNIQSRMVVPVSKCSSPRKPSMKFLIYGRTGWIGGLLGKLCEKEGIPFEYGKGRLEDRSSLIADVQSVKPTHVFNAAGVTGRPNVDWCESHKTDTIRTNVAGTLTLADVCRDHGILMMNYATGCIFEYDAAHPEGSGIGYKEEDTPNFTGSFYSKTKAMVEELLKEYDNVCTLRVRMPISSDLNNPRNFITKISRYNKVVNIPNSMTVLDELLPISIEMAKRNLRGIWNFTNPGVVSHNEILEMYKKYINPEFKWVNFTLDEQAKVIVAPRSNNEMDASKLKKEFPELLSIKDSLIKYVFEPNKKT

SEQ ID NO: 108 - DNA, 2010 bp, synthetic construct
  misc_feature 1..2010: note = coding sequence for SEQ ID NO: 107
  source 1..2010: mol_type = other DNA
atgtccgctacttacaccccaaagaacatcttgatcactggtgctgctggtttcattgcttcccacgtttgtaacagattgattagaaactacccagaatacaagattgttgttttggacaagttggattactgttctaacttgaagaacttgatcccatccaaggcttcctccaacttcaagttcgttaagggtgacattgcctctgctgacttggtcaacttcttgttgatcaccgaatccattgacactattatgcacttcgctgctcaaactcacgttgacaactctttcggtaactctttcgaattcaccaagaacaacatctacggtactcacgtcttgttggaagcctgtaaggttactggtcaaatcagaagattcatccacgtatctactgatgaagtctacggtgaaactgatgaagatgctgttgttggtaaccacgaagcttcccaattgttgccaactaacccatactctgctactaaggctggtgctgaaatgttggttatggcttacggtagatcttacggtttgccagttattactaccagaggtaacaacgtttacggtccaaaccaattcccagaaaagttgattccaaagttcatcttgttggccatgagaggtttgccattgccaattcacggtgatggttctaacgttagatcttacttgtactgtgaagatgttgctgaagctttcgaatgtattttgcacaagggtgaagttggtcacgtttacaacgttggtactaagaaggaaagaagagttattgatgttgctaaggatatttgtaagttgttctctatggacccagaaacttctatcaagttcgttgaaaacagaccattcaacgatcaaagatacttcttggatgatcaaaagttgacttctttgggttggtctgaaagaaccatctgggaagaaggtttgagaaagactattgaatggtacactcaaaacccagattggtggggtgatgtctctggtgctttgttgccacacccaagaatgttgatgatgccaggtggtagacacttcgatggttctgaagaaaacaaggctgtttcctctgtctctactaacaacattcaatccagaatggttgttccagtttccaagtgttcttctccaagaaagccatccatgaagttcttgatctacggtagaactggttggattggtggtttgttgggtaagttgtgtgaaaaggaaggtattccattcgaatacggtaagggtagattggaagatagatcttctttgattgctgatgttcaatctgttaagccaactcacgttttcaacgctgctggtgttactggtagaccaaacgttgattggtgtgaatctcacaagactgacaccattagaaccaacgttgctggtaccttgaccttggctgatgtttgtagagatcacggtatcttgatgatgaactacgctaccggttgtatcttcgaatacgatgctgctcacccagaaggttctggtattggttacaaggaagaagatactccaaacttcactggttctttctactctaagaccaaggccatggttgaagaattgttgaaggaatacgacaacgtttgtactttgagagtcagaatgccaatctcttctgacttgaacaacccaagaaacttcattaccaagatttctagatacaacaaggttgtcaacattccaaactctatgactgttttggatgaattgttgccaatttctattgaaatggctaagagaaacttgagaggtatttggaacttcactaacccaggtgttgtttctcacaacgaaattttggaaatgtacaagaagtacattaacccagaattcaagtgggttaacttcactttggatgaacaagctaaggttatcgtcgccccaagatctaacaacgaaatggatgcttctaagttgaagaaggaattcccagaattgttgtccatcaaggattctttgatcaagtacgtattcgaaccaaacaagaaaacctaa

SEQ ID NO: 109 - protein, 670 aa, Arabidopsis thaliana
MSASYTPKNILITGAAGFIASHVANRLIRSYPDYKIVVLDKLDYCSNLKNLNPSKHSPNFKFVKGDIASADLVNHLLITEGIDTIMHFAAQTHVDNSFGNSFEFTKNNIYGTHVLLEACKVTGQIRRFIHVSTDEVYGETDEDALVGNHEASQLLPTNPYSATKAGAEMLVMAYGRSYGLPVITTRGNNVYGPNQFPEKLIPKFILLAMRGQVLPIHGDGSNVRSYLYCEDVAEAFEVVLHKGEVGHVYNIGTKKERRVNDVAKDICKLFNMDPEANIKFVDNRPFNDQRYFLDDQKLKKLGWSERTTWEEGLKKTMDWYTQNPEWWGDVSGALLPHPRMLMMPGGRHFDGSEDNSLAATLSEKPSQTHMVVPSQRSNGTPQKPSLKFLIYGKTGWIGGLLGKICDKQGIAYEYGKGRLEDRSSLLQDIQSVKPTHVFNSAGVTGRPNVDWCESHKTETIRANVAGTLTLADVCREHGLLMMNFATGCIFEYDDKHPEGSGIGFKEEDTPNFTGSFYSKTKAMVEELLKEYDNVCTLRVRMPISSDLNNPRNFITKISRYNKVVNIPNSMTVLDELLPISIEMAKRNLKGIWNFTNPGVVSHNEILEMYRDYINPEFKWANFTLEEQAKVIVAPRSNNEMDASKLKKEFPELLSIKESLIKYAYGPNKKT

SEQ ID NO: 110 - DNA, 2013 bp, synthetic construct
  misc_feature 1..2013: note = coding sequence for SEQ ID NO: 109
  source 1..2013: mol_type = other DNA
atgtccgcttcttacactccaaagaacattttgatcaccggtgctgctggtttcattgcttctcacgtcgccaacagattgatcagatcttacccagattacaagatcgttgttttggacaagttggattactgttctaacttgaagaacttgaacccatctaagcactctccaaacttcaagttcgtcaagggtgatatcgcttctgctgacttggttaaccacttgttgatcactgaaggtattgacaccatcatgcacttcgctgctcaaactcacgtcgacaactccttcggtaactctttcgaattcactaagaacaacatctacggtactcacgtcttgttggaagcttgtaaggttactggtcaaattagaagattcattcacgtttctactgatgaagtttacggtgaaactgatgaagatgctttggttggtaaccacgaagcttctcaattgttgccaactaacccatactctgccactaaggctggtgctgaaatgttggttatggcttacggtagatcttacggtttgccagttattaccactagaggtaacaacgtctacggtccaaaccaattcccagaaaagttgattccaaagttcattttgttggctatgagaggtcaagttttgccaattcacggtgatggttctaacgtcagatcttacttgtactgtgaggacgttgctgaagctttcgaagttgttttgcacaagggtgaagttggtcacgtttacaacattggtactaagaaggaaagaagagttaacgatgttgccaaggacatctgtaagttgttcaacatggacccagaagctaacatcaagttcgtcgacaacagaccattcaacgatcaaagatacttcttggacgatcaaaagttgaagaagttgggttggtctgaaagaaccacttgggaagaaggtttgaagaaaactatggattggtacactcaaaacccagaatggtggggtgatgtttctggtgctttgttgccacacccaagaatgttgatgatgccaggtggtagacacttcgatggttccgaagataactctttggctgctactttgtctgaaaagccatctcaaacccacatggttgttccatctcaaagatctaacggtactccacaaaagccatctttgaagttcttgatctacggtaagaccggttggatcggtggtttgttgggtaagatctgtgataagcaaggtattgcttacgaatacggtaagggtagattggaagatagatcttctttgttgcaagatattcaatctgttaagccaacccacgttttcaactccgctggtgttactggtagaccaaacgttgactggtgtgaatctcacaagaccgaaactatcagagccaacgttgctggtactttgactttggctgatgtctgtagagaacacggtttgttgatgatgaacttcgctactggttgtatcttcgaatacgacgacaagcacccagaaggttctggtattggtttcaaggaagaagatactccaaacttcactggttctttctactctaagaccaaggccatggtcgaagaattgttgaaggaatacgacaacgtttgtactttgagagttagaatgccaatctcctctgatttgaacaacccaagaaacttcatcaccaagatctccagatacaacaaggttgttaacatcccaaactctatgactgttttggacgaattgttgccaatctccatcgaaatggctaagagaaacttgaagggtatctggaacttcactaacccaggtgttgtttctcacaacgaaatcttggaaatgtacagagactacatcaacccagaattcaagtgggctaacttcactttggaagaacaagctaaggtcattgttgctccaagatctaacaacgaaatggatgcttccaagttgaagaaggaattcccagaattgttgtctatcaaggaatctttgattaagtacgcttacggtccaaacaagaaaacctaa

SEQ ID NO: 111 - protein, 668 aa, Arabidopsis thaliana
MSDDTTYKPKNILITGAAGFIASHVANRLIRNYPDYKIVVLDKLDYCSDLKNLDPSFSSPNFKFVKGDIASDDLVNYLLITENIDTIMHFAAQTHVDNSFGNSFEFTKNNIYGTHVLLEACKVTGQIRRFIHVSTDEVYGETDEDAAVGNHEASQLLPTNPYSATKAGAEMLVMAYGRSYGLPVITTRGNNVYGPNQFPEKMIPKFILLAMSGKPLPIHGDGSNVRSYLYCEDVAEAFEVVLHKGEIGHVYNVGTKRERRVIDVARDICKLFGKDPESSIQFVENRPFNDQRYFLDDQKLKKLGWQERTNWEDGLKKTMDWYTQNPEWWGDVSGALLPHPRMLMMPGGRLSDGSSEKKDVSSNTVQTFTVVTPKNGDSGDKASLKFLIYGKTGWLGGLLGKLCEKQGITYEYGKGRLEDRASLVADIRSIKPTHVFNAAGLTGRPNVDWCESHKPETIRVNVAGTLTLADVCRENDLLMMNFATGCIFEYDATHPEGSGIGFKEEDKPNFFGSFYSKTKAMVEELLREFDNVCTLRVRMPISSDLNNPRNFITKISRYNKVVDIPNSMTVLDELLPISIEMAKRNLRGIWNFTNPGVVSHNEILEMYKNYIEPGFKWSNFTVEEQAKVIVAARSNNEMDGSKLSKEFPEMLSIKESLLKYVFEPNKRT

SEQ ID NO: 112 - DNA, 2007 bp, synthetic construct
  misc_feature 1..2007: note = coding sequence for SEQ ID NO: 111
  source 1..2007: mol_type = other DNA
atgtccgatgatactacttacaagccaaagaacattttgattactggtgctgctggtttcattgcttctcacgttgccaacagattgatcagaaactacccagattacaagatcgttgttttggacaagttggattactgttctgatttgaagaacttggatccatctttctcttctccaaacttcaagttcgtcaagggtgatatcgcttctgatgatttggttaactacttgttgatcactgaaaacattgatactatcatgcacttcgctgctcaaactcacgttgataactctttcggtaactctttcgaattcaccaagaacaacatttacggtactcacgttttgttggaagcctgtaaggttactggtcaaatcagaagattcatccacgtttctaccgatgaagtctacggtgaaaccgatgaagatgctgctgttggtaaccacgaagcttctcaattgttgccaactaacccatactctgctactaaggctggtgctgaaatgttggttatggcttacggtagatcttacggtttgccagttattactactagaggtaacaacgtttacggtccaaaccaattcccagaaaagatgattccaaagttcatcttgttggctatgtctggtaagccattgccaatccacggtgatggttctaacgtcagatcttacttgtactgtgaggacgttgctgaagctttcgaagttgttttgcacaagggtgaaatcggtcacgtctacaacgtcggtactaagagagaaagaagagttatcgatgttgctagagacatctgtaagttgttcggtaaggacccagaatcttctattcaattcgttgaaaacagaccattcaacgatcaaagatacttcttggatgatcaaaagttgaagaagttgggttggcaagaaagaactaactgggaagatggtttgaagaaaactatggactggtacactcaaaacccagaatggtggggtgatgtttctggtgctttgttgccacacccaagaatgttgatgatgccaggtggtagattgtctgatggttcttctgaaaagaaggacgtttcttctaacactgtccaaactttcactgttgttactccaaagaacggtgattctggtgacaaggcttctttgaagttcttgatctacggtaagactggttggttgggtggtttgttgggtaagttgtgtgaaaagcaaggtattacttacgaatacggtaagggtagattggaagatagagcttctttggttgctgatattagatctatcaagccaactcacgttttcaacgctgctggtttgactggtagaccaaacgttgactggtgtgaatctcacaagccagaaaccattagagttaacgtcgctggtactttgactttggctgatgtttgtagagaaaacgatttgttgatgatgaacttcgccaccggttgtatcttcgaatacgacgctactcacccagaaggttctggtatcggtttcaaggaagaagataagccaaacttcttcggttctttctactctaagaccaaggccatggttgaagaattgttgagagaattcgacaacgtttgtaccttgagagtcagaatgccaatctcctctgacttgaacaacccaagaaacttcatcactaagatctctagatacaacaaggttgttgacatcccaaactctatgaccgttttggacgaattgttgccaatctctatcgaaatggctaagagaaacttgagaggtatctggaacttcaccaacccaggtgttgtttctcacaacgaaatcttggaaatgtacaagaactacatcgaaccaggtttcaagtggtccaacttcactgttgaagaacaagctaaggtcattgttgctgctagatctaacaacgaaatggatggttctaagttgtctaaggaattcccagaaatgttgtccatcaaggaatctttgttgaagtacgtattcgaaccaaacaagagaacctaa

SEQ ID NO: 113 - protein, 482 aa, Citrus sinensis
MSKDTIVFYTSPGRGHLNSMVELGKLILTYHPCFSIDIIIPTAPFVSSAGTDDYIASVSATAPSVTFHQLPPPVSGLPDTLRSPADFPALVYELGELNNPNLHETLITISKRSNLKAFVIDFLCNPAFQVSSSTLSIPTYYYFTTAGSVLAANLYLPTLHKNTTKSFRELGSALLNFPGFPPFPARDMALPMHDREGKVYKGLVDTGIQMAKSAGVIVNTFELLEERAIKAMLEGQCTPGDTSPPLYCIGPVVGRGNGENRGRDRHECLSWLDSKPSRSVLFLCFGSLGSFSCKQLKEMAIGLERSGVKFLWVVRAPAPDSVENRSSLESLLPEGFLDRTKDRGLVVESWAPQVEVLNHESVGGFVTHCGWNSVLEGVCAGVPMLAWPLYAEQKMIRAVVVEEMKVGLAVTRSEEGDGLVSSAELEQRVSELMDSEKGRAVKERAVAMKEAAAAAMRDGGSSRVALDNLVESFKRGCIAPFG

SEQ ID NO: 114 - DNA, 1449 bp, synthetic construct
  misc_feature 1..1449: note = coding sequence for SEQ ID NO: 113
  source 1..1449: mol_type = other DNA
atgtccaaggatactattgttttctacacttctccaggtagaggtcacttgaactccatggttgaattgggtaagttgatcttgacttaccacccatgtttctccattgacatcatcatcccaaccgccccattcgtttcttctgccggtaccgatgattacatcgcctccgtttccgccactgctccatccgttaccttccaccaattgccaccaccagttagtggtttgccagacactctcagatctccagctgacttcccagccttggtttacgaattgggtgaattgaacaacccaaacttgcacgaaaccttgatcactatttccaagagatctaacttgaaggccttcgttatcgatttcttgtgtaacccagctttccaagtttcctcttctactttgtctatcccaacttactactacttcaccaccgctggttctgttttggctgctaacttgtacttgccaactttgcacaagaacaccactaagtctttcagagaattgggttctgctttgttgaacttcccaggtttcccaccattcccagctagagacatggctttgccaatgcacgatagagaaggtaaggtttacaagggtttggtcgacaccggtatccaaatggctaagtctgctggtgtaattgttaacacattcgaattgttggaggaaagagctatcaaggctatgttggaaggtcaatgtactccaggtgatacttctccaccattgtactgtatcggtccagttgttggtagaggtaacggtgaaaacagaggtagagatagacacgaatgtttgtcttggttggactctaagccatctagatctgtcttgttcttgtgtttcggttctttgggttctttctcttgtaagcaattgaaggaaatggccattggtttggaaagatctggtgttaagttcttgtgggtcgttagagctccagctccagactctgtcgaaaacagatcttctttggaatctttgttgccagaaggtttcttggatagaactaaggatagaggtttggttgttgaatcttgggctccacaagttgaagttttgaaccacgaatctgttggcggcttcgttactcactgcggttggaactctgttttggaaggtgtttgtgccggtgttccaatgttggcttggccattgtacgccgaacaaaagatgattagagctgtcgttgttgaagaaatgaaggttggtttggctgttactagatctgaagaaggtgacggtttggtttcttctgccgaattggaacaaagagtttctgaattgatggactctgaaaagggtagagctgttaaggaaagagctgttgctatgaaggaagctgctgctgctgctatgagagatggtggttcttctagagttgcgctcgacaacttggttgaaagctttaagagaggttgcatcgctccattcggttaa

SEQ ID NO: 115 - protein, 477 aa, Citrus clementina
MSEKQDHVHVAILPLPAVGHVNSMLNLAELLGHAGIKITFLNTEHYYDRVIRHSSDAFSRYMQIPGFQFKTLTDGLPRDHPRTPDKFPELVDSLNCATPPLLKEMVSDSKSPVNCIITDGYMSRAIDAAREVGVSIIYFRTISACAFWSFHCIPDIIDAGELPIKGTEDMDRLITTVPGMEGFLRCRDLPSFCRVNDPMDPHLLLFARETRLSAHADGLILNTFEDLEGPILSQIRNHSCPNIYSIGPLNAHLKVRIPEKTYSSSSLWKIDRSCMAWLDKQPKQSVIYVSFGSIAVMSRDQLIEFYYGLVHSKKNFLWVIRPDLISGKDGENQIPEELLEATKERGCIAGWVPQEEVLAHSAVGGFLTHCGWNSTLESIVAGMPMICWPSFADQQINSRFVGEVWKLGLDIKDLCDRNIVEKTVNDLMVERKEEFMESADRMANLAKKSVNKGGSSYCNLDRLVNDIKMMSSQPQNC

SEQ ID NO: 116 - DNA, 1434 bp, synthetic construct
  misc_feature 1..1434: note = coding sequence for SEQ ID NO: 115
  source 1..1434: mol_type = other DNA
atgtccgaaaagcaagatcacgttcacgttgctatcttgccattgccagccgttggtcacgtcaactccatgttgaacttggctgaattgttgggtcacgccggtatcaagattactttcttgaacaccgaacactactacgatagagtcatcagacactcttctgatgccttctctagatacatgcaaattccaggtttccaattcaagaccttgactgatggtttgccaagagatcacccaagaactccagataagttcccagaattggttgattctttgaactgtgccactccaccattgttgaaggaaatggtatctgattctaagtctccagttaactgtattatcactgatggttacatgtctagagctattgacgctgccagagaagttggtgtttccattatttacttcagaactatttctgcttgtgctttctggtctttccactgtatcccagatatcattgatgctggtgaattgccaatcaagggtactgaagatatggatagattgatcaccactgttccaggtatggaaggtttcttgagatgtagagatttgccatctttctgtagagttaacgacccaatggatccacacttgttgttgttcgctagagaaaccagattgagtgctcatgctgacggcttgatcttgaacactttcgaagatttggaaggtccaatcttgtcccaaatcagaaaccactcttgtccaaacatctactctattggtccattgaacgctcacttgaaggttagaatcccagaaaagacttactcctcttcttctttgtggaagattgacagatcttgtatggcttggttggacaagcaaccaaagcaatctgttatctacgtatctttcggttctattgctgttatgtctagagatcaattgatcgaattctactacggtttggttcactctaagaagaacttcttgtgggtcatcagaccagacttgatttctggtaaggatggtgaaaaccaaattccagaagaattgttggaagctaccaaggaaagaggttgtattgctggttgggttccacaagaagaagttttggcccactctgctgttggtggtttcttgacccactgtggttggaactctaccttggaatctatcgttgctggtatgccaatgatttgctggccaagcttcgctgatcaacagatcaactctagattcgttggtgaagtttggaagttgggtttggatatcaaggatttgtgtgatagaaacattgttgaaaagactgttaacgatttgatggttgaaagaaaggaagaattcatggaatctgctgatagaatggctaacttggctaagaagtctgttaacaagggtggttcctcttactgtaacttggacagattggttaacgatatcaagatgatgtcctctcaaccacaaaactgttaa

SEQ ID NO: 117 - protein, 354 aa, Citrus clementina
MSGSIVDGERDQSFAYANQLAMGTVLPMAMQAVYELGIFQIIDKAGPGAKLSASDIAAQLPTKNKDAPTMLDRILRLLASYSVVECSLDGSGARRRYSLNSVSKYYVPNKDGVSLGPALQMIQDKVFLESWSHLKDAILEGGIPFNRAHGMHAFEYGRVDPRFNKHFNTAMYNHTSLIMSNILESYKGFANIKQLVDVGGNLGVTLQAITSKYPYIKGINFDQPHVIEHAPLHPHIEHVAGDMFQSVPKGDAIFLKWILHDWDDEHCLKLLKNCYKSVPEDGKVIVVELMLPEVPNTSIESKSNSHIDVLMMTQNPGGKERTKHEFMTLATGAGFSGIRFDLVTGNFWVMEFYK

SEQ ID NO: 118 - DNA, 1065 bp, synthetic construct
  misc_feature 1..1065: note = coding sequence for SEQ ID NO:117
  source 1..1065: mol_type = other DNA
atgtccggttctatcgttgatggtgaaagagatcaatctttcgcttacgctaaccaattggctatgggtactgttttgccaatggccatgcaagctgtttacgaattgggtattttccaaatcatcgacaaggctggtccaggtgctaagttgtctgcttctgatattgctgcccaattgccaaccaagaacaaggacgctccaactatgctcgacagaatactaagattgttggcttcttactctgttgttgaatgttctttggatggttctggtgccagaagaagatactctttgaactctgtctccaagtactacgttccaaacaaggatggtgtctctttgggtccagctttgcaaatgattcaagacaaggttttcttggaatcttggtcccacttgaaggatgctattttggaaggtggtattccattcaacagagcccacggtatgcacgctttcgaatacggtagagttgacccaagattcaacaagcacttcaacactgctatgtacaaccacacctctttgattatgtctaacattttggaatcttacaagggtttcgccaacatcaagcaattggtcgatgttggtggtaacttgggcgtaactctccaagccatcacttccaagtacccatacattaagggtatcaacttcgaccaaccacacgttattgaacacgccccattgcacccacacattgaacacgttgctggtgatatgttccaatctgttccaaagggtgacgccattttcttgaagtggatcttgcacgattgggacgatgaacactgtttgaagttgttgaagaactgttacaagtctgttccagaagatggtaaggttatcgttgttgaattgatgttgccagaagttccaaacacttctattgaatctaagtctaactcccacattgacgttttgatgatgactcaaaacccaggtggtaaggaaagaactaagcacgaattcatgaccttggctactggtgctggtttctctggtatcagattcgatttggttactggtaacttctgggttatggaattctacaagtaa

SEQ ID NO: 119 - protein, 250 aa, Citrus sinensis
MSAANREDQEENKDRYFKAVHGNKTLLQSEKLYEYILETSVYPREPQCLKEIRELTYKHPLSPMMTSPDEAQFFSMLLKLINAKNTMEIGVFTGYSLLATALAIPDDGKILALDITKEHYEKGLPIIQKAGVAHKIDFREGPALPLLDQLIQHEKYHGTFDFVFVDADKDNYVNYHKRLIELVKVGGVIGYDNTLWGGSVVAPPDADLDEHILYLRDFVQELNKALAVDPRIEICQLSIADGVTLCRRIG

SEQ ID NO: 120 - DNA, 753 bp, synthetic construct
  misc_feature 1..753: note = coding sequence for SEQ ID NO:119
  source 1..753: mol_type = other DNA
atgtccgctgctaacagagaagatcaagaagaaaacaaggatagatacttcaaggctgttcacggtaacaagaccttgttgcaatctgaaaagttgtacgaatacatcttggaaacctctgtttacccaagagaaccacaatgtttgaaggaaatcagagaattgacttacaagcacccattgtctccaatgatgacttctccagatgaagctcaattcttctctatgttgttgaagttgatcaacgccaagaacaccatggagataggtgtattcaccggttactctttgttggccactgccttggccattccagacgatggtaagattttggccttggacatcaccaaggaacactacgaaaagggtttgccaatcattcaaaaggctggtgttgctcacaagattgacttcagagaaggtccagctttgccattattggatcaattaatccaacacgaaaagtaccatggcacattcgacttcgtctttgttgatgctgacaaggataactacgttaactaccacaagagattgattgaattggttaaggttggtggtgttattggttacgacaacaccttgtggggtggttctgttgttgctccaccagatgccgacttggatgaacacatcttgtacttgagagatttcgttcaagaattgaacaaggccttggccgttgatccaagaatagaaatttgtcaattgtccattgctgatggtgttactttgtgtagaagaattggttaa

SEQ ID NO: 121 - protein, 514 aa, Arabidopsis thaliana
MSATLFLTILLATVLFLILRIFSHRRNRSHNNRLPPGPNPWPIIGNLPHMGTKPHRTLSAMVTTYGPILHLRLGFVDVVVAASKSVAEQFLKIHDANFASRPPNSGAKHMAYNYQDLVFAPYGHRWRLLRKISSVHLFSAKALEDFKHVRQEEVGTLTRELVRVGTKPVNLGQLVNMCVVNALGREMIGRRLFGADADHKADEFRSMVTEMMALAGVFNIGDFVPSLDWLDLQGVAGKMKRLHKRFDAFLSSILKEHEMNGQDQKHTDMLSTLISLKGTDLDGDGGSLTDTEIKALLLNMFTAGTDTSASTVDWAIAELIRHPDIMVKAQEELDIVVGRDRPVNESDIAQLPYLQAVIKENFRLHPPTPLSLPHIASESCEINGYHIPKGSTLLTNIWAIARDPDQWSDPLAFKPERFLPGGEKSGVDVKGSDFELIPFGAGRRICAGLSLGLRTIQFLTATLVQGFDWELAGGVTPEKLNMEESYGLTLQRAVPLVVHPKPRLAPNVYGLGSG

SEQ ID NO: 122 - DNA, 1545 bp, synthetic construct
  misc_feature 1..1545: note = coding sequence for SEQ ID NO:121
  source 1..1545: mol_type = other DNA
atgtccgctactttgttcttgactatcttgttggctactgttttgttcttgatcttgagaatcttctctcacagaagaaacagatctcacaacaacagattgccaccaggtccaaacccatggccaatcatcggtaacttgccacacatgggtactaagccacacagaactttgtctgctatggttactacttacggtccaatcttgcacttgagattgggtttcgttgacgttgttgttgctgcttctaagtctgttgctgaacaattcttgaagatccacgacgctaacttcgcttctagaccaccaaactctggtgctaagcacatggcttacaactaccaagacttggttttcgctccatacggtcacagatggagattgttgagaaagatctcttctgttcacttgttctctgctaaggctttggaagatttcaagcacgttagacaagaagaagttggtactttgactagagaattggttagagttggtactaagccagttaacttgggtcaattggttaacatgtgtgttgttaacgctttgggtagagaaatgatcggtagaagattgttcggtgctgacgctgaccacaaggctgacgaattcagatctatggttactgaaatgatggctttggctggtgttttcaacatcggtgacttcgttccatctttggactggttggacttgcaaggtgttgctggtaagatgaagagattgcacaagagattcgacgctttcttgtcatctatcttgaaggaacacgaaatgaacggtcaagaccaaaagcacactgacatgttgtctactttgatctctttgaagggtactgacttggacggtgacggtggttctttgactgacactgaaatcaaggctttgttgttgaacatgttcactgctggtactgacacttctgcttctactgttgactgggctatcgctgaattgatcagacacccagacatcatggttaaggctcaagaagaattggacatcgttgttggtagagacagaccagttaacgaatctgacatcgctcaattgccatacttgcaagctgttatcaaggaaaacttcagattgcacccaccaactccattgtctttgccacacatcgcttctgaatcttgtgaaatcaacggttaccacatcccaaagggttctactttgttgactaacatctgggctatcgctagggacccagaccaatggtctgacccattggctttcaagccagaaagattcttgccaggtggtgaaaagtctggtgttgacgttaagggttctgacttcgaattgatcccattcggtgctggtagaagaatctgtgctggtttgtctttgggtttgagaactatccaattcttgactgctactttggttcaaggtttcgactgggaattggctggtggtgttactccagaaaagttgaacatggaagaatcttacggtttgactttgcaaagagctgttccattggttgttcacccaaagccaagattggctccaaacgtttacggtttgggttctggttaa

SEQ ID NO: 123 - protein, 571 aa, Arabidopsis thaliana
MSVLQQQTHFLTKKIDQEDEEEEPSHDFIFRSKLPDIFIPNHLPLTDYVFQRFSGDGDGDSSTTCIIDGATGRILTYADVQINMRRIATGIHRLGIRHGDVVMLLLPNSPEFALSFLAVAYLGAVSTTANPFYTQPEIAKQAKASAAKMIITKKCLVDKLTNLKNDGVLIVCLDDDGDNGVVSSSDDGCVSFTELTQADETELLKPKISPEDTVAMPYSSGTTGLPKGVMITHKGLVTSIAQKVDGENPNLNFTANDVILCFLPMFHIYALDALMLSAMRTGAALLIVPRFELNLVMELIQRYKVTVVPVAPPVVLAFIKSPETERYDLSSVRIMLSGAATLKKELEDAVRLKFPNAIFGQGYGMTESGTVAKSLAFAKNPFKTKSGACGTVIRNAEMKVVDTETGISLPRNKSGEICVRGHQLMKGYLNDPEATARTIDKDGWLHTGDIGFVDDDDEIFIVDRLKELIKFKGYQVAPAELEALLISHPSIDDAAVVAMKDEVADEVPVAFVARSQGSQLTEDDVKSYVNKQVVHYKRIKMVFFIEVIPKAVSGKILRKDLRAKLETMCSK

SEQ ID NO: 124 - DNA, 1716 bp, synthetic construct
  misc_feature 1..1716: note = coding sequence for SEQ ID NO:123
  source 1..1716: mol_type = other DNA
atgtccgttttgcaacaacaaactcacttcttgactaagaagatcgatcaagaagatgaagaagaagaaccatctcacgatttcattttcagatctaagttgccagatatcttcatcccaaaccacttgccattgaccgattacgtattccaaagattctccggtgatggtgacggtgattcctctactacctgtatcatcgacggtgccactggccgtatcctcacctacgccgatgttcaaatcaacatgagaagaatcgctaccggtatccacagattgggtatcagacacggtgacgtcgttatgttgttgttgccaaactctccagaattcgctttgtctttcttggccgttgcttacttgggtgccgtttccactaccgctaacccattctacactcaaccagaaatcgctaagcaagctaaggcctccgccgctaagatgatcatcactaagaaatgcctggtcgataagttgactaacttgaagaacgacggtgttttgatcgtttgtttggacgatgacggtgacaatggcgttgtgagctcttctgatgatggttgtgtttctttcactgaattgactcaagctgacgaaactgaattgttgaagccaaagatctctccagaagatactgttgctatgccatactcttccggcactactggtttaccaaagggtgttatgattactcacaagggtttggttacttctatcgctcaaaaggtcgacggtgaaaacccaaacttgaacttcactgccaacgacgtcatcttgtgtttcttgccaatgttccacatttacgctttggacgctttgatgttgtctgctatgagaaccggtgctgctttgttgatcgttccaagattcgaattgaacttggttatggaattgattcaaagatacaaggtcactgttgttccagttgctccaccagttgttttggctttcattaagtccccagaaactgaaagatacgacttatcttctgttagaattatgttgtctggtgctgctactttgaagaaggaattggaagatgccgttagattgaagttcccaaacgccattttcggtcaaggttacggtatgaccgaatccggtactgttgctaagtcgctcgcgttcgctaagaacccattcaagaccaagtccggtgcttgtggtactgttatcagaaacgccgaaatgaaggttgtcgataccgaaaccggtatctccttgccaagaaacaagtctggtgaaatctgtgtcagaggtcaccaattgatgaagggttacttgaacgatccagaagctactgctagaaccatcgacaaggacggttggttgcacactggtgatattggtttcgttgatgatgatgatgaaatcttcattgttgatagattgaaggaattgatcaagttcaagggttaccaagttgctccagctgaattggaagctttgttgatttctcacccatctatcgatgatgccgctgttgttgctatgaaggatgaagttgctgatgaagttccagttgctttcgttgctagatctcaaggttctcaattgactgaagatgatgtcaagtcttacgttaacaagcaagttgttcactacaagagaattaagatggttttcttcatcgaagttatcccaaaggctgtttctggtaagattttgagaaaggatttgagagctaagttggaaaccatgtgttctaagtaa

SEQ ID NO: 125 - protein, 561 aa, Citrus clementina
MSISIATKKPELSLDISSPAPPAPSNEKIATHIFKSKLPDIPISNHLPLHTYCFQDRLSDDPCLIVGLTGKTYSYAETHLICRKTAAGLSNLGIKKGDVIMILLQNCAEFVFSFMGASMIGAVTTTANPFYTSAEILKQFRTSGAKLIITMSQYVDRLPKTDKDFTVITIDAPPENCLHFTVLSEADEDQIPEVAIEPDDPVALPFSSGTTGLPKGVVLTHKSLITSVAQQVDGENPNLYLTNGDVVLCVLPLFHIYSLNSVLLCSLRAGAGVLLMQKFEIGALLELIQRHRVSVAAVVPPLVLALAKNPMVADYDLSSIRVVLSGAAPLGKELEDALRSRVPQAILGQGYGMTEAGPVLSMCLGFAKQPFPTKSGSCGTVVRNAELKVIDPEIGASLPHNQPGEICIRGPQIMKGYLNDPEATAATIDVEGWLHTGDIGYVDDDDEVFIVDRVKEIIKFKGFQVPPAEIEALLLSHPSIGDAAVVPQKDEVAGEVPVAFVVRSNGFELTEEAIKEYIAKQVVFYKRLHKIYFVHAIPKSPSGKILRKDLRAKLASSMPLN

SEQ ID NO: 126 - DNA, 1686 bp, synthetic construct
  misc_feature 1..1686: note = coding sequence for SEQ ID NO:125
  source 1..1686: mol_type = other DNA
atgtccatctctatcgccactaagaagccagaattgtctttggatatctcttctccagctccaccagctccatccaacgaaaagatcgccacccacattttcaagtctaagttgccagatatcccaatctccaaccacttgccattgcacacttactgtttccaagatagattgtctgacgacccatgtttgatcgttggtttgactggtaagacttactcttacgccgaaactcacttgatctgtagaaagaccgctgccggtttgtctaacttgggtatcaagaagggtgacgttatcatgatcttgttgcaaaactgtgccgaattcgttttctctttcatgggtgcttccatgatcggtgctgtcactactactgccaacccattctacacttctgctgaaatcttgaagcaattcagaacctctggtgccaagttgattatcactatgtctcaatacgtcgacagattgccaaagactgacaaggatttcactgttatcactatcgatgccccaccagaaaactgtttgcacttcactgttttgtctgaagctgatgaagatcaaatcccagaagttgccatcgaaccagacgatccagttgctttgccattctcttctggtaccactggtttgccaaagggtgttgttttgactcataagagcttgatcacctctgttgctcaacaagttgacggtgaaaacccaaacttgtacttgactaacggtgacgttgttttgtgtgttttgccattgttccacatttactctttgaactctgttttgttgtgttctttgagagctggtgctggtgttttgttgatgcaaaagttcgaaatcggtgctttgttggaattgatccaaagacacagagtttctgttgctgctgttgttccaccattggttttggctttggccaagaacccaatggttgctgattacgacttgtcatctatcagagttgttttgtccggtgctgctccattgggtaaggaattggaagatgctttgagatctagagtcccacaagctatcttgggtcaaggttacggtatgactgaagctggtccagttttgtctatgtgtttgggtttcgctaagcaaccattcccaactaagtctggttcttgtggtaccgttgttagaaacgctgaattgaaggttattgacccagaaattggtgcctccttgccacacaaccaaccaggtgaaatttgtatcagaggtccacaaattatgaagggttacttgaacgatccagaagctactgctgctactatcgacgttgaaggttggttgcacactggtgacatcggttacgttgatgatgatgatgaagttttcatcgtcgatagagtcaaggaaatcatcaagttcaagggtttccaagttccaccagctgaaattgaagccttgttgttgtctcacccatctattggtgatgctgctgttgttccacaaaaggatgaagttgcgggtgaagttccagttgcgttcgttgttcgttcgaacggcttcgaattgaccgaggaagccattaaggaatacatcgctaagcaagttgttttctacaagagattgcacaagatttacttcgttcacgctattccaaagtctccatccggtaagattttgagaaaggatttgagagccaagttggcctcttctatgccattgaactaa

SEQ ID NO: 127 - protein, 365 aa, Angelica archangelica
MSAPTTITALAQEKTLNLAFVRDEDERPKVAYNQFSNEIPIISLAGMDDDTGRRPQICRKIVEAFEDWGIFQVVDHGIDGTLISEMTRLSREFFALPAEEKLRYDTTGGKRGGFTISTHLQGDDVKDWREFVTYFSYPIDDRDYSRWPDKPQGWRSTTEVYSEKLMVLGAKLLEVLSEAMGLEKEALTKACVNMEQKVLINYYPTCPEPDLTLGVRRHTDPGTITILLQDMVGGLQATRDGGKTWITVQPVEGAFVVNLGDHGHYLSNGRFKNADHQAVVNSTSSRLSIATFQNPAQNAIVYPLRIREGEKAVLDEAITYAEMYKKNMTKHIEVATLKKLAKEKRLQEEKAKLETESKSADGISA

SEQ ID NO: 128 - DNA, 1098 bp, synthetic construct
  misc_feature 1..1098: note = coding sequence for SEQ ID NO:127
  source 1..1098: mol_type = other DNA
atgtccgctccaactactatcactgctttggctcaagaaaagactttgaacttggctttcgttagagatgaagatgaaagaccaaaggttgcttacaaccaattctctaacgaaatcccaatcatctctttggctggtatggacgacgacactggtaggaggccacaaatctgtagaaagatcgttgaagctttcgaagattggggtatcttccaagttgttgaccacggtatcgacggtactttgatctctgaaatgactagattgtctagagaattcttcgctttgccagctgaagaaaagttgagatacgacactactggtggtaagagaggtggtttcactatctctactcacttgcaaggtgacgacgttaaggactggagagaattcgttacttacttctcttacccaatcgacgacagagactactctagatggccagacaagccacaaggttggagatctactactgaagtttactctgaaaagttgatggttttgggtgctaagttgttggaagttttgtctgaagctatgggtttggaaaaggaagctttgactaaggcttgtgttaacatggaacaaaaggttttgatcaactactacccaacttgtccagaaccagacttgactttgggtgttaggagacacactgacccaggtactatcactatcttgttgcaagacatggttggtggtttgcaagctactagagatggtggtaagacttggatcactgttcaaccagttgaaggtgctttcgttgttaacttgggtgaccacggtcactacttgtctaacggtagattcaagaacgctgaccaccaagctgttgttaactctacttcttctagattgtctatcgctactttccaaaacccagctcaaaacgctatcgtttacccattgagaatcagagaaggtgaaaaggctgttttggacgaagctatcacttacgctgaaatgtacaagaagaacatgactaagcacatcgaagttgctactttgaagaagttggctaaggaaaagagattgcaagaagaaaaggctaagttggaaactgaatctaagtctgctgacggtatctctgcttaa

SEQ ID NO: 129 - protein, 514 aa, Cynara cardunculus var. scolymus
MSQVHETLTPAVIAAVVLSSVFLYLLFKKKQNHRLPPSPPSLPIIGHLHHLGPLIHQSFHHLSTRYGPLIHLRLGSVPCIVASTPDLARDFLKTNELAFSSRKHSLAIDHITYGVAFAFAPYGPYWKFIKKMSTVELLGNQNLGHFLPIRTHEIQELLQTLTEKAKRRESVNLTEELLKLTNNVICQMMMSIRCSGTNSEADEAKNLVREVTQIFGEFNVSDFIWFCKNIDLQGFKKRYTDTHKRYDALLEKIIFEREEKRRKEGKREDGKGKDFLDMLLDVLEDAKAEIKITRDHIKALILDFFTAATDTTAIALEWMLVELISNPKVLEIAREEIDQVVGNERLVQESDAPNLPYIQAIIKEALRLHPPIPMLIRKSIEDVSVQGYDIPAGTMLFVNIWSIGRNPKYWESPLEFKPHRFLEDDPVKKSLDIKGQSFQLLPFGTGRRGCPGINLAMRELPVVIAGLIQCFEWNVNGKQVLDMDERAGLTAPRAADFVCVPSVRENSPMMFTST

SEQ ID NO: 130 - DNA, 1545 bp, synthetic construct
  misc_feature 1..1545: note = coding sequence for SEQ ID NO:129
  source 1..1545: mol_type = other DNA
atgtcccaagttcacgaaactttgactccagctgttatcgctgctgttgttttgtcatctgttttcttgtacttgttgttcaagaagaagcaaaaccacagattgccaccatctccaccatctttgccaatcatcggtcacttgcaccacttgggtccattgatccaccaatctttccaccacttgtctactagatacggtccattgatccacttgagattgggttctgttccatgtatcgttgcttctactccagacttggctagagacttcttgaaaactaacgaattggctttctcttctagaaagcactctttggctatcgaccacatcacttacggtgttgctttcgctttcgctccatacggtccatactggaagttcatcaagaagatgtctactgttgaattgttgggtaaccaaaacttgggtcacttcttgccaatcagaactcacgaaatccaagaattgttgcaaactttgactgaaaaggctaagagaagagaatctgttaacttgactgaagaattgttgaagttgactaacaacgttatctgtcaaatgatgatgtctatcagatgttctggtactaactctgaagctgacgaagctaagaacttggttagagaagttactcaaatcttcggtgaattcaacgtttctgacttcatctggttctgtaagaacatcgacttgcaaggtttcaagaagagatacactgacactcacaagagatacgacgctttgttggaaaagatcatcttcgaaagagaagaaaagagaagaaaggaaggtaagagagaagatggtaagggtaaggacttcttggacatgttgttggacgttttggaagatgctaaggctgaaatcaagatcactagagatcacatcaaggctttgatcttggacttcttcactgctgctactgacactactgctatcgctttggaatggatgttggttgaattgatctctaacccaaaggttttggaaatcgctagagaagaaatcgaccaagttgttggtaacgaaagattggttcaagaatctgacgctccaaacttgccatacatccaagctatcatcaaggaagctttgagattgcacccaccaatcccaatgttgatcagaaagtctatcgaagatgtttctgttcaaggttacgacatcccagctggtactatgttgttcgttaacatctggtctatcggtagaaacccaaagtactgggaatctccattggaattcaagccacacagattcttggaagatgacccagttaagaagtctttggacatcaagggtcaatctttccaattgttgccattcggtactggtagaagaggttgtccaggtatcaacttggctatgagagaattgccagttgttatcgctggtttgatccaatgtttcgaatggaacgttaacggtaagcaagttttggacatggacgaaagagctggtttgactgctccaagagctgctgacttcgtttgtgttccatctgttagagaaaactctccaatgatgttcacttctacttaa

SEQ ID NO: 131 - protein, 507 aa, Perilla frutescens var. crispa
MSALYAALFLLSAAVVRSVLDRKRGRPPYPPGPFPLPIIGHLHLLGPRLHQTFHDLSQRYGPLMQLRLGSIRCVIAASPELAKECLKTHELVFSSRKHSTAIDIVTYDSSFAFSPYGPYWKFIKKLCTYELLGARNLAHFQPIRTLEVKSFLQILMRKGESGESFNVTEELVKLTSNVISHMMLSIRCSETESEAEAARTVIREVTQIFGEFDVSDIIWLCKNFDFQGIRKRSEDIQRRYDALLEKIITDREKQRRTHGGGGGGGEAKDFLDMFLDIMESGKAEVKFTREHLKALILDFFTAGTDTTAIVCEWAIAEVINNPNVLKKAQEEIANIVGFDRILQESDAPNLPYLQALIKETFRLHPPIPMLARKSISDCVIDGYMIPANTLLFVNLWSMGRNPKIWDYPTAFQPERFLEKEKAAIDVKGQHFELLPFGTGRRGCPGMLLAIQEVVIIIGTMIQCFDWKLPDGSGHVDMAERPGLTAPRETDLFCRVVPRVDPLVVSTQ

SEQ ID NO: 132 - DNA, 1524 bp, synthetic construct
  misc_feature 1..1524: note = coding sequence for SEQ ID NO:131
  source 1..1524: mol_type = other DNA
atgtccgctttgtacgctgctttgttcttgttgtctgctgctgttgttagatctgttttggacagaaagagaggtagaccaccatacccaccaggtccattcccattgccaatcatcggtcacttgcacttgttgggtccaagattgcaccaaactttccacgacttgtctcaaagatacggtccattgatgcaattgagattgggttctatcagatgtgttatcgctgcttctccagaattggctaaggaatgtttgaaaactcacgaattggttttctcttctagaaagcactctactgctatcgacatcgttacttacgactcttctttcgctttctctccatacggtccatactggaagttcatcaagaagttgtgtacttacgaattgttgggtgctagaaacttggctcacttccaaccaatcagaactttggaagttaagtctttcttgcaaatcttgatgagaaagggtgaatctggtgaatctttcaacgttactgaagaattggttaagttgacttctaacgttatctctcacatgatgttgtctatcagatgttctgaaactgaatctgaagctgaagctgctagaactgttatcagagaagttactcaaatcttcggtgaattcgacgtttctgacatcatctggttgtgtaagaacttcgacttccaaggtatcagaaagagatctgaagatatccaaagaagatacgacgctttgttggaaaagatcatcactgacagagaaaagcaaagaagaactcacggtggtggtggtggtggtggtgaagctaaggacttcttggacatgttcttggacatcatggaatctggtaaggctgaagttaagttcactagagaacacttgaaggctttgatcttggacttcttcactgctggtactgacactactgctatcgtttgtgaatgggctatcgctgaagttatcaacaacccaaacgttttgaagaaggctcaagaagaaatcgctaacatcgttggtttcgacagaatcttgcaagaatctgacgctccaaacttgccatacttgcaagctttgatcaaggaaactttcagattgcacccaccaatcccaatgttggctagaaagtctatctctgactgtgttatcgacggttacatgatcccagctaacactttgttgttcgttaacttgtggtctatgggtagaaacccaaagatctgggactacccaactgctttccaaccagaaagattcttggaaaaggaaaaggctgctatcgacgttaagggtcaacacttcgaattgttgccattcggtactggtagaagaggttgtccaggtatgttgttggctatccaagaagttgttatcatcatcggtactatgatccaatgtttcgactggaagttgccagacggttctggtcacgttgacatggctgaaagaccaggtttgactgctccaagagaaactgacttgttctgtagagttgttccaagagttgacccattggttgtttctactcaataa

SEQ ID NO: 133 - protein, 515 aa, Dahlia pinnata
MSNTLLVLQMVIPAIIAFVIFHLLFFKSKPNRRLPPSPPSLPIIGHLHHLGPLIHQSFNRLSARYGPLIHLRLGSVSCVVADAPDLAQELLQKNDLAFANRKHTLAIDHVTYGVAFAFAPYGPYWRFIKKMSTVELLGIQNLGHFLPIRTQEIHGLLLTLTEKSKQNESVNMTNELLKLSNNIICQMMMGIRCSGNKTEAEEAKNLVREVTTIFGEFNVSDFIWFCKKLDLQGFKKRYEDIRTRYDALLERIIFAREEMRKEGKGMEDGKGKDFLDMLLDVLEDDKAEIKITRNHIKALILDFVTAGTDTTAVIIEWTLVELIKNPMVMEKAKQELDEVVGNTRLVEESDAPKLPYIQAIIKEAFRLHPPIPMIIRKSNENVSVKSGYEIPAGSILFVNNWSIGRNPKYWESPLEFKPDRFLKEGVLKPSLDIRGQNFQILPFGTGRRSCPGINMAMRQLPVVVAILIQCFEWTVNDKQVLNMDERGGLTTPRATDLVCFPLLRKNSPHSMFTSV

SEQ ID NO: 134 - DNA, 1548 bp, synthetic construct
  misc_feature 1..1548: note = coding sequence for SEQ ID NO:133
  source 1..1548: mol_type = other DNA
atgtccaacactttgttggttttgcaaatggttatcccagctatcatcgctttcgttatcttccacttgttgttcttcaagtctaagccaaacagaagattgccaccatctccaccatctttgccaatcatcggtcacttgcaccacttgggtccattgatccaccaatctttcaacagattgtctgctagatacggtccattgatccacttgagattgggttctgtttcttgtgttgttgctgacgctccagacttggctcaagaattgttgcaaaagaacgacttggctttcgctaacagaaagcacactttggctatcgaccacgttacttacggtgttgctttcgctttcgctccatacggtccatactggagattcatcaagaagatgtctactgttgaattgttgggtatccaaaacttgggtcacttcttgccaatcagaactcaagaaatccacggtttgttgttgactttgactgaaaagtctaagcaaaacgaatctgttaacatgactaacgaattgttgaagttgtctaacaacatcatctgtcaaatgatgatgggtatcagatgttctggtaacaagactgaagctgaagaagctaagaacttggttagagaagttactactatcttcggtgaattcaacgtttctgacttcatctggttctgtaagaagttggacttgcaaggtttcaagaagagatacgaagatatcagaactagatacgacgctttgttggaaagaatcatcttcgctagagaagaaatgagaaaggaaggtaagggtatggaagatggtaagggtaaggacttcttggacatgttgttggacgttttggaagatgacaaggctgaaatcaagatcactagaaaccacatcaaggctttgatcttggacttcgttactgctggtactgacactactgctgttatcatcgaatggactttggttgaattgatcaagaacccaatggttatggaaaaggctaagcaagaattggacgaagttgttggtaacactagattggttgaagaatctgacgctccaaagttgccatacatccaagctatcatcaaggaagctttcagattgcacccaccaatcccaatgatcatcagaaagtctaacgaaaacgtttctgttaagtctggttacgaaatcccagctggttctatcttgttcgttaacaactggtctatcggtagaaacccaaagtactgggaatctccattggaattcaagccagacagattcttgaaggaaggtgttttgaagccatctttggacatcagaggtcaaaacttccaaatcttgccattcggtactggtagaagatcttgtccaggtatcaacatggctatgagacaattgccagttgttgttgctatcttgatccaatgtttcgaatggactgttaacgacaagcaagttttgaacatggacgaaagaggtggtttgactactccaagagctactgacttggtttgtttcccattgttgagaaagaactctccacactctatgttcacttctgtttaa

SEQ ID NO: 135 - protein, 515 aa, Callistephus chinensis
MSNIFEVFQSVSPAIIAIFFISSLFIYLVLIRNQKSLSLPPSPPALPIIGHLHHLGPLIHHSFHDLSTRYGPLIHLRLGSVPCVVASTPDLARDFLKTNELAFSSRKHSLAIDHVTYGVSFAFAPYGPYWKFIKKTSIVELLGNQNLSNFLPIRTQEVHELLQTLMVKSKKNESVNLSEELLKLTNNVICQMMMSIRCSGTNNEADEAKNLVREVTKIFGEFNISDFICLFKNIDLQGFKKRYVDTHTRYNALLEKMIFEREEKRKQKKSEDGKGKDFLDILLDVLEDENAEIKITRDHIKALILDFFTAATDTTAISIEWTLVELTNNPKVLENARKEIAEVVGDERLVQESDIPNLPYIQAIIKETLRMHPPIPMVIRKSIDNVTVQGYDIRAGTMLFVNIWSIGRNPLYWESPLEFKPHRFLDGHARNLDVKGQCFQLLPFGTGRRGCPGISLAMRELPVVIAGLIQCFEWNANDKEVLSMDERAGLTAPRAVDLEFVPLMRQNCPNIFVSA

SEQ ID NO: 136 - DNA, 1548 bp, synthetic construct
  misc_feature 1..1548: note = coding sequence for SEQ D NO 135
  source 1..1548: mol_type = other DNA
atgtccaacatcttcgaagttttccaatctgtttctccagctatcatcgctatcttcttcatctcttctttgttcatctacttggttttgatcagaaaccaaaagtctttgtctttgccaccatctccaccagctttgccaatcatcggtcacttgcaccacttgggtccattgatccaccactctttccacgacttgtctactagatacggtccattgatccacttgagattgggttctgttccatgtgttgttgcttctactccagacttggctagagacttcttgaaaactaacgaattggctttctcttctagaaagcactctttggctatcgaccacgttacttacggtgtttctttcgctttcgctccatacggtccatactggaagttcatcaagaaaacttctatcgttgaattgttgggtaaccaaaacttgtctaacttcttgccaatcagaactcaagaagttcacgaattgttgcaaactttgatggttaagtctaagaagaacgaatctgttaacttgtctgaagaattgttgaagttgactaacaacgttatctgtcaaatgatgatgtctatcagatgttctggtactaacaacgaagctgacgaagctaagaacttggttagagaagttactaagatcttcggtgaattcaacatctctgacttcatctgtttgttcaagaacatcgacttgcaaggtttcaagaagagatacgttgacactcacactagatacaacgctttgttggaaaagatgatcttcgaaagagaagaaaagagaaagcaaaagaagtctgaagatggtaagggtaaggacttcttggacatcttgttggacgttttggaagatgaaaacgctgaaatcaagatcactagagatcacatcaaggctttgatcttggacttcttcactgctgctactgacactactgctatctctatcgaatggactttggttgaattgactaacaacccaaaggttttggaaaacgctagaaaggaaatcgctgaagttgttggtgacgaaagattggttcaagaatctgacatcccaaacttgccatacatccaagctatcatcaaggaaactttgagaatgcacccaccaatcccaatggttatcagaaagtctatcgacaacgttactgttcaaggttacgacatcagagctggtactatgttgttcgttaacatctggtctatcggtagaaacccattgtactgggaatctccattggaattcaagccacacagattcttggacggtcacgctagaaacttggacgttaagggtcaatgtttccaattgttgccattcggtactggtagaagaggttgtccaggtatctctttggctatgagagaattgccagttgttatcgctggtttgatccaatgtttcgaatggaacgctaacgacaaggaagttttgtctatggacgaaagagctggtttgactgctccaagagctgttgacttggaattcgttccattgatgagacaaaactgtccaaacatcttcgtttctgcttaa

SEQ ID NO: 137 - protein, 356 aa, Apium graveolens
MSAPSTITALSQEKTLNLDFVRDEDERPKVAYNQFSNEVPIISLAGLDDDSNGRRAEICRKIVEAFEEWGIFQVVDHGIDSGLISEMSRLSREFFALPAEEKLVYDTTGEKKGGFTISTHLQGDDVRDWREFVTYFSYPISARDYSRWPKKPEGWRSTTEVYSEKLMVLGAKLLEVLSEAMGLEKEALTKACVEMEQKVLINYYPTCPEPDLTLGVRRHTDPGTITILLQDMVGGLQATRDGGKTWITVQPVEGAFVVNLGDHGHYLSNGRFRNADHQAVVNSTSTRLSIATFQNPAQNAIVYPLKIREGEKAILDEAITYAEMYKKNMTKHIAVATQKKLAKEKRLQDEKAKMKI

SEQ ID NO: 138 - DNA, 1071 bp, synthetic construct
  misc_feature 1..1071: note = coding sequence for SEQ ID NO: 137
  source 1..1071: mol_type = other DNA
atgtccgctccatctactatcactgctttgtctcaagaaaagactttgaacttggacttcgttagagatgaagatgaaagaccaaaggttgcttacaaccaattctctaacgaagttccaatcatctctttggctggtttggacgacgactctaacggtagaagagctgaaatctgtagaaagatcgttgaagctttcgaagaatggggtatcttccaagttgttgaccacggtatcgactctggtttgatctctgaaatgtctagattgtctagagaattcttcgctttgccagctgaagaaaagttggtttacgacactactggtgaaaagaagggtggtttcactatctctactcacttgcaaggtgacgacgttagagactggagagaattcgttacttacttctcttacccaatctctgctagagactactctagatggccaaagaagccagaaggttggagatctactactgaagtttactctgaaaagttgatggttttgggtgctaagttgttggaagttttgtctgaagctatgggtttggaaaaggaagctttgactaaggcttgtgttgaaatggaacaaaaggttttgatcaactactacccaacttgtccagaaccagacttgactttgggtgttaggagacacactgacccaggtactatcactatcttgttgcaagacatggttggtggtttgcaagctactagagatggtggtaagacttggatcactgttcaaccagttgaaggtgctttcgttgttaacttgggtgaccacggtcactacttgtctaacggtagattcagaaacgctgaccaccaagctgttgttaactctacttctactagattgtctatcgctactttccaaaacccagctcaaaacgctatcgtttacccattgaagatcagagaaggtgaaaaggctatcttggacgaagctatcacttacgctgaaatgtacaagaagaacatgactaagcacatcgctgttgctactcaaaagaagttggctaaggaaaagagattgcaagacgaaaaggctaagatgaagatctaa

SEQ ID NO: 139 - protein, 521 aa, Medicago truncatula
MSEPLLLAFTLFLSSLICYIIFQPILNRHKNLPPSPLFKLPIIGHMHMLGPLLHHSFDRLSQKYGPIFSLNFGSVLCVVASTPHYAKQILQINEHAFNCRNESTAIKRLTYEASLAFAPYGEYWRFIKKLSMNELLGSRSISSFQHLRLQETHNLLKLFADKAKNYEAVNVTQELLKLSNNVISKMMLGEAEEARDVVRDVTEIFGEFNVSDFIWLFKKLDLQGFGKRIEDLFMRFDTLVERIISKREELRKNKGRKENKGEQGAEFRDFLDILLDCAEDQNSEIKVQRVHIKALIMDFFTAGTDTTSISTEWALVELMNNPSLLQKAREEIDNVVGKNRLVDESDGPNLPYIQAIIKETFRLHPPVPMVTRRCVTQCKIENYVIPENSLIFVNNWAMGRNSAYWDKPLEFNPERFLKNSTNSNGVIDVRGQNFQILPFGSGRRMCPGVTLAMQEVPALLGAIIQCFDFNFVGPKGEILKGGDIVIDVNERPGLTAPRVHDLVCVPVERFACGGPLQSLGC

SEQ ID NO: 140 - DNA, 1566 bp, synthetic construct
  misc_feature 1..1566: note = coding sequence for SEQ ID NO: 139
  source 1..1566: mol_type = other DNA
atgtccgaaccattgttgttggctttcactttgttcttgtcatctttgatctgttacatcatcttccaaccaatcttgaacagacacaagaacttgccaccatctccattgttcaagttgccaatcatcggtcacatgcacatgttgggtccattgttgcaccactctttcgacagattgtctcaaaagtacggtccaatcttctctttgaacttcggttctgttttgtgtgttgttgcttctactccacactacgctaagcaaatcttgcaaatcaacgaacacgctttcaactgtagaaacgaatctactgctatcaagagattgacttacgaagcttctttggctttcgctccatacggtgaatactggagattcatcaagaagttgtctatgaacgaattgttgggttctagatctatctcttctttccaacacttgagattgcaagaaactcacaacttgttgaagttgttcgctgacaaggctaagaactacgaagctgttaacgttactcaagaattgttgaagttgtctaacaacgttatctctaagatgatgttgggtgaagctgaagaagctagagatgttgttagagatgttactgaaatcttcggtgaattcaacgtttctgacttcatctggttgttcaagaagttggacttgcaaggtttcggtaagagaatcgaagatttgttcatgagattcgacactttggttgaaagaatcatctctaagagagaagaattgagaaagaacaagggtagaaaggaaaacaagggtgaacaaggtgctgaattcagagacttcttggacatcttgttggactgtgctgaagatcaaaactctgaaatcaaggttcaaagagttcacatcaaggctttgatcatggacttcttcactgctggtactgacactacttctatctctactgaatgggctttggttgaattgatgaacaacccatctttgttgcaaaaggctagagaagaaatcgacaacgttgttggtaagaacagattggttgacgaatctgacggtccaaacttgccatacatccaagctatcatcaaggaaactttcagattgcacccaccagttccaatggttactagaagatgtgttactcaatgtaagatcgaaaactacgttatcccagaaaactctttgatcttcgttaacaactgggctatgggtagaaactctgcttactgggacaagccattggaattcaacccagaaagattcttgaagaactctactaactctaacggtgttatcgacgttagaggtcaaaacttccaaatcttgccattcggttctggtagaagaatgtgtccaggtgttactttggctatgcaagaagttccagctttgttgggtgctatcatccaatgtttcgacttcaacttcgttggtccaaagggtgaaatcttgaagggtggtgacatcgttatcgacgttaacgaaagaccaggtttgactgctccaagagttcacgacttggtttgtgttccagttgaaagattcgcttgtggtggtccattgcaatctttgggttgttaa

SEQ ID NO: 141 - protein, 366 aa, Cuminum cyminum
MSAPTTITALAQEKTLNSDFVRDEDERPKVAYNQFSTEIPIISLAGIDDDSKGRRPEVCRKIVEAFEDWGIFQVVDHGVDSALISEMSRLSREFFALPAEEKLRYDTTGGKRGGFTISTHQQGDDVRDWREFVTYFSYPVDARDYSRWPEKPEGWRSVTEVYSEKLMVLGAKLLEVLSEAMGLDKGALTKACVNMEQKVLINYYPTCPEPDLTLGVRRHTDPGTITILLQDMVGGLQATRDGGKTWITVQPVEGVFVVNLGDHGHYLSNGRFKNADHQAVVNSTSSRLSIATFQNPAQNAIVYPLKIREGEKPILEEAITYAEMYKKNMTKHIEVATQKKLAKEKRLQEEKAKLETKTKSADGILA

SEQ ID NO: 142 - DNA, 1101 bp, synthetic construct
  misc_feature 1..1101: note = coding sequence for SEQ ID NO: 141
  source 1..1101: mol_type = other DNA
atgtccgctccaactactatcactgctttggctcaagaaaagactttgaactctgacttcgttagagatgaagatgaaagaccaaaggttgcttacaaccaattctctactgaaatcccaatcatctctttggctggtatcgacgacgactctaagggtaggaggccagaagtttgtagaaagatcgttgaagctttcgaagattggggtatcttccaagttgttgaccacggtgttgactctgctttgatctctgaaatgtctagattgtctagagaattcttcgctttgccagctgaagaaaagttgagatacgacactactggtggtaagagaggtggtttcactatctctactcaccaacaaggtgacgacgttagagactggagagaattcgttacttacttctcttacccagttgacgctagagactactctagatggccagaaaagccagaaggttggagatctgttactgaagtttactctgaaaagttgatggttttgggtgctaagttgttggaagttttgtctgaagctatgggtttggacaagggtgctttgactaaggcttgtgttaacatggaacaaaaggttttgatcaactactacccaacttgtccagaaccagacttgactttgggtgttaggagacacactgacccaggtactatcactatcttgttgcaagacatggttggtggtttgcaagctactagggacggtggtaagacttggatcactgttcaaccagttgaaggtgttttcgttgttaacttgggtgaccacggtcactacttgtctaacggtagattcaagaacgctgaccaccaagctgttgttaactctacttcttctagattgtctatcgctactttccaaaacccagctcaaaacgctatcgtttacccattgaagatcagagaaggtgaaaagccaatcttggaagaagctatcacttacgctgaaatgtacaagaagaacatgactaagcacatcgaagttgctactcaaaagaagttggctaaggaaaagagattgcaagaagaaaaggctaagttggaaactaagactaagtctgctgacggtatcttggcttaa

SEQ ID NO: 143 - protein, 356 aa, Aethusa cynapium
MSAPTTITALSQEKSLNLDFVRDEDERPKVAYNQFSNEIPIISLAGMDDDSNGRRPEICRKIVEAFEDWGIFQVVDHGIDKGLISQMSRLSREFFALPAEEKLRYDTTGGKRGGFTISTHLQGDDVKDWREFVTYFSYPIEDRDYSRWPEKPEGWRSTTEVYSEKLMVLGAKLLEVLSEAMGLEKEALTKACVNMEQKVLINYYPTCPEPDLTLGVRRHTDPGTITILLQDMVGGLQATRDGGKTWITVQPVEGAFVVNLGDHGHYLSNGRFKNADHQAVVNSTSSRLSIATFQNPAQNAIVYPLKIREGEKAILDEAITYAEMYKKNMTKHIEVAALKKLAKEKRLQDEKAKLEM

SEQ ID NO: 144 - DNA, 1071 bp, synthetic construct
  misc_feature 1..1071: note = coding sequence for SEQ ID NO: 143
  source 1..1071: mol_type = other DNA
atgtccgctccaactactatcactgctttgtctcaagaaaagtctttgaacttggacttcgttagagatgaagatgaaagaccaaaggttgcttacaaccaattctctaacgaaatcccaatcatctctttggctggtatggacgacgactctaacggtaggaggccagaaatctgtagaaagatcgttgaagctttcgaagattggggtatcttccaagttgttgaccacggtatcgacaagggtttgatctctcaaatgtctagattgtctagagaattcttcgctttgccagctgaagaaaagttgagatacgacactactggtggtaagagaggtggtttcactatctctactcacttgcaaggtgacgacgttaaggactggagagaattcgttacttacttctcttacccaatcgaagatagagactactctagatggccagaaaagccagaaggttggagatctactactgaagtttactctgaaaagttgatggttttgggtgctaagttgttggaagttttgtctgaagctatgggtttggaaaaggaagctttgactaaggcttgtgttaacatggaacaaaaggttttgatcaactactacccaacttgtccagaaccagacttgactttgggtgttaggagacacactgacccaggtactatcactatcttgttgcaagacatggttggtggtttgcaagctactagggacggtggtaagacttggatcactgttcaaccagttgaaggtgctttcgttgttaacttgggtgaccacggtcactacttgtctaacggtagattcaagaacgctgaccaccaagctgttgttaactctacttcttctagattgtctatcgctactttccaaaacccagctcaaaacgctatcgtttacccattgaagatcagagaaggtgaaaaggctatcttggacgaagctatcacttacgctgaaatgtacaagaagaacatgactaagcacatcgaagttgctgctttgaagaagttggctaaggaaaagagattgcaagacgaaaaggctaagttggaaatgtaa

SEQ ID NO: 145 - protein, 366 aa, Conium maculatum
MSAPTTITALAQEKTLNLAFVRDEDERPKVAYNEFSNEIPIISLAGLENDSDGRRPEICRKIVEAFENWGIFQVVDHGIDSALISEMSRLSREFFALPAEEKLRYDTTGGKRGGFTISTHLQGDDVRDWREFVTYFSYPIDARDYSRWPDKPEGWRSITEVYSERLMVLGAKLLEVLSEAMGLEKEALTKACVNMEQKVLINYYPTCPEPDLTLGVRRHTDPGTITVLLQDMVGGLQATRDGGKTWITVQPVEGAFVVNLGDHGHYLSNGRFKNADHQAVVNSSSSRLSIATFQNPAQNAIVYPLKIREGEKAILDEAITYAEMYKKNMTKHIEVATLKKLAKEKRLQDEKANMEKKSKSAHGISA

SEQ ID NO: 146 - DNA, 1101 bp, synthetic construct
  misc_feature 1..1101: note = coding sequence for SEQ ID NO: 145
  source 1..1101: mol_type = other DNA
atgtccgctccaactactatcactgctttggctcaagaaaagactttgaacttggctttcgttagagatgaagatgaaagaccaaaggttgcttacaacgaattctctaacgaaatcccaatcatctctttggctggtttggaaaacgactctgacggtagaaggccagaaatctgtagaaagatcgttgaagctttcgaaaactggggtatcttccaagttgttgaccacggtatcgactctgctttgatctctgaaatgtctagattgtctagagaattcttcgctttgccagctgaagaaaagttgagatacgacactactggtggtaagagaggtggtttcactatctctactcacttgcaaggtgacgacgttagagactggagagaattcgttacttacttctcttacccaatcgacgctagagactactctagatggccagacaagccagaaggttggagatctatcactgaagtttactctgaaagattgatggttttgggtgctaagttgttggaagttttgtctgaagctatgggtttggaaaaggaagctttgactaaggcttgtgttaacatggaacaaaaggttttgatcaactactacccaacttgtccagaaccagacttgactttgggtgttagaaggcacactgacccaggtactatcactgttttgttgcaagacatggttggtggtttgcaagctactagagatggtggtaagacttggatcactgttcaaccagttgaaggtgctttcgttgttaacttgggtgaccacggtcactacttgtctaacggtagattcaagaacgctgaccaccaagctgttgttaactcttcttcttctagattgtctatcgctactttccaaaacccagctcaaaacgctatcgtttacccattgaagatcagagaaggtgaaaaggctatcttggacgaagctatcacttacgctgaaatgtacaagaagaacatgactaagcacatcgaagttgctactttgaagaagttggctaaggaaaagagattgcaagacgaaaaggctaacatggaaaagaagtctaagtctgctcacggtatctctgcttaa

SEQ ID NO: 147 - protein, 535 aa, Camellia sinensis
MSFDLISIATLFFVIISTTILLLSINHFKKPPHLRRRLSLPPTPFALPIIGHLHLLGPIIHRSFHDLSSRYGPLFHLRLGSVPCFVVSTPELAKEFLLTHELKFSSRRDSIAIQRLTYDSAFAFAPYGPYWKFLKKLCTCDLLGARSINHFLPTRTRELHCFVRLLIDKAVACEPVNITKELSTLANNIISQMMIGVRCSGTTGEAEEATTLAREVTKIFGEFNVSDFMWVIRNFDLQGFRKRVEDIYTRYDALLERIITNREEVREKNVQERKLGVGEGHHVKDFLDLLLDVLEEDHSEINFSRDNIKGLILDFFTAGTDTSSIAIEWALAELINNPRVLQKAQEEIDNVVGKHRLVSESDGPNLPYIQAIIREALRLHPPVPLITRKSIEDCMIQGYNIPANSMLFVNVWSLARNPKYWDSPLDFLPERFLRPEKGGPVGPTDVKGQHFQLLPFGTGRRGCPGTSLAMQELPAMLAAMIQCFEWKVVNQSGDVMNGDGALDMTEQPGMTAPRAHDLVCMPIPRIDQLYALLDP

SEQ ID NO: 148 - DNA, 1608 bp, synthetic construct
  misc_feature 1..1608: note = coding sequence for SEQ ID NO:147
  source 1..1608: mol_type = other DNA
atgtccttcgacttgatctctatcgctactttgttcttcgttatcatctctactactatcttgttgttgtctatcaaccacttcaagaagccaccacacttgagaagaagattgtctttgccaccaactccattcgctttgccaatcatcggtcacttgcacttgttgggtccaatcatccacagatctttccacgacttgtcatctagatacggtccattgttccacttgagattgggttctgttccatgtttcgttgtttctactccagaattggctaaggaattcttgttgactcacgaattgaagttctcttctagaagagactctatcgctatccaaagattgacttacgactctgctttcgctttcgctccatacggtccatactggaagttcttgaagaagttgtgtacttgtgacttgttgggtgctagatctatcaaccacttcttgccaactagaactagagaattgcactgtttcgttagattgttgatcgacaaggctgttgcttgtgaaccagttaacatcactaaggaattgtctactttggctaacaacatcatctctcaaatgatgatcggtgttagatgttctggtactactggtgaagctgaagaagctactactttggctagagaagttactaagatcttcggtgaattcaacgtttctgacttcatgtgggttatcagaaacttcgacttgcaaggtttcagaaagagagttgaagatatctacactagatacgacgctttgttggaaagaatcatcactaacagagaagaagttagagaaaagaacgttcaagaaagaaagttgggtgttggtgaaggtcaccacgttaaggacttcttggacttgttgttggacgttttggaagaagatcactctgaaatcaacttctctagagacaacatcaagggtttgatcttggacttcttcactgctggtactgacacttcttctatcgctatcgaatgggctttggctgaattgatcaacaacccaagagttttgcaaaaggctcaagaagaaatcgacaacgttgttggtaagcacagattggtttctgaatctgacggtccaaacttgccatacatccaagctatcatcagagaagctttgagattgcacccaccagttccattgatcactagaaagtctatcgaagattgtatgatccaaggttacaacatcccagctaactctatgttgttcgttaacgtttggtctttggctagaaacccaaagtactgggactctccattggacttcttgccagaaagattcttgaggccagaaaagggtggtccagttggtccaactgacgttaagggtcaacacttccaattgttgccattcggtactggtagaagaggttgtccaggtacttctttggctatgcaagaattgccagctatgttggctgctatgatccaatgtttcgaatggaaggttgttaaccaatctggtgacgttatgaacggtgacggtgctttggacatgactgaacaaccaggtatgactgctccaagagctcacgacttggtttgtatgccaatcccaagaatcgaccaattgtacgctttgttggacccataa

SEQ ID NO: 149 - protein, 517 aa, Saussurea medusa
MSQVLQTLTPAVIAAVLLSSLFLYLLIKKNQNHRLPPSPPSLPIIGHLHHLGPLIHQSFHHLSTKYGPLIHLRLGSVTCVVASTPDLARDFLKTNELAFSSRKHSLAIDHITYGVAFAFAPYGPYWKFIKKMSTVELLGNQNLGHFLPIRTQEIHELLHTLMNKAKKRESVNLTEELLKLTNNVICQMMMSIRCSGTNSEADEAKNLVREVTQIFGEFNVSDFIWFCKNIDLQGFKKRYEDTHRRYDVLLEKIILEREEERRKEGKREDGNKGKDFLDMLLDVLEDGKAEIQITRDHIKALILDFFTAATDTTAIALEWMLVELIRNPKVLEIAREEIDHIIGNERLVQESDIPNLPYIQAIIKETLRLHPPIPMLIRKSIENVSVQGYDIPAGTMLFVNIWSIGRNPKYWESPLEFKPHRFLEEDNALKSSFDIKGQNFQLLPFGTGRRGCPGINLAMKELPVVIAGLIQCFEWNINEKQVLDMDERAGLTAPRAADFVCVPSIREDSPKSFITST

SEQ ID NO: 150 - DNA, 1554 bp, synthetic construct
  misc_feature 1..1554: note = coding sequence for SEQ ID NO: 149
  source 1..1554: mol_type = other DNA
atgtcccaagttttgcaaactttgactccagctgttatcgctgctgttttgttgtcatctttgttcttgtacttgttgatcaagaagaaccaaaaccacagattgccaccatctccaccatctttgccaatcatcggtcacttgcaccacttgggtccattgatccaccaatctttccaccacttgtctactaagtacggtccattgatccacttgagattgggttctgttacttgtgttgttgcttctactccagacttggctagagacttcttgaaaactaacgaattggctttctcttctagaaagcactctttggctatcgaccacatcacttacggtgttgctttcgctttcgctccatacggtccatactggaagttcatcaagaagatgtctactgttgaattgttgggtaaccaaaacttgggtcacttcttgccaatcagaactcaagaaatccacgaattgttgcacactttgatgaacaaggctaagaagagagaatctgttaacttgactgaagaattgttgaagttgactaacaacgttatctgtcaaatgatgatgtctatcagatgttctggtactaactctgaagctgacgaagctaagaacttggttagagaagttactcaaatcttcggtgaattcaacgtttctgacttcatctggttctgtaagaacatcgacttgcaaggtttcaagaagagatacgaagatactcacagaagatacgacgttttgttggaaaagatcatcttggaaagagaagaagaaagaagaaaggaaggtaagagagaagatggtaacaagggtaaggacttcttggacatgttgttggacgttttggaagatggtaaggctgaaatccaaatcactagagatcacatcaaggctttgatcttggacttcttcactgctgctactgacactactgctatcgctttggaatggatgttggttgaattgatcagaaacccaaaggttttggaaatcgctagagaagaaatcgaccacatcatcggtaacgaaagattggttcaagaatctgacatcccaaacttgccatacatccaagctatcatcaaggaaactttgagattgcacccaccaatcccaatgttgatcagaaagtctatcgaaaacgtttctgttcaaggttacgacatcccagctggtactatgttgttcgttaacatctggtctatcggtagaaacccaaagtactgggaatctccattggaattcaagccacacagattcttggaagaagataacgctttgaagtcatctttcgacatcaagggtcaaaacttccaattgttgccattcggtactggtagaagaggttgtccaggtatcaacttggctatgaaggaattgccagttgttatcgctggtttgatccaatgtttcgaatggaacatcaacgaaaagcaagttttggacatggacgaaagagctggtttgactgctccaagagctgctgacttcgtttgtgttccatctatcagagaagattctccaaagtctttcatcacttctacttaa

SEQ ID NO: 151 - protein, 510 aa, Plectranthus barbatus
MSDHVEAALFAAIFLLSAALLNHLLTGKRRQNAYPPGPFPLPIIGHLHLLGPRLHHTFHDLTQRYGPLMQVRLGSIRCVIAATPELAKEFLKTSELVFSARKHSTAIDIVTYESSFAFSPYGPYWKYIKKLCTYELLGARNLNHFLPIRTIEVKTFLEALMQKGKTGERLNVTEELVKLTSNVISQMMLSIRCSGTEGETEAVRTVIREVTQIFGEFDVADIIWFCKNFDFQGIRKRSEDIQRRYDALLEKIITDREKQRRTQHGGEAKDFLDMFLDIMKSGKAEVNFTRDHLKALILDFFTAGTDTTAIVVGWAIAELINNPNVLKKAQAEIDKVVGLHRILQESDGPNLPYLNAVIKETFRLHPPIPMLSRKSISDCVIDGYTIPANTLLFVNIWSMGRNPKIWDNPMAFRPERFLEKEKTGIDIKGQHFELLPFGTGRRGCPGMLLAIREVVVIIGTVIQCFDWKLPVDDVSGLVDMTERPGLTAPRADDLICRVVPRVDPLVVSGH

SEQ ID NO: 152 - DNA, 1533 bp, synthetic construct
  misc_feature 1..1533: note = coding sequence for SEQ ID NO: 151
  source 1..1533: mol_type = other DNA
atgtccgaccacgttgaagctgctttgttcgctgctatcttcttgttgtctgctgctttgttgaaccacttgttgactggtaagagaaggcaaaacgcttacccaccaggtccattcccattgccaatcatcggtcacttgcacttgttgggtccaagattgcaccacactttccacgacttgactcaaagatacggtccattgatgcaagttagattgggttctatcagatgtgttatcgctgctactccagaattggctaaggaattcttgaaaacttctgaattggttttctctgctagaaagcactctactgctatcgacatcgttacttacgaatcttctttcgctttctctccatacggtccatactggaagtacatcaagaagttgtgtacttacgaattgttgggtgctagaaacttgaaccacttcttgccaatcagaactatcgaagttaagactttcttggaagctttgatgcaaaagggtaagactggtgaaagattgaacgttactgaagaattggttaagttgacttctaacgttatctctcaaatgatgttgtctatcagatgttctggtactgaaggtgaaactgaagctgttagaactgttatcagagaagttactcaaatcttcggtgaattcgacgttgctgacatcatctggttctgtaagaacttcgacttccaaggtatcagaaagagatctgaagatatccaaagaagatacgacgctttgttggaaaagatcatcactgacagagaaaagcaaagaagaactcaacacggtggtgaagctaaggacttcttggacatgttcttggacatcatgaagtctggtaaggctgaagttaacttcactagagatcacttgaaggctttgatcttggacttcttcactgctggtactgacactactgctatcgttgttggttgggctatcgctgaattgatcaacaacccaaacgttttgaagaaggctcaagctgaaatcgacaaggttgttggtttgcacagaatcttgcaagaatctgacggtccaaacttgccatacttgaacgctgttatcaaggaaactttcagattgcacccaccaatcccaatgttgtctagaaagtctatctctgactgtgttatcgacggttacactatcccagctaacactttgttgttcgttaacatctggtctatgggtagaaacccaaagatctgggacaacccaatggctttcagaccagaaagattcttggaaaaggaaaagactggtatcgacatcaagggtcaacacttcgaattgttgccattcggtactggtagaagaggttgtccaggtatgttgttggctatcagagaagttgttgttatcatcggtactgttatccaatgtttcgactggaagttgccagttgacgacgtttctggtttggttgacatgactgaaagaccaggtttgactgctccaagagctgacgacttgatctgtagagttgttccaagagttgacccattggttgtttctggtcactaa

SEQ ID NO: 153 - protein, 506 aa, Scutellaria baicalensis
MSEVTLNVALLLLSAAVCLMVFTGKRRRRLPNPPGPFPLPLIGNLNLVSPRLHHTFHMLAQRYGPIMKFRLGSIPCLVVSTPELAKDILKTHELIFSSRVKSTAIDIVTYGVSFAFSPYGPYWKYIKKLCTYELLGSRMLNHFEPLRALEVREFLKDVMAMGKAGKSFNVTEELMKLTSNVMSNMMLSIRAAESEEQAEVARTLIREVSQLFGEFDFGDMLWFCKSFDFQGIKKRSKDIKVRYDALLEKILTDRENVRRQNGVVEPKDMLDMFLDIMEGGKTDVEFTREHLKAVILDFLTAGTDTTAITVEWVLAELMNSPKAMKKAQDEMDRVVGRERMMAESDAPNLPYFLAIIKETFRLHPPIPLIIRRSIEDCVIDGYHIPADTLAFINVWSMGRNEKYWDSPLSFRPERFLEGDNAAIDIKGMHFELLPFGSGRRGCPGMLSAIQEVLIIAGTVIQCFDWEQADGSGRVDMSERPGLTTPREIDLVCRVVPRVDERVISGH

SEQ ID NO: 154 - DNA, 1521 bp, synthetic construct
  misc_feature 1..1521: note = coding sequence for SEQ ID NO:153
  source 1..1521: mol_type = other DNA
atgtccgaagttactttgaacgttgctttgttgttgttgtctgctgctgtttgtttgatggttttcactggtaagagaagaagaagattgccaaacccaccaggtccattcccattgccattgatcggtaacttgaacttggtttctccaagattgcaccacactttccacatgttggctcaaagatacggtccaatcatgaagttcagattgggttctatcccatgtttggttgtttctactccagaattggctaaggacatcttgaaaactcacgaattgatcttctcttctagagttaagtctactgctatcgacatcgttacttacggtgtttctttcgctttctctccatacggtccatactggaagtacatcaagaagttgtgtacttacgaattgttgggttctagaatgttgaaccacttcgaaccattgagagctttggaagttagagaattcttgaaggacgttatggctatgggtaaggctggtaagtctttcaacgttactgaagaattgatgaagttgacttctaacgttatgtctaacatgatgttgtctatcagagctgctgaatctgaagaacaagctgaagttgctagaactttgatcagagaagtttctcaattgttcggtgaattcgacttcggtgacatgttgtggttctgtaagtctttcgacttccaaggtatcaagaagagatctaaggacatcaaggttagatacgacgctttgttggaaaagatcttgactgacagagaaaacgttaggagacaaaacggtgttgttgaaccaaaggacatgttggacatgttcttggacatcatggaaggtggtaagactgacgttgaattcactagagaacacttgaaggctgttatcttggacttcttgactgctggtactgacactactgctatcactgttgaatgggttttggctgaattgatgaactctccaaaggctatgaagaaggctcaagacgaaatggacagagttgttggtagagaaagaatgatggctgaatctgacgctccaaacttgccatacttcttggctatcatcaaggaaactttcagattgcacccaccaatcccattgatcatcagaagatctatcgaagattgtgttatcgacggttaccacatcccagctgacactttggctttcatcaacgtttggtctatgggtagaaacgaaaagtactgggactctccattgtctttcagaccagaaagattcttggaaggtgacaacgctgctatcgacatcaagggtatgcacttcgaattgttgccattcggttctggtagaagaggttgtccaggtatgttgtctgctatccaagaagttttgatcatcgctggtactgttatccaatgtttcgactgggaacaagctgacggttctggtagagttgacatgtctgaaagaccaggtttgactactccaagagaaatcgacttggtttgtagagttgttccaagagttgacgaaagagttatctctggtcactaa

SEQ ID NO: 155 - protein, 510 aa, Dorcoceras hygrometricum
MSDLVQITLSAALLLLSAAFLHTIFATKRRRLSPPPGPLALPIIGHLHLLGPRLHQTFHDLSLRHGPIFNLRLGSVACAVVSTPELAKECLKTHELVFSSRKHSTAIDIVTYDSSFAFSPYGPYWKYIKKLCTYELLGARNLLHFQPIRTLEVNSFVGTLMNKAESGESFNVTEELVKLTSNVISHMMLGIRCSGTEGEAEAARTVIREVTQIFGEFDVADIIWFCKNFDFQGIRKRSEDIQRRYDALLEKIITDREELRRSHGGAAGEARDFLDMFLDIMEGGKSEVTFTREHLKALILDFFTAGTDTTAIVTEWAISELINNPKVLEKAQQEIDKVIGSGRLVQESDAPNLPYLMAVIKETFRLHPPIPMLSRKSISDCVIDGYDVPAKSLLFVNIWSMGRNPKIWESPLEFRPERFLEREKSSIDIKGQHFELLPFGTGRRGCPGMLLGIQEVVIIIGTMVQCFDWKLSDGSGQVDMTERPGLTAPRAHDLFCRVVPRINPVVVSGN

SEQ ID NO: 156 - DNA, 1533 bp, synthetic construct
  misc_feature 1..1533: note = coding sequence for SEQ ID NO: 155
  source 1..1533: mol_type = other DNA
atgtccgacttggttcaaatcactttgtctgctgctttgttgttgttgtctgctgctttcttgcacactatcttcgctactaagagaagaagattgtctccaccaccaggtccattggctttgccaatcatcggtcacttgcacttgttgggtccaagattgcaccaaactttccacgacttgtctttgagacacggtccaatcttcaacttgagattgggttctgttgcttgtgctgttgtttctactccagaattggctaaggaatgtttgaaaactcacgaattggttttctcttctagaaagcactctactgctatcgacatcgttacttacgactcttctttcgctttctctccatacggtccatactggaagtacatcaagaagttgtgtacttacgaattgttgggtgctagaaacttgttgcacttccaaccaatcagaactttggaagttaactctttcgttggtactttgatgaacaaggctgaatctggtgaatctttcaacgttactgaagaattggttaagttgacttctaacgttatctctcacatgatgttgggtatcagatgttctggtactgaaggtgaagctgaagctgctagaactgttatcagagaagttactcaaatcttcggtgaattcgacgttgctgacatcatctggttctgtaagaacttcgacttccaaggtatcagaaagagatctgaagatatccaaagaagatacgacgctttgttggaaaagatcatcactgacagagaagaattgagaagatctcacggtggtgctgctggtgaagctagagacttcttggacatgttcttggacatcatggaaggtggtaagtctgaagttactttcactagagaacacttgaaggctttgatcttggacttcttcactgctggtactgacactactgctatcgttactgaatgggctatctctgaattgatcaacaacccaaaggttttggaaaaggctcaacaagaaatcgacaaggttatcggttctggtagattggttcaagaatctgacgctccaaacttgccatacttgatggctgttatcaaggaaactttcagattgcacccaccaatcccaatgttgtctagaaagtctatctctgactgtgttatcgacggttacgacgttccagctaagtctttgttgttcgttaacatctggtctatgggtagaaacccaaagatctgggaatctccattggaattcagaccagaaagattcttggaaagagaaaagtcatctatcgacatcaagggtcaacacttcgaattgttgccattcggtactggtagaagaggttgtccaggtatgttgttgggtatccaagaagttgttatcatcatcggtactatggttcaatgtttcgactggaagttgtctgacggttctggtcaagttgacatgactgaaagaccaggtttgactgctccaagagctcacgacttgttctgtagagttgttccaagaatcaacccagttgttgtttctggtaactaa

SEQ ID NO: 157 - protein, 507 aa, Antirrhinum majus
MSSTLVYSTLFILSTLLLTLLTRTRRKTRPPGPLALPLIGHLHLLGPKLHHTFHQFSQRYGPLIQLYLGSVPCVVASTPELAREFLKTHELDFSSRKHSTAIDIVTYDSSFAFAPYGPYWKFIKKLCTYELLGARNLSHFQPIRALEVNSFLRILYEKTEQKQSVNVTEELVKLTSNVISNMMLGIRCSGTEGEAEVARTVIREVTQIFGEFDVSEIVWFCKNLDLQGIRKRSEDIRRRYDALLEKIISDRERLRLRGGGGGGGGEVKDFLDMLLDVMESEKSEVEFTREHLKALILDFFTAGTDTTAITTEWAIAELISNPNVLKKAQEEMDKVIGSQRLLQESDAPNLPYLNAIIKETFRLHPPIPMLTRKSISDVVVNGYTIPAKTLLFVNLWSMGRNPNYWENPMEFRPERFLEKGTGSIDVKGQHFELLPFGTGRRGCPGMLLGMQELFSIIGAMVQCFDWKLPDGVKSVDMTERPGLTAPRANDLVCQLVPRIDPVVVSGP

SEQ ID NO: 158 - DNA, 1524 bp, synthetic construct
  misc_feature 1..1524: note = coding sequence for SEQ ID NO: 157
  source 1..1524: mol_type = other DNA
atgtcctctactttggtttactctactttgttcatcttgtctactttgttgttgactttgttgactagaactagaagaaagactagaccaccaggtccattggctttgccattgatcggtcacttgcacttgttgggtccaaagttgcaccacactttccaccaattctctcaaagatacggtccattgatccaattgtacttgggttctgttccatgtgttgttgcttctactccagaattggctagagaattcttgaaaactcacgaattggacttctcttctagaaagcactctactgctatcgacatcgttacttacgactcttctttcgctttcgctccatacggtccatactggaagttcatcaagaagttgtgtacttacgaattgttgggtgctagaaacttgtctcacttccaaccaatcagagctttggaagttaactctttcttgagaatcttgtacgaaaagactgaacaaaagcaatctgttaacgttactgaagaattggttaagttgacttctaacgttatctctaacatgatgttgggtatcagatgttctggtactgaaggtgaagctgaagttgctagaactgttatcagagaagttactcaaatcttcggtgaattcgacgtttctgaaatcgtttggttctgtaagaacttggacttgcaaggtatcagaaagagatctgaagatatcagaagaagatacgacgctttgttggaaaagatcatctctgacagagaaagattgagattgagaggtggtggtggtggtggtggtggtgaagttaaggacttcttggacatgttgttggacgttatggaatctgaaaagtctgaagttgaattcactagagaacacttgaaggctttgatcttggacttcttcactgctggtactgacactactgctatcactactgaatgggctatcgctgaattgatctctaacccaaacgttttgaagaaggctcaagaagaaatggacaaggttatcggttctcaaagattgttgcaagaatctgacgctccaaacttgccatacttgaacgctatcatcaaggaaactttcagattgcacccaccaatcccaatgttgactagaaagtctatctctgacgttgttgttaacggttacactatcccagctaagactttgttgttcgttaacttgtggtctatgggtagaaacccaaactactgggaaaacccaatggaattcagaccagaaagattcttggaaaagggtactggttctatcgacgttaagggtcaacacttcgaattgttgccattcggtactggtagaagaggttgtccaggtatgttgttgggtatgcaagaattgttctctatcatcggtgctatggttcaatgtttcgactggaagttgccagacggtgttaagtctgttgacatgactgaaagaccaggtttgactgctccaagagctaacgacttggtttgtcaattggttccaagaatcgacccagttgttgtttctggtccataa

SEQ ID NO: 159 - protein, 511 aa, Erythranthe lewisii
MSNQTMDLPEICLYAVILFVSTLLILGIYKRKRSHIPSPPGPFALPVIGHLHLLGPRIHHTFHDLSQRYGPLFQLSLGSVRCVVVSTPELAREFLKTHELVFSSRKHTTAIDIVTYESSFAFSPYGPYWKYIKKLCTYELLGARNLANFEPVRNVEIKDFLKVMSNKANTGEIVNVTEELVKLTSNVISHMMLGIRCSGTEGEAEAARNVIRDVTQIFGEFDVSDIIWFCKNFDLQGIRRRSEDIQKRYDGLLEKIITDREKTRGGGGGGVKDFLDMLLDVMDSKNSDVKFTREHLKALILDFFTAGTDTTAIAVEWSIAELLRNPKVIKKAQQEIDNVVGSQRLLQESDAPKLPYIMAIIKETFRLHPPIPMISRKSVSDCAINGCMIRANTLLFVNIWSIGRNPMYWERPMEFRPERFLDPGCGSIDVKGQNFELMPFGTGRRGCPGMLLAMQELVAIIGAMVQCFEWQLPDDSQDVDMTERPGLTAPRANDLFCRVVPRVDVAVVSGN

SEQ ID NO: 160 - DNA, 1536 bp, synthetic construct
  misc_feature 1..1536: note = coding sequence for SEQ ID NO: 159
  source 1..1536: mol_type = other DNA
atgtccaaccaaactatggacttgccagaaatctgtttgtacgctgttatcttgttcgtttctactttgttgatcttgggtatctacaagagaaagagatctcacatcccatctccaccaggtccattcgctttgccagttatcggtcacttgcacttgttgggtccaagaatccaccacactttccacgacttgtctcaaagatacggtccattgttccaattgtctttgggttctgttagatgtgttgttgtttctactccagaattggctagagaattcttgaaaactcacgaattggttttctcttctagaaagcacactactgctatcgacatcgttacttacgaatcttctttcgctttctctccatacggtccatactggaagtacatcaagaagttgtgtacttacgaattgttgggtgctagaaacttggctaacttcgaaccagttagaaacgttgaaatcaaggacttcttgaaggttatgtctaacaaggctaacactggtgaaatcgttaacgttactgaagaattggttaagttgacttctaacgttatctctcacatgatgttgggtatcagatgttctggtactgaaggtgaagctgaagctgctagaaacgttatcagagatgttactcaaatcttcggtgaattcgacgtttctgacatcatctggttctgtaagaacttcgacttgcaaggtatcagaagaagatctgaagatatccaaaagagatacgacggtttgttggaaaagatcatcactgacagagaaaagactagaggtggtggtggtggtggtgttaaggacttcttggacatgttgttggacgttatggactctaagaactctgacgttaagttcactagagaacacttgaaggctttgatcttggacttcttcactgctggtactgacactactgctatcgctgttgaatggtctatcgctgaattgttgagaaacccaaaggttatcaagaaggctcaacaagaaatcgacaacgttgttggttctcaaagattgttgcaagaatctgacgctccaaagttgccatacatcatggctatcatcaaggaaactttcagattgcacccaccaatcccaatgatctctagaaagtctgtttctgactgtgctatcaacggttgtatgatcagagctaacactttgttgttcgttaacatctggtctatcggtagaaacccaatgtactgggaaagaccaatggaattcagaccagaaagattcttggacccaggttgtggttctatcgacgttaagggtcaaaacttcgaattgatgccattcggtactggtagaagaggttgtccaggtatgttgttggctatgcaagaattggttgctatcatcggtgctatggttcaatgtttcgaatggcaattgccagacgactctcaagacgttgacatgactgaaagaccaggtttgactgctccaagagctaacgacttgttctgtagagttgttccaagagttgacgttgctgttgtttctggtaactaa